(19) United States
(12) Patent Application Publication
(10) Pub. No.: US 2025/0223268 A1
(43) Pub. Date: Jul. 10, 2025

(54) 4-Methylsulfonylbenzamide Compound, Preparation Method, Herbicidal Composition And Use

(71) Applicant: Jiangsu Flag Chemical Industry Co., Ltd., Nanjing (CN)

(72) Inventors: Pu Zhang, Nanjing (CN); Wei Zhao, Nanjing (CN); Xu Xu, Nanjing (CN); Dan Xu, Nanjing (CN); Long Bu, Nanjing (CN); Kaicheng Yao, Nanjing (CN); Yaojun Wu, Nanjing (CN); Congqiang Bai, Nanjing (CN); Jian Jiao, Nanjing (CN)

(73) Assignee: Jiangsu Flag Chemical Industry Co., Ltd., Nanjing (CN)

(21) Appl. No.: 19/093,424

(22) Filed: Mar. 28, 2025

Related U.S. Application Data
(63) Continuation of application No. PCT/CN2023/123065, filed on Oct. 2, 2023.

(30) Foreign Application Priority Data
Oct. 5, 2022 (CN) 202211218419.5

(51) Int. Cl. C07D 257/06; A01N 43/653; A01N 43/713; A01N 43/82; A01P 13/00; C07D 249/14; C07D 271/08; C07D 271/113

(52) U.S. Cl. CPC C07D 257/06; A01N 43/653; A01N 43/713; A01N 43/82; A01P 13/00; C07D 249/14; C07D 271/08; C07D 271/113

(57) ABSTRACT

The present invention relates to a class of 4-methanesulfonylbenzamide compounds with sulfur-containing substituents at the 3-position, represented by general formula (I), including their stereoisomers, agriculturally acceptable salts, preparation methods, herbicidal compositions, and their use in the field of plant protection, wherein R₁, R₂, X, Q, and n are as defined herein.

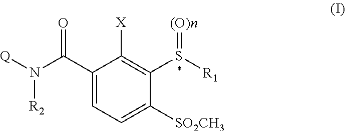

### CROSS-REFERENCE TO RELATED APPLICATIONS

The present application is a continuation of PCT Application No. PCT/CN2023/123065, filed on Oct. 2, 2023, which claims priority to Chinese Application No. 202211218419.5, filed on Oct. 5, 2022. The entire contents of these applications are incorporated herein by reference.

### TECHNICAL FIELD

The present invention pertains to the field of pesticidal herbicides, specifically relating to a class of 4-methanesulfonylbenzamide compounds, their preparation methods, herbicidal compositions, and applications.

### BACKGROUND

Chemical weed control using herbicides remains the most economical and effective approach for weed management. However, prolonged and excessive use of herbicides with a single active ingredient or mode of action often leads to issues such as weed tolerance and resistance evolution. Developing novel herbicide varieties is therefore a critical strategy to address these challenges. Patents including WO2014086746A1, WO2016146561A1, WO2014086734A1, WO2013017559A1, WO2017144402A1, WO2012126932A1, WO2013087577A1, WO2012028579A1, and WO2011035874A1 disclose certain aryl carboxamide compounds and their utility as herbicides. Nevertheless, existing aryl carboxamide compounds still exhibit limitations in herbicidal efficacy, crop safety, and resistance management. Consequently, there is an urgent market demand for novel herbicides that combine robust herbicidal activity, enhanced crop safety, and effective control of resistant weed populations.

### SUMMARY OF THE INVENTION

The technical problem addressed by the present invention is to provide novel 4-methanesulfonylbenzamide compounds, their preparation methods, herbicidal compositions, and applications. These compounds demonstrate superior herbicidal activity, improved crop safety, and effective resistance management capabilities against weeds.

The technical solution to the aforementioned problem is outlined as follows:

The first objective of the present invention is to provide a 4-methylsulfonylbenzamide compound of formula (I), stereoisomer thereof or agriculturally acceptable salt thereof

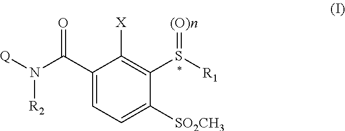

wherein
Q represents Q1, Q2, Q3 or Q4, as shown below:

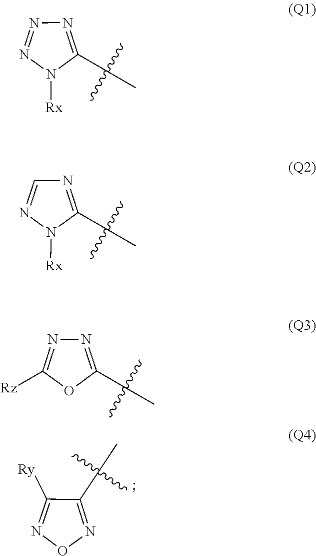

X represents fluorine, chlorine, bromine, CN, methylsulfonyl or nitro;
R₁ represents C₃₋₆ alkyl, C₃₋₆ cycloalkyl, C₃₋₆ cycloalkyl-C₁₋₆ alkyl, C₁₋₆ alkyl substituted by aromatic ring, C₁₋₆ alkyl substituted by substituted aromatic ring, halogenated C₁₋₆ alkyl, C₂₋₆ alkenyl, C₂₋₆ alkynyl, NC—C₁₋₆ alkyl, halogenated-C₂₋₆ alkenyl, C₂₋₆ alkyl-O—C₁₋₆ alkyl, halogenated C₁₋₆ alkyl-O—C₁₋₆ alkyl; R₂ represents hydrogen, C₁₋₆ alkyl, halogenated C₁₋₆ alkyl, C₃₋₆ cycloalkyl, C₂₋₆ alkenyl, halogenated-C₂₋₆ alkenyl, C₂₋₆ alkynyl, halogenated-C₃₋₆ alkynyl, C₃₋₆ cycloalkyl, halogenated-C₃₋₆ cycloalkyl, C₃₋₆ cycloalkyl-C₁₋₆ alkyl, halogenated-C₃₋₆ cycloalkyl-C₁₋₆ alkyl, Ra(O)C—C₁₋₆ alkyl, RaO(O)C—C₁₋₆ alkyl, (Ra)₂N(O)C—C₁₋₆ alkyl, NC—C₁₋₆ alkyl, RaO—C₁₋₆ alkyl, Ra(O)CO—C₁₋₆ alkyl, Rb(O)₂SO—C₁₋₆ alkyl, (Ra)₂N—C₁₋₆ alkyl, Ra(O)C(Ra)N—C₁₋₆ alkyl, Rb(O)₂S(Ra)N—C₁₋₆ alkyl, Rb(O)ₙS—C₁₋₆ alkyl, RaO(O)₂S—C₁₋₆ alkyl, (Ra)₂N(O)₂S—C₁₋₆ alkyl, Ra(O)C, RaO(O)C, (Ra)₂N(O)C, RaO, (Ra)₂N, RbO(O)C(Ra)N, (Ra)₂N(O)C(Ra)N, Rb(O)₂S or benzyl substituted by 1, 2 or 3 groups selected from methyl, ethyl, methoxy, nitro, trifluoromethyl and halogen;
Rx represents hydrogen, C₁₋₆ alkyl, halogenated-C₁₋₆ alkyl, C₂₋₆ alkenyl, halogenated-C₂₋₆ alkenyl, C₂₋₆ alkynyl or halogenated-C₃₋₆ alkynyl, wherein the above six groups are substituted by 0, 1, 2 or 3 groups selected from nitro, cyano, (Rf)₃Si, (ReO)₂(O)P, Re(O)ₙS, (Ra)₂N, RaO, Ra(O)C, RaO(O)C, Ra(O)CO, RbO(O)CO, Ra(O)C(Ra)N, Rb(O)₂S(Ra)N, C₃₋₆ cycloalkyl, heteroaryl, heterocyclic group and phenyl, wherein cycloalkyl, heteroaryl, heterocyclic group and phenyl are substituted by 1, 2 or 3 groups selected from C₁₋₆ alkyl, halogenated-C₁₋₆ alkyl, C₁₋₆ alkoxy, halogenated-C₁₋₆ alkoxy and halogen, and the heterocyclic group has n oxo groups;
or Rx represents C₃₋₇ cycloalkyl, heteroaryl, heterocyclic group or phenyl, wherein the above four groups are substituted by 0, 1, 2 or 3 groups selected from halogen, nitro, cyano, C₁₋₆ alkyl, halogenated-C₁₋₆ alkyl, C₃₋₆ cycloalkyl, C₁₋₆ alkyl-S(O)ₙ, C₁₋₆ alkoxy, halogenated-C₁₋₆ alkoxy and C₁₋₆ alkoxy-C₁₋₄ alkyl;
Ry represents hydrogen, C₁₋₆ alkyl, halogenated C₁₋₆ alkyl, C₂₋₆ alkenyl, halogenated C₂₋₆ alkenyl, C₂₋₆-alkynyl, halogenated C₃₋₆ alkynyl, C₃₋₆ cycloalkyl, C₁₋₆ alkoxy, halogenated C₁₋₆ alkoxy, C₂₋₆ alkenoxy, C₂₋₆ alkynoxy, cyano, nitro, methylthio, methylsulfinyl, methylsulfonyl, acetylamino, benzoylamino, methoxycarbonyl, ethoxycarbonyl, methoxycarbonylmethyl, ethoxycarbonylmethyl, benzoyl, methylcarbonyl, piperidinylcarbonyl, trifluoromethylcarbonyl, halogen, amino, aminocarbonyl, methylaminocarbonyl, dimethylaminocarbonyl, methoxymethyl; or Ry represents heteroaryl, heterocyclic group or phenyl, each of heteroaryl, heterocyclic group and phenyl is substituted by 1, 2 or 3 groups selected from C₁₋₆ alkyl, halogenated-C₁₋₆ alkyl, C₁₋₆ alkoxy, halogenated-C₁₋₆ alkoxy and halogen, and the heterocyclic group has n oxo groups;
Rz represents hydrogen, C₁₋₆ alkyl, RaO—C₁₋₆ alkyl, RgCH₂, C₃₋₇ cycloalkyl, halogenated-C₁₋₆ alkyl, C₂₋₆ alkenyl, halogenated-C₂₋₆ alkenyl, C₂₋₆ alkynyl, halogenated-C₃₋₆ alkynyl, RaO, Ra(H)N, methoxycarbonyl, ethoxycarbonyl, methylcarbonyl, dimethylamino, trifluoromethylcarbonyl, acetylamino, methylthio, methylsulfinyl, methylsulfonyl; or Rz represents heteroaryl, heterocyclic group, benzyl or phenyl, each of heteroaryl, heterocyclic group, benzyl and phenyl is substituted by 1, 2 or 3 groups selected from halogen, nitro, cyano, C₁₋₆ alkyl, halogenated-C₁₋₆ alkyl, C₃₋₆ cycloalkyl, C₁₋₆ alkyl-S(O)ₙ, C₁₋₆ alkoxy, halogenated-C₁₋₆ alkoxy and C₁₋₆ alkoxy-C₁₋₄ alkyl, wherein the heterocyclic group has n oxo groups;
Ra represents hydrogen, C₁₋₆ alkyl, halogenated C₁₋₆ alkyl, C₂₋₆ alkenyl, halogenated C₂₋₆ alkenyl, C₂₋₆-alkynyl, halogenated C₃₋₆ alkynyl, C₃₋₆ cycloalkyl, C₃₋₆ cycloalkenyl, halogenated-C₃₋₆ cycloalkyl, C₃₋₆ cycloalkyl-C₁₋₆ alkyl, C₁₋₆ alkyl-O—C₁₋₆ alkyl, cycloalkyl-C₁₋₆ alkyl-O—C₁₋₆ alkyl, phenyl, phenyl-C₁₋₆ alkyl, heteroaryl, heteroaryl-C₁₋₆ alkyl, heterocyclic group, heterocyclic group-C₁₋₆ alkyl, phenyl-O—C₁₋₆ alkyl, heteroaryl-O—C₁₋₆ alkyl, heterocyclic group —O—C₁₋₆ alkyl, phenyl-N(Rc)-C₁₋₆ alkyl, heteroaryl-N(Rc)-C₁₋₆ alkyl, heterocyclic group-N(Rc)-C₁₋₆ alkyl, phenyl-S(O)ₙ—C₁₋₆ alkyl, heteroaryl-S(O)ₙ—C₁₋₆ alkyl or heterocyclic group-S(O)ₙ—C₁₋₆ alkyl, wherein the mentioned last fifteen groups in each case are substituted by s groups selected from nitro, halogen, cyano, thiocyano, C₁₋₆ alkyl, halogenated-C₁₋₆ alkyl, C₃₋₆ cycloalkyl, RcO(O)C, (Rc)₂N(O)C, RcO, (Rc)₂N, Rd(O)ₙS, RcO(O)₂S, (Rc)₂N(O)₂S and RcO—C₁₋₆ alkyl, and the heterocyclic group has n oxo groups;
Rb represents C₁₋₆ alkyl, halogenated C₁₋₆ alkyl, C₂₋₆ alkenyl, halogenated C₂₋₆ alkenyl, C₂₋₆ alkynyl, halogenated C₃₋₆ alkynyl, C₃₋₆ cycloalkyl, C₃₋₆ cycloalkenyl, halogenated-C₃₋₆ cycloalkyl, C₃₋₆ cycloalkyl-C₁₋₆ alkyl, C₁₋₆ alkyl-O—C₁₋₆ alkyl, cycloalkyl-C₁₋₆ alkyl-O—C₁₋₆ alkyl, phenyl, phenyl-C₁₋₆ alkyl, heteroaryl, heteroaryl-C₁₋₆ alkyl, heterocyclic group, heterocyclic group-C₁₋₆ alkyl, phenyl-O—C₁₋₆ alkyl, heteroaryl-O—C₁₋₆ alkyl, heterocyclic group —O—C₁₋₆ alkyl, phenyl-N(Rc)-C₁₋₆ alkyl, heteroaryl-N(Rc)-C₁₋₆ alkyl, heterocyclic group-N(Rc)-C₁₋₆ alkyl, phenyl-S(O)ₙ—C₁₋₆ alkyl, heteroaryl-S(O)ₙ—C₁₋₆ alkyl or heterocyclic group-S(O)ₙ—C₁₋₆ alkyl, wherein the mentioned last fifteen groups in each case are substituted by 1, 2 or 3 groups selected from nitro, halogen, cyano, thiocyano, C₁₋₆ alkyl, halogenated-C₁₋₆ alkyl, C₃₋₆ cycloalkyl, RcO(O)C, (Rc)₂N(O)C, RcO, (Rc)₂N, Rd(O)ₙS, RcO(O)₂S, (Rc)₂N(O)₂S and RcO—C₁₋₆ alkyl, and the heterocyclic group has n oxo groups;
Rc represents hydrogen, C₁₋₆ alkyl, halogenated-C₁₋₆ alkyl, C₂₋₆ alkenyl, C₂₋₆ alkynyl, C₃₋₆ cycloalkyl, C₃₋₆ cycloalkyl-C₁₋₆ alkyl or phenyl;
Rd represents C₁₋₆ alkyl, halogenated-C₁₋₆ alkyl, C₂₋₆ alkenyl, C₂₋₆ alkynyl, C₃₋₆ cycloalkyl, C₃₋₆ cycloalkyl-C₁₋₆ alkyl or phenyl;
Re represents hydrogen or C₁₋₄ alkyl;
Rf represents C₁₋₄ alkyl;
Rg represents acetoxy, acetamido, N-methylacetamido, benzoyloxy, benzamido, N-methylbenzamido, methoxycarbonyl, ethoxycarbonyl, benzoyl, methylcarbonyl, piperidinylcarbonyl, morpholinylcarbonyl, trifluoromethylcarbonyl, aminocarbonyl, methylaminocarbonyl, dimethylaminocarbonyl, C₃₋₆ cycloalkyl; or Rg represents heteroaryl or heterocyclic group, which in each case is substituted by s groups selected from methyl, ethyl, methoxy, trifluoromethyl and halogen;
n represents 0, 1 or 2;
when n represents 1, the sulfur atom connected thereto may be selected from either an R configuration or an S configuration, or a mixture of the two, and the ratio of R to S in the mixture is 1:99 to 99:1;
s represents 0, 1, 2 or 3.

In preferred embodiment of the present invention, said 4-methylsulfonylbenzamides, stereoisomers thereof, or agriculturally acceptable salts thereof:

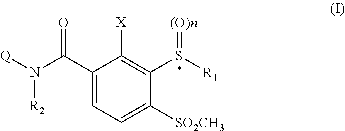

wherein
Q represents Q1, Q2, Q3 or Q4, as shown below:

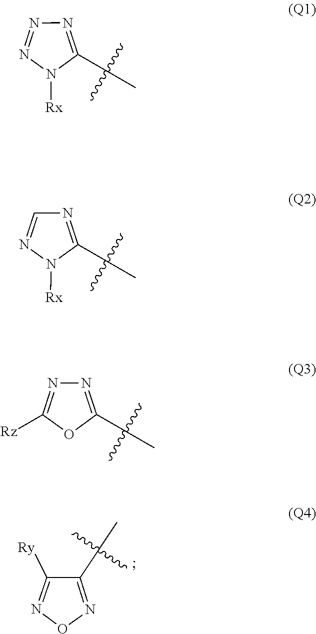

X represents chlorine;
R₁ represents C₃₋₆ alkyl, C₃₋₆ cycloalkyl, halogenated C₃₋₆ cycloalkyl, C₃₋₆ cycloalkylmethyl, methyl substituted by aromatic ring, halogenated C₃₋₄ alkyl, C₂₋₄ alkenyl, C₂₋₄ alkynyl, NC—C₁₋₄ alkyl, halogenated-C₂₋₄ alkenyl, C₁₋₂ alkyl-O—C₁₋₂ alkyl or halogenated C₁₋₂ alkyl-O—C₁₋₂ alkyl;
R₂ represents hydrogen, C₁₋₃ alkyl, halogenated C₁₋₃ alkyl, cyclopropyl, halogenated cyclopropyl, C₂₋₃ alkenyl, halogenated-C₂₋₃ alkenyl, C₂₋₃ alkynyl, halogenated C₂₋₃ alkynyl, or C₁₋₆ alkyl-O—C₁₋₆ alkyl;
Rx represents hydrogen, C₁₋₃ alkyl, halogenated-C₁₋₃ alkyl, C₂₋₃ alkenyl, halogenated C₂₋₃ alkenyl, C₂₋₃ alkynyl, halogenated C₂₋₃ alkynyl, cyclopropyl or halogenated cyclopropyl;
Ry represents hydrogen, C₁₋₃ alkyl, halogenated-C₁₋₃ alkyl, C₂₋₃ alkenyl, halogenated C₂₋₃ alkenyl, C₂₋₃ alkynyl, halogenated C₂₋₃ alkynyl, cyclopropyl or halogenated cyclopropyl;
Rz represents hydrogen, C₁₋₃ alkyl, halogenated-C₁₋₃ alkyl, C₂₋₃ alkenyl, halogenated C₂₋₃ alkenyl, C₂₋₃ alkynyl, halogenated C₂₋₃ alkynyl, cyclopropyl or halogenated cyclopropyl;
n represents 0, 1 or 2;
when n represents 1, the sulfur atom connected thereto may be selected from either an R configuration or an S configuration, or a mixture of the two, and the ratio of R to S in the mixture is 1:99 to 99:1.

In preferred embodiment of the present invention, said 4-methylsulfonylbenzamides, stereoisomers thereof, or agriculturally acceptable salts thereof:

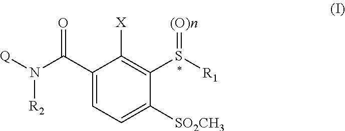

wherein
Q represents Q1, Q2, Q3 or Q4, as shown below:

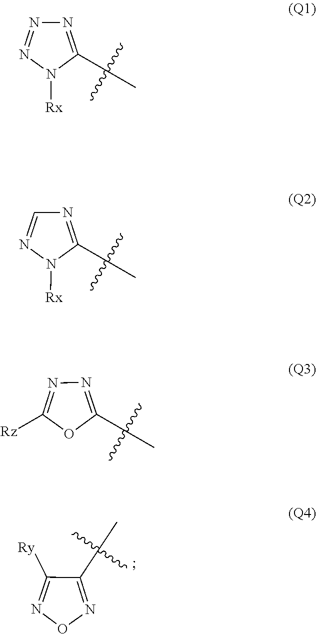

X represents chlorine;
R₁ represents CH₂CH₂CH₃, CH(CH₃)₂, CH₂CH₂CH₂CH₃, CH(CH₃)CH₂CH₃, cyclopropylmethyl, cyclopropyl, cyclobutyl, cyclopentyl, cyclohexyl, CH₂CF₃, CH₂CH═CH₂, CH₂CH₂Cl, CH₂CH(CH₃)₂, C(CH₃)₃, CH₂CH₂CH₂CH₂CH₃, CH(CH₃)CH₂CH₂CH₃, CH₂CH₂CH(CH₃)₂, C(CH₃)₂CH₂CH₃, CH₂CH(CH₃)CH₂CH₃, CH₂C₆H₅, CH(CH₃)C₆H₅, CH₂CF₂H, CH₂CFH₂, CH(CH₃)CH═CH₂, (E)-CH₂CH═CHCH₃, (Z)—CH₂CH═CHCH₃, CH₂CH═C(CH₃)₂, propargyl, but-3-en-2-yl, but-2-en-1-yl, CF₂CF₃, CF₂CF₂CF₃, CH₂CF₂CF₃, CH₂CH₂CF₃, CH₂CH₂CF₂H, CH₂CH₂CFH₂, CF₂CFHCF₃, CH₂CN, CH₂CH₂CN, CH(CH₃)CN, CH(CH₃)CH₂CN, CHClCH₃, (E)-CH₂CH═CHCl, (Z)—CH₂CH═CHCl, CH₂CH═CCl₂, (E)-CH₂CH═CHOCH₃, (Z)—CH₂CH═CHOCH₃, CH₂CH₂OCF₃, CH₂CH₂OCF₂H, CH₂CH₂OCFH₂, CH₂OCH₃, CH₂CF₂H, or CH₂CFH₂;
R₂ represents hydrogen, methyl, ethyl, CH(CH₃)₂, or C₁₋₆ alkyl-O—C₁₋₆ alkyl;
Rx represents hydrogen, methyl, or ethyl;
Ry represents hydrogen, methyl, ethyl, isopropyl, cyclopropyl, CH₂CH₂CH₃, or CN;
Rz represents hydrogen, methyl, ethyl, isopropyl, cyclopropyl, CH₂CH₂CH₃, CH₂CH₂CH₃, CF₃, cyclobutyl, furan-2-yl, C₆H₅, 4-fluorophenyl, CH₃CH₂OCO;
n represents 0, 1 or 2;
when n represents 1, the sulfur atom connected thereto may be selected from either an R configuration or an S configuration, or a mixture of the two, and the ratio of R to S in the mixture is 1:99 to 99:1

As the preferred specific compound structure of this invention, said 4-methylsulfonylbenzamide analogs, stereoisomers thereof, or agriculturally acceptable salts thereof, are selected from Table 1.

TABLE 1

| Compound NO. | Q | R_{X}/R_{Y}/R_{Z} | R₁ | R₂ | X | n | 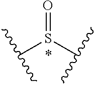 [SMILES: *S(*)=O] Configuration |
|---|---|---|---|---|---|---|---|
| 1-1 | Q1 | CH₃ | CH₂CH₂CH₃ | H | Cl | 0 | — |
| 1-2 | Q1 | CH₃ | CH₂CH₂CH₃ | H | Cl | 1 | racemate |
| 1-3 | Q1 | CH₃ | CH₂CH₂CH₃ | H | Cl | 1 | isomer1 |
| 1-4 | Q1 | CH₃ | CH₂CH₂CH₃ | H | Cl | 1 | isomer2 |
| 1-5 | Q1 | CH₃ | CH₂CH₂CH₃ | H | Cl | 2 | — |
| 1-6 | Q1 | CH₃ | CH(CH₃)₂ | H | Cl | 0 | — |
| 1-7 | Q1 | CH₃ | CH(CH₃)₂ | H | Cl | 1 | racemate |
| 1-8 | Q1 | CH₃ | CH(CH₃)₂ | H | Cl | 1 | isomer1 |
| 1-9 | Q1 | CH₃ | CH(CH₃)₂ | H | Cl | 1 | isomer2 |
| 1-10 | Q1 | CH₃ | CH(CH₃)₂ | H | Cl | 2 | — |
| 1-11 | Q1 | CH₃ | CH₂CH₂CH₂CH₃ | H | Cl | | — |
| 1-12 | Q1 | CH₃ | CH₂CH₂CH₂CH₃ | H | Cl | 1 | racemate |
| 1-13 | Q1 | CH₃ | CH₂CH₂CH₂CH₃ | H | Cl | 1 | isomer1 |
| 1-14 | Q1 | CH₃ | CH₂CH₂CH₂CH₃ | H | Cl | 1 | isomer2 |
| 1-15 | Q1 | CH₃ | CH₂CH₂CH₂CH₃ | H | Cl | 2 | — |
| 1-16 | Q1 | CH₃ | CH(CH₃)CH₂CH₃ | H | Cl | 0 | racemate |
| 1-17 | Q1 | CH₃ | CH(CH₃)CH₂CH₃ | H | Cl | 0 | isomer1 |
| 1-18 | Q1 | CH₃ | CH(CH₃)CH₂CH₃ | H | Cl | 0 | isomer2 |
| 1-19 | Q1 | CH₃ | CH(CH₃)CH₂CH₃ | H | Cl | 1 | mixture |
| 1-20 | Q1 | CH₃ | CH(CH₃)CH₂CH₃ | H | Cl | 1 | isomer1 |
| 1-21 | Q1 | CH₃ | CH(CH₃)CH₂CH₃ | H | Cl | 1 | isomer2 |
| 1-22 | Q1 | CH₃ | CH(CH₃)CH₂CH₃ | H | Cl | 1 | isomer3 |
| 1-23 | Q1 | CH₃ | CH(CH₃)CH₂CH₃ | H | Cl | 1 | isomer4 |
| 1-24 | Q1 | CH₃ | CH(CH₃)CH₂CH₃ | H | Cl | 2 | racemate |
| 1-25 | Q1 | CH₃ | CH(CH₃)CH₂CH₃ | H | Cl | 2 | isomer1 |
| 1-26 | Q1 | CH₃ | CH(CH₃)CH₂CH₃ | H | Cl | 2 | isomer2 |
| 1-27 | Q1 | CH₃ | cyclopropylmethyl | H | Cl | 0 | — |
| 1-28 | Q1 | CH₃ | cyclopropylmethyl | H | Cl | 1 | racemate |
| 1-29 | Q1 | CH₃ | cyclopropylmethyl | H | Cl | 1 | isomer1 |
| 1-30 | Q1 | CH₃ | cyclopropylmethyl | H | Cl | 1 | isomer2 |
| 1-31 | Q1 | CH₃ | cyclopropylmethyl | H | Cl | 2 | — |
| 1-32 | Q1 | CH₃ | cyclopropyl | H | Cl | 0 | — |
| 1-33 | Q1 | CH₃ | cyclopropyl | H | Cl | 1 | racemate |
| 1-34 | Q1 | CH₃ | cyclopropyl | H | Cl | 1 | isomer1 |
| 1-35 | Q1 | CH₃ | cyclopropyl | H | Cl | 1 | isomer2 |
| 1-36 | Q1 | CH₃ | cyclopropyl | H | Cl | 2 | — |
| 1-37 | Q1 | CH₃ | cyclobutyl | H | Cl | 0 | — |
| 1-38 | Q1 | CH₃ | cyclobutyl | H | Cl | 1 | racemate |
| 1-39 | Q1 | CH₃ | cyclobutyl | H | Cl | 1 | isomer1 |
| 1-40 | Q1 | CH₃ | cyclobutyl | H | Cl | 1 | isomer2 |
| 1-41 | Q1 | CH₃ | cyclobutyl | H | Cl | 2 | — |
| 1-42 | Q1 | CH₃ | cyclopentyl | H | Cl | 0 | — |
| 1-43 | Q1 | CH₃ | cyclopentyl | H | Cl | 1 | racemate |
| 1-44 | Q1 | CH₃ | cyclopentyl | H | Cl | 1 | isomer1 |
| 1-45 | Q1 | CH₃ | cyclopentyl | H | Cl | 1 | isomer2 |
| 1-46 | Q1 | CH₃ | cyclopentyl | H | Cl | 2 | — |
| 1-47 | Q1 | CH₃ | cyclohexyl | H | Cl | 0 | — |
| 1-48 | Q1 | CH₃ | cyclohexyl | H | Cl | 1 | racemate |
| 1-49 | Q1 | CH₃ | cyclohexyl | H | Cl | 1 | isomer1 |
| 1-50 | Q1 | CH₃ | cyclohexyl | H | Cl | 1 | isomer2 |
| 1-51 | Q1 | CH₃ | cyclohexyl | H | Cl | 2 | — |
| 1-52 | Q1 | CH₃ | CH₂CF₃ | H | Cl | 0 | — |
| 1-53 | Q1 | CH₃ | CH₂CF₃ | H | Cl | 1 | racemate |
| 1-54 | Q1 | CH₃ | CH₂CF₃ | H | Cl | 1 | isomer1 |
| 1-55 | Q1 | CH₃ | CH₂CF₃ | H | Cl | 1 | isomer2 |
| 1-56 | Q1 | CH₃ | CH₂CF₃ | H | Cl | 2 | — |
| 1-57 | Q1 | CH₃ | CH₂CF₂H | H | Cl | 0 | — |
| 1-58 | Q1 | CH₃ | CH₂CF₂H | H | Cl | 1 | racemate |
| 1-59 | Q1 | CH₃ | CH₂CF₂H | H | Cl | 1 | isomer1 |
| 1-60 | Q1 | CH₃ | CH₂CF₂H | H | Cl | 1 | isomer2 |
| 1-61 | Q1 | CH₃ | CH₂CF₂H | H | Cl | 2 | — |
| 1-62 | Q1 | CH₃ | CH₂CN | H | Cl | 0 | — |
| 1-63 | Q1 | CH₃ | CH₂CN | H | Cl | 1 | racemate |
| 1-64 | Q1 | CH₃ | CH₂CN | H | Cl | 1 | isomer1 |
| 1-65 | Q1 | CH₃ | CH₂CN | H | Cl | 1 | isomer2 |
| 1-66 | Q1 | CH₃ | CH₂CN | H | Cl | 2 | — |
| 1-67 | Q1 | CH₃ | CH₂CH═CH₂ | H | Cl | 0 | — |
| 1-68 | Q1 | CH₃ | CH₂CH═CH₂ | H | Cl | 1 | racemate |
| 1-69 | Q1 | CH₃ | CH₂CH═CH₂ | H | Cl | 1 | isomer1 |
| 1-70 | Q1 | CH₃ | CH₂CH═CH₂ | H | Cl | 1 | isomer2 |
| 1-71 | Q1 | CH₃ | CH₂CH═CH₂ | H | Cl | 2 | — |
| 1-72 | Q1 | CH₃ | CH₂CH₂Cl | H | Cl | 0 | — |
| 1-73 | Q1 | CH₃ | CH₂CH₂Cl | H | Cl | 1 | racemate |
| 1-74 | Q1 | CH₃ | CH₂CH₂Cl | H | Cl | 1 | isomer1 |
| 1-75 | Q1 | CH₃ | CH₂CH₂Cl | H | Cl | 1 | isomer2 |
| 1-76 | Q1 | CH₃ | CH₂CH₂Cl | H | Cl | 2 | — |
| 1-77 | Q1 | H | CH₂CH₂CH₃ | H | Cl | 0 | — |
| 1-78 | Q1 | H | CH₂CH₂CH₃ | H | Cl | 1 | racemate |
| 1-79 | Q1 | H | CH₂CH₂CH₃ | H | Cl | 1 | isomer1 |
| 1-80 | Q1 | H | CH₂CH₂CH₃ | H | Cl | 1 | isomer2 |
| 1-81 | Q1 | H | CH₂CH₂CH₃ | H | Cl | 2 | — |
| 1-82 | Q1 | H | CH(CH₃)₂ | H | Cl | 0 | — |
| 1-83 | Q1 | H | CH(CH₃)₂ | H | Cl | 1 | racemate |
| 1-84 | Q1 | H | CH(CH₃)₂ | H | Cl | 1 | isomer1 |
| 1-85 | Q1 | H | CH(CH₃)₂ | H | Cl | 1 | isomer2 |
| 1-86 | Q1 | H | CH(CH₃)₂ | H | Cl | 2 | — |
| 1-87 | Q1 | CH₂CH₃ | CH₂CH₂CH₃ | H | Cl | 0 | — |
| 1-88 | Q1 | CH₂CH₃ | CH₂CH₂CH₃ | H | Cl | 1 | racemate |
| 1-89 | Q1 | CH₂CH₃ | CH₂CH₂CH₃ | H | Cl | 1 | isomer1 |
| 1-90 | Q1 | CH₂CH₃ | CH₂CH₂CH₃ | H | Cl | 1 | isomer2 |
| 1-91 | Q1 | CH₂CH₃ | CH₂CH₂CH₃ | H | Cl | 2 | — |
| 1-92 | Q1 | CH₂CH₃ | CH(CH₃)₂ | H | Cl | 0 | — |
| 1-93 | Q1 | CH₂CH₃ | CH(CH₃)₂ | H | Cl | 1 | racemate |
| 1-94 | Q1 | CH₂CH₃ | CH(CH₃)₂ | H | Cl | 1 | isomer1 |
| 1-95 | Q1 | CH₂CH₃ | CH(CH₃)₂ | H | Cl | 1 | isomer2 |
| 1-96 | Q1 | CH₂CH₃ | CH(CH₃)₂ | H | Cl | 2 | — |
| 1-97 | Q1 | CH₃ | CH₂CH₂CH₃ | CH₃ | Cl | 0 | — |
| 1-98 | Q1 | CH₃ | CH₂CH₂CH₃ | CH₃ | Cl | 1 | racemate |
| 1-99 | Q1 | CH₃ | CH₂CH₂CH₃ | CH₃ | Cl | 1 | isomer1 |
| 1-100 | Q1 | CH₃ | CH₂CH₂CH₃ | CH₃ | Cl | 1 | isomer2 |
| 1-101 | Q1 | CH₃ | CH₂CH₂CH₃ | CH₃ | Cl | 2 | — |
| 1-102 | Q1 | CH₃ | CH(CH₃)₂ | CH₃ | Cl | 0 | — |
| 1-103 | Q1 | CH₃ | CH(CH₃)₂ | CH₃ | Cl | 1 | racemate |
| 1-104 | Q1 | CH₃ | CH(CH₃)₂ | CH₃ | Cl | 1 | isomer1 |
| 1-105 | Q1 | CH₃ | CH(CH₃)₂ | CH₃ | Cl | 1 | isomer2 |
| 1-106 | Q1 | CH₃ | CH(CH₃)₂ | CH₃ | Cl | 2 | — |
| 1-107 | Q1 | CH₃ | CH₂CH₂CH₂CH₃ | CH₃ | Cl | 0 | — |
| 1-108 | Q1 | CH₃ | CH₂CH₂CH₂CH₃ | CH₃ | Cl | 1 | racemate |
| 1-109 | Q1 | CH₃ | CH₂CH₂CH₂CH₃ | CH₃ | Cl | 1 | isomer1 |
| 1-110 | Q1 | CH₃ | CH₂CH₂CH₂CH₃ | CH₃ | Cl | 1 | isomer2 |
| 1-111 | Q1 | CH₃ | CH₂CH₂CH₂CH₃ | CH₃ | Cl | 2 | — |
| 1-112 | Q1 | CH₃ | CH(CH₃)CH₂CH₃ | CH₃ | Cl | 0 | racemate |
| 1-113 | Q1 | CH₃ | CH(CH₃)CH₂CH₃ | CH₃ | Cl | 0 | isomer1 |
| 1-114 | Q1 | CH₃ | CH(CH₃)CH₂CH₃ | CH₃ | Cl | 0 | isomer2 |
| 1-115 | Q1 | CH₃ | CH(CH₃)CH₂CH₃ | CH₃ | Cl | 1 | mixture |
| 1-116 | Q1 | CH₃ | CH(CH₃)CH₂CH₃ | CH₃ | Cl | 1 | isomer1 |
| 1-117 | Q1 | CH₃ | CH(CH₃)CH₂CH₃ | CH₃ | Cl | 1 | isomer2 |
| 1-118 | Q1 | CH₃ | CH(CH₃)CH₂CH₃ | CH₃ | Cl | 1 | isomer3 |
| 1-119 | Q1 | CH₃ | CH(CH₃)CH₂CH₃ | CH₃ | Cl | 1 | isomer4 |
| 1-120 | Q1 | CH₃ | CH(CH₃)CH₂CH₃ | CH₃ | Cl | 2 | racemate |
| 1-121 | Q1 | CH₃ | CH(CH₃)CH₂CH₃ | CH₃ | Cl | 2 | isomer1 |
| 1-122 | Q1 | CH₃ | CH(CH₃)CH₂CH₃ | CH₃ | Cl | 2 | isomer2 |
| 1-123 | Q1 | CH₃ | CH₂CH(CH₃)₂ | CH₃ | Cl | 0 | — |
| 1-124 | Q1 | CH₃ | CH₂CH(CH₃)₂ | CH₃ | Cl | 1 | racemate |
| 1-125 | Q1 | CH₃ | CH₂CH(CH₃)₂ | CH₃ | Cl | 1 | isomer1 |
| 1-126 | Q1 | CH₃ | CH₂CH(CH₃)₂ | CH₃ | Cl | 1 | isomer2 |
| 1-127 | Q1 | CH₃ | CH₂CH(CH₃)₂ | CH₃ | Cl | 2 | — |
| 1-128 | Q1 | CH₃ | cyclopropylmethyl | CH₃ | Cl | 0 | — |
| 1-129 | Q1 | CH₃ | cyclopropylmethyl | CH₃ | Cl | 1 | racemate |
| 1-130 | Q1 | CH₃ | cyclopropylmethyl | CH₃ | Cl | 1 | isomer1 |
| 1-131 | Q1 | CH₃ | cyclopropylmethyl | CH₃ | Cl | 1 | isomer2 |
| 1-132 | Q1 | CH₃ | cyclopropylmethyl | CH₃ | Cl | 2 | — |
| 1-133 | Q1 | CH₃ | cyclopropyl | CH₃ | Cl | 0 | — |
| 1-134 | Q1 | CH₃ | cyclopropyl | CH₃ | Cl | 1 | racemate |
| 1-135 | Q1 | CH₃ | cyclopropyl | CH₃ | Cl | 1 | isomer1 |
| 1-136 | Q1 | CH₃ | cyclopropyl | CH₃ | Cl | 1 | isomer2 |
| 1-137 | Q1 | CH₃ | cyclopropyl | CH₃ | Cl | 2 | — |
| 1-138 | Q1 | CH₃ | cyclobutyl | CH₃ | Cl | 0 | — |
| 1-139 | Q1 | CH₃ | cyclobutyl | CH₃ | Cl | 1 | racemate |
| 1-140 | Q1 | CH₃ | cyclobutyl | CH₃ | Cl | 1 | isomer1 |
| 1-141 | Q1 | CH₃ | cyclobutyl | CH₃ | Cl | 1 | isomer2 |
| 1-142 | Q1 | CH₃ | cyclobutyl | CH₃ | Cl | 2 | — |
| 1-143 | Q1 | CH₃ | cyclopentyl | CH₃ | Cl | 0 | — |
| 1-144 | Q1 | CH₃ | cyclopentyl | CH₃ | Cl | 1 | racemate |
| 1-145 | Q1 | CH₃ | cyclopentyl | CH₃ | Cl | 1 | isomer1 |
| 1-146 | Q1 | CH₃ | cyclopentyl | CH₃ | Cl | 1 | isomer2 |
| 1-147 | Q1 | CH₃ | cyclopentyl | CH₃ | Cl | 2 | — |
| 1-148 | Q1 | CH₃ | cyclohexyl | CH₃ | Cl | 0 | — |
| 1-149 | Q1 | CH₃ | cyclohexyl | CH₃ | Cl | 1 | racemate |
| 1-150 | Q1 | CH₃ | cyclohexyl | CH₃ | Cl | 1 | isomer1 |
| 1-151 | Q1 | CH₃ | cyclohexyl | CH₃ | Cl | 1 | isomer2 |
| 1-152 | Q1 | CH₃ | cyclohexyl | CH₃ | Cl | 2 | — |
| 1-153 | Q1 | CH₃ | CH₂CH₂CH₃ | CH₂CH₃ | Cl | 0 | — |
| 1-154 | Q1 | CH₃ | CH₂CH₂CH₃ | CH₂CH₃ | Cl | 1 | racemate |
| 1-155 | Q1 | CH₃ | CH₂CH₂CH₃ | CH₂CH₃ | Cl | 1 | isomer1 |
| 1-156 | Q1 | CH₃ | CH₂CH₂CH₃ | CH₂CH₃ | Cl | 1 | isomer2 |
| 1-157 | Q1 | CH₃ | CH₂CH₂CH₃ | CH₂CH₃ | Cl | 2 | — |
| 1-158 | Q1 | CH₃ | CH(CH₃)₂ | CH₂CH₃ | Cl | 0 | — |
| 1-159 | Q1 | CH₃ | CH(CH₃)₂ | CH₂CH₃ | Cl | 1 | racemate |
| 1-160 | Q1 | CH₃ | CH(CH₃)₂ | CH₂CH₃ | Cl | 1 | isomer 1 |
| 1-161 | Q1 | CH₃ | CH(CH₃)₂ | CH₂CH₃ | Cl | 1 | isomer2 |
| 1-162 | Q1 | CH₃ | CH(CH₃)₂ | CH₂CH₃ | Cl | 2 | — |
| 1-163 | Q1 | CH₃ | CH₂CH₂CH₃ | CH₂CH₂OCH₃ | Cl | 0 | — |
| 1-164 | Q1 | CH₃ | CH₂CH₂CH₃ | CH₂CH₂OCH₃ | Cl | 1 | racemate |
| 1-165 | Q1 | CH₃ | CH₂CH₂CH₃ | CH₂CH₂OCH₃ | Cl | 1 | isomer1 |
| 1-166 | Q1 | CH₃ | CH₂CH₂CH₃ | CH₂CH₂OCH₃ | Cl | 1 | isomer2 |
| 1-167 | Q1 | CH₃ | CH₂CH₂CH₃ | CH₂CH₂OCH₃ | Cl | 2 | — |
| 1-168 | Q1 | CH₃ | CH(CH₃)₂ | CH₂CH₂OCH₃ | Cl | 0 | — |
| 1-169 | Q1 | CH₃ | CH(CH₃)₂ | CH₂CH₂OCH₃ | Cl | 1 | racemate |
| 1-170 | Q1 | CH₃ | CH(CH₃)₂ | CH₂CH₂OCH₃ | Cl | 1 | isomer1 |
| 1-171 | Q1 | CH₃ | CH(CH₃)₂ | CH₂CH₂OCH₃ | Cl | 1 | isomer2 |
| 1-172 | Q1 | CH₃ | CH(CH₃)₂ | CH₂CH₂OCH₃ | Cl | 2 | — |
| 1-173 | Q1 | CH₃ | cyclobutylmethyl | H | Cl | 0 | — |
| 1-174 | Q1 | CH₃ | cyclobutylmethyl | H | Cl | 1 | racemate |
| 1-175 | Q1 | CH₃ | cyclobutylmethyl | H | Cl | 1 | isomer1 |
| 1-176 | Q1 | CH₃ | cyclobutylmethyl | H | Cl | 1 | isomer2 |
| 1-177 | Q1 | CH₃ | cyclobutylmethyl | H | Cl | 2 | — |
| 1-178 | Q1 | CH₃ | cyclopentylmethyl | H | Cl | 0 | — |
| 1-179 | Q1 | CH₃ | cyclopentylmethyl | H | Cl | 1 | racemate |
| 1-180 | Q1 | CH₃ | cyclopentylmethyl | H | Cl | 1 | isomer1 |
| 1-181 | Q1 | CH₃ | cyclopentylmethyl | H | Cl | 1 | isomer2 |
| 1-182 | Q1 | CH₃ | cyclopentylmethyl | H | Cl | 2 | — |
| 1-183 | Q1 | CH₃ | 3,3-difluorocyclobutyl | H | Cl | 0 | — |
| 1-184 | Q1 | CH₃ | 3,3-difluorocyclobutyl | H | Cl | 1 | racemate |
| 1-185 | Q1 | CH₃ | 3,3-difluorocyclobutyl | H | Cl | 1 | isomer 1 |
| 1-186 | Q1 | CH₃ | 3,3-difluorocyclobutyl | H | Cl | 1 | isomer2 |
| 1-187 | Q1 | CH₃ | 3,3-difluorocyclobutyl | H | Cl | 2 | — |
| 2-1 | Q2 | CH₃ | CH₂CH₂CH₃ | H | Cl | 0 | — |
| 2-2 | Q2 | CH₃ | CH₂CH₂CH₃ | H | Cl | 1 | racemate |
| 2-3 | Q2 | CH₃ | CH₂CH₂CH₃ | H | Cl | 1 | isomer1 |
| 2-4 | Q2 | CH₃ | CH₂CH₂CH₃ | H | Cl | 1 | isomer2 |
| 2-5 | Q2 | CH₃ | CH₂CH₂CH₃ | H | Cl | 2 | — |
| 2-6 | Q2 | CH₃ | CH(CH₃)₂ | H | Cl | 0 | — |
| 2-7 | Q2 | CH₃ | CH(CH₃)₂ | H | Cl | 1 | racemate |
| 2-8 | Q2 | CH₃ | CH(CH₃)₂ | H | Cl | 1 | isomer1 |
| 2-9 | Q2 | CH₃ | CH(CH₃)₂ | H | Cl | 1 | isomer2 |
| 2-10 | Q2 | CH₃ | CH(CH₃)₂ | H | Cl | 2 | — |
| 2-11 | Q2 | CH₃ | CH₂CH₂CH₂CH₃ | H | Cl | 0 | — |
| 2-12 | Q2 | CH₃ | CH₂CH₂CH₂CH₃ | H | Cl | 1 | racemate |
| 2-13 | Q2 | CH₃ | CH₂CH₂CH₂CH₃ | H | Cl | 1 | isomer1 |
| 2-14 | Q2 | CH₃ | CH₂CH₂CH₂CH₃ | H | Cl | 1 | isomer2 |
| 2-15 | Q2 | CH₃ | CH₂CH₂CH₂CH₃ | H | Cl | 2 | — |
| 2-16 | Q2 | CH₃ | CH(CH₃)CH₂CH₃ | H | Cl | 0 | racemate |
| 2-17 | Q2 | CH₃ | CH(CH₃)CH₂CH₃ | H | Cl | 0 | isomer1 |
| 2-18 | Q2 | CH₃ | CH(CH₃)CH₂CH₃ | H | Cl | 0 | isomer2 |
| 2-19 | Q2 | CH₃ | CH(CH₃)CH₂CH₃ | H | Cl | 1 | mixture |
| 2-20 | Q2 | CH₃ | CH(CH₃)CH₂CH₃ | H | Cl | 1 | isomer1 |
| 2-21 | Q2 | CH₃ | CH(CH₃)CH₂CH₃ | H | Cl | 1 | isomer2 |
| 2-22 | Q2 | CH₃ | CH(CH₃)CH₂CH₃ | H | Cl | 1 | isomer3 |
| 2-23 | Q2 | CH₃ | CH(CH₃)CH₂CH₃ | H | Cl | 1 | isomer4 |
| 2-24 | Q2 | CH₃ | CH(CH₃)CH₂CH₃ | H | Cl | 2 | racemate |
| 2-25 | Q2 | CH₃ | CH(CH₃)CH₂CH₃ | H | Cl | 2 | isomer1 |
| 2-26 | Q2 | CH₃ | CH(CH₃)CH₂CH₃ | H | Cl | 2 | isomer2 |
| 2-27 | Q2 | CH₃ | cyclopropylmethyl | H | Cl | 0 | — |
| 2-28 | Q2 | CH₃ | cyclopropylmethyl | H | Cl | 1 | racemate |
| 2-29 | Q2 | CH₃ | cyclopropylmethyl | H | Cl | 1 | isomer1 |
| 2-30 | Q2 | CH₃ | cyclopropylmethyl | H | Cl | 1 | isomer2 |
| 2-31 | Q2 | CH₃ | cyclopropylmethyl | H | Cl | 2 | — |
| 2-32 | Q2 | CH₃ | cyclopropyl | H | Cl | 0 | — |
| 2-33 | Q2 | CH₃ | cyclopropyl | H | Cl | 1 | racemate |
| 2-34 | Q2 | CH₃ | cyclopropyl | H | Cl | 1 | isomer1 |
| 2-35 | Q2 | CH₃ | cyclopropyl | H | Cl | 1 | isomer2 |
| 2-36 | Q2 | CH₃ | cyclopropyl | H | Cl | 2 | — |
| 2-37 | Q2 | CH₃ | cyclobutyl | H | Cl | 0 | — |
| 2-38 | Q2 | CH₃ | cyclobutyl | H | Cl | 1 | racemate |
| 2-39 | Q2 | CH₃ | cyclobutyl | H | Cl | 1 | isomer1 |
| 2-40 | Q2 | CH₃ | cyclobutyl | H | Cl | 1 | isomer2 |
| 2-41 | Q2 | CH₃ | cyclobutyl | H | Cl | 2 | — |
| 2-42 | Q2 | CH₃ | cyclopentyl | H | Cl | 0 | — |
| 2-43 | Q2 | CH₃ | cyclopentyl | H | Cl | 1 | racemate |
| 2-44 | Q2 | CH₃ | cyclopentyl | H | Cl | 1 | isomer1 |
| 2-45 | Q2 | CH₃ | cyclopentyl | H | Cl | 1 | isomer2 |
| 2-46 | Q2 | CH₃ | cyclopentyl | H | Cl | 2 | — |
| 2-47 | Q2 | CH₃ | cyclohexyl | H | Cl | 0 | — |
| 2-48 | Q2 | CH₃ | cyclohexyl | H | Cl | 1 | racemate |
| 2-49 | Q2 | CH₃ | cyclohexyl | H | Cl | 1 | isomer1 |
| 2-50 | Q2 | CH₃ | cyclohexyl | H | Cl | 1 | isomer2 |
| 2-51 | Q2 | CH₃ | cyclohexyl | H | Cl | 2 | — |
| 2-52 | Q2 | CH₃ | CH₂CF₃ | H | Cl | 0 | — |
| 2-53 | Q2 | CH₃ | CH₂CF₃ | H | Cl | 1 | racemate |
| 2-54 | Q2 | CH₃ | CH₂CF₃ | H | Cl | 1 | isomer1 |
| 2-55 | Q2 | CH₃ | CH₂CF₃ | H | Cl | 1 | isomer2 |
| 2-56 | Q2 | CH₃ | CH₂CF₃ | H | Cl | 2 | — |
| 2-57 | Q2 | CH₃ | CH₂CF₂H | H | Cl | 0 | — |
| 2-58 | Q2 | CH₃ | CH₂CF₂H | H | Cl | 1 | racemate |
| 2-59 | Q2 | CH₃ | CH₂CF₂H | H | Cl | 1 | isomer1 |
| 2-60 | Q2 | CH₃ | CH₂CF₂H | H | Cl | 1 | isomer2 |
| 2-61 | Q2 | CH₃ | CH₂CF₂H | H | Cl | 2 | — |
| 2-62 | Q2 | CH₃ | CH₂CN | H | Cl | 0 | — |
| 2-63 | Q2 | CH₃ | CH₂CN | H | Cl | 1 | racemate |
| 2-64 | Q2 | CH₃ | CH₂CN | H | Cl | 1 | isomer1 |
| 2-65 | Q2 | CH₃ | CH₂CN | H | Cl | 1 | isomer2 |
| 2-66 | Q2 | CH₃ | CH₂CN | H | Cl | 2 | — |
| 2-67 | Q2 | CH₃ | CH₂CH═CH₂ | H | Cl | 0 | — |
| 2-68 | Q2 | CH₃ | CH₂CH═CH₂ | H | Cl | 1 | racemate |
| 2-69 | Q2 | CH₃ | CH₂CH═CH₂ | H | Cl | 1 | isomer1 |
| 2-70 | Q2 | CH₃ | CH₂CH═CH₂ | H | Cl | 1 | isomer2 |
| 2-71 | Q2 | CH₃ | CH₂CH═CH₂ | H | Cl | 2 | — |
| 2-72 | Q2 | CH₃ | CH₂CH₂Cl | H | Cl | 0 | — |
| 2-73 | Q2 | CH₃ | CH₂CH₂Cl | H | Cl | 1 | racemate |
| 2-74 | Q2 | CH₃ | CH₂CH₂Cl | H | Cl | 1 | isomer1 |
| 2-75 | Q2 | CH₃ | CH₂CH₂Cl | H | Cl | 1 | isomer2 |
| 2-76 | Q2 | CH₃ | CH₂CH₂Cl | H | Cl | 2 | — |
| 2-77 | Q2 | CH₃ | CH₂CH₂CH₃ | CH₃ | Cl | 0 | — |
| 2-78 | Q2 | CH₃ | CH₂CH₂CH₃ | CH₃ | Cl | 1 | racemate |
| 2-79 | Q2 | CH₃ | CH₂CH₂CH₃ | CH₃ | Cl | 1 | isomer1 |
| 2-80 | Q2 | CH₃ | CH₂CH₂CH₃ | CH₃ | Cl | 1 | isomer2 |
| 2-81 | Q2 | CH₃ | CH₂CH₂CH₃ | CH₃ | Cl | 2 | — |
| 2-82 | Q2 | CH₃ | CH(CH₃)₂ | CH₃ | Cl | 0 | — |
| 2-83 | Q2 | CH₃ | CH(CH₃)₂ | CH₃ | Cl | 1 | racemate |
| 2-84 | Q2 | CH₃ | CH(CH₃)₂ | CH₃ | Cl | 1 | isomer1 |
| 2-85 | Q2 | CH₃ | CH(CH₃)₂ | CH₃ | Cl | 1 | isomer2 |
| 2-86 | Q2 | CH₃ | CH(CH₃)₂ | CH₃ | Cl | 2 | — |
| 2-87 | Q2 | CH₃ | CH₂CH₂CH₃ | CH₂CH₃ | Cl | 0 | — |
| 2-88 | Q2 | CH₃ | CH₂CH₂CH₃ | CH₂CH₃ | Cl | 1 | racemate |
| 2-89 | Q2 | CH₃ | CH₂CH₂CH₃ | CH₂CH₃ | Cl | 1 | isomer1 |
| 2-90 | Q2 | CH₃ | CH₂CH₂CH₃ | CH₂CH₃ | Cl | 1 | isomer2 |
| 2-91 | Q2 | CH₃ | CH₂CH₂CH₃ | CH₂CH₃ | Cl | 2 | — |
| 2-92 | Q2 | CH₃ | CH(CH₃)₂ | CH₂CH₃ | Cl | 0 | — |
| 2-93 | Q2 | CH₃ | CH(CH₃)₂ | CH₂CH₃ | Cl | 1 | racemate |
| 2-94 | Q2 | CH₃ | CH(CH₃)₂ | CH₂CH₃ | Cl | 1 | isomer1 |
| 2-95 | Q2 | CH₃ | CH(CH₃)₂ | CH₂CH₃ | Cl | 1 | isomer2 |
| 2-96 | Q2 | CH₃ | CH(CH₃)₂ | CH₂CH₃ | Cl | 2 | — |
| 2-97 | Q2 | CH₃ | cyclobutyl | CH₂CH₂OCH₃ | Cl | 0 | — |
| 2-98 | Q2 | CH₃ | cyclobutyl | CH₂CH₂OCH₃ | Cl | 1 | racemate |
| 2-99 | Q2 | CH₃ | cyclobutyl | CH₂CH₂OCH₃ | Cl | 1 | isomer1 |
| 2-100 | Q2 | CH₃ | cyclobutyl | CH₂CH₂OCH₃ | Cl | 1 | isomer2 |
| 2-101 | Q2 | CH₃ | cyclobutyl | CH₂CH₂OCH₃ | Cl | 2 | — |
| 2-102 | Q2 | CH₃ | cyclobutylmethyl | H | Cl | 0 | — |
| 2-103 | Q2 | CH₃ | cyclobutylmethyl | H | Cl | 1 | racemate |
| 2-104 | Q2 | CH₃ | cyclobutylmethyl | H | Cl | 1 | isomer1 |
| 2-105 | Q2 | CH₃ | cyclobutylmethyl | H | Cl | 1 | isomer2 |
| 2-106 | Q2 | CH₃ | cyclobutylmethyl | H | Cl | 2 | — |
| 2-107 | Q2 | CH₃ | cyclopentylmethyl | H | Cl | 0 | — |
| 2-108 | Q2 | CH₃ | cyclopentylmethyl | H | Cl | 1 | racemate |
| 2-109 | Q2 | CH₃ | cyclopentylmethyl | H | Cl | 1 | isomer1 |
| 2-110 | Q2 | CH₃ | cyclopentylmethyl | H | Cl | 1 | isomer2 |
| 2-111 | Q2 | CH₃ | cyclopentylmethyl | H | Cl | 2 | — |
| 2-112 | Q2 | CH₃ | 3,3-difluorocyclobutyl | H | Cl | 0 | — |
| 2-113 | Q2 | CH₃ | 3,3-difluorocyclobutyl | H | Cl | 1 | racemate |
| 2-114 | Q2 | CH₃ | 3,3-difluorocyclobutyl | H | Cl | 1 | isomer1 |
| 2-115 | Q2 | CH₃ | 3,3-difluorocyclobutyl | H | Cl | 1 | isomer2 |
| 2-116 | Q2 | CH₃ | 3,3-difluorocyclobutyl | H | Cl | 2 | — |
| 3-1 | Q3 | CH₃ | CH₂CH₂CH₃ | H | Cl | 0 | — |
| 3-2 | Q3 | CH₃ | CH₂CH₂CH₃ | H | Cl | 1 | racemate |
| 3-3 | Q3 | CH₃ | CH₂CH₂CH₃ | H | Cl | 1 | isomer1 |
| 3-4 | Q3 | CH₃ | CH₂CH₂CH₃ | H | Cl | 1 | isomer2 |
| 3-5 | Q3 | CH₃ | CH₂CH₂CH₃ | H | Cl | 2 | — |
| 3-6 | Q3 | CH₃ | CH(CH₃)₂ | H | Cl | 0 | — |
| 3-7 | Q3 | CH₃ | CH(CH₃)₂ | H | Cl | 1 | racemate |
| 3-8 | Q3 | CH₃ | CH(CH₃)₂ | H | Cl | 1 | isomer1 |
| 3-9 | Q3 | CH₃ | CH(CH₃)₂ | H | Cl | 1 | isomer2 |
| 3-10 | Q3 | CH₃ | CH(CH₃)₂ | H | Cl | 2 | — |
| 3-11 | Q3 | CH₃ | CH₂CH₂CH₂CH₃ | H | Cl | 0 | — |
| 3-12 | Q3 | CH₃ | CH₂CH₂CH₂CH₃ | H | Cl | 1 | racemate |
| 3-13 | Q3 | CH₃ | CH₂CH₂CH₂CH₃ | H | Cl | 1 | isomer 1 |
| 3-14 | Q3 | CH₃ | CH₂CH₂CH₂CH₃ | H | Cl | 1 | isomer2 |
| 3-15 | Q3 | CH₃ | CH₂CH₂CH₂CH₃ | H | Cl | 2 | — |
| 3-16 | Q3 | CH₃ | CH(CH₃)CH₂CH₃ | H | Cl | 0 | racemate |
| 3-17 | Q3 | CH₃ | CH(CH₃)CH₂CH₃ | H | Cl | 0 | isomer1 |
| 3-18 | Q3 | CH₃ | CH(CH₃)CH₂CH₃ | H | Cl | 0 | isomer2 |
| 3-19 | Q3 | CH₃ | CH(CH₃)CH₂CH₃ | H | Cl | 1 | mixture |
| 3-20 | Q3 | CH₃ | CH(CH₃)CH₂CH₃ | H | Cl | 1 | isomer1 |
| 3-21 | Q3 | CH₃ | CH(CH₃)CH₂CH₃ | H | Cl | 1 | isomer2 |
| 3-22 | Q3 | CH₃ | CH(CH₃)CH₂CH₃ | H | Cl | 1 | isomer3 |
| 3-23 | Q3 | CH₃ | CH(CH₃)CH₂CH₃ | H | Cl | 1 | isomer4 |
| 3-24 | Q3 | CH₃ | CH(CH₃)CH₂CH₃ | H | Cl | 2 | racemate |
| 3-25 | Q3 | CH₃ | CH(CH₃)CH₂CH₃ | H | Cl | 2 | isomer1 |
| 3-26 | Q3 | CH₃ | CH(CH₃)CH₂CH₃ | H | Cl | 2 | isomer2 |
| 3-27 | Q3 | CH₃ | CH₂CH(CH₃)₂ | H | Cl | 0 | — |
| 3-28 | Q3 | CH₃ | CH₂CH(CH₃)₂ | H | Cl | 1 | racemate |
| 3-29 | Q3 | CH₃ | CH₂CH(CH₃)₂ | H | Cl | 1 | isomer1 |
| 3-30 | Q3 | CH₃ | CH₂CH(CH₃)₂ | H | Cl | 1 | isomer2 |
| 3-31 | Q3 | CH₃ | CH₂CH(CH₃)₂ | H | Cl | 2 | — |
| 3-32 | Q3 | CH₃ | C(CH₃)₃ | H | Cl | 0 | — |
| 3-33 | Q3 | CH₃ | C(CH₃)₃ | H | Cl | 1 | racemate |
| 3-34 | Q3 | CH₃ | C(CH₃)₃ | H | Cl | 1 | isomer1 |
| 3-35 | Q3 | CH₃ | C(CH₃)₃ | H | Cl | 1 | isomer2 |
| 3-36 | Q3 | CH₃ | C(CH₃)₃ | H | Cl | 2 | — |
| 3-37 | Q3 | CH₃ | cyclopropylmethyl | H | Cl | 0 | — |
| 3-38 | Q3 | CH₃ | cyclopropylmethyl | H | Cl | 1 | racemate |
| 3-39 | Q3 | CH₃ | cyclopropylmethyl | H | Cl | 1 | isomer 1 |
| 3-40 | Q3 | CH₃ | cyclopropylmethyl | H | Cl | 1 | isomer2 |
| 3-41 | Q3 | CH₃ | cyclopropylmethyl | H | Cl | 2 | — |
| 3-42 | Q3 | CH₃ | CH₂CH₂CH₂CH₂CH₃ | H | Cl | 0 | — |
| 3-43 | Q3 | CH₃ | CH₂CH₂CH₂CH₂CH₃ | H | Cl | 1 | racemate |
| 3-44 | Q3 | CH₃ | CH₂CH₂CH₂CH₂CH₃ | H | Cl | 1 | isomer 1 |
| 3-45 | Q3 | CH₃ | CH₂CH₂CH₂CH₂CH₃ | H | Cl | 1 | isomer2 |
| 3-46 | Q3 | CH₃ | CH₂CH₂CH₂CH₂CH₃ | H | Cl | 2 | — |
| 3-47 | Q3 | CH₃ | CH(CH₃)CH₂CH₂CH₃ | H | Cl | 0 | — |
| 3-48 | Q3 | CH₃ | CH(CH₃)CH₂CH₂CH₃ | H | Cl | 1 | racemate |
| 3-49 | Q3 | CH₃ | CH(CH₃)CH₂CH₂CH₃ | H | Cl | 1 | isomer1 |
| 3-50 | Q3 | CH₃ | CH(CH₃)CH₂CH₂CH₃ | H | Cl | 1 | isomer2 |
| 3-51 | Q3 | CH₃ | CH(CH₃)CH₂CH₂CH₃ | H | Cl | 2 | — |
| 3-52 | Q3 | CH₃ | CH₂CH₂CH(CH₃)₂ | H | Cl | 0 | — |
| 3-53 | Q3 | CH₃ | CH₂CH₂CH(CH₃)₂ | H | Cl | 1 | racemate |
| 3-54 | Q3 | CH₃ | CH₂CH₂CH(CH₃)₂ | H | Cl | 1 | isomer1 |
| 3-55 | Q3 | CH₃ | CH₂CH₂CH(CH₃)₂ | H | Cl | 1 | isomer2 |
| 3-56 | Q3 | CH₃ | CH₂CH₂CH(CH₃)₂ | H | Cl | 2 | — |
| 3-57 | Q3 | CH₃ | C(CH₃)₂CH₂CH₃ | H | Cl | 0 | — |
| 3-58 | Q3 | CH₃ | C(CH₃)₂CH₂CH₃ | H | Cl | 1 | racemate |
| 3-59 | Q3 | CH₃ | C(CH₃)₂CH₂CH₃ | H | Cl | 1 | isomer1 |
| 3-60 | Q3 | CH₃ | C(CH₃)₂CH₂CH₃ | H | Cl | 1 | isomer2 |
| 3-61 | Q3 | CH₃ | C(CH₃)₂CH₂CH₃ | H | Cl | 2 | — |
| 3-62 | Q3 | CH₃ | CH₂CH(CH₃)CH₂CH₃ | H | Cl | 0 | — |
| 3-63 | Q3 | CH₃ | CH₂CH(CH₃)CH₂CH₃ | H | Cl | 1 | racemate |
| 3-64 | Q3 | CH₃ | CH₂CH(CH₃)CH₂CH₃ | H | Cl | 1 | isomer 1 |
| 3-65 | Q3 | CH₃ | CH₂CH(CH₃)CH₂CH₃ | H | Cl | 1 | isomer2 |
| 3-66 | Q3 | CH₃ | CH₂CH(CH₃)CH₂CH₃ | H | Cl | 2 | — |
| 3-67 | Q3 | CH₃ | CH₂C₆H₅ | H | Cl | 0 | — |
| 3-68 | Q3 | CH₃ | CH₂C₆H₅ | H | Cl | 1 | racemate |
| 3-69 | Q3 | CH₃ | CH₂C₆H₅ | H | Cl | 1 | isomer 1 |
| 3-70 | Q3 | CH₃ | CH₂C₆H₅ | H | Cl | 1 | isomer2 |
| 3-71 | Q3 | CH₃ | CH₂C₆H₅ | H | Cl | 2 | — |
| 3-72 | Q3 | CH₃ | CH(CH₃)C₆H₅ | H | Cl | 0 | — |
| 3-73 | Q3 | CH₃ | CH(CH₃)C₆H₅ | H | Cl | 1 | racemate |
| 3-74 | Q3 | CH₃ | CH(CH₃)C₆H₅ | H | Cl | 1 | isomer1 |
| 3-75 | Q3 | CH₃ | CH(CH₃)C₆H₅ | H | Cl | 1 | isomer2 |
| 3-76 | Q3 | CH₃ | CH(CH₃)C₆H₅ | H | Cl | 2 | — |
| 3-77 | Q3 | CH₃ | cyclopropyl | H | Cl | 0 | — |
| 3-78 | Q3 | CH₃ | cyclopropyl | H | Cl | 1 | racemate |
| 3-79 | Q3 | CH₃ | cyclopropyl | H | Cl | 1 | isomer1 |
| 3-80 | Q3 | CH₃ | cyclopropyl | H | Cl | 1 | isomer2 |
| 3-81 | Q3 | CH₃ | cyclopropyl | H | Cl | 2 | — |
| 3-82 | Q3 | CH₃ | cyclobutyl | H | Cl | 0 | — |
| 3-83 | Q3 | CH₃ | cyclobutyl | H | Cl | 1 | racemate |
| 3-84 | Q3 | CH₃ | cyclobutyl | H | Cl | 1 | isomer 1 |
| 3-85 | Q3 | CH₃ | cyclobutyl | H | Cl | 1 | isomer2 |
| 3-86 | Q3 | CH₃ | cyclobutyl | H | Cl | 2 | — |
| 3-87 | Q3 | CH₃ | cyclopentyl | H | Cl | 0 | — |
| 3-88 | Q3 | CH₃ | cyclopentyl | H | Cl | 1 | racemate |
| 3-89 | Q3 | CH₃ | cyclopentyl | H | Cl | 1 | isomer1 |
| 3-90 | Q3 | CH₃ | cyclopentyl | H | Cl | 1 | isomer2 |
| 3-91 | Q3 | CH₃ | cyclopentyl | H | Cl | 2 | — |
| 3-92 | Q3 | CH₃ | cyclohexyl | H | Cl | 0 | — |
| 3-93 | Q3 | CH₃ | cyclohexyl | H | Cl | 1 | racemate |
| 3-94 | Q3 | CH₃ | cyclohexyl | H | Cl | 1 | isomer1 |
| 3-95 | Q3 | CH₃ | cyclohexyl | H | Cl | 1 | isomer2 |
| 3-96 | Q3 | CH₃ | cyclohexyl | H | Cl | 2 | — |
| 3-97 | Q3 | CH₃ | CH₂CF₃ | H | Cl | 0 | — |
| 3-98 | Q3 | CH₃ | CH₂CF₃ | H | Cl | 1 | racemate |
| 3-99 | Q3 | CH₃ | CH₂CF₃ | H | Cl | 1 | isomer1 |
| 3-100 | Q3 | CH₃ | CH₂CF₃ | H | Cl | 1 | isomer2 |
| 3-101 | Q3 | CH₃ | CH₂CF₃ | H | Cl | 2 | — |
| 3-102 | Q3 | CH₃ | CH₂CF₂H | H | Cl | 0 | — |
| 3-103 | Q3 | CH₃ | CH₂CF₂H | H | Cl | 1 | racemate |
| 3-104 | Q3 | CH₃ | CH₂CF₂H | H | Cl | 1 | isomer1 |
| 3-105 | Q3 | CH₃ | CH₂CF₂H | H | Cl | 1 | isomer2 |
| 3-106 | Q3 | CH₃ | CH₂CF₂H | H | Cl | 2 | — |
| 3-107 | Q3 | CH₃ | CH₂CFH₂ | H | Cl | 0 | — |
| 3-108 | Q3 | CH₃ | CH₂CFH₂ | H | Cl | 1 | racemate |
| 3-109 | Q3 | CH₃ | CH₂CFH₂ | H | Cl | 1 | isomer1 |
| 3-110 | Q3 | CH₃ | CH₂CFH₂ | H | Cl | 1 | isomer2 |
| 3-111 | Q3 | CH₃ | CH₂CFH₂ | H | Cl | 2 | — |
| 3-112 | Q3 | CH₃ | CH₂CH═CH₂ | H | Cl | 0 | — |
| 3-113 | Q3 | CH₃ | CH₂CH═CH₂ | H | Cl | 1 | racemate |
| 3-114 | Q3 | CH₃ | CH₂CH═CH₂ | H | Cl | 1 | isomer1 |
| 3-115 | Q3 | CH₃ | CH₂CH═CH₂ | H | Cl | 1 | isomer2 |
| 3-116 | Q3 | CH₃ | CH₂CH═CH₂ | H | Cl | 2 | — |
| 3-117 | Q3 | CH₃ | CH(CH₃)CH═CH₂ | H | Cl | 0 | — |
| 3-118 | Q3 | CH₃ | CH(CH₃)CH═CH₂ | H | Cl | 1 | racemate |
| 3-119 | Q3 | CH₃ | CH(CH₃)CH═CH₂ | H | Cl | 1 | isomer1 |
| 3-120 | Q3 | CH₃ | CH(CH₃)CH═CH₂ | H | Cl | 1 | isomer2 |
| 3-121 | Q3 | CH₃ | CH(CH₃)CH═CH₂ | H | Cl | 2 | — |
| 3-122 | Q3 | CH₃ | (E)—CH₂CH═CHCH₃ | H | Cl | 0 | — |
| 3-123 | Q3 | CH₃ | (E)—CH₂CH═CHCH₃ | H | Cl | 1 | racemate |
| 3-124 | Q3 | CH₃ | (E)—CH₂CH═CHCH₃ | H | Cl | 1 | isomer1 |
| 3-125 | Q3 | CH₃ | (E)—CH₂CH═CHCH₃ | H | Cl | 1 | isomer2 |
| 3-126 | Q3 | CH₃ | (E)—CH₂CH═CHCH₃ | H | Cl | 2 | — |
| 3-127 | Q3 | CH₃ | (Z)—CH₂CH═CHCH₃ | H | Cl | 0 | — |
| 3-128 | Q3 | CH₃ | (Z)—CH₂CH═CHCH | H | Cl | 1 | racemate |
| 3-129 | Q3 | CH₃ | (Z)—CH₂CH═CHCH₃ | H | Cl | 1 | isomer1 |
| 3-130 | Q3 | CH₃ | (Z)—CH₂CH═CHCH₃ | H | Cl | 1 | isomer2 |
| 3-131 | Q3 | CH₃ | (Z)—CH₂CH═CHCH₃ | H | Cl | 2 | — |
| 3-132 | Q3 | CH₃ | CH₂CH═C(CH₃)₂ | H | Cl | 0 | — |
| 3-133 | Q3 | CH₃ | CH₂CH═C(CH₃)₂ | H | Cl | 1 | racemate |
| 3-134 | Q3 | CH₃ | CH₂CH═C(CH₃)₂ | H | Cl | 1 | isomer1 |
| 3-135 | Q3 | CH₃ | CH₂CH═C(CH₃)₂ | H | Cl | 1 | isomer2 |
| 3-136 | Q3 | CH₃ | CH₂CH═C(CH₃)₂ | H | Cl | 2 | — |
| 3-137 | Q3 | CH₃ | CH(CH₃)CH═CH₂ | H | Cl | 0 | — |
| 3-138 | Q3 | CH₃ | CH(CH₃)CH═CH₂ | H | Cl | 1 | racemate |
| 3-139 | Q3 | CH₃ | CH(CH₃)CH═CH₂ | H | Cl | 1 | isomer1 |
| 3-140 | Q3 | CH₃ | CH(CH₃)CH═CH₂ | H | Cl | 1 | isomer2 |
| 3-141 | Q3 | CH₃ | CH(CH₃)CH═CH₂ | H | Cl | 2 | — |
| 3-142 | Q3 | CH₃ | CH₂C(CH₃)═CH₂ | H | Cl | 0 | — |
| 3-143 | Q3 | CH₃ | CH₂C(CH₃)═CH₂ | H | Cl | 1 | racemate |
| 3-144 | Q3 | CH₃ | CH₂C(CH₃)═CH₂ | H | Cl | 1 | isomer 1 |
| 3-145 | Q3 | CH₃ | CH₂C(CH₃)═CH₂ | H | Cl | 1 | isomer2 |
| 3-146 | Q3 | CH₃ | CH₂C(CH₃)═CH₂ | H | Cl | 2 | — |
| 3-147 | Q3 | CH₃ | prop-2-yn-1-yl | H | Cl | 0 | — |
| 3-148 | Q3 | CH₃ | prop-2-yn-1-yl | H | Cl | 1 | racemate |
| 3-149 | Q3 | CH₃ | prop-2-yn-1-yl | H | Cl | 1 | isomer1 |
| 3-150 | Q3 | CH₃ | prop-2-yn-1-yl | H | Cl | 1 | isomer2 |
| 3-151 | Q3 | CH₃ | prop-2-yn-1-yl | H | Cl | 2 | — |
| 3-152 | Q3 | CH₃ | but-3-en-2-yl | H | Cl | 0 | — |
| 3-153 | Q3 | CH₃ | but-3-en-2-yl | H | Cl | 1 | racemate |
| 3-154 | Q3 | CH₃ | but-3-en-2-yl | H | Cl | 1 | isomer1 |
| 3-155 | Q3 | CH₃ | but-3-en-2-yl | H | Cl | 1 | isomer2 |
| 3-156 | Q3 | CH₃ | but-3-en-2-yl | H | Cl | 2 | — |
| 3-157 | Q3 | CH₃ | But-2-en-1-yl | H | Cl | 0 | — |
| 3-158 | Q3 | CH₃ | But-2-en-1-yl | H | Cl | 1 | racemate |
| 3-159 | Q3 | CH₃ | But-2-en-1-yl | H | Cl | 1 | isomer1 |
| 3-160 | Q3 | CH₃ | But-2-en-1-yl | H | Cl | 1 | isomer2 |
| 3-161 | Q3 | CH₃ | But-2-en-1-yl | H | Cl | 2 | — |
| 3-162 | Q3 | CH₃ | CF₂CF₃ | H | Cl | 0 | — |
| 3-163 | Q3 | CH₃ | CF₂CF₃ | H | Cl | 1 | racemate |
| 3-164 | Q3 | CH₃ | CF₂CF₃ | H | Cl | 1 | isomer1 |
| 3-165 | Q3 | CH₃ | CF₂CF₃ | H | Cl | 1 | isomer2 |
| 3-166 | Q3 | CH₃ | CF₂CF₃ | H | Cl | 2 | — |
| 3-167 | Q3 | CH₃ | CF₂CF₂CF₃ | H | Cl | 0 | — |
| 3-168 | Q3 | CH₃ | CF₂CF₂CF₃ | H | Cl | 1 | racemate |
| 3-169 | Q3 | CH₃ | CF₂CF₂CF₃ | H | Cl | 1 | isomer1 |
| 3-170 | Q3 | CH₃ | CF₂CF₂CF₃ | H | Cl | 1 | isomer2 |
| 3-171 | Q3 | CH₃ | CF₂CF₂CF₃ | H | Cl | 2 | — |
| 3-172 | Q3 | CH₃ | CH₂CF₂CF₃ | H | Cl | 0 | — |
| 3-173 | Q3 | CH₃ | CH₂CF₂CF₃ | H | Cl | 1 | racemate |
| 3-174 | Q3 | CH₃ | CH₂CF₂CF₃ | H | Cl | 1 | isomer1 |
| 3-175 | Q3 | CH₃ | CH₂CF₂CF₃ | H | Cl | 1 | isomer2 |
| 3-176 | Q3 | CH₃ | CH₂CF₂CF₃ | H | Cl | 2 | — |
| 3-177 | Q3 | CH₃ | CH₂CH₂CF₃ | H | Cl | 0 | — |
| 3-178 | Q3 | CH₃ | CH₂CH₂CF₃ | H | Cl | 1 | racemate |
| 3-179 | Q3 | CH₃ | CH₂CH₂CF₃ | H | Cl | 1 | isomer1 |
| 3-180 | Q3 | CH₃ | CH₂CH₂CF₃ | H | Cl | 1 | isomer2 |
| 3-181 | Q3 | CH₃ | CH₂CH₂CF₃ | H | Cl | 2 | — |
| 3-182 | Q3 | CH₃ | CH₂CH₂CF₂H | H | Cl | 0 | — |
| 3-183 | Q3 | CH₃ | CH₂CH₂CF₂H | H | Cl | 1 | racemate |
| 3-184 | Q3 | CH₃ | CH₂CH₂CF₂H | H | Cl | 1 | isomer1 |
| 3-185 | Q3 | CH₃ | CH₂CH₂CF₂H | H | Cl | 1 | isomer2 |
| 3-186 | Q3 | CH₃ | CH₂CH₂CF₂H | H | Cl | 2 | — |
| 3-187 | Q3 | CH₃ | CH₂CH₂CFH₂ | H | Cl | 0 | — |
| 3-188 | Q3 | CH₃ | CH₂CH₂CFH₂ | H | Cl | 1 | racemate |
| 3-189 | Q3 | CH₃ | CH₂CH₂CFH₂ | H | Cl | 1 | isomer1 |
| 3-190 | Q3 | CH₃ | CH₂CH₂CFH₂ | H | Cl | 1 | isomer2 |
| 3-191 | Q3 | CH₃ | CH₂CH₂CFH₂ | H | Cl | 2 | — |
| 3-192 | Q3 | CH₃ | CH₂CN | H | Cl | 0 | — |
| 3-193 | Q3 | CH₃ | CH₂CN | H | Cl | 1 | racemate |
| 3-194 | Q3 | CH₃ | CH₂CN | H | Cl | 1 | isomer1 |
| 3-195 | Q3 | CH₃ | CH₂CN | H | Cl | 1 | isomer2 |
| 3-196 | Q3 | CH₃ | CH₂CN | H | Cl | 2 | — |
| 3-197 | Q3 | CH₃ | CH₂CH₂CN | H | Cl | 0 | — |
| 3-198 | Q3 | CH₃ | CH₂CH₂CN | H | Cl | 1 | racemate |
| 3-199 | Q3 | CH₃ | CH₂CH₂CN | H | Cl | 1 | isomer1 |
| 3-200 | Q3 | CH₃ | CH₂CH₂CN | H | Cl | 1 | isomer2 |
| 3-201 | Q3 | CH₃ | CH₂CH₂CN | H | Cl | 2 | — |
| 3-202 | Q3 | CH₃ | CH(CH₃)CN | H | Cl | 0 | — |
| 3-203 | Q3 | CH₃ | CH(CH₃)CN | H | Cl | 1 | racemate |
| 3-204 | Q3 | CH₃ | CH(CH₃)CN | H | Cl | 1 | isomer1 |
| 3-205 | Q3 | CH₃ | CH(CH₃)CN | H | Cl | 1 | isomer2 |
| 3-206 | Q3 | CH₃ | CH(CH₃)CN | H | Cl | 2 | — |
| 3-207 | Q3 | CH₃ | CH(CH₃)CH₂CN | H | Cl | 0 | — |
| 3-208 | Q3 | CH₃ | CH(CH₃)CH₂CN | H | Cl | 1 | racemate |
| 3-209 | Q3 | CH₃ | CH(CH₃)CH₂CN | H | Cl | 1 | isomer1 |
| 3-210 | Q3 | CH₃ | CH(CH₃)CH₂CN | H | Cl | 1 | isomer2 |
| 3-211 | Q3 | CH₃ | CH(CH₃)CH₂CN | H | Cl | 2 | — |
| 3-212 | Q3 | CH₃ | CH₂CH₂Cl | H | Cl | 0 | — |
| 3-213 | Q3 | CH₃ | CH₂CH₂Cl | H | Cl | 1 | racemate |
| 3-214 | Q3 | CH₃ | CH₂CH₂Cl | H | Cl | 1 | isomer1 |
| 3-215 | Q3 | CH₃ | CH₂CH₂Cl | H | Cl | 1 | isomer2 |
| 3-216 | Q3 | CH₃ | CH₂CH₂Cl | H | Cl | 2 | — |
| 3-217 | Q3 | CH₃ | CHClCH₃ | H | Cl | 0 | — |
| 3-218 | Q3 | CH₃ | CHClCH₃ | H | Cl | 1 | racemate |
| 3-219 | Q3 | CH₃ | CHClCH₃ | H | Cl | 1 | isomer1 |
| 3-220 | Q3 | CH₃ | CHClCH₃ | H | Cl | 1 | isomer2 |
| 3-221 | Q3 | CH₃ | CHClCH₃ | H | Cl | 2 | — |
| 3-222 | Q3 | CH₃ | (E)—CH₂CH═CHCl | H | Cl | 0 | — |
| 3-223 | Q3 | CH₃ | (E)—CH₂CH═CHCl | H | Cl | 1 | racemate |
| 3-224 | Q3 | CH₃ | (E)—CH₂CH═CHCl | H | Cl | 1 | isomer1 |
| 3-225 | Q3 | CH₃ | (E)—CH₂CH═CHCl | H | Cl | 1 | isomer2 |
| 3-226 | Q3 | CH₃ | (E)—CH₂CH═CHCl | H | Cl | 2 | — |
| 3-227 | Q3 | CH₃ | (Z)—CH₂CH═CHCl | H | Cl | 0 | — |
| 3-228 | Q3 | CH₃ | (Z)—CH₂CH═CHCl | H | Cl | 1 | racemate |
| 3-229 | Q3 | CH₃ | (Z)—CH₂CH═CHCl | H | Cl | 1 | isomer 1 |
| 3-230 | Q3 | CH₃ | (Z)—CH₂CH═CHCl | H | Cl | 1 | isomer2 |
| 3-231 | Q3 | CH₃ | (Z)—CH₂CH═CHCl | H | Cl | 2 | — |
| 3-232 | Q3 | CH₃ | CH₂CH═CCl₂ | H | Cl | 0 | — |
| 3-233 | Q3 | CH₃ | CH₂CH═CCl₂ | H | Cl | 1 | racemate |
| 3-234 | Q3 | CH₃ | CH₂CH═CCl₂ | H | Cl | 1 | isomer1 |
| 3-235 | Q3 | CH₃ | CH₂CH═CCl₂ | H | Cl | 1 | isomer2 |
| 3-236 | Q3 | CH₃ | CH₂CH═CCl₂ | H | Cl | 2 | — |
| 3-237 | Q3 | CH₃ | (E)—CH₂CH═CHOCH₃ | H | Cl | 0 | — |
| 3-238 | Q3 | CH₃ | (E)—CH₂CH═CHOCH₃ | H | Cl | 1 | racemate |
| 3-239 | Q3 | CH₃ | (E)—CH₂CH═CHOCH₃ | H | Cl | 1 | isomer1 |
| 3-240 | Q3 | CH₃ | (E)—CH₂CH═CHOCH₃ | H | Cl | 1 | isomer2 |
| 3-241 | Q3 | CH₃ | (E)—CH₂CH═CHOCH₃ | H | Cl | 2 | — |
| 3-242 | Q3 | CH₃ | (Z)—CH₂CH═CHOCH₃ | H | Cl | 0 | — |
| 3-243 | Q3 | CH₃ | (Z)—CH₂CH═CHOCH₃ | H | Cl | 1 | racemate |
| 3-244 | Q3 | CH₃ | (Z)—CH₂CH═CHOCH₃ | H | Cl | 1 | isomer1 |
| 3-245 | Q3 | CH₃ | (Z)—CH₂CH═CHOCH₃ | H | Cl | 1 | isomer2 |
| 3-246 | Q3 | CH₃ | (Z)—CH₂CH═CHOCH₃ | H | Cl | 2 | — |
| 3-247 | Q3 | CH₃ | CH₂CH₂OCF₃ | H | Cl | 0 | — |
| 3-248 | Q3 | CH₃ | CH₂CH₂OCF₃ | H | Cl | 1 | racemate |
| 3-249 | Q3 | CH₃ | CH₂CH₂OCF₃ | H | Cl | 1 | isomer1 |
| 3-250 | Q3 | CH₃ | CH₂CH₂OCF₃ | H | Cl | 1 | isomer2 |
| 3-251 | Q3 | CH₃ | CH₂CH₂OCF₃ | H | Cl | 2 | — |
| 3-252 | Q3 | CH₃ | CH₂CH₂OCF₂H | H | Cl | 0 | — |
| 3-253 | Q3 | CH₃ | CH₂CH₂OCF₂H | H | Cl | 1 | racemate |
| 3-254 | Q3 | CH₃ | CH₂CH₂OCF₂H | H | Cl | 1 | isomer 1 |
| 3-255 | Q3 | CH₃ | CH₂CH₂OCF₂H | H | Cl | 1 | isomer2 |
| 3-256 | Q3 | CH₃ | CH₂CH₂OCF₂H | H | Cl | 2 | — |
| 3-257 | Q3 | CH₃ | CH₂CH₂OCFH₂ | H | Cl | 0 | — |
| 3-258 | Q3 | CH₃ | CH₂CH₂OCFH₂ | H | Cl | 1 | racemate |
| 3-259 | Q3 | CH₃ | CH₂CH₂OCFH₂ | H | Cl | 1 | isomer1 |
| 3-260 | Q3 | CH₃ | CH₂CH₂OCFH₂ | H | Cl | 1 | isomer2 |
| 3-261 | Q3 | CH₃ | CH₂CH₂OCFH₂ | H | Cl | 2 | — |
| 3-262 | Q3 | CH₃ | CH₂OCH₃ | H | Cl | 0 | — |
| 3-263 | Q3 | CH₃ | CH₂OCH₃ | H | Cl | 1 | racemate |
| 3-264 | Q3 | CH₃ | CH₂OCH₃ | H | Cl | 1 | isomer 1 |
| 3-265 | Q3 | CH₃ | CH₂OCH₃ | H | Cl | 1 | isomer2 |
| 3-266 | Q3 | CH₃ | CH₂OCH₃ | H | Cl | 2 | — |
| 3-267 | Q3 | H | CH₂CH₂CH₃ | H | Cl | 0 | — |
| 3-268 | Q3 | H | CH₂CH₂CH₃ | H | Cl | 1 | racemate |
| 3-269 | Q3 | H | CH₂CH₂CH₃ | H | Cl | 1 | isomer1 |
| 3-270 | Q3 | H | CH₂CH₂CH₃ | H | Cl | 1 | isomer2 |
| 3-271 | Q3 | H | CH₂CH₂CH₃ | H | Cl | 2 | — |
| 3-272 | Q3 | H | CH(CH₃)₂ | H | Cl | 0 | — |
| 3-273 | Q3 | H | CH(CH₃)₂ | H | Cl | 1 | racemate |
| 3-274 | Q3 | H | CH(CH₃)₂ | H | Cl | 1 | isomer 1 |
| 3-275 | Q3 | H | CH(CH₃)₂ | H | Cl | 1 | isomer2 |
| 3-276 | Q3 | H | CH(CH₃)₂ | H | Cl | 2 | — |
| 3-277 | Q3 | H | CH₂CH₂CH₂CH₃ | H | Cl | 0 | — |
| 3-278 | Q3 | H | CH₂CH₂CH₂CH₃ | H | Cl | 1 | racemate |
| 3-279 | Q3 | H | CH₂CH₂CH₂CH₃ | H | Cl | 1 | isomer1 |
| 3-280 | Q3 | H | CH₂CH₂CH₂CH₃ | H | Cl | 1 | isomer2 |
| 3-281 | Q3 | H | CH₂CH₂CH₂CH₃ | H | Cl | 2 | — |
| 3-282 | Q3 | H | CH(CH₃)CH₂CH₃ | H | Cl | 0 | racemate |
| 3-283 | Q3 | H | CH(CH₃)CH₂CH₃ | H | Cl | 0 | isomer1 |
| 3-284 | Q3 | H | CH(CH₃)CH₂CH₃ | H | Cl | 0 | isomer2 |
| 3-285 | Q3 | H | CH(CH₃)CH₂CH₃ | H | Cl | 1 | mixture |
| 3-286 | Q3 | H | CH(CH₃)CH₂CH₃ | H | Cl | 1 | isomer1 |
| 3-287 | Q3 | H | CH(CH₃)CH₂CH₃ | H | Cl | 1 | isomer2 |
| 3-288 | Q3 | H | CH(CH₃)CH₂CH₃ | H | Cl | 1 | isomer3 |
| 3-289 | Q3 | H | CH(CH₃)CH₂CH₃ | H | Cl | 1 | isomer4 |
| 3-290 | Q3 | H | CH(CH₃)CH₂CH₃ | H | Cl | 2 | racemate |
| 3-291 | Q3 | H | CH(CH₃)CH₂CH₃ | H | Cl | 2 | isomer1 |
| 3-292 | Q3 | H | CH(CH₃)CH₂CH₃ | H | Cl | 2 | isomer2 |
| 3-293 | Q3 | H | CH₂CH(CH₃)₂ | H | Cl | 0 | — |
| 3-294 | Q3 | H | CH₂CH(CH₃)₂ | H | Cl | 1 | racemate |
| 3-295 | Q3 | H | CH₂CH(CH₃)₂ | H | Cl | 1 | isomer1 |
| 3-296 | Q3 | H | CH₂CH(CH₃)₂ | H | Cl | 1 | isomer2 |
| 3-297 | Q3 | H | CH₂CH(CH₃)₂ | H | Cl | 2 | — |
| 3-298 | Q3 | H | cyclopropylmethyl | H | Cl | 0 | — |
| 3-299 | Q3 | H | cyclopropylmethyl | H | Cl | 1 | racemate |
| 3-300 | Q3 | H | cyclopropylmethyl | H | Cl | 1 | isomer1 |
| 3-301 | Q3 | H | cyclopropylmethyl | H | Cl | 1 | isomer2 |
| 3-302 | Q3 | H | cyclopropylmethyl | H | Cl | 2 | — |
| 3-303 | Q3 | H | cyclopropyl | H | Cl | 0 | — |
| 3-304 | Q3 | H | cyclopropyl | H | Cl | 1 | racemate |
| 3-305 | Q3 | H | cyclopropyl | H | Cl | 1 | isomer1 |
| 3-306 | Q3 | H | cyclopropyl | H | Cl | 1 | isomer2 |
| 3-307 | Q3 | H | cyclopropyl | H | Cl | 2 | — |
| 3-308 | Q3 | H | cyclobutyl | H | Cl | 0 | — |
| 3-309 | Q3 | H | cyclobutyl | H | Cl | 1 | racemate |
| 3-310 | Q3 | H | cyclobutyl | H | Cl | 1 | isomer1 |
| 3-311 | Q3 | H | cyclobutyl | H | Cl | 1 | isomer2 |
| 3-312 | Q3 | H | cyclobutyl | H | Cl | 2 | — |
| 3-313 | Q3 | H | cyclopentyl | H | Cl | 0 | — |
| 3-314 | Q3 | H | cyclopentyl | H | Cl | 1 | racemate |
| 3-315 | Q3 | H | cyclopentyl | H | Cl | 1 | isomer1 |
| 3-316 | Q3 | H | cyclopentyl | H | Cl | 1 | isomer2 |
| 3-317 | Q3 | H | cyclopentyl | H | Cl | 2 | — |
| 3-318 | Q3 | H | cyclohexyl | H | Cl | 0 | — |
| 3-319 | Q3 | H | cyclohexyl | H | Cl | 1 | racemate |
| 3-320 | Q3 | H | cyclohexyl | H | Cl | 1 | isomer1 |
| 3-321 | Q3 | H | cyclohexyl | H | Cl | 1 | isomer2 |
| 3-322 | Q3 | H | cyclohexyl | H | Cl | 2 | — |
| 3-323 | Q3 | H | CH₂CF₃ | H | Cl | 0 | — |
| 3-324 | Q3 | H | CH₂CF₃ | H | Cl | 1 | racemate |
| 3-325 | Q3 | H | CH₂CF₃ | H | Cl | 1 | isomer1 |
| 3-326 | Q3 | H | CH₂CF₃ | H | Cl | 1 | isomer2 |
| 3-327 | Q3 | H | CH₂CF₃ | H | Cl | 2 | — |
| 3-328 | Q3 | H | CH₂CF₂H | H | Cl | 0 | — |
| 3-329 | Q3 | H | CH₂CF₂H | H | Cl | 1 | racemate |
| 3-330 | Q3 | H | CH₂CF₂H | H | Cl | 1 | isomer1 |
| 3-331 | Q3 | H | CH₂CF₂H | H | Cl | 1 | isomer2 |
| 3-332 | Q3 | H | CH₂CF₂H | H | Cl | 2 | — |
| 3-333 | Q3 | H | CH₂CFH₂ | H | Cl | 0 | — |
| 3-334 | Q3 | H | CH₂CFH₂ | H | Cl | 1 | racemate |
| 3-335 | Q3 | H | CH₂CFH₂ | H | Cl | 1 | isomer1 |
| 3-336 | Q3 | H | CH₂CFH₂ | H | Cl | 1 | isomer2 |
| 3-337 | Q3 | H | CH₂CFH₂ | H | Cl | 2 | — |
| 3-338 | Q3 | H | CH₂CH═CH₂ | H | Cl | 0 | — |
| 3-339 | Q3 | H | CH₂CH═CH₂ | H | Cl | 1 | racemate |
| 3-340 | Q3 | H | CH₂CH═CH₂ | H | Cl | 1 | isomer1 |
| 3-341 | Q3 | H | CH₂CH═CH₂ | H | Cl | 1 | isomer2 |
| 3-342 | Q3 | H | CH₂CH═CH₂ | H | Cl | 2 | — |
| 3-343 | Q3 | H | CF₂CF₃ | H | Cl | 0 | — |
| 3-344 | Q3 | H | CF₂CF₃ | H | Cl | 1 | racemate |
| 3-345 | Q3 | H | CF₂CF₃ | H | Cl | 1 | isomer1 |
| 3-346 | Q3 | H | CF₂CF₃ | H | Cl | 1 | isomer2 |
| 3-347 | Q3 | H | CF₂CF₃ | H | Cl | 2 | — |
| 3-348 | Q3 | H | CF₂CF₂CF₃ | H | Cl | 0 | — |
| 3-349 | Q3 | H | CF₂CF₂CF₃ | H | Cl | 1 | racemate |
| 3-350 | Q3 | H | CF₂CF₂CF₃ | H | Cl | 1 | isomer1 |
| 3-351 | Q3 | H | CF₂CF₂CF₃ | H | Cl | 1 | isomer2 |
| 3-352 | Q3 | H | CF₂CF₂CF₃ | H | Cl | 2 | — |
| 3-353 | Q3 | H | CH₂CH₂CF₃ | H | Cl | 0 | — |
| 3-354 | Q3 | H | CH₂CH₂CF₃ | H | Cl | 1 | racemate |
| 3-355 | Q3 | H | CH₂CH₂CF₃ | H | Cl | 1 | isomer1 |
| 3-356 | Q3 | H | CH₂CH₂CF₃ | H | Cl | 1 | isomer2 |
| 3-357 | Q3 | H | CH₂CH₂CF₃ | H | Cl | 2 | — |
| 3-358 | Q3 | H | CH₂CH₂CF₂H | H | Cl | 0 | — |
| 3-359 | Q3 | H | CH₂CH₂CF₂H | H | Cl | 1 | racemate |
| 3-360 | Q3 | H | CH₂CH₂CF₂H | H | Cl | 1 | isomer1 |
| 3-361 | Q3 | H | CH₂CH₂CF₂H | H | Cl | 1 | isomer2 |
| 3-362 | Q3 | H | CH₂CH₂CF₂H | H | Cl | 2 | — |
| 3-363 | Q3 | H | CH₂CH₂CFH₂ | H | Cl | 0 | — |
| 3-364 | Q3 | H | CH₂CH₂CFH₂ | H | Cl | 1 | racemate |
| 3-365 | Q3 | H | CH₂CH₂CFH₂ | H | Cl | 1 | isomer1 |
| 3-366 | Q3 | H | CH₂CH₂CFH₂ | H | Cl | 1 | isomer2 |
| 3-367 | Q3 | H | CH₂CH₂CFH₂ | H | Cl | 2 | — |
| 3-368 | Q3 | H | CH₂CN | H | Cl | 0 | — |
| 3-369 | Q3 | H | CH₂CN | H | Cl | 1 | racemate |
| 3-370 | Q3 | H | CH₂CN | H | Cl | 1 | isomer1 |
| 3-371 | Q3 | H | CH₂CN | H | Cl | 1 | isomer2 |
| 3-372 | Q3 | H | CH₂CN | H | Cl | 2 | — |
| 3-373 | Q3 | H | CH₂CH₂CN | H | Cl | 0 | — |
| 3-374 | Q3 | H | CH₂CH₂CN | H | Cl | 1 | racemate |
| 3-375 | Q3 | H | CH₂CH₂CN | H | Cl | 1 | isomer1 |
| 3-376 | Q3 | H | CH₂CH₂CN | H | Cl | 1 | isomer2 |
| 3-377 | Q3 | H | CH₂CH₂CN | H | Cl | 2 | — |
| 3-378 | Q3 | H | CH₂CH₂Cl | H | Cl | 0 | — |
| 3-379 | Q3 | H | CH₂CH₂Cl | H | Cl | 1 | racemate |
| 3-380 | Q3 | H | CH₂CH₂Cl | H | Cl | 1 | isomer1 |
| 3-381 | Q3 | H | CH₂CH₂Cl | H | Cl | 1 | isomer2 |
| 3-382 | Q3 | H | CH₂CH₂Cl | H | Cl | 2 | — |
| 3-383 | Q3 | CH₂CH₃ | CH₂CH₂CH₃ | H | Cl | 0 | — |
| 3-384 | Q3 | CH₂CH₃ | CH₂CH₂CH₃ | H | Cl | 1 | racemate |
| 3-385 | Q3 | CH₂CH₃ | CH₂CH₂CH₃ | H | Cl | 1 | isomer1 |
| 3-386 | Q3 | CH₂CH₃ | CH₂CH₂CH₃ | H | Cl | 1 | isomer2 |
| 3-387 | Q3 | CH₂CH₃ | CH₂CH₂CH₃ | H | Cl | 2 | — |
| 3-388 | Q3 | CH₂CH₃ | CH(CH₃)₂ | H | Cl | 0 | — |
| 3-389 | Q3 | CH₂CH₃ | CH(CH₃)₂ | H | Cl | 1 | racemate |
| 3-390 | Q3 | CH₂CH₃ | CH(CH₃)₂ | H | Cl | 1 | isomer1 |
| 3-391 | Q3 | CH₂CH₃ | CH(CH₃)₂ | H | Cl | 1 | isomer2 |
| 3-392 | Q3 | CH₂CH₃ | CH(CH₃)₂ | H | Cl | 2 | — |
| 3-393 | Q3 | CH₂CH₃ | CH₂CH₂CH₂CH₃ | H | Cl | 0 | — |
| 3-394 | Q3 | CH₂CH₃ | CH₂CH₂CH₂CH₃ | H | Cl | 1 | racemate |
| 3-395 | Q3 | CH₂CH₃ | CH₂CH₂CH₂CH₃ | H | Cl | 1 | isomer1 |
| 3-396 | Q3 | CH₂CH₃ | CH₂CH₂CH₂CH₃ | H | Cl | 1 | isomer2 |
| 3-397 | Q3 | CH₂CH₃ | CH₂CH₂CH₂CH₃ | H | Cl | 2 | — |
| 3-398 | Q3 | CH₂CH₃ | CH(CH₃)CH₂CH₃ | H | Cl | 0 | racemate |
| 3-399 | Q3 | CH₂CH₃ | CH(CH₃)CH₂CH₃ | H | Cl | 0 | isomer1 |
| 3-400 | Q3 | CH₂CH₃ | CH(CH₃)CH₂CH₃ | H | Cl | 0 | isomer2 |
| 3-401 | Q3 | CH₂CH₃ | CH(CH₃)CH₂CH₃ | H | Cl | 1 | mixture |
| 3-402 | Q3 | CH₂CH₃ | CH(CH₃)CH₂CH₃ | H | Cl | 1 | isomer1 |
| 3-403 | Q3 | CH₂CH₃ | CH(CH₃)CH₂CH₃ | H | Cl | 1 | isomer2 |
| 3-404 | Q3 | CH₂CH₃ | CH(CH₃)CH₂CH₃ | H | Cl | 1 | isomer3 |
| 3-405 | Q3 | CH₂CH₃ | CH(CH₃)CH₂CH₃ | H | Cl | 1 | isomer4 |
| 3-406 | Q3 | CH₂CH₃ | CH(CH₃)CH₂CH₃ | H | Cl | 2 | racemate |
| 3-407 | Q3 | CH₂CH₃ | CH(CH₃)CH₂CH₃ | H | Cl | 2 | isomer1 |
| 3-408 | Q3 | CH₂CH₃ | CH(CH₃)CH₂CH₃ | H | Cl | 2 | isomer2 |
| 3-409 | Q3 | CH₂CH₃ | CH₂CH(CH₃)₂ | H | Cl | 0 | — |
| 3-410 | Q3 | CH₂CH₃ | CH₂CH(CH₃)₂ | H | Cl | 1 | racemate |
| 3-411 | Q3 | CH₂CH₃ | CH₂CH(CH₃)₂ | H | Cl | 1 | isomer1 |
| 3-412 | Q3 | CH₂CH₃ | CH₂CH(CH₃)₂ | H | Cl | 1 | isomer2 |
| 3-413 | Q3 | CH₂CH₃ | CH₂CH(CH₃)₂ | H | Cl | 2 | — |
| 3-414 | Q3 | CH₂CH₃ | cyclopropylmethyl | H | Cl | 0 | — |
| 3-415 | Q3 | CH₂CH₃ | cyclopropylmethyl | H | Cl | 1 | racemate |
| 3-416 | Q3 | CH₂CH₃ | cyclopropylmethyl | H | Cl | 1 | isomer1 |
| 3-417 | Q3 | CH₂CH₃ | cyclopropylmethyl | H | Cl | 1 | isomer2 |
| 3-418 | Q3 | CH₂CH₃ | cyclopropylmethyl | H | Cl | 2 | — |
| 3-419 | Q3 | CH₂CH₃ | cyclopropyl | H | Cl | 0 | — |
| 3-420 | Q3 | CH₂CH₃ | cyclopropyl | H | Cl | 1 | racemate |
| 3-421 | Q3 | CH₂CH₃ | cyclopropyl | H | Cl | 1 | isomer1 |
| 3-422 | Q3 | CH₂CH₃ | cyclopropyl | H | Cl | 1 | isomer2 |
| 3-423 | Q3 | CH₂CH₃ | cyclopropyl | H | Cl | 2 | — |
| 3-424 | Q3 | CH₂CH₃ | cyclobutyl | H | Cl | 0 | — |
| 3-425 | Q3 | CH₂CH₃ | cyclobutyl | H | Cl | 1 | racemate |
| 3-426 | Q3 | CH₂CH₃ | cyclobutyl | H | Cl | 1 | isomer1 |
| 3-427 | Q3 | CH₂CH₃ | cyclobutyl | H | Cl | 1 | isomer2 |
| 3-428 | Q3 | CH₂CH₃ | cyclobutyl | H | Cl | 2 | — |
| 3-429 | Q3 | CH₂CH₃ | cyclopentyl | H | Cl | 0 | — |
| 3-430 | Q3 | CH₂CH₃ | cyclopentyl | H | Cl | 1 | racemate |
| 3-431 | Q3 | CH₂CH₃ | cyclopentyl | H | Cl | 1 | isomer1 |
| 3-432 | Q3 | CH₂CH₃ | cyclopentyl | H | Cl | 1 | isomer2 |
| 3-433 | Q3 | CH₂CH₃ | cyclopentyl | H | Cl | 2 | — |
| 3-434 | Q3 | CH₂CH₃ | cyclohexyl | H | Cl | 0 | — |
| 3-435 | Q3 | CH₂CH₃ | cyclohexyl | H | Cl | 1 | racemate |
| 3-436 | Q3 | CH₂CH₃ | cyclohexyl | H | Cl | 1 | isomer1 |
| 3-437 | Q3 | CH₂CH₃ | cyclohexyl | H | Cl | 1 | isomer2 |
| 3-438 | Q3 | CH₂CH₃ | cyclohexyl | H | Cl | 2 | — |
| 3-439 | Q3 | CH₂CH₃ | CH₂CF₃ | H | Cl | 0 | — |
| 3-440 | Q3 | CH₂CH₃ | CH₂CF₃ | H | Cl | 1 | racemate |
| 3-441 | Q3 | CH₂CH₃ | CH₂CF₃ | H | Cl | 1 | isomer1 |
| 3-442 | Q3 | CH₂CH₃ | CH₂CF₃ | H | Cl | 1 | isomer2 |
| 3-443 | Q3 | CH₂CH₃ | CH₂CF₃ | H | Cl | 2 | — |
| 3-444 | Q3 | CH₂CH₃ | CH₂CF₂H | H | Cl | 0 | — |
| 3-445 | Q3 | CH₂CH₃ | CH₂CF₂H | H | Cl | 1 | racemate |
| 3-446 | Q3 | CH₂CH₃ | CH₂CF₂H | H | Cl | 1 | isomer1 |
| 3-447 | Q3 | CH₂CH₃ | CH₂CF₂H | H | Cl | 1 | isomer2 |
| 3-448 | Q3 | CH₂CH₃ | CH₂CF₂H | H | Cl | 2 | — |
| 3-449 | Q3 | CH₂CH₃ | CH₂CFH₂ | H | Cl | 0 | — |
| 3-450 | Q3 | CH₂CH₃ | CH₂CFH₂ | H | Cl | 1 | racemate |
| 3-451 | Q3 | CH₂CH₃ | CH₂CFH₂ | H | Cl | 1 | isomer1 |
| 3-452 | Q3 | CH₂CH₃ | CH₂CFH₂ | H | Cl | 1 | isomer2 |
| 3-453 | Q3 | CH₂CH₃ | CH₂CFH₂ | H | Cl | 2 | — |
| 3-454 | Q3 | CH₂CH₃ | CH₂CH═CH₂ | H | Cl | 0 | — |
| 3-455 | Q3 | CH₂CH₃ | CH₂CH═CH₂ | H | Cl | 1 | racemate |
| 3-456 | Q3 | CH₂CH₃ | CH₂CH═CH₂ | H | Cl | 1 | isomer 1 |
| 3-457 | Q3 | CH₂CH₃ | CH₂CH═CH₂ | H | Cl | 1 | isomer2 |
| 3-458 | Q3 | CH₂CH₃ | CH₂CH═CH₂ | H | Cl | 2 | — |
| 3-459 | Q3 | CH₂CH₃ | CF₂CF₃ | H | Cl | 0 | — |
| 3-460 | Q3 | CH₂CH₃ | CF₂CF₃ | H | Cl | 1 | racemate |
| 3-461 | Q3 | CH₂CH₃ | CF₂CF₃ | H | Cl | 1 | isomer1 |
| 3-462 | Q3 | CH₂CH₃ | CF₂CF₃ | H | Cl | 1 | isomer2 |
| 3-463 | Q3 | CH₂CH₃ | CF₂CF₃ | H | Cl | 2 | — |
| 3-464 | Q3 | CH₂CH₃ | CF₂CF₂CF₃ | H | Cl | 0 | — |
| 3-465 | Q3 | CH₂CH₃ | CF₂CF₂CF₃ | H | Cl | 1 | racemate |
| 3-466 | Q3 | CH₂CH₃ | CF₂CF₂CF₃ | H | Cl | 1 | isomer1 |
| 3-467 | Q3 | CH₂CH₃ | CF₂CF₂CF₃ | H | Cl | 1 | isomer2 |
| 3-468 | Q3 | CH₂CH₃ | CF₂CF₂CF₃ | H | Cl | 2 | — |
| 3-469 | Q3 | CH₂CH₃ | CH₂CH₂CF₃ | H | Cl | 0 | — |
| 3-470 | Q3 | CH₂CH₃ | CH₂CH₂CF₃ | H | Cl | 1 | racemate |
| 3-471 | Q3 | CH₂CH₃ | CH₂CH₂CF₃ | H | Cl | 1 | isomer1 |
| 3-472 | Q3 | CH₂CH₃ | CH₂CH₂CF₃ | H | Cl | 1 | isomer2 |
| 3-473 | Q3 | CH₂CH₃ | CH₂CH₂CF₃ | H | Cl | 2 | — |
| 3-474 | Q3 | CH₂CH₃ | CH₂CH₂CF₂H | H | Cl | 0 | — |
| 3-475 | Q3 | CH₂CH₃ | CH₂CH₂CF₂H | H | Cl | 1 | racemate |
| 3-476 | Q3 | CH₂CH₃ | CH₂CH₂CF₂H | H | Cl | 1 | isomer1 |
| 3-477 | Q3 | CH₂CH₃ | CH₂CH₂CF₂H | H | Cl | 1 | isomer2 |
| 3-478 | Q3 | CH₂CH₃ | CH₂CH₂CF₂H | H | Cl | 2 | — |
| 3-479 | Q3 | CH₂CH₃ | CH₂CH₂CFH₂ | H | Cl | 0 | — |
| 3-480 | Q3 | CH₂CH₃ | CH₂CH₂CFH₂ | H | Cl | 1 | racemate |
| 3-481 | Q3 | CH₂CH₃ | CH₂CH₂CFH₂ | H | Cl | 1 | isomer1 |
| 3-482 | Q3 | CH₂CH₃ | CH₂CH₂CFH₂ | H | Cl | 1 | isomer2 |
| 3-483 | Q3 | CH₂CH₃ | CH₂CH₂CFH₂ | H | Cl | 2 | — |
| 3-484 | Q3 | CH₂CH₃ | CH₂CN | H | Cl | 0 | — |
| 3-485 | Q3 | CH₂CH₃ | CH₂CN | H | Cl | 1 | racemate |
| 3-486 | Q3 | CH₂CH₃ | CH₂CN | H | Cl | 1 | isomer1 |
| 3-487 | Q3 | CH₂CH₃ | CH₂CN | H | Cl | 1 | isomer2 |
| 3-488 | Q3 | CH₂CH₃ | CH₂CN | H | Cl | 2 | — |
| 3-489 | Q3 | CH₂CH₃ | CH₂CH₂CN | H | Cl | 0 | — |
| 3-490 | Q3 | CH₂CH₃ | CH₂CH₂CN | H | Cl | 1 | racemate |
| 3-491 | Q3 | CH₂CH₃ | CH₂CH₂CN | H | Cl | 1 | isomer1 |
| 3-492 | Q3 | CH₂CH₃ | CH₂CH₂CN | H | Cl | 1 | isomer2 |
| 3-493 | Q3 | CH₂CH₃ | CH₂CH₂CN | H | Cl | 2 | — |
| 3-494 | Q3 | CH₂CH₃ | CH₂CH₂Cl | H | Cl | 0 | — |
| 3-495 | Q3 | CH₂CH₃ | CH₂CH₂Cl | H | Cl | 1 | racemate |
| 3-496 | Q3 | CH₂CH₃ | CH₂CH₂Cl | H | Cl | 1 | isomer1 |
| 3-497 | Q3 | CH₂CH₃ | CH₂CH₂Cl | H | Cl | 1 | isomer2 |
| 3-498 | Q3 | CH₂CH₃ | CH₂CH₂Cl | H | Cl | 2 | — |
| 3-499 | Q3 | CH(CH₃)₂ | CH₂CH₂CH₃ | H | Cl | 0 | — |
| 3-500 | Q3 | CH(CH₃)₂ | CH₂CH₂CH₃ | H | Cl | 1 | racemate |
| 3-501 | Q3 | CH(CH₃)₂ | CH₂CH₂CH₃ | H | Cl | 1 | isomer1 |
| 3-502 | Q3 | CH(CH₃)₂ | CH₂CH₂CH₃ | H | Cl | 1 | isomer2 |
| 3-503 | Q3 | CH(CH₃)₂ | CH₂CH₂CH₃ | H | Cl | 2 | — |
| 3-504 | Q3 | CH(CH₃)₂ | CH(CH₃)₂ | H | Cl | 0 | — |
| 3-505 | Q3 | CH(CH₃)₂ | CH(CH₃)₂ | H | Cl | 1 | racemate |
| 3-506 | Q3 | CH(CH₃)₂ | CH(CH₃)₂ | H | Cl | 1 | isomer1 |
| 3-507 | Q3 | CH(CH₃)₂ | CH(CH₃)₂ | H | Cl | 1 | isomer2 |
| 3-508 | Q3 | CH(CH₃)₂ | CH(CH₃)₂ | H | Cl | 2 | — |
| 3-509 | Q3 | CH(CH₃)₂ | CH₂CH₂CH₂CH₃ | H | Cl | 0 | — |
| 3-510 | Q3 | CH(CH₃)₂ | CH₂CH₂CH₂CH₃ | H | Cl | 1 | racemate |
| 3-511 | Q3 | CH(CH₃)₂ | CH₂CH₂CH₂CH₃ | H | Cl | 1 | isomer1 |
| 3-512 | Q3 | CH(CH₃)₂ | CH₂CH₂CH₂CH₃ | H | Cl | 1 | isomer2 |
| 3-513 | Q3 | CH(CH₃)₂ | CH₂CH₂CH₂CH₃ | H | Cl | 2 | — |
| 3-514 | Q3 | CH(CH₃)₂ | CH(CH₃)CH₂CH₃ | H | Cl | 0 | racemate |
| 3-515 | Q3 | CH(CH₃)₂ | CH(CH₃)CH₂CH₃ | H | Cl | 0 | isomer1 |
| 3-516 | Q3 | CH(CH₃)₂ | CH(CH₃)CH₂CH₃ | H | Cl | 0 | isomer2 |
| 3-517 | Q3 | CH(CH₃)₂ | CH(CH₃)CH₂CH₃ | H | Cl | 1 | mixture |
| 3-518 | Q3 | CH(CH₃)₂ | CH(CH₃)CH₂CH₃ | H | Cl | 1 | isomer1 |
| 3-519 | Q3 | CH(CH₃)₂ | CH(CH₃)CH₂CH₃ | H | Cl | 1 | isomer2 |
| 3-520 | Q3 | CH(CH₃)₂ | CH(CH₃)CH₂CH₃ | H | Cl | 1 | isomer3 |
| 3-521 | Q3 | CH(CH₃)₂ | CH(CH₃)CH₂CH₃ | H | Cl | 1 | isomer4 |
| 3-522 | Q3 | CH(CH₃)₂ | CH(CH₃)CH₂CH₃ | H | Cl | 2 | racemate |
| 3-523 | Q3 | CH(CH₃)₂ | CH(CH₃)CH₂CH₃ | H | Cl | 2 | isomer1 |
| 3-524 | Q3 | CH(CH₃)₂ | CH(CH₃)CH₂CH₃ | H | Cl | 2 | isomer2 |
| 3-525 | Q3 | CH(CH₃)₂ | CH₂CH(CH₃)₂ | H | Cl | 0 | — |
| 3-526 | Q3 | CH(CH₃)₂ | CH₂CH(CH₃)₂ | H | Cl | 1 | racemate |
| 3-527 | Q3 | CH(CH₃)₂ | CH₂CH(CH₃)₂ | H | Cl | 1 | isomer1 |
| 3-528 | Q3 | CH(CH₃)₂ | CH₂CH(CH₃)₂ | H | Cl | 1 | isomer2 |
| 3-529 | Q3 | CH(CH₃)₂ | CH₂CH(CH₃)₂ | H | Cl | 2 | — |
| 3-530 | Q3 | CH(CH₃)₂ | cyclopropylmethyl | H | Cl | 0 | — |
| 3-531 | Q3 | CH(CH₃)₂ | cyclopropylmethyl | H | Cl | 1 | racemate |
| 3-532 | Q3 | CH(CH₃)₂ | cyclopropylmethyl | H | Cl | 1 | isomer 1 |
| 3-533 | Q3 | CH(CH₃)₂ | cyclopropylmethyl | H | Cl | 1 | isomer2 |
| 3-534 | Q3 | CH(CH₃)₂ | cyclopropylmethyl | H | Cl | 2 | — |
| 3-535 | Q3 | CH(CH₃)₂ | cyclopropyl | H | Cl | 0 | — |
| 3-536 | Q3 | CH(CH₃)₂ | cyclopropyl | H | Cl | 1 | racemate |
| 3-537 | Q3 | CH(CH₃)₂ | cyclopropyl | H | Cl | 1 | isomer1 |
| 3-538 | Q3 | CH(CH₃)₂ | cyclopropyl | H | Cl | 1 | isomer2 |
| 3-539 | Q3 | CH(CH₃)₂ | cyclopropyl | H | Cl | 2 | — |
| 3-540 | Q3 | CH(CH₃)₂ | cyclobutyl | H | Cl | 0 | — |
| 3-541 | Q3 | CH(CH₃)₂ | cyclobutyl | H | Cl | 1 | racemate |
| 3-542 | Q3 | CH(CH₃)₂ | cyclobutyl | H | Cl | 1 | isomer1 |
| 3-543 | Q3 | CH(CH₃)₂ | cyclobutyl | H | Cl | 1 | isomer2 |
| 3-544 | Q3 | CH(CH₃)₂ | cyclobutyl | H | Cl | 2 | — |
| 3-545 | Q3 | CH(CH₃)₂ | cyclopentyl | H | Cl | 0 | — |
| 3-546 | Q3 | CH(CH₃)₂ | cyclopentyl | H | Cl | 1 | racemate |
| 3-547 | Q3 | CH(CH₃)₂ | cyclopentyl | H | Cl | 1 | isomer1 |
| 3-548 | Q3 | CH(CH₃)₂ | cyclopentyl | H | Cl | 1 | isomer2 |
| 3-549 | Q3 | CH(CH₃)₂ | cyclopentyl | H | Cl | 2 | — |
| 3-550 | Q3 | CH(CH₃)₂ | cyclohexyl | H | Cl | 0 | — |
| 3-551 | Q3 | CH(CH₃)₂ | cyclohexyl | H | Cl | 1 | racemate |
| 3-552 | Q3 | CH(CH₃)₂ | cyclohexyl | H | Cl | 1 | isomer1 |
| 3-553 | Q3 | CH(CH₃)₂ | cyclohexyl | H | Cl | 1 | isomer2 |
| 3-554 | Q3 | CH(CH₃)₂ | cyclohexyl | H | Cl | 2 | — |
| 3-555 | Q3 | CH(CH₃)₂ | CH₂CF₃ | H | Cl | 0 | — |
| 3-556 | Q3 | CH(CH₃)₂ | CH₂CF₃ | H | Cl | 1 | racemate |
| 3-557 | Q3 | CH(CH₃)₂ | CH₂CF₃ | H | Cl | 1 | isomer1 |
| 3-558 | Q3 | CH(CH₃)₂ | CH₂CF₃ | H | Cl | 1 | isomer2 |
| 3-559 | Q3 | CH(CH₃)₂ | CH₂CF₃ | H | Cl | 2 | — |
| 3-560 | Q3 | CH(CH₃)₂ | CH₂CF₂H | H | Cl | 0 | — |
| 3-561 | Q3 | CH(CH₃)₂ | CH₂CF₂H | H | Cl | 1 | racemate |
| 3-562 | Q3 | CH(CH₃)₂ | CH₂CF₂H | H | Cl | 1 | isomer1 |
| 3-563 | Q3 | CH(CH₃)₂ | CH₂CF₂H | H | Cl | 1 | isomer2 |
| 3-564 | Q3 | CH(CH₃)₂ | CH₂CF₂H | H | Cl | 2 | — |
| 3-565 | Q3 | CH(CH₃)₂ | CH₂CFH₂ | H | Cl | 0 | — |
| 3-566 | Q3 | CH(CH₃)₂ | CH₂CFH₂ | H | Cl | 1 | racemate |
| 3-567 | Q3 | CH(CH₃)₂ | CH₂CFH₂ | H | Cl | 1 | isomer1 |
| 3-568 | Q3 | CH(CH₃)₂ | CH₂CFH₂ | H | Cl | 1 | isomer2 |
| 3-569 | Q3 | CH(CH₃)₂ | CH₂CFH₂ | H | Cl | 2 | — |
| 3-570 | Q3 | CH(CH₃)₂ | CH₂CH═CH₂ | H | Cl | 0 | — |
| 3-571 | Q3 | CH(CH₃)₂ | CH₂CH═CH₂ | H | Cl | 1 | racemate |
| 3-572 | Q3 | CH(CH₃)₂ | CH₂CH═CH₂ | H | Cl | 1 | isomer1 |
| 3-573 | Q3 | CH(CH₃)₂ | CH₂CH═CH₂ | H | Cl | 1 | isomer2 |
| 3-574 | Q3 | CH(CH₃)₂ | CH₂CH═CH₂ | H | Cl | 2 | — |
| 3-575 | Q3 | CH(CH₃)₂ | CF₂CF₃ | H | Cl | 0 | — |
| 3-576 | Q3 | CH(CH₃)₂ | CF₂CF₃ | H | Cl | 1 | racemate |
| 3-577 | Q3 | CH(CH₃)₂ | CF₂CF₃ | H | Cl | 1 | isomer1 |
| 3-578 | Q3 | CH(CH₃)₂ | CF₂CF₃ | H | Cl | 1 | isomer2 |
| 3-579 | Q3 | CH(CH₃)₂ | CF₂CF₃ | H | Cl | 2 | — |
| 3-580 | Q3 | CH(CH₃)₂ | CF₂CF₂CF₃ | H | Cl | 0 | — |
| 3-581 | Q3 | CH(CH₃)₂ | CF₂CF₂CF₃ | H | Cl | 1 | racemate |
| 3-582 | Q3 | CH(CH₃)₂ | CF₂CF₂CF₃ | H | Cl | 1 | isomer1 |
| 3-583 | Q3 | CH(CH₃)₂ | CF₂CF₂CF₃ | H | Cl | 1 | isomer2 |
| 3-584 | Q3 | CH(CH₃)₂ | CF₂CF₂CF₃ | H | Cl | 2 | — |
| 3-585 | Q3 | CH(CH₃)₂ | CH₂CH₂CF₃ | H | Cl | 0 | — |
| 3-586 | Q3 | CH(CH₃)₂ | CH₂CH₂CF₃ | H | Cl | 1 | racemate |
| 3-587 | Q3 | CH(CH₃)₂ | CH₂CH₂CF₃ | H | Cl | 1 | isomer1 |
| 3-588 | Q3 | CH(CH₃)₂ | CH₂CH₂CF₃ | H | Cl | 1 | isomer2 |
| 3-589 | Q3 | CH(CH₃)₂ | CH₂CH₂CF₃ | H | Cl | 2 | — |
| 3-590 | Q3 | CH(CH₃)₂ | CH₂CH₂CF₂H | H | Cl | 0 | — |
| 3-591 | Q3 | CH(CH₃)₂ | CH₂CH₂CF₂H | H | Cl | 1 | racemate |
| 3-592 | Q3 | CH(CH₃)₂ | CH₂CH₂CF₂H | H | Cl | 1 | isomer1 |
| 3-593 | Q3 | CH(CH₃)₂ | CH₂CH₂CF₂H | H | Cl | 1 | isomer2 |
| 3-594 | Q3 | CH(CH₃)₂ | CH₂CH₂CF₂H | H | Cl | 2 | — |
| 3-595 | Q3 | CH(CH₃)₂ | CH₂CH₂CFH₂ | H | Cl | 0 | — |
| 3-596 | Q3 | CH(CH₃)₂ | CH₂CH₂CFH₂ | H | Cl | 1 | racemate |
| 3-597 | Q3 | CH(CH₃)₂ | CH₂CH₂CFH₂ | H | Cl | 1 | isomer1 |
| 3-598 | Q3 | CH(CH₃)₂ | CH₂CH₂CFH₂ | H | Cl | 1 | isomer2 |
| 3-599 | Q3 | CH(CH₃)₂ | CH₂CH₂CFH₂ | H | Cl | 2 | — |
| 3-600 | Q3 | CH(CH₃)₂ | CH₂CN | H | Cl | 0 | — |
| 3-601 | Q3 | CH(CH₃)₂ | CH₂CN | H | Cl | 1 | racemate |
| 3-602 | Q3 | CH(CH₃)₂ | CH₂CN | H | Cl | 1 | isomer1 |
| 3-603 | Q3 | CH(CH₃)₂ | CH₂CN | H | Cl | 1 | isomer2 |
| 3-604 | Q3 | CH(CH₃)₂ | CH₂CN | H | Cl | 2 | — |
| 3-605 | Q3 | CH(CH₃)₂ | CH₂CH₂CN | H | Cl | 0 | — |
| 3-606 | Q3 | CH(CH₃)₂ | CH₂CH₂CN | H | Cl | 1 | racemate |
| 3-607 | Q3 | CH(CH₃)₂ | CH₂CH₂CN | H | Cl | 1 | isomer1 |
| 3-608 | Q3 | CH(CH₃)₂ | CH₂CH₂CN | H | Cl | 1 | isomer2 |
| 3-609 | Q3 | CH(CH₃)₂ | CH₂CH₂CN | H | Cl | 2 | — |
| 3-610 | Q3 | CH(CH₃)₂ | CH₂CH₂Cl | H | Cl | 0 | — |
| 3-611 | Q3 | CH(CH₃)₂ | CH₂CH₂Cl | H | Cl | 1 | racemate |
| 3-612 | Q3 | CH(CH₃)₂ | CH₂CH₂Cl | H | Cl | 1 | isomer1 |
| 3-613 | Q3 | CH(CH₃)₂ | CH₂CH₂Cl | H | Cl | 1 | isomer2 |
| 3-614 | Q3 | CH(CH₃)₂ | CH₂CH₂Cl | H | Cl | 2 | — |
| 3-615 | Q3 | cyclopropyl | CH₂CH₂CH₃ | H | Cl | 0 | — |
| 3-616 | Q3 | cyclopropyl | CH₂CH₂CH₃ | H | Cl | 1 | racemate |
| 3-617 | Q3 | cyclopropyl | CH₂CH₂CH₃ | H | Cl | 1 | isomer1 |
| 3-618 | Q3 | cyclopropyl | CH₂CH₂CH₃ | H | Cl | 1 | isomer2 |
| 3-619 | Q3 | cyclopropyl | CH₂CH₂CH₃ | H | Cl | 2 | — |
| 3-620 | Q3 | cyclopropyl | CH(CH₃)₂ | H | Cl | 0 | — |
| 3-621 | Q3 | cyclopropyl | CH(CH₃)₂ | H | Cl | 1 | racemate |
| 3-622 | Q3 | cyclopropyl | CH(CH₃)₂ | H | Cl | 1 | isomer1 |
| 3-623 | Q3 | cyclopropyl | CH(CH₃)₂ | H | Cl | 1 | isomer2 |
| 3-624 | Q3 | cyclopropyl | CH(CH₃)₂ | H | Cl | 2 | — |
| 3-625 | Q3 | cyclopropyl | CH₂CH₂CH₂CH₃ | H | Cl | 0 | — |
| 3-626 | Q3 | cyclopropyl | CH₂CH₂CH₂CH₃ | H | Cl | 1 | racemate |
| 3-627 | Q3 | cyclopropyl | CH₂CH₂CH₂CH₃ | H | Cl | 1 | isomer1 |
| 3-628 | Q3 | cyclopropyl | CH₂CH₂CH₂CH₃ | H | Cl | 1 | isomer2 |
| 3-629 | Q3 | cyclopropyl | CH₂CH₂CH₂CH₃ | H | Cl | 2 | — |
| 3-630 | Q3 | cyclopropyl | CH(CH₃)CH₂CH₃ | H | Cl | 0 | racemate |
| 3-631 | Q3 | cyclopropyl | CH(CH₃)CH₂CH₃ | H | Cl | 0 | isomer1 |
| 3-632 | Q3 | cyclopropyl | CH(CH₃)CH₂CH₃ | H | Cl | 0 | isomer2 |
| 3-633 | Q3 | cyclopropyl | CH(CH₃)CH₂CH₃ | H | Cl | 1 | mixture |
| 3-634 | Q3 | cyclopropyl | CH(CH₃)CH₂CH₃ | H | Cl | 1 | isomer1 |
| 3-635 | Q3 | cyclopropyl | CH(CH₃)CH₂CH₃ | H | Cl | 1 | isomer2 |
| 3-636 | Q3 | cyclopropyl | CH(CH₃)CH₂CH₃ | H | Cl | 1 | isomer3 |
| 3-637 | Q3 | cyclopropyl | CH(CH₃)CH₂CH₃ | H | Cl | 1 | isomer4 |
| 3-638 | Q3 | cyclopropyl | CH(CH₃)CH₂CH₃ | H | Cl | 2 | racemate |
| 3-639 | Q3 | cyclopropyl | CH(CH₃)CH₂CH₃ | H | Cl | 2 | isomer1 |
| 3-640 | Q3 | cyclopropyl | CH(CH₃)CH₂CH₃ | H | Cl | 2 | isomer2 |
| 3-641 | Q3 | cyclopropyl | CH₂CH(CH₃)₂ | H | Cl | 0 | — |
| 3-642 | Q3 | cyclopropyl | CH₂CH(CH₃)₂ | H | Cl | 1 | racemate |
| 3-643 | Q3 | cyclopropyl | CH₂CH(CH₃)₂ | H | Cl | 1 | isomer1 |
| 3-644 | Q3 | cyclopropyl | CH₂CH(CH₃)₂ | H | Cl | 1 | isomer2 |
| 3-645 | Q3 | cyclopropyl | CH₂CH(CH₃)₂ | H | Cl | 2 | — |
| 3-646 | Q3 | cyclopropyl | cyclopropylmethyl | H | Cl | 0 | — |
| 3-647 | Q3 | cyclopropyl | cyclopropylmethyl | H | Cl | 1 | racemate |
| 3-648 | Q3 | cyclopropyl | cyclopropylmethyl | H | Cl | 1 | isomer1 |
| 3-649 | Q3 | cyclopropyl | cyclopropylmethyl | H | Cl | 1 | isomer2 |
| 3-650 | Q3 | cyclopropyl | cyclopropylmethyl | H | Cl | 2 | — |
| 3-651 | Q3 | cyclopropyl | cyclopropyl | H | Cl | 0 | — |
| 3-652 | Q3 | cyclopropyl | cyclopropyl | H | Cl | 1 | racemate |
| 3-653 | Q3 | cyclopropyl | cyclopropyl | H | Cl | 1 | isomer1 |
| 3-654 | Q3 | cyclopropyl | cyclopropyl | H | Cl | 1 | isomer2 |
| 3-655 | Q3 | cyclopropyl | cyclopropyl | H | Cl | 2 | — |
| 3-656 | Q3 | cyclopropyl | cyclobutyl | H | Cl | 0 | — |
| 3-657 | Q3 | cyclopropyl | cyclobutyl | H | Cl | 1 | racemate |
| 3-658 | Q3 | cyclopropyl | cyclobutyl | H | Cl | 1 | isomer1 |
| 3-659 | Q3 | cyclopropyl | cyclobutyl | H | Cl | 1 | isomer2 |
| 3-660 | Q3 | cyclopropyl | cyclobutyl | H | Cl | 2 | — |
| 3-661 | Q3 | cyclopropyl | cyclopentyl | H | Cl | 0 | — |
| 3-662 | Q3 | cyclopropyl | cyclopentyl | H | Cl | 1 | racemate |
| 3-663 | Q3 | cyclopropyl | cyclopentyl | H | Cl | 1 | isomer1 |
| 3-664 | Q3 | cyclopropyl | cyclopentyl | H | Cl | 1 | isomer2 |
| 3-665 | Q3 | cyclopropyl | cyclopentyl | H | Cl | 2 | — |
| 3-666 | Q3 | cyclopropyl | cyclohexyl | H | Cl | 0 | — |
| 3-667 | Q3 | cyclopropyl | cyclohexyl | H | Cl | 1 | racemate |
| 3-668 | Q3 | cyclopropyl | cyclohexyl | H | Cl | 1 | isomer1 |
| 3-669 | Q3 | cyclopropyl | cyclohexyl | H | Cl | 1 | isomer2 |
| 3-670 | Q3 | cyclopropyl | cyclohexyl | H | Cl | 2 | — |
| 3-671 | Q3 | cyclopropyl | CH₂CF₃ | H | Cl | 0 | — |
| 3-672 | Q3 | cyclopropyl | CH₂CF₃ | H | Cl | 1 | racemate |
| 3-673 | Q3 | cyclopropyl | CH₂CF₃ | H | Cl | 1 | isomer1 |
| 3-674 | Q3 | cyclopropyl | CH₂CF₃ | H | Cl | 1 | isomer2 |
| 3-675 | Q3 | cyclopropyl | CH₂CF₃ | H | Cl | 2 | — |
| 3-676 | Q3 | cyclopropyl | CH₂CF₂H | H | Cl | 0 | — |
| 3-677 | Q3 | cyclopropyl | CH₂CF₂H | H | Cl | 1 | racemate |
| 3-678 | Q3 | cyclopropyl | CH₂CF₂H | H | Cl | 1 | isomer1 |
| 3-679 | Q3 | cyclopropyl | CH₂CF₂H | H | Cl | 1 | isomer2 |
| 3-680 | Q3 | cyclopropyl | CH₂CF₂H | H | Cl | 2 | — |
| 3-681 | Q3 | cyclopropyl | CH₂CFH₂ | H | Cl | 0 | — |
| 3-682 | Q3 | cyclopropyl | CH₂CFH₂ | H | Cl | 1 | racemate |
| 3-683 | Q3 | cyclopropyl | CH₂CFH₂ | H | Cl | 1 | isomer1 |
| 3-684 | Q3 | cyclopropyl | CH₂CFH₂ | H | Cl | 1 | isomer2 |
| 3-685 | Q3 | cyclopropyl | CH₂CFH₂ | H | Cl | 2 | — |
| 3-686 | Q3 | cyclopropyl | CH₂CH═CH₂ | H | Cl | 0 | — |
| 3-687 | Q3 | cyclopropyl | CH₂CH═CH₂ | H | Cl | 1 | racemate |
| 3-688 | Q3 | cyclopropyl | CH₂CH═CH₂ | H | Cl | 1 | isomer1 |
| 3-689 | Q3 | cyclopropyl | CH₂CH═CH₂ | H | Cl | 1 | isomer2 |
| 3-690 | Q3 | cyclopropyl | CH₂CH═CH₂ | H | Cl | 2 | — |
| 3-691 | Q3 | cyclopropyl | CF₂CF₃ | H | Cl | 0 | — |
| 3-692 | Q3 | cyclopropyl | CF₂CF₃ | H | Cl | 1 | racemate |
| 3-693 | Q3 | cyclopropyl | CF₂CF₃ | H | Cl | 1 | isomer1 |
| 3-694 | Q3 | cyclopropyl | CF₂CF₃ | H | Cl | 1 | isomer2 |
| 3-695 | Q3 | cyclopropyl | CF₂CF₃ | H | Cl | 2 | — |
| 3-696 | Q3 | cyclopropyl | CF₂CF₂CF₃ | H | Cl | 0 | — |
| 3-697 | Q3 | cyclopropyl | CF₂CF₂CF₃ | H | Cl | 1 | racemate |
| 3-698 | Q3 | cyclopropyl | CF₂CF₂CF₃ | H | Cl | 1 | isomer1 |
| 3-699 | Q3 | cyclopropyl | CF₂CF₂CF₃ | H | Cl | 1 | isomer2 |
| 3-700 | Q3 | cyclopropyl | CF₂CF₂CF₃ | H | Cl | 2 | — |
| 3-701 | Q3 | cyclopropyl | CH₂CH₂CF₃ | H | Cl | 0 | — |
| 3-702 | Q3 | cyclopropyl | CH₂CH₂CF₃ | H | Cl | 1 | racemate |
| 3-703 | Q3 | cyclopropyl | CH₂CH₂CF₃ | H | Cl | 1 | isomer1 |
| 3-704 | Q3 | cyclopropyl | CH₂CH₂CF₃ | H | Cl | 1 | isomer2 |
| 3-705 | Q3 | cyclopropyl | CH₂CH₂CF₃ | H | Cl | 2 | — |
| 3-706 | Q3 | cyclopropyl | CH₂CH₂CF₂H | H | Cl | 0 | — |
| 3-707 | Q3 | cyclopropyl | CH₂CH₂CF₂H | H | Cl | 1 | racemate |
| 3-708 | Q3 | cyclopropyl | CH₂CH₂CF₂H | H | Cl | 1 | isomer1 |
| 3-709 | Q3 | cyclopropyl | CH₂CH₂CF₂H | H | Cl | 1 | isomer2 |
| 3-710 | Q3 | cyclopropyl | CH₂CH₂CF₂H | H | Cl | 2 | — |
| 3-711 | Q3 | cyclopropyl | CH₂CH₂CFH₂ | H | Cl | 0 | — |
| 3-712 | Q3 | cyclopropyl | CH₂CH₂CFH₂ | H | Cl | 1 | racemate |
| 3-713 | Q3 | cyclopropyl | CH₂CH₂CFH₂ | H | Cl | 1 | isomer1 |
| 3-714 | Q3 | cyclopropyl | CH₂CH₂CFH₂ | H | Cl | 1 | isomer2 |
| 3-715 | Q3 | cyclopropyl | CH₂CH₂CFH₂ | H | Cl | 2 | — |
| 3-716 | Q3 | cyclopropyl | CH₂CN | H | Cl | 0 | — |
| 3-717 | Q3 | cyclopropyl | CH₂CN | H | Cl | 1 | racemate |
| 3-718 | Q3 | cyclopropyl | CH₂CN | H | Cl | 1 | isomer1 |
| 3-719 | Q3 | cyclopropyl | CH₂CN | H | Cl | 1 | isomer2 |
| 3-720 | Q3 | cyclopropyl | CH₂CN | H | Cl | 2 | — |
| 3-721 | Q3 | cyclopropyl | CH₂CH₂CN | H | Cl | 0 | — |
| 3-722 | Q3 | cyclopropyl | CH₂CH₂CN | H | Cl | 1 | racemate |
| 3-723 | Q3 | cyclopropyl | CH₂CH₂CN | H | Cl | 1 | isomer1 |
| 3-724 | Q3 | cyclopropyl | CH₂CH₂CN | H | Cl | 1 | isomer2 |
| 3-725 | Q3 | cyclopropyl | CH₂CH₂CN | H | Cl | 2 | — |
| 3-726 | Q3 | cyclopropyl | CH₂CH₂Cl | H | Cl | 0 | — |
| 3-727 | Q3 | cyclopropyl | CH₂CH₂Cl | H | Cl | 1 | racemate |
| 3-728 | Q3 | cyclopropyl | CH₂CH₂Cl | H | Cl | 1 | isomer1 |
| 3-729 | Q3 | cyclopropyl | CH₂CH₂Cl | H | Cl | 1 | isomer2 |
| 3-730 | Q3 | cyclopropyl | CH₂CH₂Cl | H | Cl | 2 | — |
| 3-731 | Q3 | CH₂CH₂CH₃ | CH₂CH₂CH₃ | H | Cl | 0 | — |
| 3-732 | Q3 | CH₂CH₂CH₃ | CH₂CH₂CH₃ | H | Cl | 1 | racemate |
| 3-733 | Q3 | CH₂CH₂CH₃ | CH₂CH₂CH₃ | H | Cl | 1 | isomer1 |
| 3-734 | Q3 | CH₂CH₂CH₃ | CH₂CH₂CH₃ | H | Cl | 1 | isomer2 |
| 3-735 | Q3 | CH₂CH₂CH₃ | CH₂CH₂CH₃ | H | Cl | 2 | — |
| 3-736 | Q3 | CH₂CH₂CH₃ | CH(CH₃)₂ | H | Cl | 0 | — |
| 3-737 | Q3 | CH₂CH₂CH₃ | CH(CH₃)₂ | H | Cl | 1 | racemate |
| 3-738 | Q3 | CH₂CH₂CH₃ | CH(CH₃)₂ | H | Cl | 1 | isomer1 |
| 3-739 | Q3 | CH₂CH₂CH₃ | CH(CH₃)₂ | H | Cl | 1 | isomer2 |
| 3-740 | Q3 | CH₂CH₂CH₃ | CH(CH₃)₂ | H | Cl | 2 | — |
| 3-741 | Q3 | C(CH₃)₃ | CH₂CH₂CH₃ | H | Cl | 0 | — |
| 3-742 | Q3 | C(CH₃)₃ | CH₂CH₂CH₃ | H | Cl | 1 | racemate |
| 3-743 | Q3 | C(CH₃)₃ | CH₂CH₂CH₃ | H | Cl | 1 | isomer1 |
| 3-744 | Q3 | C(CH₃)₃ | CH₂CH₂CH₃ | H | Cl | 1 | isomer2 |
| 3-745 | Q3 | C(CH₃)₃ | CH₂CH₂CH₃ | H | Cl | 2 | — |
| 3-746 | Q3 | C(CH₃)₃ | CH(CH₃)₂ | H | Cl | 0 | — |
| 3-747 | Q3 | C(CH₃)₃ | CH(CH₃)₂ | H | Cl | 1 | racemate |
| 3-748 | Q3 | C(CH₃)₃ | CH(CH₃)₂ | H | Cl | 1 | isomer1 |
| 3-749 | Q3 | C(CH₃)₃ | CH(CH₃)₂ | H | Cl | 1 | isomer2 |
| 3-750 | Q3 | C(CH₃)₃ | CH(CH₃)₂ | H | Cl | 2 | — |
| 3-751 | Q3 | CF₃ | CH₂CH₂CH₃ | H | Cl | 0 | — |
| 3-752 | Q3 | CF₃ | CH₂CH₂CH₃ | H | Cl | 1 | racemate |
| 3-753 | Q3 | CF₃ | CH₂CH₂CH₃ | H | Cl | 1 | isomer1 |
| 3-754 | Q3 | CF₃ | CH₂CH₂CH₃ | H | Cl | 1 | isomer2 |
| 3-755 | Q3 | CF₃ | CH₂CH₂CH₃ | H | Cl | 2 | — |
| 3-756 | Q3 | CF₃ | CH(CH₃)₂ | H | Cl | 0 | — |
| 3-757 | Q3 | CF₃ | CH(CH₃)₂ | H | Cl | 1 | racemate |
| 3-758 | Q3 | CF₃ | CH(CH₃)₂ | H | Cl | 1 | isomer1 |
| 3-759 | Q3 | CF₃ | CH(CH₃)₂ | H | Cl | 1 | isomer2 |
| 3-760 | Q3 | CF₃ | CH(CH₃)₂ | H | Cl | 2 | — |
| 3-761 | Q3 | cyclobutyl | CH(CH₃)₂ | H | Cl | 0 | — |
| 3-762 | Q3 | cyclobutyl | CH(CH₃)₂ | H | Cl | 1 | racemate |
| 3-763 | Q3 | cyclobutyl | CH(CH₃)₂ | H | Cl | 1 | isomer1 |
| 3-764 | Q3 | cyclobutyl | CH(CH₃)₂ | H | Cl | 1 | isomer2 |
| 3-765 | Q3 | cyclobutyl | CH(CH₃)₂ | H | Cl | 2 | — |
| 3-766 | Q3 | Furan-2-yl | CH(CH₃)₂ | H | Cl | 0 | — |
| 3-767 | Q3 | Furan-2-yl | CH(CH₃)₂ | H | Cl | 1 | racemate |
| 3-768 | Q3 | Furan-2-yl | CH(CH₃)₂ | H | Cl | 1 | isomer1 |
| 3-769 | Q3 | Furan-2-yl | CH(CH₃)₂ | H | Cl | 1 | isomer2 |
| 3-770 | Q3 | Furan-2-yl | CH(CH₃)₂ | H | Cl | 2 | — |
| 3-771 | Q3 | C₆H₅ | CH(CH₃)₂ | H | Cl | 0 | — |
| 3-772 | Q3 | C₆H₅ | CH(CH₃)₂ | H | Cl | 1 | racemate |
| 3-773 | Q3 | C₆H₅ | CH(CH₃)₂ | H | Cl | 1 | isomer1 |
| 3-774 | Q3 | C₆H₅ | CH(CH₃)₂ | H | Cl | 1 | isomer2 |
| 3-775 | Q3 | C₆H₅ | CH(CH₃)₂ | H | Cl | 2 | — |
| 3-776 | Q3 | 4-fluorophenyl | CH(CH₃)₂ | H | Cl | 0 | — |
| 3-777 | Q3 | 4-fluorophenyl | CH(CH₃)₂ | H | Cl | 1 | racemate |
| 3-778 | Q3 | 4-fluorophenyl | CH(CH₃)₂ | H | Cl | 1 | isomer1 |
| 3-779 | Q3 | 4-fluorophenyl | CH(CH₃)₂ | H | Cl | 1 | isomer2 |
| 3-780 | Q3 | 4-fluorophenyl | CH(CH₃)₂ | H | Cl | 2 | — |
| 3-781 | Q3 | C₂H₅OCO | CH(CH₃)₂ | H | Cl | 0 | — |
| 3-782 | Q3 | C₂H₅OCO | CH(CH₃)₂ | H | Cl | 1 | racemate |
| 3-783 | Q3 | C₂H₅OCO | CH(CH₃)₂ | H | Cl | 1 | isomer1 |
| 3-784 | Q3 | C₂H₅OCO | CH(CH₃)₂ | H | Cl | 1 | isomer2 |
| 3-785 | Q3 | C₂H₅OCO | CH(CH₃)₂ | H | Cl | 2 | — |
| 3-786 | Q3 | CH₃ | CH₂CH₂CH₃ | CH₃ | Cl | 0 | — |
| 3-787 | Q3 | CH₃ | CH₂CH₂CH₃ | CH₃ | Cl | 1 | racemate |
| 3-788 | Q3 | CH₃ | CH₂CH₂CH₃ | CH₃ | Cl | 1 | isomer1 |
| 3-789 | Q3 | CH₃ | CH₂CH₂CH₃ | CH₃ | Cl | 1 | isomer2 |
| 3-790 | Q3 | CH₃ | CH₂CH₂CH₃ | CH₃ | Cl | 2 | — |
| 3-791 | Q3 | CH₃ | CH(CH₃)₂ | CH₃ | Cl | 0 | — |
| 3-792 | Q3 | CH₃ | CF₂CFHCF₃ | H | Cl | 0 | — |
| 3-793 | Q3 | CH₃ | CH(CH₃)₂ | CH₃ | Cl | 1 | isomer1 |
| 3-794 | Q3 | CH₃ | CH(CH₃)₂ | CH₃ | Cl | 1 | isomer2 |
| 3-795 | Q3 | H | CH₂CH₂CH₃ | CH₃ | Cl | 1 | racemate |
| 3-796 | Q3 | H | CH₂CH₂CH₃ | CH₃ | Cl | 1 | isomer1 |
| 3-797 | Q3 | H | CH₂CH₂CH₃ | CH₃ | Cl | 1 | isomer2 |
| 3-798 | Q3 | CH₂CH₃ | CH₂CH₂CH₃ | CH₃ | Cl | 1 | racemate |
| 3-799 | Q3 | CH₂CH₃ | CH₂CH₂CH₃ | CH₃ | Cl | 1 | isomer1 |
| 3-800 | Q3 | CH₂CH₃ | CH₂CH₂CH₃ | CH₃ | Cl | 1 | isomer2 |
| 3-801 | Q3 | CH(CH₃)₂ | CH₂CH₂CH₃ | CH₃ | Cl | 1 | racemate |
| 3-802 | Q3 | CH(CH₃)₂ | CH₂CH₂CH₃ | CH₃ | Cl | 1 | isomer1 |
| 3-803 | Q3 | CH(CH₃)₂ | CH₂CH₂CH₃ | CH₃ | Cl | 1 | isomer2 |
| 3-804 | Q3 | H | CH(CH₃)₂ | CH₃ | Cl | 1 | racemate |
| 3-805 | Q3 | H | CH(CH₃)₂ | CH₃ | Cl | 1 | isomer1 |
| 3-806 | Q3 | H | CH(CH₃)₂ | CH₃ | Cl | 1 | isomer2 |
| 3-807 | Q3 | CH₃ | CH(CH₃)₂ | CH₃ | Cl | 1 | racemate |
| 3-808 | Q3 | CH(CH₃)₂ | CH(CH₃)₂ | CH₃ | Cl | 1 | racemate |
| 3-809 | Q3 | cyclopropyl | CH(CH₃)₂ | CH₃ | Cl | 1 | racemate |
| 3-810 | Q3 | CH₂CH₃ | CH(CH₃)₂ | CH₃ | Cl | 1 | racemate |
| 3-811 | Q3 | CH₃ | CH(CH₃)₂ | CH₃ | Cl | 2 | — |
| 3-812 | Q3 | CH₃ | cyclopropyl | CH₃ | Cl | 1 | racemate |
| 3-813 | Q3 | CH₂CH₃ | cyclopropyl | CH₃ | Cl | 1 | racemate |
| 3-814 | Q3 | CH₃ | CH₂CH₂CH₂CH₃ | CH₃ | Cl | 0 | — |
| 3-815 | Q3 | CH₃ | CH₂CH₂CH₂CH₃ | CH₃ | Cl | 1 | racemate |
| 3-816 | Q3 | CH₃ | CH₂CH₂CH₂CH₃ | CH₃ | Cl | 2 | — |
| 3-817 | Q3 | CH₃ | CH(CH₃)CH₂CH₃ | CH₃ | Cl | 0 | racemate |
| 3-818 | Q3 | CH₃ | CH(CH₃)CH₂CH₃ | CH₃ | Cl | 0 | isomer1 |
| 3-819 | Q3 | CH₃ | CH(CH₃)CH₂CH₃ | CH₃ | Cl | 0 | isomer2 |
| 3-820 | Q3 | CH₃ | CH(CH₃)CH₂CH₃ | CH₃ | Cl | 1 | mixture |
| 3-821 | Q3 | CH₃ | CH(CH₃)CH₂CH₃ | CH₃ | Cl | 1 | isomer1 |
| 3-822 | Q3 | CH₃ | CH(CH₃)CH₂CH₃ | CH₃ | Cl | 1 | isomer2 |
| 3-823 | Q3 | CH₃ | CH(CH₃)CH₂CH₃ | CH₃ | Cl | 1 | isomer3 |
| 3-824 | Q3 | CH₃ | CH(CH₃)CH₂CH₃ | CH₃ | Cl | 1 | isomer4 |
| 3-825 | Q3 | CH₃ | CH(CH₃)CH₂CH₃ | CH₃ | Cl | 2 | racemate |
| 3-826 | Q3 | CH₃ | CH(CH₃)CH₂CH₃ | CH₃ | Cl | 2 | isomer1 |
| 3-827 | Q3 | CH₃ | CH(CH₃)CH₂CH₃ | CH₃ | Cl | 2 | isomer2 |
| 3-828 | Q3 | CH₃ | CH₂CH(CH₃)₂ | CH₃ | Cl | 0 | — |
| 3-829 | Q3 | CH₃ | CH₂CH(CH₃)₂ | CH₃ | Cl | 1 | racemate |
| 3-830 | Q3 | CH₃ | CH₂CH(CH₃)₂ | CH₃ | Cl | 2 | — |
| 3-831 | Q3 | CH₃ | CH₂CF₃ | CH₃ | Cl | 0 | — |
| 3-832 | Q3 | CH₃ | CH₂CF₃ | CH₃ | Cl | 1 | racemate |
| 3-833 | Q3 | CH₃ | CH₂CF₃ | CH₃ | Cl | 2 | — |
| 3-834 | Q3 | CH₃ | CH₂CH₂Cl | CH₃ | Cl | 0 | — |
| 3-835 | Q3 | CH₃ | CH₂CH₂Cl | CH₃ | Cl | 1 | racemate |
| 3-836 | Q3 | CH₃ | CH₂CH₂Cl | CH₃ | Cl | 2 | — |
| 3-837 | Q3 | CH₃ | CH₂CH₂CH₃ | CH₂CH₃ | Cl | 0 | — |
| 3-838 | Q3 | CH₃ | CH₂CH₂CH₃ | CH₂CH₃ | Cl | 1 | racemate |
| 3-839 | Q3 | CH₃ | CH₂CH₂CH₃ | CH₂CH₃ | Cl | 2 | — |
| 3-840 | Q3 | CH₃ | CH(CH₃)₂ | CH₂CH₃ | Cl | 0 | — |
| 3-841 | Q3 | CH₃ | CH(CH₃)₂ | CH₂CH₃ | Cl | 1 | racemate |
| 3-842 | Q3 | CH₃ | CH(CH₃)₂ | CH₂CH₃ | Cl | 2 | — |
| 3-843 | Q3 | CH₃ | CH₂CH₂CH₃ | CH(CH₃)₂ | Cl | 0 | — |
| 3-844 | Q3 | CH₃ | CH₂CH₂CH₃ | CH(CH₃)₂ | Cl | 1 | racemate |
| 3-845 | Q3 | CH₃ | CH₂CH₂CH₃ | CH(CH₃)₂ | Cl | 2 | — |
| 3-846 | Q3 | CH₃ | CH(CH₃)₂ | CH(CH₃)₂ | Cl | 0 | — |
| 3-847 | Q3 | CH₃ | CH(CH₃)₂ | CH(CH₃)₂ | Cl | 1 | racemate |
| 3-848 | Q3 | CH₃ | CH(CH₃)₂ | CH(CH₃)₂ | Cl | 2 | — |
| 3-849 | Q3 | CH₃ | CH₂CH₂CH₃ | CH₂CH₂OCH₃ | Cl | 0 | — |
| 3-850 | Q3 | CH₃ | CH₂CH₂CH₃ | CH₂CH₂OCH₃ | Cl | 1 | racemate |
| 3-851 | Q3 | CH₃ | CH₂CH₂CH₃ | CH₂CH₂OCH₃ | Cl | 2 | — |
| 3-852 | Q3 | CH₃ | cyclobutylmethyl | H | Cl | 0 | — |
| 3-853 | Q3 | CH₃ | cyclobutylmethyl | H | Cl | 1 | racemate |
| 3-854 | Q3 | CH₃ | cyclobutylmethyl | H | Cl | 1 | isomer1 |
| 3-855 | Q3 | CH₃ | cyclobutylmethyl | H | Cl | 1 | isomer2 |
| 3-856 | Q3 | CH₃ | cyclobutylmethyl | H | Cl | 2 | — |
| 3-857 | Q3 | CH₃ | cyclopentylmethyl | H | Cl | 0 | — |
| 3-858 | Q3 | CH₃ | cyclopentylmethyl | H | Cl | 1 | racemate |
| 3-859 | Q3 | CH₃ | cyclopentylmethyl | H | Cl | 1 | isomer1 |
| 3-860 | Q3 | CH₃ | cyclopentylmethyl | H | Cl | 1 | isomer2 |
| 3-861 | Q3 | CH₃ | cyclopentylmethyl | H | Cl | 2 | — |
| 3-862 | Q3 | CH₃ | 3,3-difluorocyclobutyl | H | Cl | 0 | — |
| 3-863 | Q3 | CH₃ | 3,3-difluorocyclobutyl | H | Cl | 1 | racemate |
| 3-864 | Q3 | CH₃ | 3,3-difluorocyclobutyl | H | Cl | 1 | isomer1 |
| 3-865 | Q3 | CH₃ | 3,3-difluorocyclobutyl | H | Cl | 1 | isomer2 |
| 3-866 | Q3 | CH₃ | 3,3-difluorocyclobutyl | H | Cl | 2 | — |
| 4-1 | Q4 | CH₃ | CH₂CH₂CH₃ | H | Cl | 0 | — |
| 4-2 | Q4 | CH₃ | CH₂CH₂CH₃ | H | Cl | 1 | racemate |
| 4-3 | Q4 | CH₃ | CH₂CH₂CH₃ | H | Cl | 1 | isomer1 |
| 4-4 | Q4 | CH₃ | CH₂CH₂CH₃ | H | Cl | 1 | isomer2 |
| 4-5 | Q4 | CH₃ | CH₂CH₂CH₃ | H | Cl | 2 | — |
| 4-6 | Q4 | CH₃ | CH(CH₃)₂ | H | Cl | 0 | — |
| 4-7 | Q4 | CH₃ | CH(CH₃)₂ | H | Cl | 1 | racemate |
| 4-8 | Q4 | CH₃ | CH(CH₃)₂ | H | Cl | 1 | isomer1 |
| 4-9 | Q4 | CH₃ | CH(CH₃)₂ | H | Cl | 1 | isomer2 |
| 4-10 | Q4 | CH₃ | CH(CH₃)₂ | H | Cl | 2 | — |
| 4-11 | Q4 | CH₃ | CH₂CH₂CH₂CH₃ | H | Cl | 0 | — |
| 4-12 | Q4 | CH₃ | CH₂CH₂CH₂CH₃ | H | Cl | 1 | racemate |
| 4-13 | Q4 | CH₃ | CH₂CH₂CH₂CH₃ | H | Cl | 1 | isomer1 |
| 4-14 | Q4 | CH₃ | CH₂CH₂CH₂CH₃ | H | Cl | 1 | isomer2 |
| 4-15 | Q4 | CH₃ | CH₂CH₂CH₂CH₃ | H | Cl | 2 | — |
| 4-16 | Q4 | CH₃ | CH(CH₃)CH₂CH₃ | H | Cl | 0 | racemate |
| 4-17 | Q4 | CH₃ | CH(CH₃)CH₂CH₃ | H | Cl | 0 | isomer1 |
| 4-18 | Q4 | CH₃ | CH(CH₃)CH₂CH₃ | H | Cl | 0 | isomer2 |
| 4-19 | Q4 | CH₃ | CH(CH₃)CH₂CH₃ | H | Cl | 1 | mixture |
| 4-20 | Q4 | CH₃ | CH(CH₃)CH₂CH₃ | H | Cl | 1 | isomer1 |
| 4-21 | Q4 | CH₃ | CH(CH₃)CH₂CH₃ | H | Cl | 1 | isomer2 |
| 4-22 | Q4 | CH₃ | CH(CH₃)CH₂CH₃ | H | Cl | 1 | isomer3 |
| 4-23 | Q4 | CH₃ | CH(CH₃)CH₂CH₃ | H | Cl | 1 | isomer4 |
| 4-24 | Q4 | CH₃ | CH(CH₃)CH₂CH₃ | H | Cl | 2 | racemate |
| 4-25 | Q4 | CH₃ | CH(CH₃)CH₂CH₃ | H | Cl | 2 | isomer1 |
| 4-26 | Q4 | CH₃ | CH(CH₃)CH₂CH₃ | H | Cl | 2 | isomer2 |
| 4-27 | Q4 | CH₃ | CH₂CH(CH₃)₂ | H | Cl | 0 | — |
| 4-28 | Q4 | CH₃ | CH₂CH(CH₃)₂ | H | Cl | 1 | racemate |
| 4-29 | Q4 | CH₃ | CH₂CH(CH₃)₂ | H | Cl | 1 | isomer1 |
| 4-30 | Q4 | CH₃ | CH₂CH(CH₃)₂ | H | Cl | 1 | isomer2 |
| 4-31 | Q4 | CH₃ | CH₂CH(CH₃)₂ | H | Cl | 2 | — |
| 4-32 | Q4 | CH₃ | cyclopropylmethyl | H | Cl | 0 | — |
| 4-33 | Q4 | CH₃ | cyclopropylmethyl | H | Cl | 1 | racemate |
| 4-34 | Q4 | CH₃ | cyclopropylmethyl | H | Cl | 1 | isomer1 |
| 4-35 | Q4 | CH₃ | cyclopropylmethyl | H | Cl | 1 | isomer2 |
| 4-36 | Q4 | CH₃ | cyclopropylmethyl | H | Cl | 2 | — |
| 4-37 | Q4 | CH₃ | cyclopropyl | H | Cl | 0 | — |
| 4-38 | Q4 | CH₃ | cyclopropyl | H | Cl | 1 | racemate |
| 4-39 | Q4 | CH₃ | cyclopropyl | H | Cl | 1 | isomer1 |
| 4-40 | Q4 | CH₃ | cyclopropyl | H | Cl | 1 | isomer2 |
| 4-41 | Q4 | CH₃ | cyclopropyl | H | Cl | 2 | — |
| 4-42 | Q4 | CH₃ | cyclobutyl | H | Cl | 0 | — |
| 4-43 | Q4 | CH₃ | cyclobutyl | H | Cl | 1 | racemate |
| 4-44 | Q4 | CH₃ | cyclobutyl | H | Cl | 1 | isomer1 |
| 4-45 | Q4 | CH₃ | cyclobutyl | H | Cl | 1 | isomer2 |
| 4-46 | Q4 | CH₃ | cyclobutyl | H | Cl | 2 | — |
| 4-47 | Q4 | CH₃ | cyclopentyl | H | Cl | 0 | — |
| 4-48 | Q4 | CH₃ | cyclopentyl | H | Cl | 1 | racemate |
| 4-49 | Q4 | CH₃ | cyclopentyl | H | Cl | 1 | isomer1 |
| 4-50 | Q4 | CH₃ | cyclopentyl | H | Cl | 1 | isomer2 |
| 4-51 | Q4 | CH₃ | cyclopentyl | H | Cl | 2 | — |
| 4-52 | Q4 | CH₃ | cyclohexyl | H | Cl | 0 | — |
| 4-53 | Q4 | CH₃ | cyclohexyl | H | Cl | 1 | racemate |
| 4-54 | Q4 | CH₃ | cyclohexyl | H | Cl | 1 | isomer1 |
| 4-55 | Q4 | CH₃ | cyclohexyl | H | Cl | 1 | isomer2 |
| 4-56 | Q4 | CH₃ | cyclohexyl | H | Cl | 2 | — |
| 4-57 | Q4 | CH₃ | CH₂CF₃ | H | Cl | 0 | — |
| 4-58 | Q4 | CH₃ | CH₂CF₃ | H | Cl | 1 | racemate |
| 4-59 | Q4 | CH₃ | CH₂CF₃ | H | Cl | 1 | isomer1 |
| 4-60 | Q4 | CH₃ | CH₂CF₃ | H | Cl | 1 | isomer2 |
| 4-61 | Q4 | CH₃ | CH₂CF₃ | H | Cl | 2 | — |
| 4-62 | Q4 | CH₃ | CH₂CF₂H | H | Cl | 0 | — |
| 4-63 | Q4 | CH₃ | CH₂CF₂H | H | Cl | 1 | racemate |
| 4-64 | Q4 | CH₃ | CH₂CF₂H | H | Cl | 1 | isomer1 |
| 4-65 | Q4 | CH₃ | CH₂CF₂H | H | Cl | 1 | isomer2 |
| 4-66 | Q4 | CH₃ | CH₂CF₂H | H | Cl | 2 | — |
| 4-67 | Q4 | CH₃ | CH₂CFH₂ | H | Cl | 0 | — |
| 4-68 | Q4 | CH₃ | CH₂CFH₂ | H | Cl | 1 | racemate |
| 4-69 | Q4 | CH₃ | CH₂CFH₂ | H | Cl | 1 | isomer1 |
| 4-70 | Q4 | CH₃ | CH₂CFH₂ | H | Cl | 1 | isomer2 |
| 4-71 | Q4 | CH₃ | CH₂CFH₂ | H | Cl | 2 | — |
| 4-72 | Q4 | CH₃ | CH₂CH═CH₂ | H | Cl | 0 | — |
| 4-73 | Q4 | CH₃ | CH₂CH═CH₂ | H | Cl | 1 | racemate |
| 4-74 | Q4 | CH₃ | CH₂CH═CH₂ | H | Cl | 1 | isomer1 |
| 4-75 | Q4 | CH₃ | CH₂CH═CH₂ | H | Cl | 1 | isomer2 |
| 4-76 | Q4 | CH₃ | CH₂CH═CH₂ | H | Cl | 2 | — |
| 4-77 | Q4 | CH₃ | CF₂CF₃ | H | Cl | 0 | — |
| 4-78 | Q4 | CH₃ | CF₂CF₃ | H | Cl | 1 | racemate |
| 4-79 | Q4 | CH₃ | CF₂CF₃ | H | Cl | 1 | isomer1 |
| 4-80 | Q4 | CH₃ | CF₂CF₃ | H | Cl | 1 | isomer2 |
| 4-81 | Q4 | CH₃ | CF₂CF₃ | H | Cl | 2 | — |
| 4-82 | Q4 | CH₃ | CF₂CF₂CF₃ | H | Cl | 0 | — |
| 4-83 | Q4 | CH₃ | CF₂CF₂CF₃ | H | Cl | 1 | racemate |
| 4-84 | Q4 | CH₃ | CF₂CF₂CF₃ | H | Cl | 1 | isomer1 |
| 4-85 | Q4 | CH₃ | CF₂CF₂CF₃ | H | Cl | 1 | isomer2 |
| 4-86 | Q4 | CH₃ | CF₂CF₂CF₃ | H | Cl | 2 | — |
| 4-87 | Q4 | CH₃ | CH₂CH₂CF₃ | H | Cl | 0 | — |
| 4-88 | Q4 | CH₃ | CH₂CH₂CF₃ | H | Cl | 1 | racemate |
| 4-89 | Q4 | CH₃ | CH₂CH₂CF₃ | H | Cl | 1 | isomer1 |
| 4-90 | Q4 | CH₃ | CH₂CH₂CF₃ | H | Cl | 1 | isomer2 |
| 4-91 | Q4 | CH₃ | CH₂CH₂CF₃ | H | Cl | 2 | — |
| 4-92 | Q4 | CH₃ | CH₂CH₂CF₂H | H | Cl | 0 | — |
| 4-93 | Q4 | CH₃ | CH₂CH₂CF₂H | H | Cl | 1 | racemate |
| 4-94 | Q4 | CH₃ | CH₂CH₂CF₂H | H | Cl | 1 | isomer1 |
| 4-95 | Q4 | CH₃ | CH₂CH₂CF₂H | H | Cl | 1 | isomer2 |
| 4-96 | Q4 | CH₃ | CH₂CH₂CF₂H | H | Cl | 2 | — |
| 4-97 | Q4 | CH₃ | CH₂CH₂CFH₂ | H | Cl | 0 | — |
| 4-98 | Q4 | CH₃ | CH₂CH₂CFH₂ | H | Cl | 1 | racemate |
| 4-99 | Q4 | CH₃ | CH₂CH₂CFH₂ | H | Cl | 1 | isomer1 |
| 4-100 | Q4 | CH₃ | CH₂CH₂CFH₂ | H | Cl | 1 | isomer2 |
| 4-101 | Q4 | CH₃ | CH₂CH₂CFH₂ | H | Cl | 2 | — |
| 4-102 | Q4 | CH₃ | CH₂CN | H | Cl | 0 | — |
| 4-103 | Q4 | CH₃ | CH₂CN | H | Cl | 1 | racemate |
| 4-104 | Q4 | CH₃ | CH₂CN | H | Cl | 1 | isomer1 |
| 4-105 | Q4 | CH₃ | CH₂CN | H | Cl | 1 | isomer2 |
| 4-106 | Q4 | CH₃ | CH₂CN | H | Cl | 2 | — |
| 4-107 | Q4 | CH₃ | CH₂CH₂CN | H | Cl | 0 | — |
| 4-108 | Q4 | CH₃ | CH₂CH₂CN | H | Cl | 1 | racemate |
| 4-109 | Q4 | CH₃ | CH₂CH₂CN | H | Cl | 1 | isomer1 |
| 4-110 | Q4 | CH₃ | CH₂CH₂CN | H | Cl | 1 | isomer2 |
| 4-111 | Q4 | CH₃ | CH₂CH₂CN | H | Cl | 2 | — |
| 4-112 | Q4 | CH₃ | CH₂CH₂Cl | H | Cl | 0 | — |
| 4-113 | Q4 | CH₃ | CH₂CH₂Cl | H | Cl | 1 | racemate |
| 4-114 | Q4 | CH₃ | CH₂CH₂Cl | H | Cl | 1 | isomer1 |
| 4-115 | Q4 | CH₃ | CH₂CH₂Cl | H | Cl | 1 | isomer2 |
| 4-116 | Q4 | CH₃ | CH₂CH₂Cl | H | Cl | 2 | — |
| 4-117 | Q4 | CH₂CH₃ | CH₂CH₂CH₃ | H | Cl | 0 | — |
| 4-118 | Q4 | CH₂CH₃ | CH₂CH₂CH₃ | H | Cl | 1 | racemate |
| 4-119 | Q4 | CH₂CH₃ | CH₂CH₂CH₃ | H | Cl | 1 | isomer 1 |
| 4-120 | Q4 | CH₂CH₃ | CH₂CH₂CH₃ | H | Cl | 1 | isomer2 |
| 4-121 | Q4 | CH₂CH₃ | CH₂CH₂CH₃ | H | Cl | 2 | — |
| 4-122 | Q4 | CH₂CH₃ | CH(CH₃)₂ | H | Cl | 0 | — |
| 4-123 | Q4 | CH₂CH₃ | CH(CH₃)₂ | H | Cl | 1 | racemate |
| 4-124 | Q4 | CH₂CH₃ | CH(CH₃)₂ | H | Cl | 1 | isomer1 |
| 4-125 | Q4 | CH₂CH₃ | CH(CH₃)₂ | H | Cl | 1 | isomer2 |
| 4-126 | Q4 | CH₂CH₃ | CH(CH₃)₂ | H | Cl | 2 | — |
| 4-127 | Q4 | CH(CH₃)₂ | CH(CH₃)₂ | H | Cl | 0 | — |
| 4-128 | Q4 | CH(CH₃)₂ | CH(CH₃)₂ | H | Cl | 1 | racemate |
| 4-129 | Q4 | CH(CH₃)₂ | CH(CH₃)₂ | H | Cl | 1 | isomer1 |
| 4-130 | Q4 | CH(CH₃)₂ | CH(CH₃)₂ | H | Cl | 1 | isomer2 |
| 4-131 | Q4 | CH(CH₃)₂ | CH(CH₃)₂ | H | Cl | 2 | — |
| 4-132 | Q4 | CH(CH₃)₂ | CH(CH₃)CH₂CH₃ | H | Cl | 0 | racemate |
| 4-133 | Q4 | CH(CH₃)₂ | CH(CH₃)CH₂CH₃ | H | Cl | 0 | isomer1 |
| 4-134 | Q4 | CH(CH₃)₂ | CH(CH₃)CH₂CH₃ | H | Cl | 0 | isomer2 |
| 4-135 | Q4 | CH(CH₃)₂ | CH(CH₃)CH₂CH₃ | H | Cl | 1 | mixture |
| 4-136 | Q4 | CH(CH₃)₂ | CH(CH₃)CH₂CH₃ | H | Cl | 1 | isomer1 |
| 4-137 | Q4 | CH(CH₃)₂ | CH(CH₃)CH₂CH₃ | H | Cl | 1 | isomer2 |
| 4-138 | Q4 | CH(CH₃)₂ | CH(CH₃)CH₂CH₃ | H | Cl | 1 | isomer3 |
| 4-139 | Q4 | CH(CH₃)₂ | CH(CH₃)CH₂CH₃ | H | Cl | 1 | isomer4 |
| 4-140 | Q4 | CH(CH₃)₂ | CH(CH₃)CH₂CH₃ | H | Cl | 2 | racemate |
| 4-141 | Q4 | CH(CH₃)₂ | CH(CH₃)CH₂CH₃ | H | Cl | 2 | isomer1 |
| 4-142 | Q4 | CH(CH₃)₂ | CH(CH₃)CH₂CH₃ | H | Cl | 2 | isomer2 |
| 4-143 | Q4 | CH₂CH₂CH₃ | CH(CH₃)₂ | H | Cl | 0 | — |
| 4-144 | Q4 | CH₂CH₂CH₃ | CH(CH₃)₂ | H | Cl | 1 | racemate |
| 4-145 | Q4 | CH₂CH₂CH₃ | CH(CH₃)₂ | H | Cl | 1 | isomer1 |
| 4-146 | Q4 | CH₂CH₂CH₃ | CH(CH₃)₂ | H | Cl | 1 | isomer2 |
| 4-147 | Q4 | CH₂CH₂CH₃ | CH(CH₃)₂ | H | Cl | 2 | — |
| 4-148 | Q4 | CH₂CH₂CH₃ | CH(CH₃)CH₂CH₃ | H | Cl | 0 | racemate |
| 4-149 | Q4 | CH₂CH₂CH₃ | CH(CH₃)CH₂CH₃ | H | Cl | 0 | isomer1 |
| 4-150 | Q4 | CH₂CH₂CH₃ | CH(CH₃)CH₂CH₃ | H | Cl | 0 | isomer2 |
| 4-151 | Q4 | CH₂CH₂CH₃ | CH(CH₃)CH₂CH₃ | H | Cl | 1 | mixture |
| 4-152 | Q4 | CH₂CH₂CH₃ | CH(CH₃)CH₂CH₃ | H | Cl | 1 | isomer1 |
| 4-153 | Q4 | CH₂CH₂CH₃ | CH(CH₃)CH₂CH₃ | H | Cl | 1 | isomer2 |
| 4-154 | Q4 | CH₂CH₂CH₃ | CH(CH₃)CH₂CH₃ | H | Cl | 1 | isomer3 |
| 4-155 | Q4 | CH₂CH₂CH₃ | CH(CH₃)CH₂CH₃ | H | Cl | 1 | isomer4 |
| 4-156 | Q4 | CH₂CH₂CH₃ | CH(CH₃)CH₂CH₃ | H | Cl | 2 | racemate |
| 4-157 | Q4 | CH₂CH₂CH₃ | CH(CH₃)CH₂CH₃ | H | Cl | 2 | isomer1 |
| 4-158 | Q4 | CH₂CH₂CH₃ | CH(CH₃)CH₂CH₃ | H | Cl | 2 | isomer2 |
| 4-159 | Q4 | CN | CH₂CH₂CH₃ | H | Cl | 0 | — |
| 4-160 | Q4 | CN | CH₂CH₂CH₃ | H | Cl | 1 | racemate |
| 4-161 | Q4 | CN | CH₂CH₂CH₃ | H | Cl | 1 | isomer 1 |
| 4-162 | Q4 | CN | CH₂CH₂CH₃ | H | Cl | 1 | isomer2 |
| 4-163 | Q4 | CN | CH₂CH₂CH₃ | H | Cl | 2 | — |
| 4-164 | Q4 | CN | CH(CH₃)₂ | H | Cl | 0 | — |
| 4-165 | Q4 | CN | CH(CH₃)₂ | H | Cl | 1 | racemate |
| 4-166 | Q4 | CN | CH(CH₃)₂ | H | Cl | 1 | isomer1 |
| 4-167 | Q4 | CN | CH(CH₃)₂ | H | Cl | 1 | isomer2 |
| 4-168 | Q4 | CN | CH(CH₃)₂ | H | Cl | 2 | — |
| 4-169 | Q4 | CN | CH₂CH₂CH₂CH₃ | H | Cl | 0 | — |
| 4-170 | Q4 | CN | CH₂CH₂CH₂CH₃ | H | Cl | 1 | racemate |
| 4-171 | Q4 | CN | CH₂CH₂CH₂CH₃ | H | Cl | 1 | isomer1 |
| 4-172 | Q4 | CN | CH₂CH₂CH₂CH₃ | H | Cl | 1 | isomer2 |
| 4-173 | Q4 | CN | CH₂CH₂CH₂CH₃ | H | Cl | 2 | — |
| 4-174 | Q4 | CN | CH(CH₃)CH₂CH₃ | H | Cl | 0 | racemate |
| 4-175 | Q4 | CN | CH(CH₃)CH₂CH₃ | H | Cl | 0 | isomer1 |
| 4-176 | Q4 | CN | CH(CH₃)CH₂CH₃ | H | Cl | 0 | isomer2 |
| 4-177 | Q4 | CN | CH(CH₃)CH₂CH₃ | H | Cl | 1 | mixture |
| 4-178 | Q4 | CN | CH(CH₃)CH₂CH₃ | H | Cl | 1 | isomer1 |
| 4-179 | Q4 | CN | CH(CH₃)CH₂CH₃ | H | Cl | 1 | isomer2 |
| 4-180 | Q4 | CN | CH(CH₃)CH₂CH₃ | H | Cl | 1 | isomer3 |
| 4-181 | Q4 | CN | CH(CH₃)CH₂CH₃ | H | Cl | 1 | isomer4 |
| 4-182 | Q4 | CN | CH(CH₃)CH₂CH₃ | H | Cl | 2 | racemate |
| 4-183 | Q4 | CN | CH(CH₃)CH₂CH₃ | H | Cl | 2 | isomer1 |
| 4-184 | Q4 | CN | CH(CH₃)CH₂CH₃ | H | Cl | 2 | isomer2 |
| 4-185 | Q4 | CN | CH₂CH(CH₃)₂ | H | Cl | 0 | — |
| 4-186 | Q4 | CN | CH₂CH(CH₃)₂ | H | Cl | 1 | racemate |
| 4-187 | Q4 | CN | CH₂CH(CH₃)₂ | H | Cl | 1 | isomer1 |
| 4-188 | Q4 | CN | CH₂CH(CH₃)₂ | H | Cl | 1 | isomer2 |
| 4-189 | Q4 | CN | CH₂CH(CH₃)₂ | H | Cl | 2 | — |
| 4-190 | Q4 | CN | cyclopropylmethyl | H | Cl | 0 | — |
| 4-191 | Q4 | CN | cyclopropylmethyl | H | Cl | 1 | racemate |
| 4-192 | Q4 | CN | cyclopropylmethyl | H | Cl | 1 | isomer1 |
| 4-193 | Q4 | CN | cyclopropylmethyl | H | Cl | 1 | isomer2 |
| 4-194 | Q4 | CN | cyclopropylmethyl | H | Cl | 2 | — |
| 4-195 | Q4 | CN | cyclopropyl | H | Cl | 0 | — |
| 4-196 | Q4 | CN | cyclopropyl | H | Cl | 1 | racemate |
| 4-197 | Q4 | CN | cyclopropyl | H | Cl | 1 | isomer1 |
| 4-198 | Q4 | CN | cyclopropyl | H | Cl | 1 | isomer2 |
| 4-199 | Q4 | CN | cyclopropyl | H | Cl | 2 | — |
| 4-200 | Q4 | CN | cyclobutyl | H | Cl | 0 | — |
| 4-201 | Q4 | CN | cyclobutyl | H | Cl | 1 | racemate |
| 4-202 | Q4 | CN | cyclobutyl | H | Cl | 1 | isomer1 |
| 4-203 | Q4 | CN | cyclobutyl | H | Cl | 1 | isomer2 |
| 4-204 | Q4 | CN | cyclobutyl | H | Cl | 2 | — |
| 4-205 | Q4 | CN | cyclopentyl | H | Cl | 0 | — |
| 4-206 | Q4 | CN | cyclopentyl | H | Cl | 1 | racemate |
| 4-207 | Q4 | CN | cyclopentyl | H | Cl | 1 | isomer1 |
| 4-208 | Q4 | CN | cyclopentyl | H | Cl | 1 | isomer2 |
| 4-209 | Q4 | CN | cyclopentyl | H | Cl | 2 | — |
| 4-210 | Q4 | CN | cyclohexyl | H | Cl | 0 | — |
| 4-211 | Q4 | CN | cyclohexyl | H | Cl | 1 | racemate |
| 4-212 | Q4 | CN | cyclohexyl | H | Cl | 1 | isomer1 |
| 4-213 | Q4 | CN | cyclohexyl | H | Cl | 1 | isomer2 |
| 4-214 | Q4 | CN | cyclohexyl | H | Cl | 2 | — |
| 4-215 | Q4 | cyclopropyl | CH₂CH₂CH₃ | H | Cl | 0 | — |
| 4-216 | Q4 | cyclopropyl | CH₂CH₂CH₃ | H | Cl | 1 | racemate |
| 4-217 | Q4 | cyclopropyl | CH₂CH₂CH₃ | H | Cl | 1 | isomer1 |
| 4-218 | Q4 | cyclopropyl | CH₂CH₂CH₃ | H | Cl | 1 | isomer2 |
| 4-219 | Q4 | cyclopropyl | CH₂CH₂CH₃ | H | Cl | 2 | — |
| 4-220 | Q4 | cyclopropyl | CH(CH₃)₂ | H | Cl | 0 | — |
| 4-221 | Q4 | cyclopropyl | CH(CH₃)₂ | H | Cl | 1 | racemate |
| 4-222 | Q4 | cyclopropyl | CH(CH₃)₂ | H | Cl | 1 | isomer1 |
| 4-223 | Q4 | cyclopropyl | CH(CH₃)₂ | H | Cl | 1 | isomer2 |
| 4-224 | Q4 | cyclopropyl | CH(CH₃)₂ | H | Cl | 2 | — |
| 4-225 | Q4 | cyclopropyl | CH₂CH₂CH₂CH₃ | H | Cl | 0 | — |
| 4-226 | Q4 | cyclopropyl | CH₂CH₂CH₂CH₃ | H | Cl | 1 | racemate |
| 4-227 | Q4 | cyclopropyl | CH₂CH₂CH₂CH₃ | H | Cl | 1 | isomer1 |
| 4-228 | Q4 | cyclopropyl | CH₂CH₂CH₂CH₃ | H | Cl | 1 | isomer2 |
| 4-229 | Q4 | cyclopropyl | CH₂CH₂CH₂CH₃ | H | Cl | 2 | — |
| 4-230 | Q4 | cyclopropyl | CH(CH₃)CH₂CH₃ | H | Cl | 0 | racemate |
| 4-231 | Q4 | cyclopropyl | CH(CH₃)CH₂CH₃ | H | Cl | 0 | isomer 1 |
| 4-232 | Q4 | cyclopropyl | CH(CH₃)CH₂CH₃ | H | Cl | 0 | isomer2 |
| 4-233 | Q4 | cyclopropyl | CH(CH₃)CH₂CH₃ | H | Cl | 1 | mixture |
| 4-234 | Q4 | cyclopropyl | CH(CH₃)CH₂CH₃ | H | Cl | 1 | isomer1 |
| 4-235 | Q4 | cyclopropyl | CH(CH₃)CH₂CH₃ | H | Cl | 1 | isomer2 |
| 4-236 | Q4 | cyclopropyl | CH(CH₃)CH₂CH₃ | H | Cl | 1 | isomer3 |
| 4-237 | Q4 | cyclopropyl | CH(CH₃)CH₂CH₃ | H | Cl | 1 | isomer4 |
| 4-238 | Q4 | cyclopropyl | CH(CH₃)CH₂CH₃ | H | Cl | 2 | racemate |
| 4-239 | Q4 | cyclopropyl | CH(CH₃)CH₂CH₃ | H | Cl | 2 | isomer1 |
| 4-240 | Q4 | cyclopropyl | CH(CH₃)CH₂CH₃ | H | Cl | 2 | isomer2 |
| 4-241 | Q4 | cyclopropyl | CH₂CH(CH₃)₂ | H | Cl | 0 | — |
| 4-242 | Q4 | cyclopropyl | CH₂CH(CH₃)₂ | H | Cl | 1 | racemate |
| 4-243 | Q4 | cyclopropyl | CH₂CH(CH₃)₂ | H | Cl | 1 | isomer1 |
| 4-244 | Q4 | cyclopropyl | CH₂CH(CH₃)₂ | H | Cl | 1 | isomer2 |
| 4-245 | Q4 | cyclopropyl | CH₂CH(CH₃)₂ | H | Cl | 2 | — |
| 4-246 | Q4 | cyclopropyl | cyclopropylmethyl | H | Cl | 0 | — |
| 4-247 | Q4 | cyclopropyl | cyclopropylmethyl | H | Cl | 1 | racemate |
| 4-248 | Q4 | cyclopropyl | cyclopropylmethyl | H | Cl | 1 | isomer1 |
| 4-249 | Q4 | cyclopropyl | cyclopropylmethyl | H | Cl | 1 | isomer2 |
| 4-250 | Q4 | cyclopropyl | cyclopropylmethyl | H | Cl | 2 | — |
| 4-251 | Q4 | cyclopropyl | cyclopropyl | H | Cl | 0 | — |
| 4-252 | Q4 | cyclopropyl | cyclopropyl | H | Cl | 1 | racemate |
| 4-253 | Q4 | cyclopropyl | cyclopropyl | H | Cl | 1 | isomer1 |
| 4-254 | Q4 | cyclopropyl | cyclopropyl | H | Cl | 1 | isomer2 |
| 4-255 | Q4 | cyclopropyl | cyclopropyl | H | Cl | 2 | — |
| 4-256 | Q4 | cyclopropyl | cyclobutyl | H | Cl | 0 | — |
| 4-257 | Q4 | cyclopropyl | cyclobutyl | H | Cl | 1 | racemate |
| 4-258 | Q4 | cyclopropyl | cyclobutyl | H | Cl | 1 | isomer1 |
| 4-259 | Q4 | cyclopropyl | cyclobutyl | H | Cl | 1 | isomer2 |
| 4-260 | Q4 | cyclopropyl | cyclobutyl | H | Cl | 2 | — |
| 4-261 | Q4 | cyclopropyl | cyclopentyl | H | Cl | 0 | — |
| 4-262 | Q4 | cyclopropyl | cyclopentyl | H | Cl | 1 | racemate |
| 4-263 | Q4 | cyclopropyl | cyclopentyl | H | Cl | 1 | isomer1 |
| 4-264 | Q4 | cyclopropyl | cyclopentyl | H | Cl | 1 | isomer2 |
| 4-265 | Q4 | cyclopropyl | cyclopentyl | H | Cl | 2 | — |
| 4-266 | Q4 | cyclopropyl | cyclohexyl | H | Cl | 0 | — |
| 4-267 | Q4 | cyclopropyl | cyclohexyl | H | Cl | 1 | racemate |
| 4-268 | Q4 | cyclopropyl | cyclohexyl | H | Cl | 1 | isomer1 |
| 4-269 | Q4 | cyclopropyl | cyclohexyl | H | Cl | 1 | isomer2 |
| 4-270 | Q4 | cyclopropyl | cyclohexyl | H | Cl | 2 | — |
| 4-271 | Q4 | CH₃ | CH₂CH₂CH₃ | CH₃ | Cl | 0 | — |
| 4-272 | Q4 | CH₃ | CH₂CH₂CH₃ | CH₃ | Cl | 1 | racemate |
| 4-273 | Q4 | CH₃ | CH₂CH₂CH₃ | CH₃ | Cl | 1 | isomer1 |
| 4-274 | Q4 | CH₃ | CH₂CH₂CH₃ | CH₃ | Cl | 1 | isomer2 |
| 4-275 | Q4 | CH₃ | CH₂CH₂CH₃ | CH₃ | Cl | 2 | — |
| 4-276 | Q4 | CH₃ | CH(CH₃)₂ | CH₃ | Cl | 0 | — |
| 4-277 | Q4 | CH₃ | CH(CH₃)₂ | CH₃ | Cl | 1 | racemate |
| 4-278 | Q4 | CH₃ | CH(CH₃)₂ | CH₃ | Cl | 1 | isomer1 |
| 4-279 | Q4 | CH₃ | CH(CH₃)₂ | CH₃ | Cl | 1 | isomer2 |
| 4-280 | Q4 | CH₃ | CH(CH₃)₂ | CH₃ | Cl | 2 | — |
| 4-281 | Q4 | CH₃ | CH₂CH₂CH₃ | CH₂CH₃ | Cl | 0 | — |
| 4-282 | Q4 | CH₃ | CH₂CH₂CH₃ | CH₂CH₃ | Cl | 1 | racemate |
| 4-283 | Q4 | CH₃ | CH₂CH₂CH₃ | CH₂CH₃ | Cl | 1 | isomer1 |
| 4-284 | Q4 | CH₃ | CH₂CH₂CH₃ | CH₂CH₃ | Cl | 1 | isomer2 |
| 4-285 | Q4 | CH₃ | CH₂CH₂CH₃ | CH₂CH₃ | Cl | 2 | — |
| 4-286 | Q4 | CH₃ | CH(CH₃)₂ | CH₂CH₃ | Cl | 0 | — |
| 4-287 | Q4 | CH₃ | CH(CH₃)₂ | CH₂CH₃ | Cl | 1 | racemate |
| 4-288 | Q4 | CH₃ | CH(CH₃)₂ | CH₂CH₃ | Cl | 1 | isomer1 |
| 4-289 | Q4 | CH₃ | CH(CH₃)₂ | CH₂CH₃ | Cl | 1 | isomer2 |
| 4-290 | Q4 | CH₃ | CH(CH₃)₂ | CH₂CH₃ | Cl | 2 | — |
| 4-291 | Q4 | CH₃ | cyclobutyl | CH₂CH₂OCH₃ | Cl | 0 | — |
| 4-292 | Q4 | CH₃ | cyclobutyl | CH₂CH₂OCH₃ | Cl | 1 | racemate |
| 4-293 | Q4 | CH₃ | cyclobutyl | CH₂CH₂OCH₃ | Cl | 1 | isomer 1 |
| 4-294 | Q4 | CH₃ | cyclobutyl | CH₂CH₂OCH₃ | Cl | 1 | isomer2 |
| 4-295 | Q4 | CH₃ | cyclobutyl | CH₂CH₂OCH₃ | Cl | 2 | — |
| 4-296 | Q4 | CH₃ | cyclobutylmethyl | H | Cl | 0 | — |
| 4-297 | Q4 | CH₃ | cyclobutylmethyl | H | Cl | 1 | racemate |
| 4-298 | Q4 | CH₃ | cyclobutylmethyl | H | Cl | 1 | isomer1 |
| 4-299 | Q4 | CH₃ | cyclobutylmethyl | H | Cl | 1 | isomer2 |
| 4-300 | Q4 | CH₃ | cyclobutylmethyl | H | Cl | 2 | — |
| 4-301 | Q4 | CH₃ | cyclopentylmethyl | H | Cl | 0 | — |
| 4-302 | Q4 | CH₃ | cyclopentylmethyl | H | Cl | 1 | racemate |
| 4-303 | Q4 | CH₃ | cyclopentylmethyl | H | Cl | 1 | isomer1 |
| 4-304 | Q4 | CH₃ | cyclopentylmethyl | H | Cl | 1 | isomer2 |
| 4-305 | Q4 | CH₃ | cyclopentylmethyl | H | Cl | 2 | — |
| 4-306 | Q4 | CH₃ | 3,3-difluorocyclobutyl | H | Cl | 0 | — |
| 4-307 | Q4 | CH₃ | 3,3-difluorocyclobutyl | H | Cl | 1 | racemate |
| 4-308 | Q4 | CH₃ | 3,3-difluorocyclobutyl | H | Cl | 1 | isomer1 |
| 4-309 | Q4 | CH₃ | 3,3-difluorocyclobutyl | H | Cl | 1 | isomer2 |
| 4-310 | Q4 | CH₃ | 3,3-difluorocyclobutyl | H | Cl | 2 | — |

The present invention also provides a first method of preparing said 4-methylsulfonylbenzamide analogs, stereoisomers thereof, or agriculturally acceptable salts thereof, comprising the following steps:
(1) conducting a substitution reaction of a compound of formula (VI) with different substituted thiols or thiolates to give a compound of formula (V);
(2) conducting a condensation reaction between the compound of formula (V) and a compound of formula (IV) to give a compound of formula (III);
(3) conducting an oxidation reaction of the compound of formula (III) to give a compound of formula (II); and
(4) conducting a substitution reaction of the compound of formula (II) to give the 4-methylsulfonylbenzamide compound of formula (I);

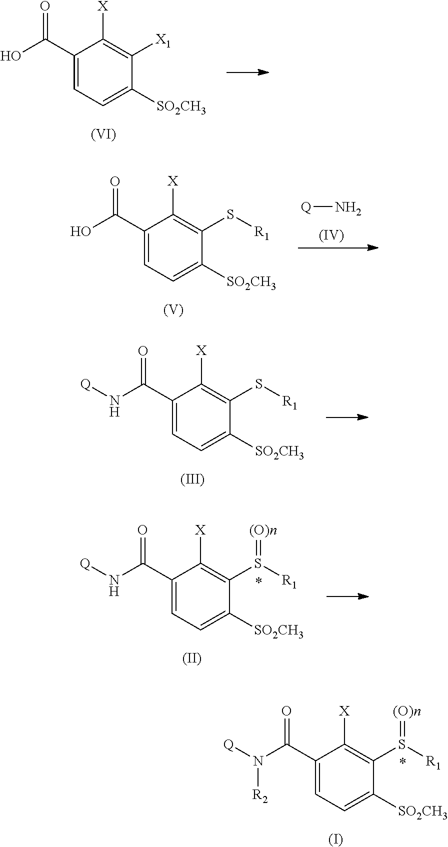

wherein
Xᵢ is selected from fluorine or chlorine;
R₁, R₂, X, and Q are defined as before, and n represents 1 or 2.

The present invention also provides a second method of preparing said 4-methylsulfonylbenzamide analogs, their stereoisomers, or agriculturally acceptable salts thereof, comprising the following steps:
(1) conducting a substitution reaction of a compound of formula (VI) with hydrosulfide salt to give a thiophenol compound of formula (VII);
(2) conducting a substitution reaction of the compound of formula (VII) with a compound of formula (VIII) to give a compound of formula (V);
(3) conducting a condensation reaction between the compound of formula (V) and a compound of formula (IV) to give a compound of formula (III);
(4) conducting an oxidation reaction of the compound of formula (III) to give a compound of formula (II); and
(5) conducting a substitution reaction of the compound of formula (II) to give the 4-methylsulfonylbenzamide compound of formula (I);

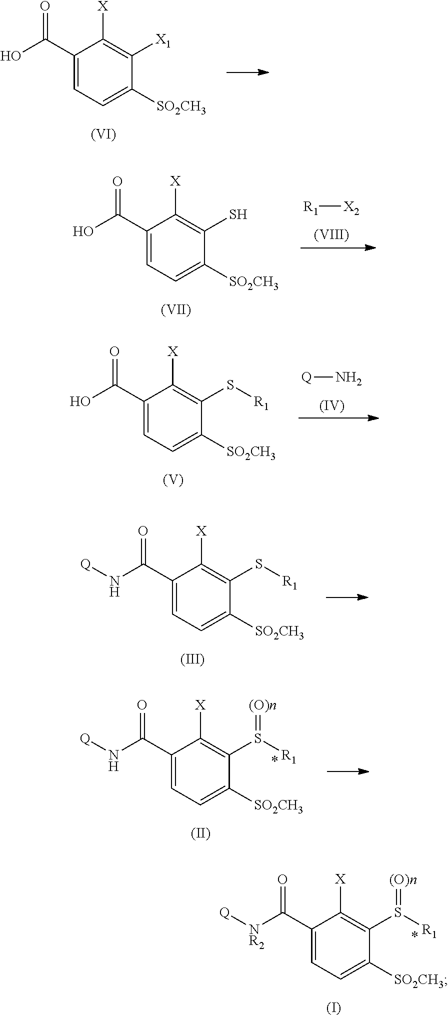

wherein
X₁ is selected from fluorine or chlorine;
X₂ is selected from chlorine, bromine, iodine, MsO (methanesulfonyloxy), TfO (trifluoromethanesulfonyloxy) or TsO (p-toluenesulfonyloxy);
R₁, R₂, X, and Q have the meaning as mentioned above.

The present invention also provides a herbicidal composition, the comprising at least one of the 4-methylsulfonylbenzamide compound, stereoisomer thereof or salt thereof according to anyone of claims 1-3 with the compound of formula (I) as an active ingredient, wherein the weight percentage of the active ingredient in the composition is 0.1% to 99.9%. Preferably, one or more additional herbicides and/or safeners. More preferably, agrochemically acceptable formulation aids.

The present invention also provides a method of controlling weeds comprising applying a herbicidally effective amount of said 4-methanesulfonylbenzamides, stereoisomers thereof, or agriculturally acceptable salts thereof, or said herbicidal compositions, to a plant or to a weed area.

The present invention also provides the use of said 4-methylsulfonylbenzamides, stereoisomers thereof or agriculturally acceptable salts thereof or said herbicidal compositions for the control of weeds.

The present invention also provides the use of said 4-methylsulfonylbenzamide analogs, stereoisomers thereof, or agriculturally acceptable salts thereof, or said herbicidal compositions for controlling weeds.

The present invention also provides the use of said 4-methylsulfonylbenzamides, stereoisomers thereof, or agriculturally acceptable salts thereof, or said herbicidal compositions for the control of weeds in a useful crop, said useful crop being a genetically modified crop or a genome editing technology treated crop.

The inventive compounds of the formula (I) (and/or salts thereof), referred to collectively as “compounds of the invention” hereinafter, have excellent herbicidal efficacy against a broad spectrum of economically important monocotyledonous and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

The compounds of the invention can be selective in crops of useful plants and can also be employed as nonselective herbicides.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) or salts thereof in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What has been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377A) or of the sulfonylurea type (EP 0257993 A, U.S. Pat. No. 5,013,659) or to combinations or mixtures of these herbicides through “gene stacking’, such as transgenic crop plants, for example corn or soya with the trade name or the designation Optimum™ GAT™ (Glyphosate ALS Tolerant),
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A),
genetically modified plants having reduced photorespiration, which have higher yields and higher stress tolerance (EP 0305398 A),
transgenic crop plants which produce pharmaceutically or diagnostically important proteins (‘molecular pharming”),
transgenic crop plants which feature higher yields or better quality,
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. For the connection of the DNA fragments to one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, NY; or Winnacker “Gene and Klone”, VCH Weinheim, 2nd edition, 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Thus, transgenic plants can be obtained whose properties are altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients, or to any desired combitions of these active ingredients.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as corn or soya with the trade name or the designation Optimum™ GAT™ (glyphosate ALS tolerant), for example.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Ktichler, “Chemische Technologic” [Chemical Technology], Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The necessary formulation assistants, such as inert materials, surfactants, solvents and further additives, are likewise known and are described, for example, in: Watkins, “Handbook of Insecticide Dust Diluents and Carriers”, 2nd ed., Darland Books, Caldwell N.J., H. v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schonfeldt, “Grenzflachenaktive Athylenoxidaddukte” [Interface-active Ethylene Oxide Adducts], Wiss. Verlagsgesell., Stuttgart 1976, WinnackerKéchler, “Chemische Technologic”, Volume 7, C. Hanser Verlag Munich, 4th ed. 1986.

On the basis of these formulations, it is also possible to produce combinations with other active ingredients, for example insecticides, acaricides, herbicides, fungicides, and also with safeners, fertilizers and/or growth regulators, for example in the form of a finished formulation or as a tank mix.

Active ingredients which can be employed in combination with the compounds of the invention in mixed formulations or in a tank mix are, for example, known active ingredients which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as described, for example, in Weed Research 26 (1986) 441-445 or “The Pesticide Manual”, 16th edition, The British Crop Protection Council and the Royal Soc. of Chemistry, 2006 and the literature cited therein. Known herbicides or plant growth regulators which can be combined with the compounds of the invention are, for example, the following, where said active ingredients are designated either with their “common name” in accordance with the International Organization for Standardization (ISO) or with the chemical name or with the code number. They always encompass all the use forms, for example acids, salts, esters and also all isomeric forms such as stereoisomers and optical isomers, even if they are not mentioned explicitly.

Examples of Such Herbicidal Mixing Partners are:
acetochlor, acifluorfen, acifluorfen-sodium, aclonifen, alachlor, allidochlor, alloxydim, alloxydim-sodium, ametryn, amicarbazone, amidochlor, amidosulfuron, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methy]l, bensulide, bentazone, benzobicy don, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, 3-[5-chloro-4-(trifluoromethyl)pyridin-2-yl]-4-hydroxy-1-methylimidazolidin-2-one, cinidon, cinidon-ethyl, cinmethy lin, cinosulfuron, clacyfos, clethodim, clodinafop, clodinafoppropargyl, clomazone, clomeprop, clopyralid, cloransulam, cloransulam-methyl, cumyluron, cyanamide, cyanazine, cycloate, cyclopyranil, cyclopyrimorate, cyclosulfamuron, cycloxydim, cyhalofop, cyhalofop-butyl, cyprazine, 2,4-D, 2,4-D-butotyl, -butyl, -dimethylammonium, -diolamin, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimetham etryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-G-fluoropropy])-4,5-dihydro-5-oxo-1H-tetrazol-1-yl]-phenyl]ethanesulfonamide, F-7967, i.e. 3-[7-chloro-5-fluoro-2-(trifluoromethy])-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-Pethyl, fenoxasulfone, fenquinotrione, fentrazamide, flamprop, flamprop-M-isopropyl], flamprop-M-methyl, flazasulfuron, florasulam, florpyrauxifen, florpyrauxifen-benzyl, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)-O-ethyl isopropylphosphoramidothioate, halauxifen, halauxifen-methy], halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphorylethyl (2,4-dichlorophenoxy)acetate, 4-hydroxy-1-methoxy-5-methyl-3-[4-(trifluoromethyl)pyridin-2-yl]imidazolidin-2-one, 4-hydroxy-1-methyl-3-[4-(trifluoromethy])pyridin-2-yl]imidazolidin-2-one, imazamethabenz, imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, ie. 3-({[5-(difluoromethyl)-1-methyl-3-(trifluoromethyl)-1Hpyrazol-4-yl]methyl}sulfonyl)-5,5-dimethyl-4,5-dihydro-1, 2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPBmethyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, ie. N-[3-chloro-4-(1-methylethy])phenyl|]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxotrione (lancotrione), oxyfluorfen, paraquat, paraquat dichloride, pebulate, pendimethalin, penoxsulam, pentachlorophenol, pentoxazone, pethoxamid, petroleum oils, phenmedipham, picloram, picolinafen, pinoxaden, piperophos, pretilachlor, primisulfuron, primisulfuron-methy]l, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, sulcotrion, sulfentrazone, sulfometuron, sulfometuron-methy], sulfosulfuron, SYN-523, SYP-249, i.e. 1-ethoxy-3-methyl-1-oxobut-3-en2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methy]l, tritosulfuron, urea sulfate, vernolate, 7J-0862, i.e. 3,4-dichloro-N-{2-[(4,6-dimethoxypyrimidin-2-yloxy]benzyl}aniline, and the following compounds:

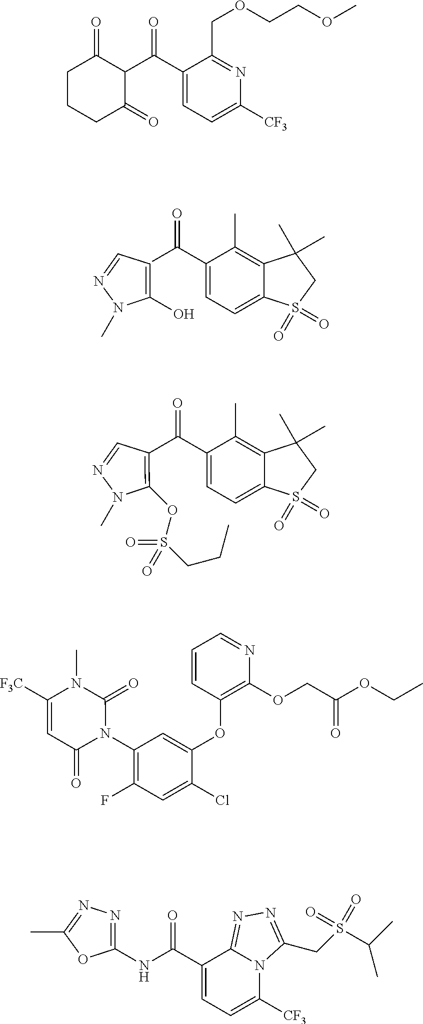

In the context of the present description, if an abbreviation of a generic name of active compound is used, it includes in each case all conventional derivatives thereof, such as esters and salts as well as isomers, in particular optical isomers, in particular one or more commercially available forms thereof. If the generic name denotes an ester or a salt, it also includes in each case all other conventional derivatives, such as other esters and salts, free acids and neutral compounds, as well as isomers, in particular optical isomers, in particular one or more commercially available forms thereof. The chemical name given to a compound means at least one compound encompassed by the generic name, and generally the preferred compound.

For use, the formulations which are present in commercially available form are, if appropriate, diluted in the customary manner, for example using water in the case of wettable powders, emulsifiable concentrates, dispersions and water-dispersible granules. Products in the form of dusts, granules for soil application or broadcasting and sprayable solutions are usually not further diluted with other inert substances prior to use. The application rate of the compounds of the formula (I) required varies with the external conditions, such as temperature, humidity, the nature of the herbicide used and the like. It can vary within wide limits, for example between 0.001 and 1.0 kg a.i/ha or more of active substance, but it is preferably between 0.005 and 750 g a.i/ha, in particular between 0.005 and 500 g a.i./ha.

The term “aryl” refers to a 6 to 14 membered all-carbon monocyclic ring or polycyclic fused ring (i.e. each ring in the system shares an adjacent pair of carbon atoms with another ring in the system) having a conjugated π-electron system, preferably a 6 to 10 membered aryl, for example, phenyl and naphthyl. The aryl is more preferably phenyl. The aryl ring can be fused to the ring of heteroaryl, heterocyclyl or cycloalkyl, wherein the ring bound to the parent structure is aryl ring. The aryl can be substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, carboxy and alkoxycarbonyl.

The term “heteroaryl” refers to a 5 to 14 membered heteroaromatic system having 1 to 4 heteroatoms selected from the group consisting of oxygen, sulfur and nitrogen. The heteroaryl is preferably a 5 to 10 membered heteroaryl, more preferably a 5 or 6 membered heteroaryl, for example imidazolyl, furanyl, thienyl, thiazolyl, pyrazolyl, oxazolyl, pyrrolyl, triazolyl, tetrazolyl, pyridyl, pyrimidinyl, thiadiazolyl, oxadiazolyl, pyrazinyl and the like, preferably oxazolyl, oxadiazolyl, tetrazolyl, triazolyl, thienyl, imidazolyl, pyridyl, pyrazolyl, pyrimidinyl and thiazolyl, and more preferably oxazolyl, oxadiazolyl, tetrazolyl, triazolyl, thienyl, pyridyl, thiazolyl and pyrimidinyl. The heteroaryl ring can be fused to the ring of aryl, heterocyclyl or cycloalkyl, wherein the ring bound to the parent structure is heteroaryl ring. The heteroaryl can be optionally substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, carboxy and alkoxycarbonyl.

The term “cycloalkyl” refers to a saturated or partially unsaturated monocyclic or polycyclic hydrocarbon substituent group having 3 to 20 carbon atoms, preferably 3 to 8 carbon atoms, more preferably 3 to 6 carbon atoms. Non-limiting examples of monocyclic cycloalkyl include cyclopropyl, cyclobutyl, cyclopentyl, cyclopentenyl, cyclohexyl, cyclohexenyl, cyclohexadienyl, cycloheptyl, cycloheptatrienyl, cyclooctyl and the like. Polycyclic cycloalkyl includes a cycloalkyl having a spiro ring, fused ring or bridged ring. The cycloalkyl is preferably cyclopropyl, cyclobutyl, cyclohexyl, cyclopentyl and cycloheptyl. The cycloalkyl ring can be fused to the ring of aryl, heteroaryl or heterocyclyl, wherein the ring bound to the parent structure is cycloalkyl. Non-limiting examples include indanyl, tetrahydronaphthyl, benzocycloheptyl and the like. The cycloalkyl can be optionally substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy and alkoxycarbonyl.

The cycloalkyl ring can be fused to the ring of aryl, heteroaryl or heterocyclyl, wherein the ring bound to the parent structure is cycloalkyl. Non-limiting examples include indanyl, tetrahydronaphthyl, benzocycloheptyl and the like. The cycloalkyl can be optionally substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy and alkoxycarbonyl.

The term “heterocyclyl” refers to a 3 to 20 membered saturated or partially unsaturated monocyclic or polycyclic hydrocarbon group, wherein one or more ring atoms are heteroatoms selected from the group consisting of nitrogen, oxygen and S(O)ₘ (wherein m is an integer of 0 to 2), but excluding —O—O—, —O—S— or —S—S— in the ring, with the remaining ring atoms being carbon atoms. Preferably, the heterocyclyl has 3 to 12 ring atoms wherein 1 to 4 atoms are heteroatoms; more preferably, 3 to 8 ring atoms; and most preferably 3 to 8 ring atoms. Non-limiting examples of monocyclic heterocyclyl include oxetanyl, pyrrolidinyl, pyrrolidonyl, imidazolidinyl, tetrahydrofuranyl, tetrahydrothienyl, dihydroimidazolyl, dihydrofuranyl, dihydropyrazolyl, dihydropyrrolyl, piperidinyl, piperazinyl, morpholinyl, thiomorpholinyl, homopiperazinyl, pyranyl and the like, preferably oxetanyl, pyrrolidonyl, tetrahydrofuranyl, pyrazolidinyl, morpholinyl, piperazinyl and pyranyl. Polycyclic heterocyclyl includes a heterocyclyl having a spiro ring, fused ring or bridged ring. The heterocyclyl having a spiro ring, fused ring or bridged ring is optionally bonded to other group via a single bond, or further bonded to other cycloalkyl, heterocyclyl, aryl and heteroaryl via any two or more atoms on the ring. The heterocyclyl can be optionally substituted or unsubstituted. When substituted, the substituent group(s) is preferably one or more group(s) independently selected from the group consisting of alkyl, alkenyl, alkynyl, alkoxy, alkylthio, alkylamino, halogen, thiol, hydroxy, nitro, cyano, cycloalkyl, heterocyclyl, aryl, heteroaryl, cycloalkoxy, heterocycloalkoxy, cycloalkylthio, heterocyclylthio, oxo, carboxy and alkoxycarbonyl.

In the present invention, if any discrepancy exists between the nomenclature of the compounds and their structural formulas, the structural formulas shall prevail, unless the structural formulas contain obvious errors.

The advantageous effects of the invention lie in providing a class of 4-methanesulfonylbenzamide compounds with sulfur-containing substituents at the 3-position, their preparation methods, herbicidal compositions, and applications. These compounds demonstrate superior herbicidal activity and enhanced crop safety, particularly exhibiting excellent selectivity for key crops such as wheat, rice, and Zea mays. Furthermore, the preparation method for the herbicide of this invention is simple to operate, cost-efficient, and suitable for large-scale industrial production.

### DETAILED DESCRIPTION

The following Examples are provided to illustrate the invention and should not be construed as limiting its scope in any manner. The scope of protection sought by the invention is defined in the claims. Modifications or improvements made by those skilled in the art without departing from the inventive concept of the invention shall fall within the scope of the technical solutions protected by the invention.

Several methods for preparing the compounds of the invention are described in detail in the following schemes and Examples. The raw materials may be commercially available, prepared using methods documented in the literature, or synthesized as described herein. Skilled artisans will recognize that alternative synthetic routes may also be employed to produce the compounds of the invention. While specific raw materials and reaction conditions are outlined below, substitutions with analogous materials and conditions are permissible. Variations or modifications to the preparation methods (e.g., generating isomers of the compounds) are encompassed within the scope of the invention. Additionally, the described preparation methods may be further refined using conventional chemical techniques known in the art, such as protecting functional groups during reactions.

The method Examples provided below are intended to facilitate understanding of the preparation processes. The specific materials, types, and conditions employed are illustrative and not intended to restrict the reasonable scope of the invention. Reagents used in the synthesis of the compounds may be commercially sourced or readily prepared by those of ordinary skill in the art.

### Analytical Instruments Used in the Examples

### 1. High-Performance Liquid Chromatography (HPLC):

Instrument: Agilent Technologies, 1260 Infinity II
Column: Agilent Eclipse Plus C18, 3.5 μm, 4.6×100 mm
Mobile phase: A: Water+0.1% phosphoric acid; B: Acetonitrile
Temperature: 30° C.
Gradient: 10% B to 95% B over 15 min; hold at 95% B for 3 min
Flow rate: 1 mL/min

### 2. Ultra-Performance Liquid Chromatography-Tandem Mass Spectrometry (LC-MS):

Instrument: Waters ACQUITY H-Class UPLC-SQ Detector 2
Column: ACQUITY UPLC® BEH C18, 1.7 μm, 2.1×50 mm
Mobile phase: A: Water+0.2% formic acid; B: Acetonitrile
Temperature: 30° C.
Gradient: 10% B to 95% B over 5 min; hold at 95% B for 1 min
Flow rate: 0.5 mL/min
MS parameters: ESI positive/negative mode; mass range: m/z 100-800

### 3. Gas Chromatography-Tandem Mass Spectrometry (GC-MS):

Instrument: Agilent Technologies 7890B GC System-5977A MSD
Column: Agilent HP-5MS UI, 0.25 μm, 30 μm×0.250 mm
Injector temperature: 250° C.
Column flow rate: Helium, 1 mL/min
Method: Hold at 40° C. for 2 min; ramp at 20° C./min to 280° C.; hold at 280° C. for 5 min (total run time: 19 min)
MSD transfer line temperature: 280° C.
EI ion source temperature: 230° C.; MS quadrupole temperature: 150° C.; scan range: m/z 30.00-400.00

### Proton Nuclear Magnetic Resonance (¹H-NMR) Analysis

¹H-NMR chemical shifts (8) are reported relative to tetramethylsilane (TMS) as an internal standard. Measurements were conducted in deuterated chloroform (CDCl₃) using a Bruker AVANCE III HD 400 MHz spectrometer. For spectra acquired in deuterated dimethyl sulfoxide (DMSO-d₆), the solvent is explicitly noted as “(DMSO-d₆).” The abbreviations for peak multiplicity are defined as follows:
s: singlet, d: doublet, dd: double doublet, dt: double triplet, td: triple doublet, ddd: double double doublet, t: triplet, q: quartet, sep: septet, m: multiplet, brs: broad singlet For compounds with two or more stereoisomers, resolvable signals are annotated with “and” to denote distinct chemical shifts for each isomer.

### Representative Examples

Synthetic procedures for representative compounds are provided below. Methods for other compounds follow analogous protocols and are not elaborated herein.

### Intermediate 1

### 2-chloro-3-mercapto-4-(methylsulfonyl)benzoic acid

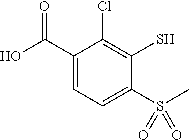

At room temperature, compound 2-chloro-3-fluoro-4-(methylsulfonyl)benzoic acid (40.00 g, 0.16 mol) and 1-methylpyrrolidin-2-one (400 mL) were added to a 1 L reaction flask. The temperature was raised to 55° C., and sodium hydrogen sulfide (63.4 g, 0.79 mol) was added in batches. After the addition was complete, the reaction was continued for 0.5 hours. Upon completion, the reaction mixture was poured into a large amount of ice water, and the pH was adjusted to 1 using hydrochloric acid. A solid precipitated, which was filtered and dried in an oven at 45° C. to yield crude Intermediate 1 (white solid, 37.60 g). This crude product was used directly in the next step without further purification. A portion of the crude Intermediate 1 mixture was subjected to reverse-phase high-pressure preparation to obtain NMR data:

¹H NMR (400 MHz, CD₃CN-d₃) δ 8.07 (d, J=8.2 Hz, 1H), 7.67 (d, J=8.3 Hz, 1H), 5.65 (brs, 1H), 3.25 (s, 3H).

### Intermediate 2

### 2-chloro-4-(methylsulfonyl)-3-(propylthio)benzoic acid

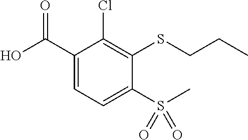 [SMILES: CCCSc1c(S(C)(=O)=O)ccc(C(=O)O)c1Cl]

At room temperature, compound 2,3-dichloro-4-(methylsulfonyl)benzoic acid (200 mg, 0.74 mmol), potassium hydroxide (166 mg, 2.96 mmol), cesium carbonate (968 mg, 4.91 mmol), dimethyl sulfoxide (4 mL), tetrabutylammonium bromide (15 mg, 0.05 mmol), and potassium iodide (15 mg, 0.09 mmol) were added to a 50 mL single-neck flask. The mixture was cooled in an ice bath to 5° C., and propanethiol (200 mg, 2.63 mmol) was slowly added dropwise. After the addition was completed, the reaction was stirred at room temperature overnight. Upon completion of the reaction, 5% dilute hydrochloric acid was added dropwise to adjust the pH to 1-2. The mixture was extracted twice with 10 mL of ethyl acetate, and the combined organic phases were washed once with saturated brine. After drying over anhydrous sodium sulfate, the solvent was removed under reduced pressure to afford the crude Intermediate 2 (yellow solid, 210 mg), which was used directly in the next step without further purification. A portion of the crude Intermediate 2 was subjected to reverse-phase high-pressure preparation to obtain NMR data:
1H NMR (400 MHz, DMSO-d6) δ 8.09 (d, J=7.0 Hz, 1H), 7.89 (d, J=7.0 Hz, 1H), 3.55 (s, 3H), 2.97 (t, J=7.4 Hz, 2H), 1.61 (q, J=7.4 Hz, 2H), 0.97 (t, J=7.3 Hz, 3H).

### Intermediate 3

### 2-chloro-4-(methylsulfonyl)-3-(propylsulfinyl)benzoic acid

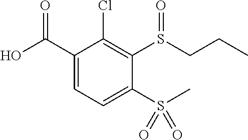 [SMILES: CCCS(=O)c1c(S(C)(=O)=O)ccc(C(=O)O)c1Cl]

At room temperature, Intermediate 2 (200 mg, 0.65 mmol) and dichloromethane (5 mL) were added to a 25 mL single-neck flask. The mixture was cooled in an ice bath to 5° C., and 85% m-chloroperbenzoic acid (131.49 mg, 0.65 mmol) was added in portions. After completion of the addition, the reaction was stirred in the ice bath for 30 minutes. Upon completion of the reaction, the mixture was filtered to remove solid impurities and washed with saturated sodium sulfite solution to eliminate peroxides. The solvent was removed under reduced pressure, and the crude product was purified by flash column chromatography on silica gel to afford Intermediate 3 (white solid, 68 mg).

¹H NMR (400 MHz, DMSO-d₆) δ 8.08 (d, J=8.1 Hz, 1H), 8.00 (d, J=8.1 Hz, 1H), 3.87-3.65 (m, 1H), 3.51 (s, 3H), 3.12-3.01 (m, 1H), 1.91-1.69 (m, 2H), 1.07 (t, J=7.4 Hz, 3H).

### Intermediate 4

### 2-chloro-4-(methylsulfonyl)-3-(propylsulfonyl)benzoic acid

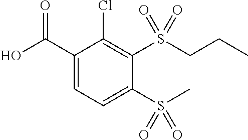 [SMILES: CCCS(=O)(=O)c1c(S(C)(=O)=O)ccc(C(=O)O)c1Cl]

At room temperature, Intermediate 2 (200 mg, 0.65 mmol), acetic acid (5 mL), and sodium tungstate (9.55 mg, 0.03 mmol) were added to a 25 mL single-necked flask. The mixture was heated to 90° C., and 30% hydrogen peroxide (1473.33 mg, 13 mmol) was added dropwise. After completion of the addition, the reaction was stirred at 90° C. for 2 hours. Upon completion, the solvent was removed under reduced pressure. The residue was dissolved in 10 mL ethyl acetate, dried over anhydrous sodium sulfate, filtered, and concentrated under reduced pressure to afford Intermediate 4 crude product (white solid, 205 mg), which was used directly in the next step without further purification. A portion of the Intermediate 4 crude mixture was subjected to reverse-phase high-pressure preparative chromatography to obtain NMR data:

¹H NMR (400 MHz, DMSO-d₆) δ 8.29 (d, J=8.2 Hz, 1H), 8.08 (d, J=8.2 Hz, 1H), 3.71-3.64 (m, 2H), 3.61 (s, 3H), 1.96-1.82 (m, 2H), 1.04 (t, J=7.4 Hz, 3H).

### Intermediate 5

### 2-chloro-3-(isopropylthio)-4-(methylsulfonyl)benzoic acid

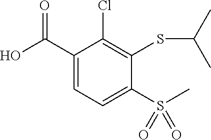 [SMILES: CC(C)Sc1c(S(C)(=O)=O)ccc(C(=O)O)c1Cl]

At room temperature, 2-chloro-3-fluoro-4-(methylsulfonyl)benzoic acid (3.50 g, 13.85 mmol), dimethyl sulfoxide (DMSO, 30 mL), and potassium hydroxide (2.30 g, 40.99 mmol) were added to a 100 mL single-necked flask. The mixture was cooled to 10° C. in an ice bath, and 2-propanethiol (2.10 g, 27.58 mmol) was added dropwise. After completion of the addition, the reaction was stirred at room temperature for 0.5 hours. Upon completion, 60 mL water was added, and the mixture was extracted with ethyl acetate (20 mL×2). The combined organic phases were washed with saturated brine, and the solvent was removed under reduced pressure to afford Intermediate 5 crude product (yellow solid, 4.14 g), which was used directly in the next step without further purification. A portion of the Intermediate 5 crude mixture was subjected to reverse-phase high-pressure preparative chromatography to obtain NMR data:

¹H NMR (400 MHz, DMSO-d₆) δ 8.17 (d, J=8.1 Hz, 1H), 7.94 (d, J=8.2 Hz, 1H), 3.84 (p, J=6.7 Hz, 1H), 3.59 (s, 3H), 1.28 (d, J=6.7 Hz, 6H).

### Intermediate 29

### methyl 2-chloro-3-mercapto-4-(methylsulfonyl)benzoate

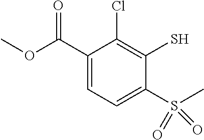

At room temperature, Intermediate 1 (5 g, 18.75 mmol) and methanol (150 mL) were added to a 250 mL single-necked flask. The mixture was cooled to −10° C., and concentrated sulfuric acid (5 mL) was slowly added dropwise. After completion of the addition, the temperature was raised to 60° C., and the reaction was stirred under reflux overnight. Upon completion, the reaction mixture was poured into 200 mL of ice-water, filtered, and dried to afford crude Intermediate 29 (off-white solid, 4.70 g), which was used directly in the next step without further purification.

### Intermediate 30

### methyl 2-chloro-3-(cyclopropylthio)-4-(methylsulfonyl)benzoate

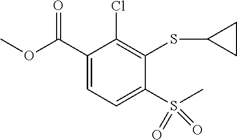 [SMILES: COC(=O)c1ccc(S(C)(=O)=O)c(SC2CC2)c1Cl]

At room temperature, Intermediate 29 (5.00 g, 17.81 mmol), 1,2-dichloroethane (45 mL), 2,2′-bipyridine (3.34 g, 21.39 mmol), copper(II) acetate (3.56 g, 17.81 mmol), and cyclopropylboronic acid (3.32 g, 38.60 mmol) were added to a 125 mL pressure-resistant bottle. The mixture was heated to 70° C. and stirred for 4 hours. After completion, 200 mL of 25% aqueous ammonia was added and stirred for 15 minutes. The mixture was extracted with dichloromethane (25 mL×3), washed twice with water, filtered to remove solid impurities, and concentrated to afford crude Intermediate 30 (white solid, 4.87 g), which was used directly in the next step without further purification.

### Intermediate 31

### 2-chloro-3-(cyclopropylthio)-4-(methylsulfonyl)benzoic acid

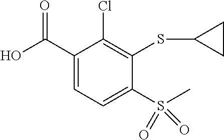 [SMILES: CS(=O)(=O)c1ccc(C(=O)O)c(Cl)c1SC1CC1]

At room temperature, Intermediate 30 (4.87 g, 14.46 mmol), tetrahydrofuran (25 mL), and water (10 mL) were added to a 50 mL single-necked flask. The mixture was cooled to 5° C., and lithium hydroxide monohydrate (1.8 g, 42.90 mmol) was slowly added. After completion of the addition, the mixture was stirred at room temperature for 2 hours. Upon completion, the solvent was removed under reduced pressure. The residue was dissolved in 30 mL of water and extracted with dichloromethane (10 mL×2). The aqueous phase was adjusted to pH 1-2 by dropwise addition of 5% dilute hydrochloric acid, dried over anhydrous sodium sulfate, extracted with ethyl acetate (15 mL×2), dried again, and concentrated to afford crude Intermediate 31 (white solid, 4.72 g), which was used directly in the next step without further purification. A portion of crude Intermediate 31 was subjected to reverse-phase high-pressure preparation for NMR analysis ¹H NMR (400 MHz, DMSO-d₆) δ 8.08 (d, J=8.2 Hz, 1H), 7.88 (d, J=8.2 Hz, 1H), 3.45 (s, 3H), 2.70-2.63 (m, 1H), 0.92-0.86 (m, 2H), 0.79-0.73 (m, 2H).

### Intermediate 32

### 2-chloro-3-(cyclopropylsulfinyl)-4-(methylsulfonyl)benzoic acid

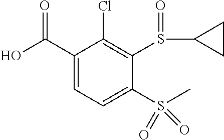 [SMILES: CS(=O)(=O)c1ccc(C(=O)O)c(Cl)c1S(=O)C1CC1]

At room temperature, Intermediate 31 (198.49 mg, 0.65 mmol), acetic acid (5 mL), and sodium tungstate dihydrate (9.55 mg, 0.03 mmol) were added to a 25 mL single-necked flask. The mixture was heated to 50° C., and 30% hydrogen peroxide (110.50 mg, 0.98 mmol) was added dropwise. After completion of the addition, the mixture was stirred at 50° C. for 2 hours. Upon completion, the solvent was removed under reduced pressure. The residue was dissolved in 10 mL of ethyl acetate, dried over anhydrous sodium sulfate, filtered, and concentrated. Reverse-phase high-pressure preparation afforded Intermediate 32 (white solid, 158.79 mg).

¹H NMR (400 MHz, DMSO-d₆) δ 8.08 (d, J=8.2 Hz, 1H), 7.89 (d, J=8.1 Hz, 1H), 3.48 (s, 3H), 3.38-3.34 (m, 1H), 1.28-1.17 (m, 2H), 1.09-0.95 (m, 2H).

### Intermediate 33

### 2-chloro-3-(cyclopropylsulfonyl)-4-(methylsulfonyl)benzoic acid

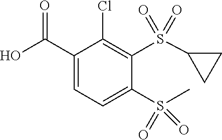 [SMILES: CS(=O)(=O)c1ccc(C(=O)O)c(Cl)c1S(=O)(=O)C1CC1]

At room temperature, Intermediate 31 (198.49 mg, 0.65 mmol) and 1,2-dichloroethane (5 mL) were added to a 25 mL single-necked flask. The mixture was heated to 50° C., and 85% m-chloroperbenzoic acid (277.13 mg, 1.36 mmol) was added in portions. After completion of the addition, the mixture was stirred for 30 minutes. Upon completion, solid impurities were removed by filtration, and the solution was washed with saturated sodium bisulfite to remove excess peroxide. The solvent was removed under reduced pressure, and purification by flash column chromatography on silica gel afforded Intermediate 33 (white solid, 158 mg).

¹H NMR (400 MHz, DMSO-d₆) δ 8.36 (d, J=8.3 Hz, 1H), 8.11 (d, J=8.3 Hz, 1H), 3.61 (s, 3H), 3.48-3.40 (m, 1H), 1.49-1.43 (m, 2H), 1.24-1.17 (m, 2H).

### Intermediate 34

### 2-chloro-3-(cyclobutylthio)-4-(methylsulfonyl)benzoic acid

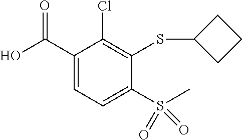 [SMILES: CS(=O)(=O)c1ccc(C(=O)O)c(Cl)c1SC1CCC1]

At room temperature, Intermediate 1 (1.50 g, 5.62 mmol), potassium carbonate (2.34 g, 16.92 mmol), and N,N-dimethylformamide (15 mL) were added to a 250 mL single-necked flask. The mixture was heated to 40° C., and bromocyclobutane (0.72 g, 5.36 mmol) was slowly added dropwise. After completion of the addition, the mixture was stirred at room temperature for 10 hours. Upon completion, 5% dilute hydrochloric acid was added dropwise to adjust the pH to 1-2. The mixture was extracted with ethyl acetate (10 mL×2), and the combined organic phase was washed once with saturated brine. The organic layer was dried over anhydrous sodium sulfate, and the solvent was removed under reduced pressure to afford crude Intermediate 34 (yellow solid, 1.91 g), which was used directly in the next step without further purification. A portion of crude Intermediate 34 was subjected to reverse-phase high-pressure preparation for NMR analysis:

¹H NMR (400 MHz, DMSO-d₆) δ 7.89 (d, J=8.2 Hz, 1H), 7.65 (d, J=8.2 Hz, 1H), 3.74 (p, J=8.2 Hz, 1H), 3.34 (s, 3H), 2.18-2.06 (m, 2H), 2.02-1.88 (m, 2H), 1.78-1.57 (m, 2H).

### Intermediate 40

### 2-chloro-3-(cyclohexylthio)-4-(methylsulfonyl)benzoic acid

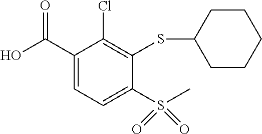 [SMILES: CS(=O)(=O)c1ccc(C(=O)O)c(Cl)c1SC1CCCCC1]

At room temperature, compound 2-chloro-3-fluoro-4-(methylsulfonyl)benzoic acid (3.50 g, 13.85 mmol), dimethyl sulfoxide (30 mL), and potassium hydroxide (2.30 g, 40.99 mmol) were added to a 100 mL single-neck flask. The mixture was cooled to 10° C. using an ice bath, followed by dropwise addition of cyclohexanethiol (3.20 g, 27.53 mmol). After completion of the addition, the reaction was stirred at room temperature for 0.5 hours. Upon completion, 60 mL of water was added, and the mixture was extracted twice with 20 mL of ethyl acetate. The combined organic phases were washed once with saturated brine, and the solvent was removed under reduced pressure to yield Intermediate 40 as a crude mixture (yellow solid, 4.30 g), which was used directly in the next step without further purification. A portion of the crude Intermediate 40 was purified by reverse-phase high-pressure preparative chromatography, and NMR data were obtained:

¹H NMR (400 MHz, DMSO-d₆) δ 8.10 (d, J=8.1 Hz, 1H), 7.86 (d, J=8.2 Hz, 1H), 3.55-3.43 (m, 4H), 1.81-1.67 (m, 4H), 1.59-1.53 (m, 1H), 1.50-1.37 (m, 2H), 1.31-1.12 (m, 3H).

### Intermediate 43

### 2-chloro-4-(methylsulfonyl)-3-((2,2,2-trifluoroethyl)thio)benzoic acid

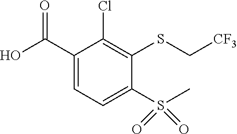 [SMILES: CS(=O)(=O)c1ccc(C(=O)O)c(Cl)c1SCC(F)(F)F]

At room temperature, Intermediate 1 (1.50 g, 5.64 mmol), potassium carbonate (2.34 g, 16.92 mmol), and N,N-dimethylformamide (15 mL) were added to a 250 mL single-neck flask. The mixture was cooled to 5° C., and 2,2,2-trifluoroethyl trifluoromethanesulfonate (1.31 g, 5.64 mmol) was slowly added dropwise. After completion of the addition, the reaction was stirred at room temperature for 2 hours. Upon completion, 5% dilute hydrochloric acid was added to adjust the pH to 1-2, and the mixture was extracted twice with 10 mL of ethyl acetate. The combined organic phases were washed once with saturated brine, dried over anhydrous sodium sulfate, and concentrated under reduced pressure to yield Intermediate 43 as a crude mixture (white solid, 2.12 g), which was used directly in the next step without further purification. A portion of the crude Intermediate 43 was purified by reverse-phase high-pressure preparative chromatography, and NMR data were obtained:

¹H NMR (400 MHz, DMSO-d₆) δ 8.10 (d, J=8.1 Hz, 1H), 7.91 (d, J=8.1 Hz, 1H), 4.01 (q, J=10.6 Hz, 2H), 3.52 (s, 3H).

### Intermediate 44

### 2-chloro-4-(methylsulfonyl)-3-((2,2,2-trifluoroethyl)sulfinyl)benzoic acid

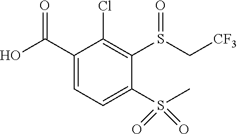 [SMILES: CS(=O)(=O)c1ccc(C(=O)O)c(Cl)c1S(=O)CC(F)(F)F]

At room temperature, Intermediate 43 (226.68 mg, 0.65 mmol) and 1,2-dichloroethane (5 mL) were added to a 25 mL single-necked flask. The mixture was heated to 50° C., and 85% m-chloroperbenzoic acid (277.13 mg, 0.72 mmol) was added portionwise. After completion of the addition, the reaction was stirred for 30 minutes. Upon completion, the mixture was filtered to remove solid impurities, washed with saturated sodium bisulfite to eliminate peroxides, and concentrated under reduced pressure to remove the solvent. Purification via high-pressure reverse-phase chromatography afforded Intermediate 44 (white solid, 181.35 mg).

### Intermediate 49

### 3-(allylthio)-2-chloro-4-(methylsulfonyl)benzoic acid

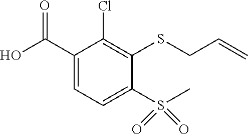 [SMILES: C=CCSc1c(S(C)(=O)=O)ccc(C(=O)O)c1Cl]

At room temperature, Intermediate 1 (1.50 g, 5.64 mmol), potassium carbonate (2.34 g, 16.92 mmol), and N,N-dimethylformamide (15 mL) were added to a 250 mL single-neck flask. The mixture was cooled to 0° C. using an ice bath, and 3-bromopropene (750.56 mg, 6.21 mmol) was slowly added dropwise. After completion of the addition, the reaction was stirred at room temperature for 1 hour. Upon completion, 5% dilute hydrochloric acid was added to adjust the pH to 1-2, and the mixture was extracted twice with 10 mL of ethyl acetate. The combined organic phases were washed once with saturated brine, dried over anhydrous sodium sulfate, and concentrated under reduced pressure to yield Intermediate 49 as a crude mixture (yellow solid, 1.65 g), which was used directly in the next step without further purification. A portion of the crude Intermediate 49 was purified by reverse-phase high-pressure preparative chromatography, and NMR data were obtained:

¹H NMR (400 MHz, DMSO-d₆) δ 8.07 (d, J=8.2 Hz, 1H), 7.86 (d, J=8.2 Hz, 1H), 5.94-5.79 (m, 1H), 5.11-4.98 (m, 2H), 3.72 (d, J=7.4 Hz, 2H), 3.53 (s, 3H).

### Intermediate 51

### 3-(allylsulfonyl)-2-chloro-4-(methylsulfonyl)benzoic acid

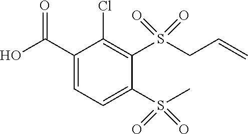 [SMILES: C=CCS(=O)(=O)c1c(S(C)(=O)=O)ccc(C(=O)O)c1Cl]

At room temperature, Intermediate 49 (199.41 mg, 0.65 mmol), acetic acid (5 mL), and sodium tungstate (9.55 mg, 0.03 mmol) were added to a 25 mL single-neck flask. The mixture was heated to 50° C., and 30% hydrogen peroxide (162.07 mg, 1.43 mmol) was added dropwise. After stirring at 50° C. for 2 hours, the solvent was removed under reduced pressure. The residue was dissolved in 10 mL of ethyl acetate, dried over anhydrous sodium sulfate, filtered, and concentrated. Reverse-phase high-pressure purification yielded Intermediate 51 (white solid, 159.53 mg).

¹H NMR (400 MHz, DMSO-d₆) CN8.33 (d, J=8.2 Hz, 1H), 8.12 (d, J=8.2 Hz, 1H), 6.01-5.86 (m, 1H), 5.52-5.42 (m, 2H), 4.50 (d, J=7.4 Hz, 2H), 3.62 (s, 3H).

The intermediates listed in Table 1-1 can be synthesized using a method analogous to the one described above for the intermediate:

TABLE 1-1

| Intermediate | | |
|---|---|---|
| NO. | Structure | ¹H NMR |
| Intermediate 1 | 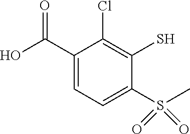 | ¹H NMR (400 MHz, CD₃CN-d₃) δ 8.07 (d, J = 8.2 Hz, 1H), 7.67 (d, J = 8.3 Hz, 1H), 5.65 (brs, 1H), 3.25 (s, 3H). |
| Intermediate 2 | 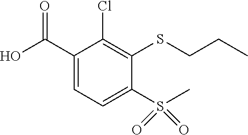 [SMILES: CCCSc1c(S(C)(=O)=O)ccc(C(=O)O)c1Cl] | ¹H NMR (400 MHz, DMSO-d₆) δ 8.09 (d, J = 7.0 Hz, 1H), 7.89 (d, J = 7.0 Hz, 1H), 3.55 (s, 3H), 2.97 (t, J = 7.4 Hz, 2H), 1.61 (q, J = 7.4 Hz, 2H), 0.97 (t, J = 7.3 Hz, 3H). |
| Intermediate 3 | 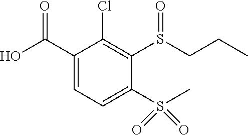 [SMILES: CCCS(=O)c1c(S(C)(=O)=O)ccc(C(=O)O)c1Cl] | ¹H NMR (400 MHz, DMSO-d₆) δ 8.08 (d, J = 8.1 Hz, 1H), 8.00 (d, J = 8.1 Hz, 1H), 3.87-3.65 (m, 1H), 3.51 (s, 3H), 3.12-3.01 (m, 1H), 1.91-1.69 (m, 2H), 1.07 (t, J = 7.4 Hz, 3H). |
| Intermediate 4 | 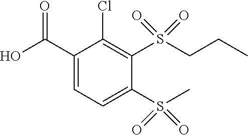 [SMILES: CCCS(=O)(=O)c1c(S(C)(=O)=O)ccc(C(=O)O)c1Cl] | ¹H NMR (400 MHz, DMSO-d₆) δ 8.29 (d, J = 8.2Hz, 1H), 8.08 (d, J = 8.2 Hz, 1H), 3.71-3.64 (m, 2H), 3.61 (s, 3H), 1.96-1.82 (m, 2H), 1.04 (t, J = 7.4 Hz, 3H). |
| Intermediate 5 | 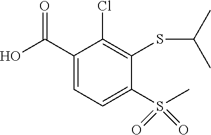 [SMILES: CC(C)Sc1c(S(C)(=O)=O)ccc(C(=O)O)c1Cl] | ¹H NMR (400 MHz, DMSO-d₆) δ 8.17 (d, J = 8.1 Hz, 1H), 7.94 (d, J = 8.2 Hz, 1H), 3.84 (p, J = 6.7 Hz, 1H), 3.59 (s, 3H), 1.28 (d, J = 6.7 Hz, 6H). |
| Intermediate 6 | 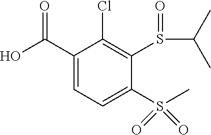 [SMILES: CC(C)S(=O)c1c(S(C)(=O)=O)ccc(C(=O)O)c1Cl] | ¹H NMR (400 MHz, DMSO-d₆) δ 8.13 (d, J = 8.1 Hz, 1H), 7.95 (d, J = 8.2 Hz, 1H), 4.13-4.07 (m, 1H), 3.49 (s, 3H), 1.44 (d, J = 6.8 Hz, 3H), 1.07 (d, J = 7.0 Hz, 3H). |
| Intermediate 7 | 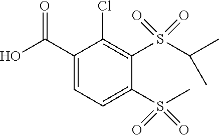 [SMILES: CC(C)S(=O)(=O)c1c(S(C)(=O)=O)ccc(C(=O)O)c1Cl] | ¹H NMR (400 MHz, DMSO-d₆) δ 8.40 (d, J = 8.3 Hz, 1H), 8.12 (d, J = 8.3 Hz, 1H), 4.25-4.10 (m, 1H), 3.59 (s, 3H), 1.33 (d, J = 6.9 Hz, 6H). |
| Intermediate 8 | 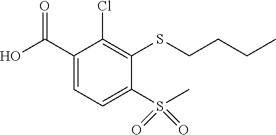 [SMILES: CCCCSc1c(S(C)(=O)=O)ccc(C(=O)O)c1Cl] | |
| Intermediate 9 | 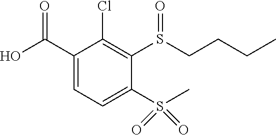 [SMILES: CCCCS(=O)c1c(S(C)(=O)=O)ccc(C(=O)O)c1Cl] | |
| Intermediate 10 | 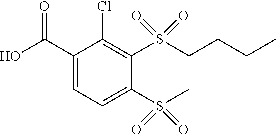 [SMILES: CCCCS(=O)(=O)c1c(S(C)(=O)=O)ccc(C(=O)O)c1Cl] | |
| Intermediate 11 | 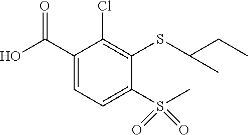 [SMILES: CCC(C)Sc1c(S(C)(=O)=O)ccc(C(=O)O)c1Cl] | ¹H NMR (400 MHz, DMSO-d₆) δ 8.11 (d, J = 8.2 Hz, 1H), 7.88 (d, J = 8.2 Hz, 1H), 3.73-3.57 (m, 1H), 3.53 (s, 3H), 1.60 (p, J = 7.2 Hz, 2H), 1.14 (d, J = 6.7 Hz, 3H), 0.96 (t, J = 7.4 Hz, 3H). |
| Intermediate 12 | 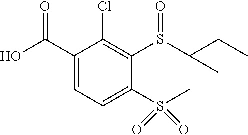 [SMILES: CCC(C)S(=O)c1c(S(C)(=O)=O)ccc(C(=O)O)c1Cl] | ¹H NMR (400 MHz, DMSO-d₆) δ 8.15 (d, J = 8.0 Hz, 1H), 8.05-7.97 (m, 1H), 4.03-3.97 (m, 0.7H), 3.94-3.83 (m, 0.3H), 3.53-3.47 (m, 3H), 2.13-1.42 (m, 2H), 1.40 (d, J = 6.9 Hz, 1H), 1.10-0.98 (m, 4H), 0.89 (t, J = 7.4 Hz, 1H). |
| Intermediate 13 | 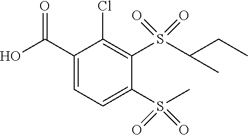 [SMILES: CCC(C)S(=O)(=O)c1c(S(C)(=O)=O)ccc(C(=O)O)c1Cl] | ¹H NMR (400 MHz, DMSO-d₆) δ 8.38 (d, J = 8.0 Hz, 1H), 8.07 (d, J = 8.2 Hz, 1H), 4.04-3.91 (m, 1H), 3.59 (s, 3H), 2.00-1.85 (m, 1H), 1.70-1.56 (m, 1H), 1.31 (d, J = 6.8 Hz, 3H), 0.97 (t, J = 7.4 Hz, 3H). |
| Intermediate 14 | 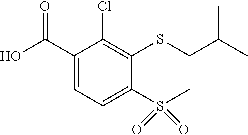 [SMILES: CC(C)CSc1c(S(C)(=O)=O)ccc(C(=O)O)c1Cl] | |
| Intermediate 15 | 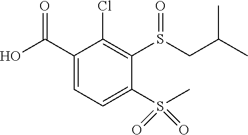 [SMILES: CC(C)CS(=O)c1c(S(C)(=O)=O)ccc(C(=O)O)c1Cl] | |
| Intermediate 16 | 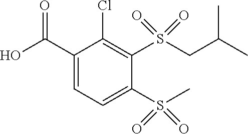 [SMILES: CC(C)CS(=O)(=O)c1c(S(C)(=O)=O)ccc(C(=O)O)c1Cl] | |
| Intermediate 17 | 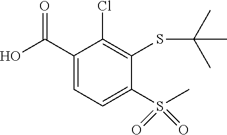 [SMILES: CC(C)(C)Sc1c(S(C)(=O)=O)ccc(C(=O)O)c1Cl] | |
| Intermediate 18 | 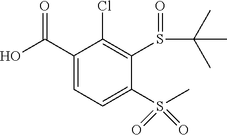 [SMILES: CC(C)(C)S(=O)c1c(S(C)(=O)=O)ccc(C(=O)O)c1Cl] | |
| Intermediate 19 | 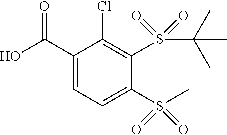 [SMILES: CC(C)(C)S(=O)(=O)c1c(S(C)(=O)=O)ccc(C(=O)O)c1Cl] | |
| Intermediate 20 | 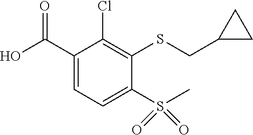 [SMILES: CS(=O)(=O)c1ccc(C(=O)O)c(Cl)c1SCC1CC1] | ¹H NMR (400 MHz, DMSO-d₆) δ 8.12 (d, J = 8.3 Hz, 1H), 7.93-7.86 (m, 1H), 3.63-3.59 (m, 3H), 3.01-2.95 (m, 2H), 1.06-1.01 (m, 1H), 0.54-0.49 (m, 2H), 0.25-0.11 (m, 2H). |
| Intermediate 21 | 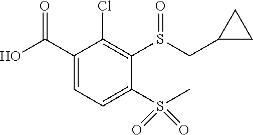 [SMILES: CS(=O)(=O)c1ccc(C(=O)O)c(Cl)c1S(=O)CC1CC1] | ¹H NMR (400 MHz, DMSO-d₆) δ 8.06 (d, J = 8.1 Hz, 1H), 7.95 (d, J = 8.1 Hz, 1H), 3.87 (dd, J = 13.1, 6.0 Hz, 1H), 3.51 (s, 3H), 2.85 (dd, J = 13.1, 9.0 Hz, 1H), 1.24-1.12 (m, 1H), 0.75-0.56 (m, 2H), 0.55-0.32 (m, 2H). |
| Intermediate 22 | 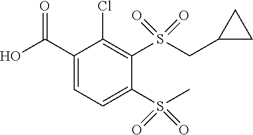 [SMILES: CS(=O)(=O)c1ccc(C(=O)O)c(Cl)c1S(=O)(=O)CC1CC1] | ¹H NMR (400 MHz, DMSO-d₆) δ 8.33 (d, J = 8.3 Hz, 1H), 8.14 (d, J = 8.3 Hz, 1H), 3.69-3.62 (m, 5H), 1.21-1.12 (m, 1H), 0.66-0.55 (m, 2H), 0.38 - 0.30 (m, 2H). |
| Intermediate 23 | 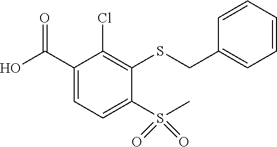 [SMILES: CS(=O)(=O)c1ccc(C(=O)O)c(Cl)c1SCc1ccccc1] | ¹H NMR (400 MHz, DMSO-d₆) δ 8.08 (d, J = 8.2 Hz, 1H), 7.90 (d, J = 8.2 Hz, 1H), 7.37-7.24 (m, 5H), 4.24 (s, 2H), 3.35 (s, 3H). |
| Intermediate 24 | 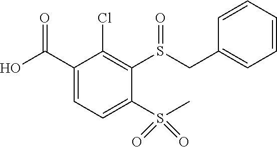 [SMILES: CS(=O)(=O)c1ccc(C(=O)O)c(Cl)c1S(=O)Cc1ccccc1] | |
| Intermediate 25 | 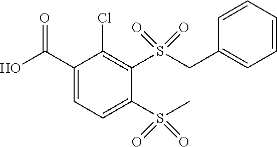 [SMILES: CS(=O)(=O)c1ccc(C(=O)O)c(Cl)c1S(=O)(=O)Cc1ccccc1] | |
| Intermediate 26 | 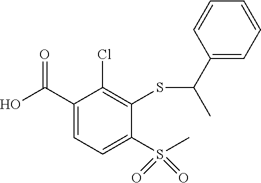 [SMILES: CC(Sc1c(S(C)(=O)=O)ccc(C(=O)O)c1Cl)c1ccccc1] | |
| Intermediate 27 | 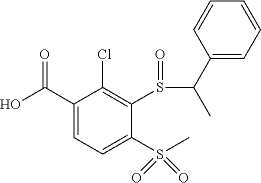 [SMILES: CC(c1ccccc1)S(=O)c1c(S(C)(=O)=O)ccc(C(=O)O)c1Cl] | |
| Intermediate 28 | 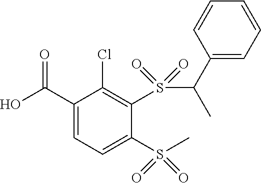 [SMILES: CC(c1ccccc1)S(=O)(=O)c1c(S(C)(=O)=O)ccc(C(=O)O)c1Cl] | |
| Intermediate 29 | 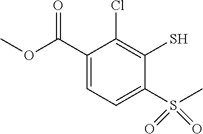 | |
| Intermediate 30 | 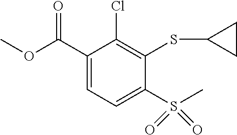 [SMILES: COC(=O)c1ccc(S(C)(=O)=O)c(SC2CC2)c1Cl] | |
| Intermediate 31 | 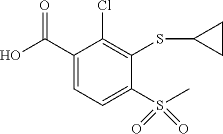 [SMILES: CS(=O)(=O)c1ccc(C(=O)O)c(Cl)c1SC1CC1] | ¹H NMR (400 MHz, DMSO-d₆) δ 8.08 (d, J = 8.2 Hz, 1H), 7.88 (d, J = 8.2 Hz, 1H), 3.45 (s, 3H), 2.70-2.63 (m, 1H), 0.92-0.86 (m, 2H), 0.79-0.73 (m, 2H). |
| Intermediate 32 | 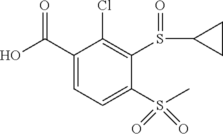 [SMILES: CS(=O)(=O)c1ccc(C(=O)O)c(Cl)c1S(=O)C1CC1] | ¹H NMR (400 MHz, DMSO-d₆) δ 8.08 (d, J = 8.2 Hz, 1H), 7.89 (d, J = 8.1 Hz, 1H), 3.48 (s, 3H), 3.38-3.34 (m, 1H), 1.28-1.17 (m, 2H), 1.09-0.95 (m, 2H). |
| Intermediate 33 | 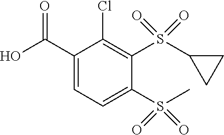 [SMILES: CS(=O)(=O)c1ccc(C(=O)O)c(Cl)c1S(=O)(=O)C1CC1] | ¹H NMR (400 MHz, DMSO-d₆) δ 8.36 (d, J = 8.3 Hz, 1H), 8.11 (d, J = 8.3 Hz, 1H), 3.61 (s, 3H), 3.48-3.40 (m, 1H), 1.49-1.43 (m, 2H), 1.24 - 1.17 (m, 2H). |
| Intermediate 34 | 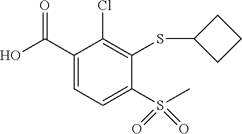 [SMILES: CS(=O)(=O)c1ccc(C(=O)O)c(Cl)c1SC1CCC1] | ¹H NMR (400 MHz, DMSO-d₆) δ 7.89 (d, J = 8.2 Hz, 1H), 7.65 (d, J = 8.2 Hz, 1H), 3.74 (p, J = 8.2 Hz, 1H), 3.34 (s, 3H), 2.18-2.06 (m, 2H), 2.02-1.88 (m, 2H), 1.78-1.57 (m, 2H). |
| Intermediate 35 | 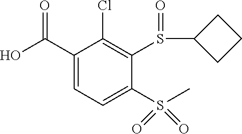 [SMILES: CS(=O)(=O)c1ccc(C(=O)O)c(Cl)c1S(=O)C1CCC1] | ¹H NMR (400 MHz, DMSO-d₆) δ 8.07 (d, J = 8.1 Hz, 1H), 7.96 (d, J = 8.1 Hz, 1H), 4.44 (p, J = 8.4 Hz, 1H), 3.50 (s, 3H), 2.80-2.66 (m, 1H), 2.35-1.85 (m, 5H). |
| Intermediate 36 | 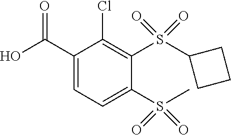 [SMILES: CS(=O)(=O)c1ccc(C(=O)O)c(Cl)c1S(=O)(=O)C1CCC1] | ¹H NMR (400 MHz, DMSO-d₆) δ 8.35 (d, J = 8.2 Hz, 1H), 8.10 (d, J = 8.2 Hz, 1H), 4.78-4.65 (m, 1H), 3.62 (s, 3H), 2.71-2.56 (m, 2H), 2.27-2.15 (m, 2H), 2.08-1.85 (m, 2H). |
| Intermediate 37 | 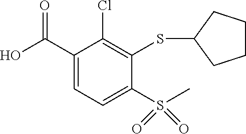 [SMILES: CS(=O)(=O)c1ccc(C(=O)O)c(Cl)c1SC1CCCC1] | ¹H NMR (400 MHz, DMSO-d₆) δ 8.10 (d, J = 8.2 Hz, 1H), 7.85 (d, J = 8.2 Hz, 1H), 3.93-3.85 (m, 1H), 3.51 (s, 3H), 1.93-1.80 (m, 2H), 1.74-1.69 (m, 2H), 1.60-1.50 (m, 4H). |
| Intermediate 38 | 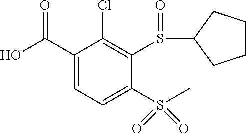 [SMILES: CS(=O)(=O)c1ccc(C(=O)O)c(Cl)c1S(=O)C1CCCC1] | ¹H NMR (400 MHz, DMSO-d₆) δ 8.13 (d, J = 8.1 Hz, 1H), 8.02 (d, J = 8.2 Hz, 1H), 4.43-4.32 (m, 1H), 3.51 (s, 3H), 2.23-2.11 (m, 1H), 2.11-1.97 (m, 1H), 1.79-1.50 (m, 6H). |
| Intermediate 39 | 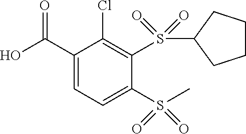 [SMILES: CS(=O)(=O)c1ccc(C(=O)O)c(Cl)c1S(=O)(=O)C1CCCC1] | ¹H NMR (400 MHz, DMSO-d₆) δ 8.41 (d, J = 8.3 Hz, 1H), 8.15 (d, J = 8.3 Hz, 1H), 4.52-4.40 (m, 1H), 3.61 (s, 3H), 2.20-2.10 (m, 2H), 1.95-1.91 (m, 2H), 1.76-1.70 (m, 2H), 1.65-1.58 (m, 2H). |
| Intermediate 40 | 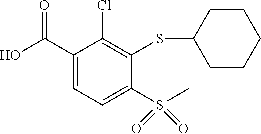 [SMILES: CS(=O)(=O)c1ccc(C(=O)O)c(Cl)c1SC1CCCCC1] | ¹H NMR (400 MHz, DMSO-d₆) δ 8.10 (d, J = 8.1 Hz, 1H), 7.86 (d, J = 8.2 Hz, 1H), 3.55-3.43 (m, 4H), 1.81-1.67 (m, 4H), 1.59-1.53 (m, 1H), 1.50-1.37 (m, 2H), 1.31-1.12 (m, 3H). |
| Intermediate 41 | 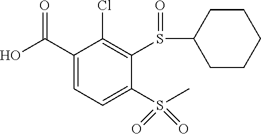 [SMILES: CS(=O)(=O)c1ccc(C(=O)O)c(Cl)c1S(=O)C1CCCCC1] | |
| Intermediate 42 | 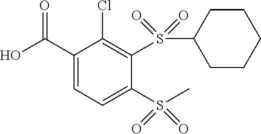 [SMILES: CS(=O)(=O)c1ccc(C(=O)O)c(Cl)c1S(=O)(=O)C1CCCCC1] | |
| Intermediate 43 | 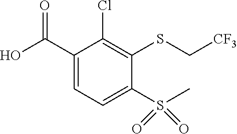 [SMILES: CS(=O)(=O)c1ccc(C(=O)O)c(Cl)c1SCC(F)(F)F] | ¹H NMR (400 MHz, DMSO-d₆) δ 8.10 (d, J = 8.1 Hz, 1H), 7.91 (d, J = 8.1 Hz, 1H), 4.01 (q, J = 10.6 Hz, 2H), 3.52 (s, 3H). |
| Intermediate 44 | 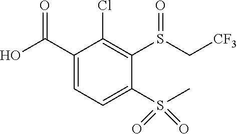 [SMILES: CS(=O)(=O)c1ccc(C(=O)O)c(Cl)c1S(=O)CC(F)(F)F] | |
| Intermediate 45 | 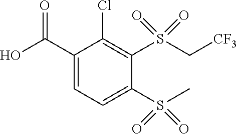 [SMILES: CS(=O)(=O)c1ccc(C(=O)O)c(Cl)c1S(=O)(=O)CC(F)(F)F] | ¹H NMR (400 MHz, DMSO-d₆) δ 8.36 (d, J = 8.3 Hz, 1H), 8.22 (d, J = 8.2 Hz, 1H), 5.06 (q, J = 9.8 Hz, 2H), 3.63 (s, 3H). |
| Intermediate 46 | 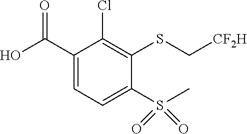 [SMILES: CS(=O)(=O)c1ccc(C(=O)O)c(Cl)c1SCC(F)F] | |
| Intermediate 47 | 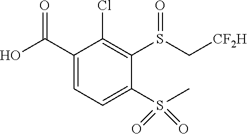 [SMILES: CS(=O)(=O)c1ccc(C(=O)O)c(Cl)c1S(=O)CC(F)F] | |
| Intermediate 48 | 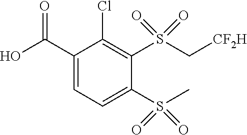 [SMILES: CS(=O)(=O)c1ccc(C(=O)O)c(Cl)c1S(=O)(=O)CC(F)F] | |
| Intermediate 49 | 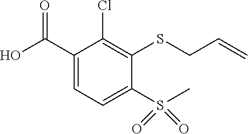 [SMILES: C=CCSc1c(S(C)(=O)=O)ccc(C(=O)O)c1Cl] | ¹H NMR (400 MHz, DMSO-d₆) δ 8.07 (d, J = 8.2 Hz, 1H), 7.86 (d, J = 8.2 Hz, 1H), 5.94-5.79 (m, 1H), 5.11-4.98 (m, 2H), 3.72 (d, J = 7.4 Hz, 2H), 3.53 (s, 3H). |
| Intermediate 50 | 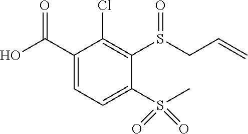 [SMILES: C=CCS(=O)c1c(S(C)(=O)=O)ccc(C(=O)O)c1Cl] | |
| Intermediate 51 | 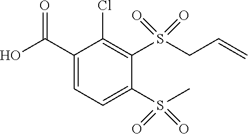 [SMILES: C=CCS(=O)(=O)c1c(S(C)(=O)=O)ccc(C(=O)O)c1Cl] | ¹H NMR (400 MHz, DMSO-d₆) δ 8.33 (d, J = 8.2 Hz, 1H), 8.12 (d, J = 8.2 Hz, 1H), 6.01-5.86 (m, 1H), 5.52-5.42 (m, 2H), 4.50 (d, J = 7.4 Hz, 2H), 3.62 (s, 3H). |
| Intermediate 52 | 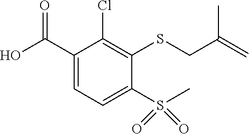 [SMILES: C=C(C)CSc1c(S(C)(=O)=O)ccc(C(=O)O)c1Cl] | ¹H NMR (400 MHz, DMSO-d₆) δ 8.06 (d, J = 7.7 Hz, 1H), 7.78 (d, J = 8.1 Hz, 1H), 4.84 (s, 1H), 4.79 (s, 1H), 3.63 (s, 2H), 3.52 (s, 3H), 1.85 (s, 3H). |
| Intermediate 53 | 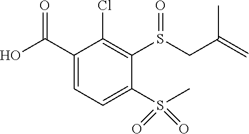 [SMILES: C=C(C)CS(=O)c1c(S(C)(=O)=O)ccc(C(=O)O)c1Cl] | |
| Intermediate 54 | 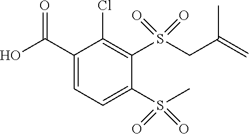 [SMILES: C=C(C)CS(=O)(=O)c1c(S(C)(=O)=O)ccc(C(=O)O)c1Cl] | |
| Intermediate 55 | 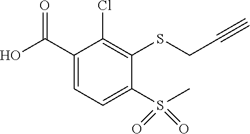 [SMILES: C#CCSc1c(S(C)(=O)=O)ccc(C(=O)O)c1Cl] | |
| Intermediate 56 | 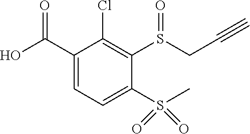 [SMILES: C#CCS(=O)c1c(S(C)(=O)=O)ccc(C(=O)O)c1Cl] | |
| Intermediate 57 | 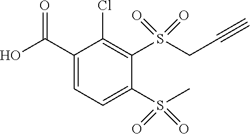 [SMILES: C#CCS(=O)(=O)c1c(S(C)(=O)=O)ccc(C(=O)O)c1Cl] | |
| Intermediate 58 | 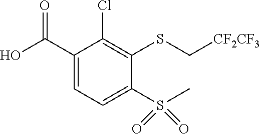 | |
| Intermediate 59 | 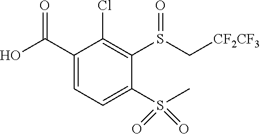 | |
| Intermediate 60 | 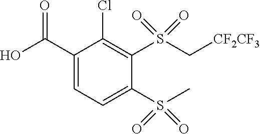 | |
| Intermediate 61 | 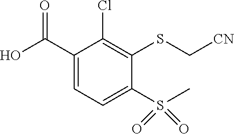 [SMILES: CS(=O)(=O)c1ccc(C(=O)O)c(Cl)c1SCC#N] | |
| Intermediate 62 | 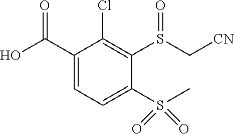 [SMILES: CS(=O)(=O)c1ccc(C(=O)O)c(Cl)c1S(=O)CC#N] | |
| Intermediate 63 | 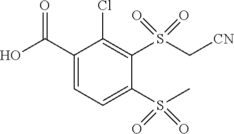 [SMILES: CS(=O)(=O)c1ccc(C(=O)O)c(Cl)c1S(=O)(=O)CC#N] | |
| Intermediate 64 | 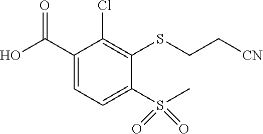 [SMILES: CS(=O)(=O)c1ccc(C(=O)O)c(Cl)c1SCCC#N] | |
| Intermediate 65 | 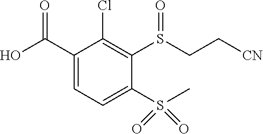 [SMILES: CS(=O)(=O)c1ccc(C(=O)O)c(Cl)c1S(=O)CCC#N] | |
| Intermediate 66 | 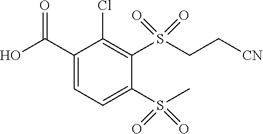 [SMILES: CS(=O)(=O)c1ccc(C(=O)O)c(Cl)c1S(=O)(=O)CCC#N] | |
| Intermediate 67 | 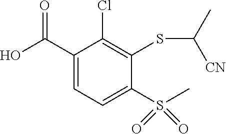 [SMILES: CC(C#N)Sc1c(S(C)(=O)=O)ccc(C(=O)O)c1Cl] | |
| Intermediate 68 | 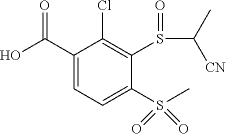 [SMILES: CC(C#N)S(=O)c1c(S(C)(=O)=O)ccc(C(=O)O)c1Cl] | |
| Intermediate 69 | 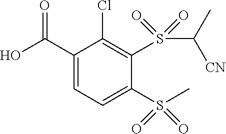 [SMILES: CC(C#N)S(=O)(=O)c1c(S(C)(=O)=O)ccc(C(=O)O)c1Cl] | |
| Intermediate 70 | 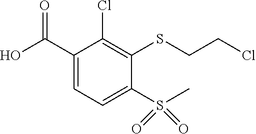 [SMILES: CS(=O)(=O)c1ccc(C(=O)O)c(Cl)c1SCCCl] | |
| Intermediate 71 | 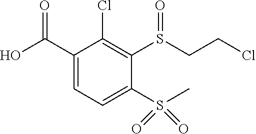 [SMILES: CS(=O)(=O)c1ccc(C(=O)O)c(Cl)c1S(=O)CCCl] | |
| Intermediate 72 | 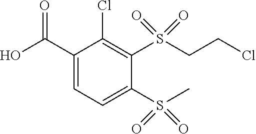 [SMILES: CS(=O)(=O)c1ccc(C(=O)O)c(Cl)c1S(=O)(=O)CCCl] | ¹H NMR (400 MHz, DMSO-d₆) δ 8.33 (d, J = 8.2 Hz, 1H), 8.17 (d, J = 8.2 Hz, 1H), 4.23 (t, J = 6.2 Hz, 2H), 4.12 (t, J = 6.2 Hz, 2H), 3.63 (s, 3H). |

### Example 1-1

### 2-chloro-N-(1-methyl-1H-tetrazol-5-yl)-4-(methylsulfonyl)-3-(propylthio)benzamide

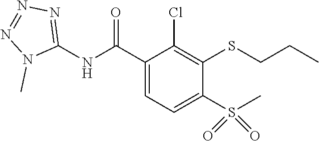 [SMILES: CCCSc1c(S(C)(=O)=O)ccc(C(=O)Nc2nnnn2C)c1Cl]

At room temperature, Intermediate 2 (120 mg, 0.39 mmol), 1-methyl-5-aminotetrazole (50.55 mg, 0.45 mmol), pyridine (4 mL), and N-methylimidazole (63.88 mg, 0.78 mmol) were added to a 50 mL single-neck flask. The mixture was cooled to 5° C. using an ice bath, and oxalyl chloride (98.75 mg, 0.78 mmol) was slowly added dropwise. After stirring at 0° C. for 30 minutes, the reaction was warmed to 55° C. and stirred for 1 hour. Upon completion, pyridine was removed by rotary evaporation. Water was added, and 5% dilute hydrochloric acid was added to adjust the pH to 1-2. The mixture was extracted twice with 15 mL of ethyl acetate, and the combined organic phases were washed once with saturated brine, dried over anhydrous sodium sulfate, and concentrated. Flash column chromatography on silica gel yielded Compound 1-1 (white solid, 90 mg). ¹H NMR (400 MHz, DMSO-d₆) δ 12.01 (s, 1H), 8.16 (d, J=8.1 Hz, 1H), 8.00 (d, J=8.1 Hz, 1H), 4.03 (s, 3H), 3.58 (s, 3H), 3.00 (t, J=7.4 Hz, 2H), 1.64 (q, J=7.4 Hz, 2H), 0.98 (t, J=7.3 Hz, 3H).

### Example 1-2

### 2-chloro-N-(1-methyl-1H-tetrazol-5-yl)-4-(methylsulfonyl)-3-(propylsulfinyl)benzamide

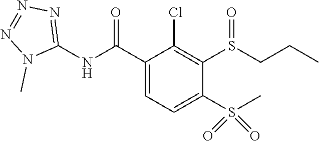 [SMILES: CCCS(=O)c1c(S(C)(=O)=O)ccc(C(=O)Nc2nnnn2C)c1Cl]

At room temperature, compound 1-1 (90 mg, 0.22 mmol) and dichloromethane (5 mL) were added to a 25 mL single-necked flask. The mixture was cooled to 5° C. in an ice bath, and 85% m-chloroperbenzoic acid (45.27 mg, 0.22 mmol) was added in portions. After complete addition, the reaction was stirred in the ice bath for 1 hour. Upon completion, solid impurities were removed by filtration. The mixture was washed with saturated sodium bisulfite to remove peroxides, and the solvent was evaporated under reduced pressure. Purification by flash silica gel column chromatography yielded compound 1-2 (white solid, 45 mg).

¹H NMR (400 MHz, DMSO-d₆) δ 12.07 (s, 1H), 8.20-8.10 (m, 2H), 4.03 (s, 3H), 3.74 (ddd, J=12.9, 7.9, 5.0 Hz, 1H), 3.55 (s, 3H), 3.08 (dt, J=12.7, 8.1 Hz, 1H), 1.91-1.74 (m, 2H), 1.08 (t, J=7.4 Hz, 3H).

### Example 1-3 1-4

1-2-Racemate (130 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-3 (59 mg, Rt=15.01 min, 100% ee, purity 99.7%, [α]_{D}²⁰=+100.30° (c 0.100, DMF)) and compound 1-4 (60 mg, Rt=11.35 min, 100% ee, purity 99.6%, [α]_{D}²⁰=−106.54° (c 0.101, DMF)) were obtained.

### Example 1-5

### 2-chloro-N-(1-methyl-1H-tetrazol-5-yl)-4-(methylsulfonyl)-3-(propylsulfonyl)benzamide

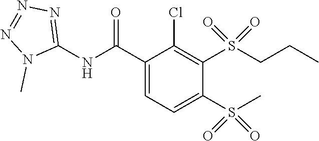 [SMILES: CCCS(=O)(=O)c1c(S(C)(=O)=O)ccc(C(=O)Nc2nnnn2C)c1Cl]

At room temperature, intermediate 4 (132.61 mg, 0.39 mmol), 1-methyl-5-aminotetrazole (50.55 mg, 0.45 mmol), pyridine (4 mL), and N-methylimidazole (63.88 mg, 0.79 mmol) were added to a 50 mL single-necked flask. The mixture was cooled to 5° C. in an ice bath, and oxalyl chloride (98.75 mg, 0.79 mmol) was added dropwise. After complete addition, the reaction was stirred at 0° C. for 30 minutes, then warmed to 55° C. and stirred for 1 hour. Upon completion, pyridine was removed by rotary evaporation. Water was added to the flask, and the pH was adjusted to 1-2 by dropwise addition of 5% dilute hydrochloric acid. The mixture was extracted twice with ethyl acetate (15 mL each). The combined organic phases were washed with saturated brine once, dried over anhydrous sodium sulfate, and concentrated under reduced pressure. Reverse-phase HPLC purification afforded compound 1-5 (white solid, 65 mg).

¹H NMR (400 MHz, DMSO-d₆) δ 12.09 (s, 1H), 8.40 (d, J=8.1 Hz, 1H), 8.31 (d, J=7.6 Hz, 1H), 4.03 (s, 3H), 3.70 (t, J=7.6 Hz, 2H), 3.66 (s, 3H), 1.96-1.84 (m, 2H), 1.06 (t, J=7.4 Hz, 3H).

### Example 1-8 1-9

1-7-Racemate (110 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-8 (46 mg, Rt=14.14 min, 100% ee, purity 99.8%, [α]_{D}²⁰=+58.73° (c 0.116, DMF)) and compound 1-9 (54 mg, Rt=9.59 min, 100% ee, purity 99.7%, [α]_{D}²⁰=−64.82° (c 0.104, DMF)) were obtained.

### Example 1-17 1-18

1-16-Racemate (430 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-17 (200 mg, Rt=24.86 min, 100% ee, purity 98.5%, [α]_{D}²⁰=+16.83° (c 0.102, DMF)) and compound 1-18 (208 mg, Rt=27.23 min, 100% ee, purity 99.1%, [α]_{D}²⁰=−19.58° (c 0.104, DMF)) were obtained.

### Example 1-2 1-21

1-19-Racemate (70 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-20 (32 mg, Rt=59.4 min, 100% ee, purity 99.9%, [α]_{D}²⁰=+60.17° (c 0.103, DMF)) and compound 1-21 (32 mg, Rt=45.8 min, 100% ee, purity 99.2%, [α]_{D}²⁰=−67.37° (c 0.100, DMF)) were obtained.

### Example 1-22 1-23

1-19-Racemate (130 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-22 (30 mg, Rt=23.1 min, 100% ee, purity 100%, [α]_{D}²⁰=+59.65° (c 0.103, DMF)) and compound 1-23 (30 mg, Rt=14.2 min, 100% ee, purity 99.9%, [α]_{D}²⁰=−65.81° (c 0.101, DMF)) were obtained.

### Example 1-25 1-26

1-24-Racemate (110 mg, 97% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 1-25 (50 mg, Rt=61.8 min, 100% ee, purity 99.7%, [α]_{D}²⁰=+1.95° (c 0.102, DMF)) and compound 1-26 (52 mg, Rt=55.5 min, 100% ee, purity 99.4%, [α]_{D}²⁰=−2.62° (c 0.107, DMF)) were obtained.

### Example 1-92

### 2-chloro-N-(1-ethyl-1H-tetrazol-5-yl)-3-(isopropylthio)-4-(methylsulfonyl)benzamide

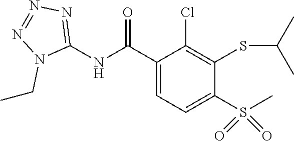 [SMILES: CCn1nnnc1NC(=O)c1ccc(S(C)(=O)=O)c(SC(C)C)c1Cl]

At room temperature, intermediate 5 (500 mg, 1.62 mmol), 1-ethyl-5-aminotetrazole (183.17 mg, 1.62 mmol), pyridine (8 mL), and N-methylimidazole (465.56 mg, 5.67 mmol) were added to a 50 mL single-necked flask. The mixture was cooled to 5° C. in an ice bath, and oxalyl chloride (98.75 mg, 0.78 mmol) was added dropwise. After complete addition, the reaction was stirred at 0° C. for 30 minutes, then warmed to 55° C. and stirred for 1 hour. Upon completion, pyridine was removed by rotary evaporation. Water was added to the flask, and the pH was adjusted to 1-2 by dropwise addition of 5% dilute hydrochloric acid. The mixture was extracted twice with ethyl acetate (20 mL each). The combined organic phases were washed with saturated brine once, dried over anhydrous sodium sulfate, and concentrated under reduced pressure. Purification by flash silica gel column chromatography yielded compound 1-92 (white solid, 470 mg).

¹H NMR (400 MHz, DMSO-d₆) δ 11.95 (s, 1H), 8.18 (d, J=8.1 Hz, 1H), 8.00 (d, J=8.2 Hz, 1H), 4.45-4.34 (m, 2H), 3.88-3.74 (m, 1H), 3.57 (s, 3H), 1.49 (t, J=7.2 Hz, 3H), 1.25 (t, J=5.9 Hz, 6H).

### Example 1-93

### 2-chloro-N-(1-ethyl-1H-tetrazol-5-yl)-3-(isopropylsulfinyl)-4-(methylsulfonyl)benzamide

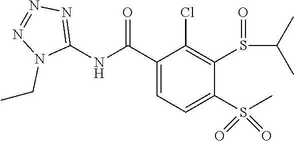 [SMILES: CCn1nnnc1NC(=O)c1ccc(S(C)(=O)=O)c(S(=O)C(C)C)c1Cl]

At room temperature, compound 1-92 (260 mg, 0.513 mmol) and dichloromethane (6 mL) were added to a 25 mL single-necked flask. The mixture was cooled to 5° C. in an ice bath, and 85% m-chloroperbenzoic acid (124.99 mg, 0.616 mmol) was added in portions. After complete addition, the reaction was stirred in the ice bath for 1 hour. Upon completion, solid impurities were removed by filtration. The mixture was washed with saturated sodium bisulfite to remove peroxides, and the solvent was evaporated under reduced pressure. Purification by flash silica gel column chromatography yielded compound 1-93 (white solid, 180 mg).

¹H NMR (400 MHz, DMSO-d₆) δ 11.99 (s, 1H), 8.22 (d, J=8.0 Hz, 1H), 8.16 (d, J=8.3 Hz, 1H), 4.44-4.32 (m, 2H), 4.18-4.07 (m, 1H), 3.53 (s, 3H), 1.52-1.41 (m, 6H), 1.12 (d, J=7.0 Hz, 3H).

### Example 1-96

### 2-chloro-N-(1-ethyl-1H-tetrazol-5-yl)-3-(isopropylsulfonyl)-4-(methylsulfonyl)benzamide

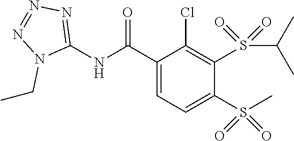 [SMILES: CCn1nnnc1NC(=O)c1ccc(S(C)(=O)=O)c(S(=O)(=O)C(C)C)c1Cl]

At room temperature, intermediate 7 (250 mg, 0.73 mmol), 1-ethyl-5-aminotetrazole (82.98 mg, 0.73 mmol), pyridine (5 mL), and N-methylimidazole (210.94 mg, 2.57 mmol) were added to a 50 mL single-necked flask. The mixture was cooled to 5° C. in an ice bath, and oxalyl chloride (232.92 mg, 1.84 mmol) was added dropwise. After complete addition, the reaction was stirred at 0° C. for 30 minutes, then warmed to 55° C. and stirred for 1 hour. Upon completion, pyridine was removed by rotary evaporation. Water was added to the flask, and the pH was adjusted to 1-2 by dropwise addition of 5% dilute hydrochloric acid. The mixture was extracted twice with ethyl acetate (15 mL each). The combined organic phases were washed with saturated brine once, dried over anhydrous sodium sulfate, and concentrated under reduced pressure. Reverse-phase HPLC purification afforded compound 1-96 (white solid, 189 mg).

¹H NMR (400 MHz, DMSO-d₆) δ 11.99 (s, 1H), 8.48 (d, J=8.2 Hz, 1H), 8.34 (d, 1H), 4.44-4.34 (m, 2H), 4.26-4.14 (m, 1H), 3.64 (s, 3H), 1.48 (t, J=7.2 Hz, 3H), 1.37 (d, J=6.8 Hz, 6H).

### Example 1-102

### 2-chloro-3-(isopropylthio)-N-methyl-N-(1-methyl-1H-tetrazol-5-yl)-4-(methylsulfonyl)benzamide

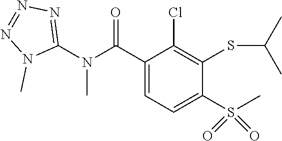 [SMILES: CC(C)Sc1c(S(C)(=O)=O)ccc(C(=O)N(C)c2nnnn2C)c1Cl]

At room temperature, Intermediate 5 (1.50 g, 4.87 mmol) and N,N-dimethylformamide (13 mL) were added to a 50 mL single-necked flask. N,N′-Carbonyldiimidazole (1.58 g, 9.74 mmol) was added in portions, and the mixture was stirred at room temperature for 2 h. Then, 1,8-diazabicyclo[5.4.0]undec-7-ene (2.52 g, 14.61 mmol) and N,1-dimethyl-1H-tetrazol-5-amine (1.00 g, 8.84 mmol) were added, and stirring continued at room temperature for 2 h. After completion, the reaction mixture was poured into 50 mL of ice-water, and a solid precipitated. The solid was filtered, washed with a small amount of water, and dried. The crude product was purified by flash silica gel column chromatography to afford Compound 1-102 (white solid, 1100 mg).

¹H NMR (400 MHz, CDCl3-d, rotameric) δ 8.24 (d, J=8.1 Hz, 0.7H), 8.02 (d, J=8.1 Hz, 0.3H), 7.61 (d, J=8.1 Hz, 0.7H), 7.49 (d, J=8.2 Hz, 0.3H), 4.03-3.92 (m, 3H), 3.86-3.75 (m, 0.7H), 3.66-3.59 (m, 0.3H), 3.46-3.39 (m, 3H), 3.30 (s, 3H), 1.27-1.23 (m, 4.2H), 1.09 (d, J=6.7 Hz, 1.8H).

### Example 1-103

### 2-chloro-3-(isopropylsulfinyl)-N-methyl-N-(1-methyl-1H-tetrazol-5-yl)-4-(methylsulfonyl)benzamide

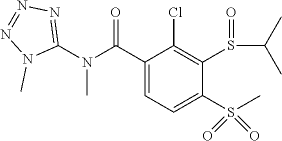 [SMILES: CC(C)S(=O)c1c(S(C)(=O)=O)ccc(C(=O)N(C)c2nnnn2C)c1Cl]

At room temperature, Compound 1-102 (300 mg, 0.74 mmol) and dichloromethane (3 mL) were added to a 25 mL single-necked flask. 85% m-chloroperoxybenzoic acid (151.13 mg, 0.74 mmol) was added in portions, and the mixture was stirred for 1 h. After completion, the reaction mixture was filtered to remove solid impurities, washed with saturated sodium bisulfite to remove excess peroxide, and concentrated under reduced pressure. The crude product was purified by flash silica gel column chromatography to afford Compound 1-103 (white solid, 201 mg).

¹H NMR (400 MHz, CDCl3-d, rotameric) δ 8.39-8.34 (m, 0.7H), 8.15 (d, J=8.0 Hz, 0.3H), 7.81-7.69 (m, 1H), 4.40-3.89 (m, 4H), 3.60-3.24 (m, 6H), 1.71-0.98 (m, 6H).

### Example 1-106

### 2-chloro-3-(isopropylsulfonyl)-N-methyl-N-(1-methyl-1H-tetrazol-5-yl)-4-(methylsulfonyl)benzamide

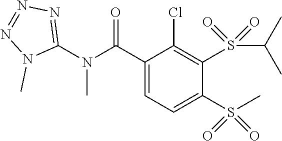 [SMILES: CC(C)S(=O)(=O)c1c(S(C)(=O)=O)ccc(C(=O)N(C)c2nnnn2C)c1Cl]

At room temperature, Compound 1-102 (305 mg, 0.76 mmol) and dichloromethane (3 mL) were added to a 25 mL single-necked flask. 85% m-chloroperoxybenzoic acid (383.70 mg, 1.89 mmol) was added in portions, and the mixture was stirred for 1 h. After completion, the reaction mixture was filtered to remove solid impurities, washed with saturated sodium bisulfite, and concentrated under reduced pressure. The crude product was purified by flash silica gel column chromatography to afford Compound 1-106 (white solid, 176 mg).

¹H NMR (400 MHz, DMSO-d₆, rotameric) δ 8.51 (d, J=8.3 Hz, 0.5H), 8.37 (d, J=8.3 Hz, 0.5H), 8.28 (d, J=8.3 Hz, 0.5H), 8.02 (d, J=8.3 Hz, 0.5H), 4.21 (p, J=6.8 Hz, 0.5H), 4.13-4.01 (m, 3.5H), 3.63 (s, 1.5H), 3.56 (s, 1.5H), 3.46 (s, 1.5H), 3.27 (s, 1.5H), 1.39 (d, 3H), 1.10-1.02 (m, 3H).

### Example 2-6

### 2-chloro-3-(isopropylthio)-N-(1-methyl-1H-1,2,4-triazol-5-yl)-4-(methylsulfonyl)benzamide

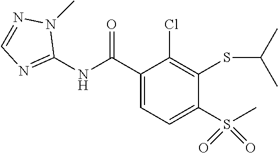 [SMILES: CC(C)Sc1c(S(C)(=O)=O)ccc(C(=O)Nc2ncnn2C)c1Cl]

At room temperature, Intermediate 5 (1.20 g, 3.88 mmol), 1-methyl-1H-1,2,4-triazol-5-amine (0.57 g, 5.83 mmol), N-methylimidazole (0.95 g, 11.64 mmol), and pyridine (15 mL) were added to a 100 mL single-necked flask. The mixture was cooled to 0° C., and thionyl chloride (1.38 g, 11.64 mmol) was added dropwise. The reaction was then warmed to room temperature and stirred overnight. After completion, pyridine was removed under reduced pressure. The residue was diluted with 10% hydrochloric acid (20 mL) and extracted with ethyl acetate (20 mL×3). The combined organic layers were washed with water and saturated sodium chloride solution, dried over anhydrous sodium sulfate, and concentrated under reduced pressure. The crude product was purified by flash silica gel column chromatography to afford Compound 2-6 (yellow solid, 1.20 g).

¹H NMR (400 MHz, DMSO-d₆) δ 11.47 (s, 1H), 8.16 (d, J=7.9 Hz, 1H), 8.01-7.87 (m, 2H), 3.90-3.81 (m, 1H), 3.80 (s, 3H), 3.56 (s, 3H), 1.26 (d, J=6.5 Hz, 6H).

### Example 2-7

### 2-chloro-3-(isopropylsulfinyl)-N-(1-methyl-1H-1,2,4-triazol-5-yl)-4-(methylsulfonyl)benzamide

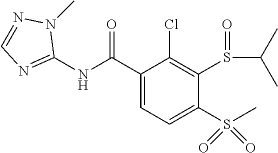 [SMILES: CC(C)S(=O)c1c(S(C)(=O)=O)ccc(C(=O)Nc2ncnn2C)c1Cl]

At room temperature, Compound 2-6 (200 mg, 0.51 mmol) and dichloromethane (5 mL) were added to a 25 mL single-necked flask. The mixture was cooled to 5° C. in an ice bath, and 85% m-chloroperoxybenzoic acid (94 mg, 0.46 mmol) was added in portions. The reaction was warmed to room temperature and stirred for 1 h. After completion, the solvent was evaporated under vacuum, and the crude product was purified by reverse-phase chromatography to afford Compound 2-7 (yellow solid, 90 mg).

¹H NMR (400 MHz, DMSO-d₆) δ 11.53 (s, 1H), 8.21 (d, J=7.9 Hz, 1H), 8.14 (d, J=3.5 Hz, 1H), 7.93 (s, 1H), 4.19-4.07 (m, 1H), 3.80 (s, 3H), 3.52 (s, 3H), 1.46 (d, J=6.7 Hz, 3H), 1.12 (d, J=6.8 Hz, 3H).

### Example 2-8 2-9

2-7-Racemate (120 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 2-8 (48 mg, Rt=21.05 min, 100% ee, purity 100%, [α]_{D}²⁰=+56.09° (c 0.103, DMF)) and compound 2-9 (58 mg, Rt=16.81 min, 100% ee, purity 100%, [α]_{D}²⁰=−58.03° (c 0.102, DMF)) were obtained.

### Example 2-10

### 2-chloro-3-(isopropylsulfonyl)-N-(1-methyl-H-1,2,4-triazol-5-yl)-4-(methylsulfonyl)benzamide

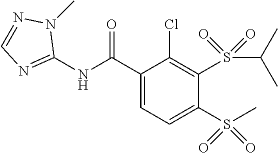 [SMILES: CC(C)S(=O)(=O)c1c(S(C)(=O)=O)ccc(C(=O)Nc2ncnn2C)c1Cl]

At room temperature, Compound 2-6 (330 mg, 0.85 mmol) and dichloromethane (10 mL) were added to a 25 mL single-necked flask. The mixture was cooled to 5° C. in an ice bath, and 85% m-chloroperoxybenzoic acid (517 mg, 2.55 mmol) was added in portions. The reaction was warmed to room temperature and stirred for 1 h. The reaction was monitored by LC-MS until completion. The solvent was evaporated under vacuum, and the crude product was purified by reverse-phase chromatography to afford Compound 2-10 (yellow solid, 130 mg).

¹H NMR (400 MHz, DMSO-d₆) δ 11.59 (s, 1H), 8.46 (d, J=8.1 Hz, 1H), 8.28 (d, J=7.8 Hz, 1H), 7.96 (s, 1H), 4.28-4.12 (m, 1H), 3.80 (s, 3H), 3.63 (s, 3H), 1.37 (d, J=6.4 Hz, 6H).

### Example 2-29 2-30

2-28-Racemate (120 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 2-29 (58 mg, Rt=27.83 min, 100% ee, purity 98.6%, [α]_{D}²⁰=+99.87° (c 0.104, DMF)) and compound 2-30 (52 mg, Rt=30.74 min, 100% ee, purity 99.1%, [α]_{D}²⁰=−98.32° (c 0.108, DMF)) were obtained.

### Example 2-82

### 2-chloro-3-(isopropylthio)-N-methyl-N-(1-methyl-1H-1,2,4-triazol-5-yl)-4-(methylsulfonyl)benzamide

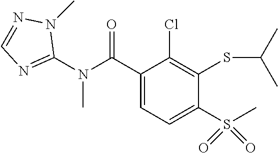 [SMILES: CC(C)Sc1c(S(C)(=O)=O)ccc(C(=O)N(C)c2ncnn2C)c1Cl]

At room temperature, compound 2-6 (1.20 g, 3.08 mmol), N,N-dimethylformamide (10 mL), and anhydrous potassium carbonate (0.64 g, 4.62 mmol) were sequentially added to a single-neck flask. The reaction system was cooled to 0° C., and methyl iodide (0.87 g, 6.16 mmol) was slowly added dropwise. The mixture was stirred at 0° C. for 30 minutes and then allowed to warm to room temperature and stirred overnight. The reaction was monitored by LC-MS until completion. The reaction mixture was poured into water (50 mL) and extracted three times with ethyl acetate. The combined organic layers were washed twice with saturated sodium chloride solution, dried over sodium sulfate, and concentrated under vacuum. Purification by column chromatography yielded compound 2-82 (white solid, 0.8 g).

¹H NMR (400 MHz, DMSO-d₆, rotameric) δ 8.21 (d, J=8.1 Hz, 0.3H), 8.02-7.94 (m, 1.3H), 7.67 (d, J=8.7 Hz, 1.4H), 3.82 (s, 2.1H), 3.81-3.79 (m, 1.2H), 3.70-3.64 (m, 0.7H), 3.57 (s, 0.9H), 3.47 (s, 2.1H), 3.37 (s, 2.1H), 3.16 (s, 0.9H), 1.25 (d, J=6.9 Hz, 1.8H), 1.10 (d, J=6.6 Hz, 4.2H).

### Example 2-83

### 2-chloro-3-(isopropylsulfinyl)-N-methyl-N-(1-methyl-1H-1,2,4-triazol-5-yl)-4-(methylsulfonyl)benzamide

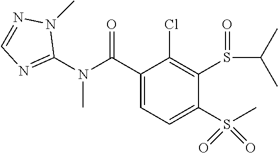 [SMILES: CC(C)S(=O)c1c(S(C)(=O)=O)ccc(C(=O)N(C)c2ncnn2C)c1Cl]

At room temperature, compound 2-82 (0.30 g, 0.74 mmol) and dichloromethane (5 mL) were sequentially added to a single-neck flask. The reaction system was cooled to 0° C., and 85% m-chloroperoxybenzoic acid (0.15 g, 0.74 mmol) was added in three batches while maintaining the temperature at 0° C. The mixture was then stirred at room temperature for 1 hour. The reaction was monitored by LC-MS until completion. The solvent was removed under vacuum, and the crude product was purified by reverse-phase chromatography to yield compound 2-83 (white solid, 250 mg).

¹H NMR (400 MHz, DMSO-d₆, rotameric) δ 8.24 (d, J=8.1 Hz, 0.3H), 8.16-7.98 (m, 1.3H), 7.84 (d, J=8.1 Hz, 0.7H), 7.69 (s, 0.7H), 4.15-4.04 (m, 0.3H), 4.10-3.89 (s, 0.7H), 3.84 (s, 2.1H), 3.81 (s, 0.9H), 3.53 (s, 0.9H), 3.46 (s, 2.1H), 3.38 (s, 2.1H), 3.20-3.19 (s, 0.9H), 1.46 (d, J=6.8 Hz, 0.9H), 1.40 (d, J=6.8 Hz, 2.1H), 1.14 (s, 0.9H), 0.91 (d, J=7.0 Hz, 2.1H).

### Example 2-86

### 2-chloro-3-(isopropylsulfonyl)-N-methyl-N-(1-methyl-1H-1,2,4-triazol-5-yl)-4-(methylsulfonyl)benzamide

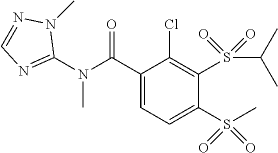 [SMILES: CC(C)S(=O)(=O)c1c(S(C)(=O)=O)ccc(C(=O)N(C)c2ncnn2C)c1Cl]

At room temperature, compound 2-82 (0.30 g, 0.74 mmol) and dichloromethane (10 mL) were sequentially added to a single-neck flask. 85% m-chloroperoxybenzoic acid (0.45 g, 2.22 mmol) was added in batches, and the mixture was stirred at room temperature for 1 hour. The reaction was monitored by LC-MS until completion. The solvent was removed under vacuum, and the crude product was purified by reverse-phase chromatography to yield compound 2-86 (white solid, 250 mg).

¹H NMR (400 MHz, DMSO-d₆, rotameric) δ 8.49 (d, J=8.3 Hz, 0.3H), 8.32-8.25 (m, 1H), 8.02-7.97 (m, 1H), 7.70 (s, 0.7H), 4.26-4.14 (m, 0.3H), 4.09-4.02 (m, 0.7H), 3.85 (s, 2.1H), 3.81 (s, 0.9H), 3.63 (s, 0.9H), 3.56 (s, 2.1H), 3.39 (s, 2.1H), 3.18 (s, 0.9H), 1.38 (d, J=6.9 Hz, 1.8H), 1.24 (d, J=6.6 Hz, 4.2H).

### Example 3-3 3-4

3-2-Racemate (120 mg, 96% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-3 (52 mg, Rt=70.26 min, 98.8% ee, purity 99.5%, [α]_{D}²⁰=+98.84° (c 0.108, DMF)) and compound 3-4 (49 mg, Rt=30.46 min, 100% ee, purity 99.6%, [α]_{D}²⁰=−101.09° (c 0.102, DMF)) were obtained.

### Example 3-6

### 2-chloro-3-(isopropylthio)-N-(5-methyl-1,3,4-oxadiazol-2-yl)-4-(methylsulfonyl)benzamide

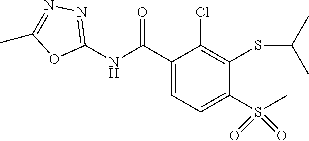 [SMILES: Cc1nnc(NC(=O)c2ccc(S(C)(=O)=O)c(SC(C)C)c2Cl)o1]

At room temperature, intermediate 5 (510 mg, 1.65 mmol), 5-methyl-1,3,4-oxadiazol-2-amine (196.39 mg, 1.98 mmol), pyridine (8 mL), and N-methylimidazole (406.44 mg, 4.95 mmol) were added to a 100 mL single-neck flask. The mixture was cooled to 5° C. in an ice bath, and thionyl chloride (588.90 mg, 4.95 mmol) was slowly added dropwise. After the addition, the mixture was stirred at room temperature for 2 hours. Upon completion, 5% dilute hydrochloric acid was added to adjust the pH to 1-2. The mixture was extracted twice with ethyl acetate (15 mL each), and the combined organic phases were washed with saturated brine and dried over anhydrous sodium sulfate. The solvent was removed under reduced pressure, and the crude product was purified by rapid silica gel column chromatography to yield compound 3-6 (white solid, 320 mg).

¹H NMR (400 MHz, DMSO-d₆) δ 12.48 (s, 1H), 8.14 (d, J=8.1 Hz, 1H), 7.87 (d, J=8.2 Hz, 1H), 3.78 (h, J=6.7 Hz, 1H), 3.55 (s, 3H), 2.48 (s, 3H), 1.24 (d, J=6.6 Hz, 6H).

### Example 3-7

### 2-chloro-3-(isopropylsulfinyl)-N-(5-methyl-1,3,4-oxadiazol-2-yl)-4-(methylsulfonyl)benzamide

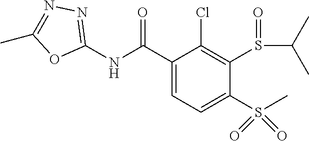 [SMILES: Cc1nnc(NC(=O)c2ccc(S(C)(=O)=O)c(S(=O)C(C)C)c2Cl)o1]

At room temperature, compound 3-6 (238 mg, 0.61 mmol) and dichloromethane (5 mL) were added to a 50 mL single-neck flask. The mixture was cooled to 5° C. in an ice bath, and 85% m-chloroperoxybenzoic acid (136.34 mg, 0.67 mmol) was added in batches. After the addition, the mixture was stirred at room temperature for 2 hours. The solvent was removed under reduced pressure, and the crude product was purified by rapid silica gel column chromatography to yield compound 3-7 (white solid, 83 mg).

¹H NMR (400 MHz, DMSO-d₆) δ 12.55 (s, 1H), 8.18 (d, J=8.1 Hz, 1H), 8.05 (d, J=8.1 Hz, 1H), 4.23-3.93 (m, 1H), 3.51 (s, 3H), 2.48 (s, 3H), 1.45 (d, J=6.8 Hz, 3H), 1.11 (d, J=7.0 Hz, 3H).

### Example 3-10

### 2-chloro-3-(isopropylsulfonyl)-N-(5-methyl-1,3,4-oxadiazol-2-yl)-4-(methylsulfonyl)benzamide

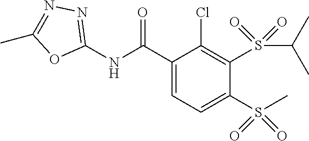 [SMILES: Cc1nnc(NC(=O)c2ccc(S(C)(=O)=O)c(S(=O)(=O)C(C)C)c2Cl)o1]

At room temperature, intermediate 7 (300 mg, 0.88 mmol), 5-methyl-1,3,4-oxadiazol-2-amine (104.69 mg, 1.06 mmol), pyridine (8 mL), and N-methylimidazole (217.59 mg, 2.65 mmol) were added to a 100 mL single-neck flask. The mixture was cooled to 5° C. in an ice bath, and thionyl chloride (315.27 mg, 2.65 mmol) was slowly added dropwise. After the addition, the mixture was stirred at room temperature for 2 hours. Upon completion, 5% dilute hydrochloric acid was added to adjust the pH to 1-2. The mixture was extracted twice with ethyl acetate (15 mL each), and the combined organic phases were washed with saturated brine and dried over anhydrous sodium sulfate. The solvent was removed under reduced pressure, and the crude product was purified by rapid silica gel column chromatography to yield compound 3-10 (white solid, 216 mg).

¹H NMR (400 MHz, DMSO-d₆) δ 12.55 (s, 1H), 8.18 (d, J=8.1 Hz, 1H), 8.05 (d, J=8.1 Hz, 1H), 4.16-4.01 (m, 1H), 3.51 (s, 3H), 2.48 (s, 3H), 1.45 (d, J=6.8 Hz, 3H), 1.11 (d, J=7.0 Hz, 3H).

### Example 3-25 3-26

3-24-Racemate (130 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-25 (28 mg, Rt=41.2 min, 100% ee, purity 95.7%, [α]_{D}²⁰=+1.97° (c 0.102, DMF)) and compound 3-26 (30 mg, Rt=30.7 min, 100% ee, purity 99.4%, [α]_{D}²⁰=−1.97° (c 0.102, DMF)) were obtained.

### Example 3-267

### 2-chloro-4-(methylsulfonyl)-N-(1,3,4-oxadiazol-2-yl)-3-(propylthio)benzamide

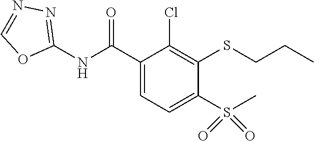 [SMILES: CCCSc1c(S(C)(=O)=O)ccc(C(=O)Nc2nnco2)c1Cl]

At room temperature, Intermediate 2 (300 mg, 0.97 mmol), 1,3,4-oxadiazol-2-amine (99.53 mg, 1.17 mmol), pyridine (6 mL), and N-methylimidazole (240.58 mg, 2.93 mmol) were added to a 100 mL single-neck flask. The mixture was cooled to 5° C. in an ice bath, and thionyl chloride (348.58 mg, 2.93 mmol) was slowly added dropwise. After complete addition, the reaction was stirred at room temperature for 2 hours. Upon completion, the pH was adjusted to 1-2 by dropwise addition of 5% dilute hydrochloric acid. The mixture was extracted twice with ethyl acetate (15 mL each), and the combined organic layers were washed once with saturated brine. The organic phase was dried over anhydrous sodium sulfate, and the solvent was removed under reduced pressure. Purification by silica gel flash column chromatography afforded Compound 3-267 (white solid, 132 mg).

¹H NMR (400 MHz, DMSO-d₆) δ 12.60 (s, 1H), 9.09 (s, 1H), 8.14 (d, J=8.1 Hz, 1H), 7.91 (d, J=8.1 Hz, 1H), 3.57 (s, 3H), 2.99 (t, J=7.4 Hz, 2H), 1.64 (h, J=7.4 Hz, 2H), 0.99 (t, J=7.3 Hz, 3H).

### Example 3-268

### 2-chloro-4-(methylsulfonyl)-N-(1,3,4-oxadiazol-2-yl)-3-(propylsulfinyl)benzamide

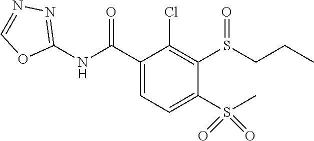 [SMILES: CCCS(=O)c1c(S(C)(=O)=O)ccc(C(=O)Nc2nnco2)c1Cl]

At room temperature, Compound 3-267 (200 mg, 0.53 mmol) and dichloromethane (5 mL) were added to a 50 mL single-neck flask. The mixture was cooled to 5° C. in an ice bath, and 85% m-chloroperbenzoic acid (119.79 mg, 0.59 mmol) was added in portions. After complete addition, the reaction was stirred at room temperature for 2 hours. Upon completion, the solvent was evaporated under reduced pressure. Purification by silica gel flash column chromatography yielded Compound 3-268 (white solid, 86 mg).

¹H NMR (400 MHz, DMSO-d₆) δ 12.69 (s, 1H), 9.08 (s, 1H), 8.12 (d, J=8.1 Hz, 1H), 8.05 (d, J=8.1 Hz, 1H), 3.79-3.66 (m, 1H), 3.54 (s, 3H), 3.15-3.01 (m, 1H), 1.90-1.73 (m, 2H), 1.08 (t, J=7.4 Hz, 3H).

### Example 3-271

### 2-chloro-4-(methylsulfonyl)-N-(1,3,4-oxadiazol-2-yl)-3-(propylsulfonyl)benzamide

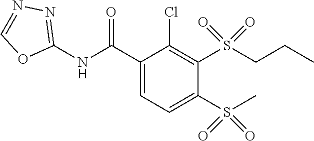 [SMILES: CCCS(=O)(=O)c1c(S(C)(=O)=O)ccc(C(=O)Nc2nnco2)c1Cl]

At room temperature, Intermediate 4 (300 mg, 0.88 mmol), 1,3,4-oxadiazol-2-amine (90.17 mg, 1.06 mmol), pyridine (8 mL), and N-methylimidazole (217.59 mg, 2.65 mmol) were added to a 100 mL single-neck flask. The mixture was cooled to 5° C. in an ice bath, and thionyl chloride (315.27 mg, 2.65 mmol) was slowly added dropwise. After complete addition, the reaction was stirred at room temperature for 2 hours. Upon completion, the pH was adjusted to 1-2 by dropwise addition of 5% dilute hydrochloric acid. The mixture was extracted twice with ethyl acetate (15 mL each), and the combined organic layers were washed once with saturated brine. The organic phase was dried over anhydrous sodium sulfate, and the solvent was removed under reduced pressure. Purification by silica gel flash column chromatography afforded Compound 3-271 (white solid, 199 mg).

¹H NMR (400 MHz, DMSO-d₆) δ 12.71 (s, 1H), 9.09 (s, 1H), 8.38 (d, J=8.2 Hz, 1H), 8.22 (d, J=8.2 Hz, 1H), 3.71-3.66 (m, 2H), 3.65 (s, 3H), 1.95-1.84 (m, 2H), 1.05 (t, J=7.4 Hz, 3H).

### Example 3-283 3-284

3-282-Racemate (400 mg, 95% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-283 (198 mg, Rt=32.8 min, 100% ee, purity 99.15%, [α]_{D}²⁰=+12.91° (c 0.115, DMF)) and compound 3-284 (183 mg, Rt=36.8 min, 98.2% ee, purity 99.2%, [α]_{D}²⁰=−13.71° (c 0.108, DMF)) were obtained.

### Example 3-286 3-287

3-285-Racemate (220 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-286 (100 mg, Rt=34.1 min, 100% ee, purity 97.8%, [α]_{D}²⁰=+78.73° (c 0.105, DMF)) and compound 3-287 (101 mg, Rt=25.3 min, 100% ee, purity 99.4%, [α]_{D}²⁰=−77.65° (c 0.109, DMF)) were obtained.

### Example 3-288 3-289

3-285-Racemate (160 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-288 (72 mg, Rt=38.8 min, 97.3% ee, purity 97.8%, [α]_{D}²⁰=+65.05° (c 0.104, DMF)) and compound 3-289 (74 mg, Rt=22.3 min, 100% ee, purity 98.8%, [α]_{D}²⁰=−80.21° (c 0.105, DMF)) were obtained.

### Example 3-388

### 2-chloro-N-(5-ethyl-1,3,4-oxadiazol-2-yl)-3-(isopropylthio)-4-(methylsulfonyl)benzamide

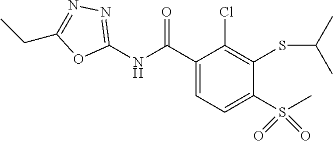 [SMILES: CCc1nnc(NC(=O)c2ccc(S(C)(=O)=O)c(SC(C)C)c2Cl)o1]

At room temperature, Intermediate 5 (300 mg, 0.97 mmol), 5-ethyl-1,3,4-oxadiazol-2-amine (131.88 mg, 1.17 mmol), pyridine (6 mL), and N-methylimidazole (240.58 mg, 2.93 mmol) were added to a 100 mL single-necked flask. The mixture was cooled to 5° C. in an ice bath, and thionyl chloride (348.58 mg, 2.93 mmol) was slowly added dropwise. After complete addition, the reaction was stirred at room temperature for 2 hours. Upon completion, 5% dilute hydrochloric acid was added to adjust the pH to 1-2. The mixture was extracted twice with 15 mL of ethyl acetate, and the combined organic phases were washed once with saturated brine. After drying over anhydrous sodium sulfate, the solvent was removed under reduced pressure. Flash silica gel column chromatography yielded compound 3-388 (white solid, 228 mg).

¹H NMR (400 MHz, DMSO-d₆) δ 12.52 (s, 1H), 8.14 (d, J=8.1 Hz, 1H), 7.88 (d, J=8.1 Hz, 1H), 3.85-3.75 (m, 1H), 3.55 (s, 3H), 2.91-2.77 (m, 2H), 1.29-1.20 (m, 9H).

### Example 3-389

### 2-chloro-N-(5-ethyl-1,3,4-oxadiazol-2-yl)-3-(isopropylsulfinyl)-4-(methylsulfonyl)benzamide

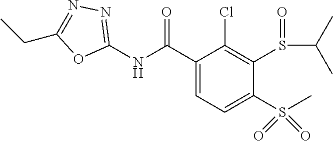 [SMILES: CCc1nnc(NC(=O)c2ccc(S(C)(=O)=O)c(S(=O)C(C)C)c2Cl)o1]

At room temperature, compound 3-388 (200 mg, 0.50 mmol) and dichloromethane (5 mL) were added to a 50 mL single-necked flask. The mixture was cooled to 5° C. in an ice bath, and 85% m-chloroperbenzoic acid (109.64 mg, 0.54 mmol) was added in batches. After complete addition, the reaction was stirred at room temperature for 2 hours. Upon completion, the solvent was removed under reduced pressure. Flash silica gel column chromatography yielded compound 3-389 (white solid, 115 mg).

¹H NMR (400 MHz, DMSO-d₆) δ 12.58 (s, 1H), 8.18 (d, J=8.1 Hz, 1H), 8.05 (d, J=8.1 Hz, 1H), 4.16-4.00 (m, 1H), 3.51 (s, 3H), 2.92-2.77 (m, 2H), 1.45 (d, J=6.8 Hz, 3H), 1.25 (t, J=7.5 Hz, 3H), 1.11 (d, J=7.0 Hz, 3H).

### Example 3-392

### 2-chloro-N-(5-ethyl-1,3,4-oxadiazol-2-yl)-3-(isopropylsulfonyl)-4-(methylsulfonyl)benzamide

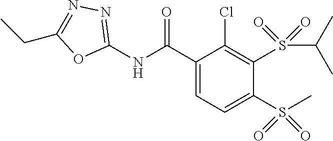 [SMILES: CCc1nnc(NC(=O)c2ccc(S(C)(=O)=O)c(S(=O)(=O)C(C)C)c2Cl)o1]

At room temperature, Intermediate 7 (300 mg, 0.88 mmol), 5-ethyl-1,3,4-oxadiazol-2-amine (118.86 mg, 1.06 mmol), pyridine (8 mL), and N-methylimidazole (217.59 mg, 2.65 mmol) were added to a 100 mL single-necked flask. The mixture was cooled to 5° C. in an ice bath, and thionyl chloride (315.27 mg, 2.65 mmol) was slowly added dropwise. After complete addition, the reaction was stirred at room temperature for 2 hours. Upon completion, 5% dilute hydrochloric acid was added to adjust the pH to 1-2. The mixture was extracted twice with 15 mL of ethyl acetate, and the combined organic phases were washed once with saturated brine. After drying over anhydrous sodium sulfate, the solvent was removed under reduced pressure. Flash silica gel column chromatography yielded compound 3-392 (white solid, 189 mg).

¹H NMR (400 MHz, DMSO-d₆) δ 8.42 (d, J=8.2 Hz, 1H), 8.16 (d, J=8.3 Hz, 1H), 4.25-4.11 (m, 1H), 3.62 (s, 3H), 2.82 (q, J=7.5 Hz, 2H), 1.35 (d, J=6.8 Hz, 6H), 1.25 (t, J=7.5 Hz, 3H).

### Example 3-441 3-442

3-440-Racemate (100 mg, 98% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-441 (43 mg, Rt=49.44 min, 97.6% ee, purity 97.5%, [α]_{D}²⁰=+64.66° (c 0.126, DMF)) and compound 3-442 (43 mg, Rt=19.91 min, 100% ee, purity 99.8%, [α]_{D}²⁰=−67.30° (c 0.106, DMF)) were obtained.

### Example 3-499

### 2-chloro-N-(5-isopropyl-1,3,4-oxadiazol-2-yl)-4-(methylsulfonyl)-3-(propylthio)benzamide

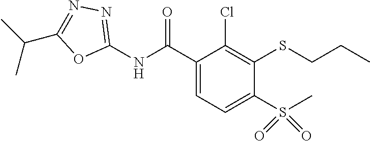 [SMILES: CCCSc1c(S(C)(=O)=O)ccc(C(=O)Nc2nnc(C(C)C)o2)c1Cl]

At room temperature, Intermediate 2 (300 mg, 0.97 mmol), 5-isopropyl-1,3,4-oxadiazol-2-amine (148.77 mg, 1.17 mmol), pyridine (6 mL), and N-methylimidazole (240.58 mg, 2.93 mmol) were added to a 100 mL single-necked flask. The mixture was cooled to 5° C. in an ice bath, and thionyl chloride (348.58 mg, 2.93 mmol) was slowly added dropwise. After completion of the addition, the reaction was stirred at room temperature for 2 hours. Upon completion, 5% dilute hydrochloric acid was added to adjust the pH to 1-2. The mixture was extracted with ethyl acetate (15 mL×2), and the combined organic phases were washed once with saturated brine. The organic layer was dried over anhydrous sodium sulfate, concentrated under reduced pressure, and purified by flash column chromatography on silica gel to afford Compound 3-499 (white solid, 219 mg).

¹H NMR (400 MHz, CDCl₃-d) δ 8.18 (d, J=8.2 Hz, 1H), 7.74 (d, J=8.1 Hz, 1H), 3.47 (s, 3H), 3.22-3.07 (m, 1H), 2.99 (t, J=7.5 Hz, 2H), 1.78-1.65 (m, 2H), 1.39 (d, J=7.0 Hz, 6H), 1.03 (t, J=7.4 Hz, 3H).

### Example 3-500

### 2-chloro-N-(5-isopropyl-1,3,4-oxadiazol-2-yl)-4-(methylsulfonyl)-3-(propylsulfinyl)benzamide

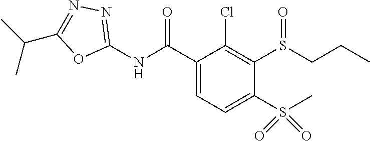 [SMILES: CCCS(=O)c1c(S(C)(=O)=O)ccc(C(=O)Nc2nnc(C(C)C)o2)c1Cl]

At room temperature, Compound 3-499 (200 mg, 0.48 mmol) and dichloromethane (5 mL) were added to a 50 mL single-necked flask. The mixture was cooled to 5° C. in an ice bath, and 85% m-chloroperbenzoic acid (mCPBA, 107.61 mg, 0.53 mmol) was added in batches. After completion of the addition, the reaction was stirred at room temperature for 2 hours. The solvent was removed under reduced pressure, and the crude product was purified by flash column chromatography on silica gel to yield Compound 3-500 (white solid, 99 mg).

¹H NMR (400 MHz, CDCl3-d) δ 8.17 (d, J=8.1 Hz, 1H), 7.88 (d, J=8.1 Hz, 1H), 3.97-3.86 (m, 1H), 3.44 (s, 3H), 3.21-3.04 (m, 2H), 2.16-1.87 (m, 2H), 1.40 (d, J=7.0 Hz, 6H), 1.16 (t, J=7.4 Hz, 3H).

### Example 3-503

### 2-chloro-N-(5-isopropyl-1,3,4-oxadiazol-2-yl)-4-(methylsulfonyl)-3-(propylsulfonyl)benzamide

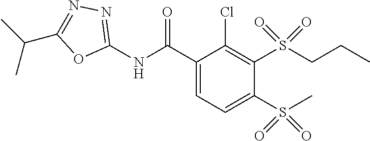 [SMILES: CCCS(=O)(=O)c1c(S(C)(=O)=O)ccc(C(=O)Nc2nnc(C(C)C)o2)c1Cl]

At room temperature, Intermediate 4 (300 mg, 0.88 mmol), 5-isopropyl-1,3,4-oxadiazol-2-amine (134.78 mg, 1.06 mmol), pyridine (8 mL), and N-methylimidazole (217.59 mg, 2.65 mmol) were added to a 100 mL single-necked flask. The mixture was cooled to 5° C. in an ice bath, and thionyl chloride (315.27 mg, 2.65 mmol) was slowly added dropwise. After completion of the addition, the reaction was stirred at room temperature for 2 hours. Upon completion, 5% dilute hydrochloric acid was added to adjust the pH to 1-2. The mixture was extracted with ethyl acetate (15 mL×2), and the combined organic phases were washed once with saturated brine. The organic layer was dried over anhydrous sodium sulfate, concentrated under reduced pressure, and purified by flash column chromatography on silica gel to afford Compound 3-503 (white solid, 212 mg).

¹H NMR (400 MHz, CDCl3-d) δ 8.39 (d, J=8.2 Hz, 1H), 7.94 (d, J=8.2 Hz, 1H), 3.67-3.57 (m, 5H), 3.22-3.07 (m, 1H), 2.14-2.02 (m, 2H), 1.41 (d, J=7.0 Hz, 6H), 1.14 (t, J=7.4 Hz, 3H).

### Example 3-542 3-543

3-541-Racemate (145 mg, 97% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-542 (75 mg, Rt=22.66 min, 100% ee, purity 98.6%, [α]_{D}²⁰=+57.50° (c 0.102, DMF)) and compound 3-543 (60 mg, Rt=15.27 min, 100% ee, purity 99.3%, [α]_{D}²⁰−=70.69° (c 0.101, DMF)) were obtained.

### Example 3-620

### 2-chloro-N-(5-cyclopropyl-1,3,4-oxadiazol-2-yl)-3-(isopropylthio)-4-(methylsulfonyl)benzamide

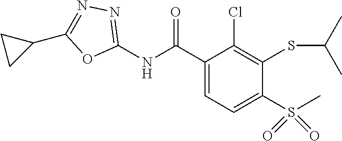 [SMILES: CC(C)Sc1c(S(C)(=O)=O)ccc(C(=O)Nc2nnc(C3CC3)o2)c1Cl]

At room temperature, Intermediate 5 (300 mg, 0.97 mmol), 5-cyclopropyl-1,3,4-oxadiazol-2-amine (146.40 mg, 1.17 mmol), pyridine (8 mL), and N-methylimidazole (240.58 mg, 2.93 mmol) were added to a 100 mL single-necked flask. The mixture was cooled to 5° C. in an ice bath, and thionyl chloride (348.58 mg, 2.93 mmol) was slowly added dropwise. After completion of the addition, the reaction was stirred at room temperature for 2 hours. Upon completion, 5% dilute hydrochloric acid was added to adjust the pH to 1-2. The mixture was extracted with ethyl acetate (15 mL×2), and the combined organic phases were washed once with saturated brine. The organic layer was dried over anhydrous sodium sulfate, concentrated under reduced pressure, and purified by flash column chromatography on silica gel to afford Compound 3-620 (white solid, 189 mg).

¹H NMR (400 MHz, DMSO-d₆) δ 12.45 (s, 1H), 8.14 (d, J=8.1 Hz, 1H), 7.87 (d, J=8.2 Hz, 1H), 3.85-3.73 (m, 1H), 3.55 (s, 3H), 2.30-2.13 (m, 1H), 1.24 (d, J=6.6 Hz, 6H), 1.17-1.07 (m, 2H), 1.05-0.90 (m, 2H).

### Example 3-621

### 2-chloro-N-(5-cyclopropyl-1,3,4-oxadiazol-2-yl)-3-(isopropylsulfinyl)-4-(methylsulfonyl)benzamide

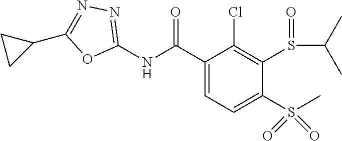 [SMILES: CC(C)S(=O)c1c(S(C)(=O)=O)ccc(C(=O)Nc2nnc(C3CC3)o2)c1Cl]

At room temperature, Compound 3-620 (150 mg, 0.36 mmol) and dichloromethane (5 mL) were added to a 50 mL single-necked flask. The mixture was cooled to 5° C. in an ice bath, and 85% m-chloroperbenzoic acid (mCPBA, 81.21 mg, 0.40 mmol) was added in batches. After completion of the addition, the reaction was stirred at room temperature for 2 hours. The solvent was removed under reduced pressure, and the crude product was purified by flash column chromatography on silica gel to yield Compound 3-621 (white solid, 78 mg).

¹H NMR (400 MHz, DMSO-d₆) δ 12.58 (s, 1H), 8.17 (d, J=8.1 Hz, 1H), 8.02 (d, J=8.2 Hz, 1H), 4.15-4.02 (m, 1H), 3.51 (s, 3H), 2.24-2.13 (m, 1H), 1.45 (d, J=6.8 Hz, 3H), 1.16-1.03 (m, 5H), 1.01-0.89 (m, 2H).

### Example 3-622 3-623

3-621-Racemate (120 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-622 (61 mg, Rt=19.36 min, 100% ee, purity 99.1%, [α]_{D}²⁰=+51.53° (c 0.111, DMF)) and compound 3-623 (52 mg, Rt=13.89 min, 100% ee, purity 99.5%, [α]_{D}²⁰=−58.27° (c 0.101, DMF)) were obtained.

### Example 3-624

### 2-chloro-N-(5-cyclopropyl-1,3,4-oxadiazol-2-yl)-3-(isopropylsulfonyl)-4-(methylsulfonyl)benzamide

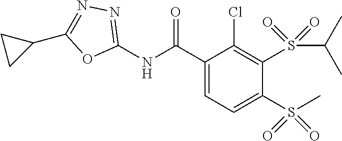 [SMILES: CC(C)S(=O)(=O)c1c(S(C)(=O)=O)ccc(C(=O)Nc2nnc(C3CC3)o2)c1Cl]

At room temperature, Intermediate 7 (300 mg, 0.67 mmol), 5-cyclopropyl-1,3,4-oxadiazol-2-amine (100.60 mg, 0.80 mmol), pyridine (8 mL), and N-methylimidazole (197.06 mg, 2.40 mmol) were added to a 100 mL single-necked flask. The mixture was cooled to 5° C. in an ice bath, and thionyl chloride (285.53 mg, 2.40 mmol) was slowly added dropwise. After completion of the addition, the reaction was stirred at room temperature for 2 hours. Upon completion, 5% dilute hydrochloric acid was added to adjust the pH to 1-2. The mixture was extracted with ethyl acetate (15 mL×2), and the combined organic phases were washed once with saturated brine. The organic layer was dried over anhydrous sodium sulfate, concentrated under reduced pressure, and purified by flash column chromatography on silica gel to afford Compound 3-624 (white solid, 163 mg).

¹H NMR (400 MHz, DMSO-d₆) δ 12.58 (s, 1H), 8.43 (d, J=8.2 Hz, 1H), 8.18 (d, J=8.2 Hz, 1H), 4.17 (p, J=6.8 Hz, 1H), 3.62 (s, 3H), 2.24-2.13 (m, 1H), 1.35 (d, J=6.8 Hz, 6H), 1.15-1.04 (m, 2H), 1.01-0.90 (m, 2H).

### Example 3-786

### 2-chloro-N-methyl-N-(5-methyl-1,3,4-oxadiazol-2-yl)-4-(methylsulfonyl)-3-(propylthio)benzamide

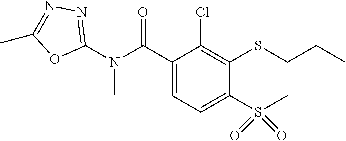 [SMILES: CCCSc1c(S(C)(=O)=O)ccc(C(=O)N(C)c2nnc(C)o2)c1Cl]

At room temperature, compound 3-2 (700 mg, 1.73 mmol), N,N-dimethylformamide (15 mL), and potassium carbonate (479.59 mg, 3.47 mmol) were added to a 50 mL single-necked flask. The mixture was cooled to 5° C. in an ice bath, and methyl iodide (731.79 mg, 5.19 mmol) was added dropwise. After completion of the addition, the reaction was stirred at room temperature for 2 hours. Upon completion, 5 mL of water was added, and the mixture was extracted with ethyl acetate (15 mL×2). The combined organic layers were washed with saturated brine once, dried over anhydrous sodium sulfate, and concentrated under reduced pressure. Purification by flash silica gel column chromatography afforded compound 3-786 (white solid, 433 mg).

¹H NMR (400 MHz, CDCl₃-d) δ 8.19 (d, J=8.1 Hz, 1H), 7.52 (d, J=8.1 Hz, 1H), 3.58 (s, 3H), 3.45 (s, 3H), 2.94 (t, J=7.5 Hz, 2H), 2.37 (s, 3H), 1.73-1.61 (m, 2H), 1.02 (t, J=7.3 Hz, 3H).

### Example 3-787

### 2-chloro-N-methyl-N-(5-methyl-1,3,4-oxadiazol-2-yl)-4-(methylsulfonyl)-3-(propylsulfinyl)benzamide

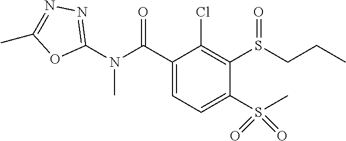 [SMILES: CCCS(=O)c1c(S(C)(=O)=O)ccc(C(=O)N(C)c2nnc(C)o2)c1Cl]

At room temperature, compound 3-786 (150 mg, 0.37 mmol) and dichloromethane (5 mL) were added to a 50 mL single-necked flask. The mixture was cooled to 5° C. in an ice bath, and 85% m-chloroperbenzoic acid (83.24 mg, 0.41 mmol) was added in portions. After completion of the addition, the reaction was stirred at room temperature for 2 hours. Upon completion, the solvent was removed under reduced pressure. Purification by flash silica gel column chromatography afforded compound 3-787 (white solid, 126 mg).

¹H NMR (400 MHz, CDCl₃-d) δ 8.15 (d, J=8.1 Hz, 1H), 7.64 (d, J=8.1 Hz, 1H), 3.80-3.76 (m, 1H), 3.58 (s, 3H), 3.41 (s, 3H), 3.11-2.99 (m, 1H), 2.42 (s, 3H), 2.07-1.83 (m, 2H), 1.14 (t, J=7.4 Hz, 3H).

### Example 3-788 3-789

3-787-Racemate (100 mg, 96% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 3-788 (46 mg, Rt=20.6 min, 97.1% ee, purity 99.1%, [α]_{D}²⁰=+143.41° (c 0.099, DMF)) and compound 3-789 (46 mg, Rt=30.1 min, 96.3% ee, purity 98.5%, [α]_{D}²⁰=−144.20° (c 0.097, DMF)) were obtained.

### Example 3-790

### 2-chloro-N-methyl-N-(5-methyl-1,3,4-oxadiazol-2-yl)-4-(methylsulfonyl)-3-(propylsulfonyl)benzamide

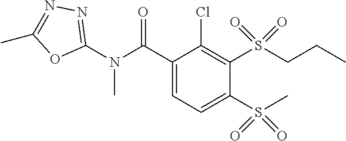 [SMILES: CCCS(=O)(=O)c1c(S(C)(=O)=O)ccc(C(=O)N(C)c2nnc(C)o2)c1Cl]

At room temperature, compound 3-786 (150 mg, 0.37 mmol) and dichloromethane (5 mL) were added to a 50 mL single-necked flask. The mixture was cooled to 5° C. in an ice bath, and 85% m-chloroperbenzoic acid (225.37 mg, 1.11 mmol) was added in portions. After completion of the addition, the reaction was stirred at room temperature for 3 hours. Upon completion, the solvent was removed under reduced pressure. Purification by flash silica gel column chromatography afforded compound 3-790 (white solid, 49 mg).

¹H NMR (400 MHz, DMSO-d₆) δ 8.32 (d, J=8.0 Hz, 1H), 8.08 (d, J=8.2 Hz, 1H), 3.69-3.60 (m, 5H), 3.49 (s, 3H), 2.33 (s, 3H), 1.93-1.77 (m, 2H), 1.02 (t, J=7.4 Hz, 3H).

### Example 4-1

### 2-chloro-N-(4-methyl-1,2,5-oxadiazol-3-yl)-4-(methylsulfonyl)-3-(propylthio)benzamide

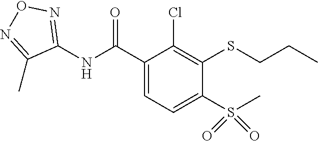 [SMILES: CCCSc1c(S(C)(=O)=O)ccc(C(=O)Nc2nonc2C)c1Cl]

At room temperature, Intermediate 2 (1.25 g, 4.06 mmol), pyridine (12 mL), 4-methyl-1,2,5-oxadiazol-3-amine (0.60 g, 6.09 mmol), and N-methylpyrazole (0.67 g, 8.12 mmol) were added sequentially to a single-necked flask. The reaction system was cooled to 0° C., and thionyl chloride (0.97 g, 8.12 mmol) was added dropwise. The mixture was stirred at 0° C. for 30 minutes and then allowed to warm to room temperature and stirred overnight. The reaction was monitored by LC-MS until completion. The solvent was removed under vacuum, and 1 M hydrochloric acid (50 mL) was added. The mixture was extracted with ethyl acetate (×3), washed with saturated sodium chloride solution (×2), dried over sodium sulfate, and concentrated under vacuum. Purification by flash silica gel column chromatography afforded compound 4-1 (white solid, 610 mg).

¹H NMR (400 MHz, CDCl3-d) δ 9.02 (s, 1H), 7.91 (d, J=8.1 Hz, 1H), 7.61 (d, J=8.2 Hz, 1H), 3.37 (s, 3H), 2.89 (t, J=7.5 Hz, 2H), 2.45 (s, 3H), 1.64 (h, J=7.4 Hz, 2H), 0.98 (t, J=7.4 Hz, 3H).

### Example 4-2

### 2-chloro-N-(4-methyl-1,2,5-oxadiazol-3-yl)-4-(methylsulfonyl)-3-(propylsulfinyl)benzamide

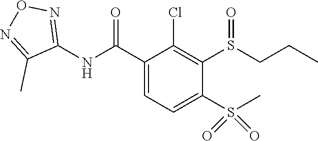 [SMILES: CCCS(=O)c1c(S(C)(=O)=O)ccc(C(=O)Nc2nonc2C)c1Cl]

At room temperature, compound 4-1 (197 mg, 0.51 mmol) and dichloromethane (5 mL) were added to a single-necked flask. The reaction system was cooled to 0° C., and 85% m-chloroperbenzoic acid (104 mg, 0.51 mmol) was added in three portions while maintaining the temperature at 0° C. The mixture was then stirred at room temperature for 1 hour. The reaction was monitored by LC-MS until completion. The solvent was removed under vacuum, and purification by flash silica gel column chromatography afforded compound 4-2 (white solid, 109 mg).

¹H NMR (400 MHz, CDCl3-d) δ 9.61 (s, 1H), 8.16 (d, J=8.1 Hz, 1H), 7.85 (d, J=8.1 Hz, 1H), 3.88-3.78 (m, 1H), 3.41 (s, 3H), 3.15-3.03 (m, 1H), 2.51 (s, 3H), 2.07-1.85 (m, 2H), 1.15 (t, J=7.4 Hz, 3H).

### Example 4-3 4-4

4-2-Racemate (130 mg, 98% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 4-3 (60 mg, Rt=19.48 min, 100% ee, purity 100%, [α]_{D}²⁰=+100.18° (c 0.108, DMF)) and compound 4-4 (58 mg, Rt=12.69 min, 100% ee, purity 100%, [α]_{D}²⁰=−111.11° (c 0.121, DMF)) were obtained.

### Example 4-5

### 2-chloro-N-(4-methyl-1,2,5-oxadiazol-3-yl)-4-(methylsulfonyl)-3-(propylsulfonyl)benzamide

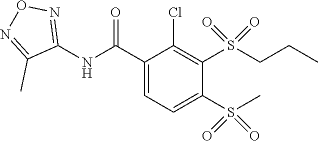 [SMILES: CCCS(=O)(=O)c1c(S(C)(=O)=O)ccc(C(=O)Nc2nonc2C)c1Cl]

At room temperature, compound 4-1 (313 mg, 0.80 mmol) and dichloromethane (10 mL) were added to a single-necked flask. 85% m-chloroperbenzoic acid (487 mg, 2.40 mmol) was added in portions, and the mixture was stirred at room temperature for 1 hour. The reaction was monitored by LC-MS until completion. The solvent was removed under vacuum, and purification by flash silica gel column chromatography afforded compound 4-5 (white solid, 128 mg).

¹H NMR (400 MHz, DMSO-d₆) δ 8.39 (d, J=8.2 Hz, 1H), 8.20 (d, J=8.2 Hz, 1H), 3.72-3.64 (m, 5H), 2.40 (s, 3H), 1.92 (q, J=7.5 Hz, 2H), 1.08 (t, J=7.4 Hz, 3H).

### Example 4-32

### 2-chloro-3-((cyclopropylmethyl)thio)-N-(4-meth 1-1,2,5-oxadiazol-3-yl)-4-(methylsulfonyl)benzamide

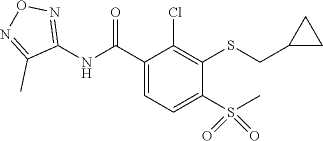 [SMILES: Cc1nonc1NC(=O)c1ccc(S(C)(=O)=O)c(SCC2CC2)c1Cl]

At room temperature, sequentially add Intermediate 20 (500 mg, 1.56 mmol), pyridine (8 mL), 4-methyl-1,2,5-oxadiazol-3-amine (232 mg, 2.34 mmol), and N-methylpyrazole (256 mg, 3.12 mmol) to a single-necked flask. Place the reaction system in a 0° C. environment, then slowly add thionyl chloride (371 mg, 3.12 mmol) dropwise while maintaining the temperature at 0° C. Stir the mixture at 0° C. for 30 minutes, then transfer the system to room temperature and stir overnight. Monitor the reaction by LC-MS until completion. Stop the reaction, remove the solvent under vacuum, add 1M hydrochloric acid (50 mL) to the system, and extract three times with ethyl acetate. Wash the combined organic layers twice with saturated sodium chloride solution and dry over sodium sulfate. Remove the solvent under vacuum and purify by silica gel column chromatography to afford Compound 4-32 (white solid, 500 mg).

¹H NMR (400 MHz, CDCl₃-d)) δ 8.72 (s, 1H), 8.08 (d, J=8.1 Hz, 1H), 7.72 (d, J=8.0 Hz, 1H), 3.49 (s, 3H), 2.95 (d, J=7.5 Hz, 2H), 2.53 (s, 3H), 1.09-1.0.5 (m, 1H), 0.65-0.54 (m, 2H), 0.33-0.21 (m, 2H).

### Example 4-33

### 2-chloro-3-((cyclopropylmethyl)sulfinyl)-N-(4-methyl-1,2,5-oxadiazol-3-yl)-4-(methylsulfonyl)benzamide

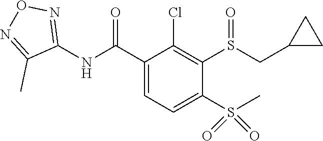 [SMILES: Cc1nonc1NC(=O)c1ccc(S(C)(=O)=O)c(S(=O)CC2CC2)c1Cl]

At room temperature, sequentially add Compound 4-32 (200 mg, 0.50 mmol) and dichloromethane (5 mL) to a single-necked flask. Place the reaction system in a 0° C. environment, then add 85% m-chloroperbenzoic acid (102 mg, 0.50 mmol) in three batches while maintaining the temperature at 0° C. Transfer the system to room temperature and stir for 1 hour. Monitor the reaction by LC-MS until completion. Stop the reaction, remove the solvent under vacuum, and purify by silica gel column chromatography to afford Compound 4-33 (brown-red solid, 120 mg).

¹H NMR (400 MHz, CDCl₃-d)) δ 10.24 (s, 1H), 8.07 (d, J=8.1 Hz, 1H), 7.83 (d, J=8.1 Hz, 1H), 3.96 (dd, J=13.2, 6.0 Hz, 1H), 3.40 (s, 3H), 2.82 (dd, J=13.2, 9.1 Hz, 1H), 2.45 (s, 3H), 2.01 (d, J=17.2 Hz, 2H), 1.24-1.20 (m, 1H), 0.82-0.68 (m, 2H).

### Example 4-36

### 2-chloro-3-((cyclopropylmethyl)sulfonyl)-N-(4-methyl-1,2,5-oxadiazol-3-yl)-4-(methylsulfonyl)benzamide

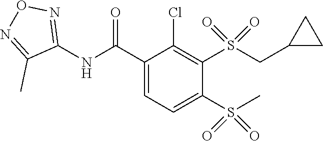 [SMILES: Cc1nonc1NC(=O)c1ccc(S(C)(=O)=O)c(S(=O)(=O)CC2CC2)c1Cl]

At room temperature, sequentially add Compound 4-32 (200 mg, 0.50 mmol) and dichloromethane (10 mL) to a single-necked flask. Add 85% m-chloroperbenzoic acid (305 mg, 1.50 mmol) in batches and stir at room temperature for 1 hour. Monitor the reaction by LC-MS until completion. Stop the reaction, remove the solvent under vacuum, and purify by silica gel column chromatography to afford Compound 4-36 (white solid, 160 mg).

¹H NMR (400 MHz, CDCl₃-d) δ 9.04 (s, 1H), 8.32 (d, J=8.1 Hz, 1H), 7.93 (d, J=8.2 Hz, 1H), 3.60 (s, 2H), 3.58 (s, 3H), 2.50 (s, 3H), 1.36-1.19 (m, 1H), 0.80-0.66 (m, 2H), 0.52-0.37 (m, 2H).

### Example 4-44 4-45

4-43-Racemate (100 mg, 98% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 4-44 (40 mg, Rt=10.59 min, 100% ee, purity 98.0%, [α]_{D}²⁰=+94.99° (c 0.099, DMF)) and compound 4-45 (48 mg, Rt=7.52 min, 100% ee, purity 99.2%, [α]_{D}²⁰=−92.68° (c 0.108, DMF)) were obtained.

### Example 4-49 4-50

4-48-Racemate (130 mg, 99% purity) was resolved via chiral HPLC (Column: CHIRALPAK® AD-H; Column Size: 3 cm×25 cm, 5 μm; Injection: 4 mL; Mobile phase: Hex:EtOH (0.1% FA)=65:35; Flow rate: 43 mL/min; Wavelength: UV 220 nm; Temperature: 25° C.; Sample solution: 6 mg/mL in Hex/EtOH; Run time: 25 mins). After concentration, compound 4-49 (54 mg, Rt=18.36 min, 100% ee, purity 96%, [α]_{D}²⁰=+42.99° (c 0.100, DMF)) and compound 4-50 (49 mg, Rt=11.70 min, 100% ee, purity 99.5%, [α]_{D}²⁰=−44.09° (c 0.104, DMF)) were obtained.

### Example 4-271

### 2-chloro-N-methyl-N-(4-methyl-1,2,5-oxadiazol-3-yl)-4-(methylsulfonyl)-3-(propylthio)benzamide

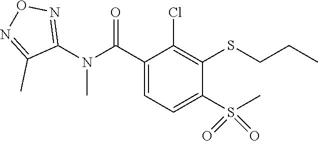 [SMILES: CCCSc1c(S(C)(=O)=O)ccc(C(=O)N(C)c2nonc2C)c1Cl]

At room temperature, sequentially add Compound 4-1 (1.20 g, 3.08 mmol), N,N-dimethylformamide (10 mL), and anhydrous potassium carbonate (0.64 g, 4.62 mmol) to a single-necked flask. Place the reaction system in a 0° C. environment, then slowly add methyl iodide (0.87 g, 6.16 mmol) dropwise while maintaining the temperature at 0° C. Stir the mixture at 0° C. for 30 minutes, then transfer the system to room temperature and stir overnight. Monitor the reaction by LC-MS until completion. Stop the reaction, pour the mixture into water (50 mL), and extract three times with ethyl acetate. Wash the combined organic layers twice with saturated sodium chloride solution and dry over sodium sulfate. Remove the solvent under vacuum and purify by silica gel column chromatography to afford Compound 4-271 (white solid, 0.8 g).

¹H NMR (400 MHz, DMSO-d₆, rotameric) δ 8.18 (d, J=8.1 Hz, 0.5H), 8.03-7.95 (m, 1H), 7.74 (d, J=8.2 Hz, 0.5H), 3.57 (s, 1.5H), 3.51 (s, 1.5H), 3.44 (s, 1.5H), 3.25 (s, 1.5H), 3.02 (t, J=7.4 Hz, 1H), 2.91-2.77 (m, 1H), 2.46 (s, 1.5H), 2.41 (s, 1.5H), 1.70-1.58 (m, 1H), 1.51-1.33 (m, 1H), 0.98 (t, J=7.3 Hz, 1.5H), 0.91 (t, J=7.3 Hz, 1.5H).

### Example 4-272

### 2-chloro-N-methyl-N-(4-methyl-1,2,5-oxadiazol-3-yl)-4-(methylsulfonyl)-3-(propylsulfinyl)benzamide

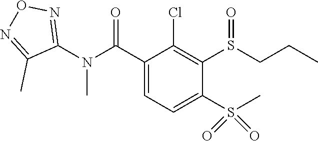 [SMILES: CCCS(=O)c1c(S(C)(=O)=O)ccc(C(=O)N(C)c2nonc2C)c1Cl]

At room temperature, sequentially add Compound 4-271 (0.30 g, 0.74 mmol) and dichloromethane (5 mL) to a single-necked flask. Place the reaction system in a 0° C. environment, then add 85% m-chloroperbenzoic acid (0.15 g, 0.74 mmol) in three batches while maintaining the temperature at 0° C. Transfer the system to room temperature and stir for 1 hour. Monitor the reaction by LC-MS until completion. Stop the reaction, remove the solvent under vacuum, and purify by silica gel column chromatography to afford Compound 4-272 (white solid, 250 mg).

¹H NMR (400 MHz, CDCl₃-d, rotameric) δ 8.18 (s, 0.6H), 8.01 (d, J=8.2 Hz, 0.4H), 7.65 (d, J=8.0 Hz, 0.6H), 7.56 (d, J=8.1 Hz, 0.4H), 3.89-3.75 (m, 0.6H), 3.70-3.56 (m, 0.4H), 3.42 (s, 1.2H), 3.37 (s, 1.8H), 3.31 (s, 1.8H), 3.29 (s, 1.2H), 3.14-3.01 (m, 0.6H), 2.98-2.87 (m, 0.4H), 2.39 (s, 1.8H), 2.36 (s, 1.2H), 2.07-1.96 (m, 0.8H), 1.90-1.76 (m, 1.2H), 1.12 (t, J=7.4 Hz, 1.8H), 1.06 (t, J=7.4 Hz, 1.2H).

### Example 4-275

### 2-chloro-N-methyl-N-(4-meth 1-1,2,5-oxadiazol-3-1)-4-methylsulfonyl)-3-(propylsulfonyl)benzamide

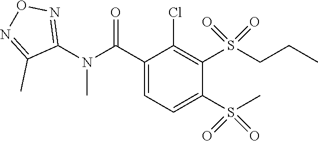 [SMILES: CCCS(=O)(=O)c1c(S(C)(=O)=O)ccc(C(=O)N(C)c2nonc2C)c1Cl]

At room temperature, sequentially add Compound 4-271 (0.30 g, 0.74 mmol) and dichloromethane (10 mL) to a single-necked flask. Add 85% m-chloroperbenzoic acid (0.45 g, 2.22 mmol) in batches and stir at room temperature for 1 hour. Monitor the reaction by LC-MS until completion. Stop the reaction, remove the solvent under vacuum, and purify by silica gel column chromatography to afford Compound 4-275 (white solid, 250 mg).

¹H NMR (400 MHz, CDCl₃-d, rotameric) δ 8.48 (d, J=8.2 Hz, 0.6H), 8.31 (d, J=8.2 Hz, 0.4H), 7.83 (d, J=8.2 Hz, 0.6H), 7.74 (d, J=8.2 Hz, 0.4H), 3.67 (td, J=7.2, 2.6 Hz, 2H), 3.62 (s, 3H), 3.56 (s, 2H), 3.53 (s, 1H), 3.49 (s, 2H), 3.35 (s, 3H), 2.46 (s, 3H), 2.44 (s, 2H), 2.15-2.10 (m, 1H), 2.03-1.91 (i, 2H), 1.17 (t, J=7.4 Hz, 3H), 1.11 (t, J=7.4 Hz, 2H).

Prepare according to a preparation embodiment similar to the one described above.

The analytical data for embodiment compounds 1-1 to 4-310 are shown in the table below

TABLE 2
Examples of compounds of formula (I)

| Compound | ¹H NMR | [α] |
|---|---|---|
| 1-1 | ¹H NMR (400 MHz, DMSO-d₆) δ 12.01 (s, 1H), 8.16 (d, J = 8.1 Hz, 1H), 8.00 | |
| | (d, J = 8.1 Hz, 1H), 4.03 (s, 3H), 3.58 (s, 3H), 3.00 (t, J = 7.4 Hz, 2H), 1.64 (q, J = 7.4 | |
| | Hz, 2H), 0.98 (t, J = 7.3 Hz, 3H). | |
| 1-2 | ¹H NMR (400 MHz, DMSO-d₆) δ 12.07 (s, 1H), 8.20-8.10 (m, 2H), 4.03 (s, | |
| | 3H), 3.79-3.70 (m, 1H), 3.55 (s, 3H), 3.13-3.04 (m, 1H), 1.91-1.74 (m, 2H), 1.08 | |
| | (t, J = 7.4 Hz, 3H). | |
| 1-3 | ¹H NMR (400 MHz, DMSO-d₆) δ 12.12 (s, 1H), 8.19-8.08 (m, 2H), 4.01 (s, | [α] |
| | 3H), 3.80-3.70 (m, 1H), 3.54 (s, 3H), 3.12-3.04 (m, 1H), 1.96-1.74 (m, 2H), 1.08 | D²⁰ = +100.30°(c |
| | (t, J = 7.4 Hz, 3H). | 0.100, DMF) |
| 1-4 | ¹H NMR (400 MHz, DMSO-d₆) δ 12.07 (s, 1H), 8.20-8.07 (m, 2H), 4.02 (s, | [α] |
| | 3H), 3.79-3.70 (m, 1H), 3.55 (s, 3H), 3.13-3.04 (m, 1H), 1.97-1.74 (m, 2H), 1.08 | D²⁰ = −106.54°(c |
| | (t, J = 7.4 Hz, 3H). | 0.101, DMF) |
| 1-5 | ¹H NMR (400 MHz, DMSO-d₆) δ 12.09 (s, 1H), 8.40 (d, J = 8.1 Hz, 1H), 8.31 | |
| | (d, J = 7.6 Hz, 1H), 4.03 (s, 3H), 3.70 (t, J = 7.6 Hz, 2H), 3.66 (s, 3H), 1.96-1.84 (m, | |
| | 2H), 1.06 (t, J = 7.4 Hz, 3H). | |
| 1-6 | ¹H NMR (400 MHz, DMSO-d₆) δ 12.04 (s, 1H), 8.18 (d, J = 8.1 Hz, 1H), 7.99 | |
| | (d, J = 8.1 Hz, 1H), 4.03 (s, 3H), 3.80 (p, J = 6.7 Hz, 1H), 3.56 (s, 3H), 1.25 (d, J = | |
| | 6.6 Hz, 6H). | |
| 1-7 | ¹H NMR (400 MHz, DMSO-d₆) δ 12.09 (s, 1H), 8.22 (d, J = 8.0 Hz, 1H), 8.15 | |
| | (d, J = 8.8 Hz, 1H), 4.16-4.05 (m, 1H), 4.02 (s, 3H), 3.53 (s, 3H), 1.46 (d, J = 6.8 | |
| | Hz, 3H), 1.12 (d, J = 7.0 Hz, 3H). | |
| 1-8 | ¹H NMR (400 MHz, DMSO-d₆) δ 12.08 (s, 1H), 8.23 (d, J = 7.9 Hz, 1H), 8.17 (d, J = | [α] |
| | 6.6 Hz, 1H), 4.18-4.06 (m, 1H), 4.03 (s, 3H), 3.53 (s, 3H), 1.46 (d, J = 6.8 Hz, 3H), | D²⁰ = +58.73°(c |
| | 1.12 (d, J = 6.9 Hz, 3H). | 0.116, DMF) |
| 1-9 | ¹H NMR (400 MHz, DMSO-d₆) δ 12.09 (s, 1H), 8.22 (d, J = 8.0 Hz, 1H), 8.15 | [α] |
| | (d, J = 8.8 Hz, 1H), 4.16-4.05 (m, 1H), 4.02 (s, 3H), 3.53 (s, 3H), 1.46 (d, J = 6.8 | D²⁰ = −64.82°(c |
| | Hz, 3H), 1.12 (d, J = 7.0 Hz, 3H). | 0.104 , DMF) |
| 1-10 | ¹H NMR (400 MHz, DMSO-d₆) δ 12.08 (s, 1H), 8.48 (d, J = 8.2 Hz, 1H), 8.32 | |
| | (d, J = 8.1 Hz, 1H), 4.28-4.12 (m, 1H), 4.03 (s, 3H), 3.64 (s, 3H), 1.37 (d, J = 6.8 | |
| | Hz, 6H). | |
| 1-16 | ¹H NMR (400 MHz, CDCl₃-d) δ 8.17 (d, J = 8.1 Hz, 1H), 7.74 (d, J = 8.1 Hz, | |
| | 1H), 4.12 (s, 3H), 3.73-3.61 (m, 1H), 3.47 (s, 3H), 1.75-1.56 (m, J = 6.9 Hz, 2H), | |
| | 1.20 (d, J = 6.7 Hz, 3H), 1.01 (t, J = 7.4 Hz, 3H). | |
| 1-17 | ¹H NMR (400 MHz, CDCl₃-d) δ 8.17 (d, J = 8.1 Hz, 1H), 7.74 (d, J = 8.1 Hz, | [α] |
| | 1H), 4.12 (s, 3H), 3.73-3.61 (m, 1H), 3.47 (s, 3H), 1.75-1.56 (m, J = 6.9 Hz, 2H), | D²⁰ = +16.83°(c |
| | 1.20 (d, J = 6.7 Hz, 3H), 1.01 (t, J = 7.4 Hz, 3H). | 0.102, DMF) |
| 1-18 | ¹H NMR (400 MHz, CDCl₃-d) δ 8.17 (d, J = 8.1 Hz, 1H), 7.74 (d, J = 8.1 Hz, | [α] |
| | 1H), 4.12 (s, 3H), 3.73-3.61 (m, 1H), 3.47 (s, 3H), 1.75-1.56 (m, J = 6.9 Hz, 2H), | D²⁰ = −19.58°(c |
| | 1.20 (d, J = 6.7 Hz, 3H), 1.01 (t, J = 7.4 Hz, 3H). | 0.104, DMF) |
| 1-19 | ¹H NMR (400 MHz, CDCl3-d) δ 11.40 (s, 1H), 8.29 (d, J = 7.9 Hz, 1H), 7.89 (d, | |
| | J = 7.9 Hz, 1H), 4.14 (s, 3H), 4.13-3.88 (m, 1H), 3.43 (d, J = 2.8 Hz, 3H), 2.24- | |
| | 1.54 (m, 2H), 1.53-0.92 (m, 6H). | |
| 1-20 | ¹H NMR (400 MHz, CDCl₃-d) δ 11.18 (s, 1H), 8.29 (d, J = 7.8 Hz, 1H), 7.88 (d, | [α] |
| | J = 7.8 Hz, 1H), 4.14 (s, 3H), 4.07-3.90 (m, 1H), 3.42 (s, 3H), 1.64-1.56 (m, 2H), | D²⁰ = +60.17°(c |
| | 1.51 (d, J = 6.9 Hz, 3H), 0.99 (t, J = 7.4 Hz, 3H). | 0.103, DMF) |
| 1-21 | ¹H NMR (400 MHz, CDCl₃-d) δ 11.23 (s, 1H), 8.30 (d, J = 7.8 Hz, 1H), 7.90 (d, | [α] |
| | J = 7.8 Hz, 1H), 4.19-4.06 (m, 4H), 3.43 (s, 3H), 2.32-2.11 (m, 1H), 1.85-1.79 | D²⁰ = −67.37°(c |
| | (m, 1H), 1.18-1.09 (m, 6H). | 0.100, DMF) |
| 1-22 | ¹H NMR (400 MHz, CDCl₃-d) δ 11.18 (s, 1H), 8.29 (d, J = 7.8 Hz, 1H), 7.89 (d, | [α] |
| | J = 8.0 Hz, 1H), 4.20-4.00 (m, 4H), 3.42 (s, 3H), 2.24-2.14 (m, 1H), 1.91-1.79 | D²⁰ = +59.65°(c |
| | (m, 1H), 1.18-1.09 (m, 6H). | 0.103, DMF) |
| 1-23 | ¹H NMR (400 MHz, CDCl₃-d) δ 11.11 (s, 1H), 8.30 (d, J = 7.6 Hz, 1H), 7.88 (d, | [α] |
| | J = 7.7 Hz, 1H), 4.14 (s, 3H), 4.05-3.84 (m, 1H), 3.42 (s, 3H), 1.64-1.56 (m, 2H), | D²⁰ = −65.81°(c |
| | 1.52 (d, J = 6.8 Hz, 3H), 0.99 (t, J = 7.4 Hz, 3H). | 0.101, DMF) |
| 1-24 | ¹H NMR (400 MHz, CDCl3-d) δ 11.23 (s, 1H), 8.52 (d, J = 7.9 Hz, 1H), 7.95 (d, | |
| | J = 8.1 Hz, 1H), 4.14 (s, 3H), 4.11-3.94 (m, 1H), 3.59 (s, 3H), 2.16-2.06 (m, 1H), | |
| | 1.85-1.65 (m, 1H), 1.44 (d, J = 6.9 Hz, 3H), 1.06 (t, J = 7.4 Hz, 3H). | |
| 1-25 | ¹H NMR (400 MHz, CDCl₃-d) δ 11.23 (s, 1H), 8.51 (d, J = 8.1 Hz, 1H), 7.96 (d, | [α] |
| | J = 8.2 Hz, 1H), 4.13 (s, 3H), 4.09-3.95 (m, 1H), 3.59 (s, 3H), 2.15-2.03 (m, 1H), | D²⁰ = +1.95°(c |
| | 1.84-1.70 (m, 1H), 1.44 (d, J = 6.9 Hz, 3H), 1.06 (t, J = 7.4 Hz, 3H). | 0.102, DMF) |
| 1-26 | ¹H NMR (400 MHz, CDCl₃-d) δ 11.22 (s, 1H), 8.52 (d, J = 8.1 Hz, 1H), 7.95 (d, | [α] |
| | J = 8.2 Hz, 1H), 4.13 (s, 3H), 4.08-3.98 (m, 1H), 3.59 (s, 3H), 2.22-2.04 (m, 1H), | D²⁰ = −2.62°(c |
| | 1.82-1.70 (m, 1H), 1.44 (d, J = 6.9 Hz, 3H), 1.06 (t, J = 7.4 Hz, 3H). | 0.107, DMF) |
| 1-27 | ¹H NMR (400 MHz, DMSO-d₆) δ 12.02 (s, 1H), 8.15 (d, J = 8.1 Hz, 1H), 8.00 | |
| | (d, J = 8.1 Hz, 1H), 4.02 (s, 3H), 3.62 (s, 3H), 2.98 (d, J = 7.4 Hz, 2H), 1.09-0.97 | |
| | (m, 0H), 0.58-0.49 (m, 2H), 0.26-0.19 (m, 2H). | |
| 1-28 | ¹H NMR (400 MHz, DMSO-d₆) δ 12.06 (s, 1H), 8.18-8.10 (m, 2H), 4.02 (s, | |
| | 3H), 3.88 (dd, J = 13.1, 6.0 Hz, 1H), 3.55 (s, 3H), 2.88 (dd, J = 13.1, 8.9 Hz, 1H), | |
| | 1.23-1.15 (m, 1H), 0.77-0.60 (m, 2H), 0.54-0.35 (m, 2H). | |
| 1-31 | ¹H NMR (400 MHz, DMSO-d₆) δ 12.09 (s, 1H), 8.41 (d, J = 8.1 Hz, 1H), 8.32 | |
| | (d, 1H), 4.03 (s, 3H), 3.69 (d, J = 7.3 Hz, 2H), 3.66 (s, 3H), 1.22-1.14 (m, 1H), | |
| | 0.67-0.59 (m, 2H), 0.41-0.32 (m, 2H). | |
| 1-32 | ¹H NMR (400 MHz, DMSO-d₆) δ 12.05 (s, 1H), 8.14 (d, J = 8.1 Hz, 1H), 8.00 | |
| | (d, J = 8.2 Hz, 1H), 4.03 (s, 3H), 3.49 (s, 3H), 2.73-2.64 (m, 1H), 0.96-0.87 (m, | |
| | 2H), 0.83-0.75 (m, 2H). | |
| 1-33 | ¹H NMR (400 MHz, DMSO-d₆) δ 12.10 (s, 1H), 8.23-8.12 (m, 2H), 4.02 (s, | |
| | 3H), 3.54 (s, 3H), 3.39-3.35 (m, 1H), 1.37-1.25 (m, 2H), 1.12-0.97 (m, 2H). | |
| 1-36 | ¹H NMR (400 MHz, DMSO-d₆) δ 12.09 (s, 1H), 8.44 (d, J = 8.2 Hz, 1H), 8.30 | |
| | (d, J = 8.3 Hz, 1H), 4.03 (s, 3H), 3.65 (s, 3H), 3.51-3.42 (m, 1H), 1.52-1.45 (m, | |
| | 2H), 1.24-1.19 (m, 2H). | |
| 1-37 | ¹H NMR (400 MHz, DMSO-d₆) δ 11.99 (s, 1H), 8.15 (d, J = 8.1 Hz, 1H), 7.97 | |
| | (d, J = 8.1 Hz, 1H), 4.03 (s, 3H), 4.00-3.90 (m, 1H), 3.56 (s, 3H), 2.39-2.27 (m, | |
| | 2H), 2.24-2.11 (m, 2H), 1.94-1.80 (m, 2H). | |
| 1-38 | ¹H NMR (400 MHz, DMSO-d₆) δ 12.05 (s, 1H), 8.19-8.07 (m, 2H), 4.51- | |
| | 4.40 (m, 1H), 4.02 (s, 3H), 3.53 (s, 3H), 2.81-2.69 (m, 1H), 2.35-2.27 (m, 1H), | |
| | 2.25-2.07 (m, 2H), 2.07-1.92 (m, 2H). | |
| 1-41 | ¹H NMR (400 MHz, DMSO-d₆) δ 12.07 (s, 1H), 8.43 (d, J = 8.2 Hz, 1H), 8.30 | |
| | (d, J = 8.2 Hz, 1H), 4.80-4.69 (m, 1H), 4.02 (s, 3H), 3.66 (s, 3H), 2.74-2.60 (m, | |
| | 2H), 2.31-2.19 (m, 2H), 2.08-1.92 (m, 2H). | |
| 1-42 | ¹H NMR (400 MHz, CDCl₃-d) δ 11.23 (s, 1H), 8.24 (d, J = 8.1 Hz, 1H), 7.74 (d, | |
| | J = 8.1 Hz, 1H), 4.15 (s, 3H), 3.95-3.87 (m, 1H), 3.46 (s, 3H), 2.00-1.91 (m, 2H), | |
| | 1.85-1.77 (m, 2H), 1.70-1.56 (m, 4H). | |
| 1-43 | ¹H NMR (400 MHz, CDCl₃-d) δ 11.47 (s, 1H), 8.27 (d, J = 7.9 Hz, 1H), 7.88 (d, | |
| | J = 7.9 Hz, 1H), 4.53-4.38 (m, 1H), 4.13 (s, 3H), 3.43 (s, 3H), 2.29 (td, J = 14.1, 7.0 | |
| | Hz, 1H), 2.15 (dt, J = 13.5, 6.8 Hz, 1H), 1.82-1.73 (m, 4H), 1.67-1.52 (m, 2H). | |
| 1-46 | ¹H NMR (400 MHz, CDCl₃-d) δ 11.15 (s, 1H), 8.54 (d, J = 8.1 Hz, 1H), 7.95 (d, | |
| | J = 8.1 Hz, 1H), 4.52-4.37 (m, 1H), 4.15 (s, 3H), 3.60 (s, 3H), 2.39-2.26 (m, 2H), | |
| | 2.02-1.94 (m, 2H), 1.93-1.84 (m, 2H), 1.73-1.65 (m, 2H). | |
| 1-52 | ¹H NMR (400 MHz, DMSO-d₆) δ 12.03 (s, 1H), 8.19 (d, J = 8.1 Hz, 1H), 8.08 | |
| | (d, J = 8.2 Hz, 1H), 4.12-4.04 (m, 2H), 4.03 (s, 3H), 3.56 (s, 3H). | |
| 1-53 | ¹H NMR (400 MHz, DMSO-d₆) δ 12.10 (s, 1H), 8.27-8.15 (m, 2H), 4.83- | |
| | 4.68 (m, 1H), 4.50-4.33 (m, 1H), 4.03 (s, 3H), 3.56 (s, 3H). | |
| 1-56 | ¹H NMR (400 MHz, DMSO-d₆) δ 12.14 (s, 1H), 8.50-8.36 (m, 2H), 5.20- | |
| | 5.06 (m, 2H), 4.03 (s, 3H), 3.66 (s, 3H). | |
| 1-57 | ¹H NMR (400 MHz, DMSO-d₆) δ 12.02 (s, 1H), 8.17 (d, J = 8.1 Hz, 1H), 8.04 | |
| | (d, J = 8.2 Hz, 1H), 6.33 (tt, J = 55.9, 4.1 Hz, 1H), 4.03 (s, 3H), 3.63-3.50 (m, 5H). | |
| 1-58 | ¹H NMR (400 MHz, DMSO-d₆) δ 11.72 (s, 1H), 8.42 (d, J = 8.2 Hz, 1H), 8.31 | |
| | (d, J = 8.2 Hz, 1H), 6.66 (tt, J = 54.0, 4.3 Hz, 1H), 4.60 (td, J = 15.0, 4.4 Hz, 2H), | |
| | 3.66 (s, 3H), 2.41 (s, 3H). | |
| 1-61 | ¹H NMR (400 MHz, DMSO-d₆) δ 12.12 (s, 1H), 8.42 (d, J = 8.1 Hz, 1H), 8.36 | |
| | (d, J = 8.4 Hz, 1H), 6.79-6.49 (m, 1H), 4.69-4.54 (m, 2H), 4.03 (s, 3H), 3.66 (s, 3H). | |
| 1-62 | ¹H NMR (400 MHz, DMSO-d₆) δ 12.09 (s, 1H), 8.20 (d, J = 8.1 Hz, 1H), 8.11 | |
| | (d, J = 8.0 Hz, 1H), 4.25 (s, 2H), 4.02 (s, 3H), 3.57 (s, 3H). | |
| 1-63 | ¹H NMR (400 MHz, DMSO-d₆) δ 8.30-8.14 (m, 2H), 4.98 (d, J = 16.1 Hz, | |
| | 1H), 4.72 (d, J = 16.1 Hz, 1H), 4.02 (s, 3H), 3.56 (s, 3H). | |
| 1-67 | ¹H NMR (400 MHz, CDCl₃-d) δ 11.15 (s, 1H), 8.14 (d, J = 8.1 Hz, 1H), 7.68 (d, | |
| | J = 8.1 Hz, 1H), 5.92-5.77 (m, 1H), 5.12-4.97 (m, 2H), 4.07 (s, 3H), 3.64 (d, J = | |
| | 7.5 Hz, 2H), 3.41 (s, 3H). | |
| 1-68 | ¹H NMR (400 MHz, CDCl₃-d) δ 11.40 (s, 1H), 8.21 (d, J = 7.9 Hz, 1H), 7.88 (d, | |
| | J = 8.0 Hz, 1H), 6.09-5.93 (m, 1H), 5.61-5.37 (m, 2H), 4.52-4.43 (m, 1H), 4.13 | |
| | (s, 3H), 4.08-3.99 (m, 1H), 3.44 (s, 3H). | |
| 1-71 | ¹H NMR (400 MHz, CDCl₃-d) δ 11.17 (brs, 1H), 8.37 (d, J = 7.9 Hz, 1H), 7.97 | |
| | (d, J = 8.0 Hz, 1H), 6.06-5.91 (m, 1H), 5.60-5.51 (m, 2H), 4.41 (d, J = 7.3 Hz, | |
| | 2H), 4.09 (s, 3H), 3.59 (s, 3H). | |
| 1-72 | ¹H NMR (400 MHz, DMSO-d₆) δ 12.00 (s, 1H), 8.17 (d, J = 8.1 Hz, 1H), 8.02 | |
| | (d, J = 8.2 Hz, 1H), 4.03 (s, 3H), 3.88 (t, J = 7.2 Hz, 2H), 3.60 (s, 3H), 3.40 (t, J = 7.2 | |
| | Hz, 2H). | |
| 1-73 | ¹H NMR (400 MHz, DMSO-d₆) δ 12.06 (s, 1H), 8.18 (s, 2H), 4.25-4.02 (m, | |
| | 7H), 3.56 (s, 3H). | |
| 1-76 | ¹H NMR (400 MHz, CCl₃D-d) δ 11.31 (s, 1H), 8.37 (d, J = 6.4 Hz, 1H), 7.99 (s, | |
| | 1H), 4.14-4.08 (m, 5H), 4.08-4.03 (m, 2H), 3.60 (s, 3H). | |
| 1-77 | ¹H NMR (400 MHz, DMSO-d₆) δ 12.74 (s, 1H), 8.14 (d, J = 8.1 Hz, 1H), 7.93 | |
| | (d, J = 8.1 Hz, 1H), 3.57 (s, 3H), 3.00 (t, J = 7.4 Hz, 2H), 1.70-1.58 (m, 2H), 0.99 (t, | |
| | J = 7.3 Hz, 3H). | |
| 1-78 | ¹H NMR (400 MHz, DMSO-d₆) δ 12.85 (s, 1H), 8.14 (d, J = 8.1 Hz, 1H), 8.08 | |
| | (d, J = 8.1 Hz, 1H), 3.80-3.69 (m, 1H), 3.55 (s, 3H), 3.15-3.02 (m, 1H), 1.96- | |
| | 1.74 (m, 2H), 1.08 (t, J = 7.4 Hz, 3H). | |
| 1-81 | ¹H NMR (400 MHz, DMSO-d₆) δ 12.80 (s, 1H), 8.40 (d, J = 8.2 Hz, 1H), 8.25 | |
| | (d, J = 8.2 Hz, 1H), 3.73-3.67 (m, 2H), 3.66 (s, 3H), 1.97-1.84 (m, 2H), 1.06 (t, J = | |
| | 7.4 Hz, 3H). | |
| 1-82 | ¹H NMR (400 MHz, DMSO-d₆) δ 12.87 (s, 1H), 8.16 (d, J = 8.1 Hz, 1H), 7.93 | |
| | (d, J = 8.1 Hz, 1H), 3.86-3.75 (m, 1H), 3.56 (s, 3H), 1.26 (d, J = 6.8 Hz, 6H). | |
| 1-83 | ¹H NMR (400 MHz, DMSO-d₆) δ 12.84 (s, 1H), 8.21 (d, J = 8.1 Hz, 1H), 8.11 | |
| | (d, J = 8.1 Hz, 1H), 4.16-4.05 (m, 1H), 3.52 (s, 3H), 1.46 (d, J = 6.8 Hz, 3H), 1.13 | |
| | (d, J = 7.0 Hz, 3H). | |
| 1-86 | ¹H NMR (400 MHz, DMSO-d₆) δ 12.76 (s, 1H), 8.46 (d, J = 8.2 Hz, 1H), 8.25 | |
| | (d, J = 8.2 Hz, 1H), 4.25-4.13 (m, 1H), 3.64 (s, 3H), 1.36 (d, J = 6.8 Hz, 6H). | |
| 1-87 | ¹H NMR (400 MHz, DMSO-d₆) δ 11.91 (s, 1H), 8.16 (d, J = 8.1 Hz, 1H), 7.99 | |
| | (d, J = 7.6 Hz, 1H), 4.38 (q, J = 7.2 Hz, 2H), 3.58 (s, 3H), 3.00 (t, J = 7.3 Hz, 2H), | |
| | 1.71-1.54 (m, 2H), 1.48 (t, J = 7.2 Hz, 3H), 0.98 (t, J = 7.3 Hz, 3H). | |
| 1-88 | ¹H NMR (400 MHz, DMSO-d₆) δ 11.98 (s, 1H), 8.23-8.05 (m, 2H), 4.39 (q, J = | |
| | 7.2 Hz, 2H), 3.83-3.67 (m, 1H), 3.55 (s, 3H), 3.14-3.02 (m, 1H), 1.97-1.70 (m, | |
| | 2H), 1.48 (t, J = 7.2 Hz, 3H), 1.09 (t, J = 7.4 Hz, 3H). | |
| 1-91 | ¹H NMR (400 MHz, DMSO-d₆) δ 12.00 (s, 1H), 8.42 (d, J = 7.6 Hz, 1H), 8.36- | |
| | 8.22 (m, 1H), 4.50-4.31 (m, 2H), 3.84-3.58 (m, 5H), 2.05-1.78 (m, 2H), 1.49 (t, | |
| | J = 6.5 Hz, 3H), 1.07 (t, J = 6.6 Hz, 3H). | |
| 1-92 | ¹H NMR (400 MHz, DMSO-d₆) δ 11.95 (s, 1H), 8.18 (d, J = 8.1 Hz, 1H), 8.00 | |
| | (d, J = 8.2 Hz, 1H), 4.45-4.34 (m, 2H), 3.88-3.74 (m, 1H), 3.57 (s, 3H), 1.49 (t, J = | |
| | 7.2 Hz, 3H), 1.25 (t, J = 5.9 Hz, 6H). | |
| 1-93 | ¹H NMR (400 MHz, DMSO-d₆) δ 11.99 (s, 1H), 8.22 (d, J = 8.0 Hz, 1H), 8.16 | |
| | (d, J = 8.3 Hz, 1H), 4.44-4.32 (m, 2H), 4.18-4.07 (m, 1H), 3.53 (s, 3H), 1.52- | |
| | 1.41 (m, 6H), 1.12 (d, J = 7.0 Hz, 3H). | |
| 1-96 | ¹H NMR (400 MHz, DMSO-d₆) δ 11.99 (s, 1H), 8.48 (d, J = 8.2 Hz, 1H), 8.34 | |
| | (d, 1H), 4.44-4.34 (m, 2H), 4.26-4.14 (m, 1H), 3.64 (s, 3H), 1.48 (t, J = 7.2 Hz, | |
| | 3H), 1.37 (d, J = 6.8 Hz, 6H). | |
| 1-97 | ¹H NMR (400 MHz, DMSO-d₆ rotameric) δ 8.19 (d, J = 8.1 Hz, 0.5H), 8.04 (d, J = | |
| | 8.1 Hz, 0.5H), 7.95 (d, J = 8.2 Hz, 0.5H), 7.70 (d, J = 8.2 Hz, 0.5H), 4.09 (s, 1.5H), | |
| | 4.03 (s, 1.5H), 3.58 (s, 1.5H), 3.49 (s, 1.5H), 3.44 (s, 1.5H), 3.25 (s, 1.5H), 3.02 (t, J = | |
| | 7.4 Hz, 1H), 2.90-2.81 (m, 1H), 1.64 (q, J = 7.3 Hz, 1H), 1.50-1.37 (m, 1H), 0.98 | |
| | (t, J = 7.3 Hz, 1.5H), 0.92 (t, J = 7.3 Hz, 1.5H). | |
| 1-98 | ¹H NMR (400 MHz, CDCl₃-d rotameric) δ 8.27 (d, J = 7.9 Hz, 0.7H), 8.05 (d, J = | |
| | 8.2 Hz, 0.3H), 7.74 (d, J = 8.0 Hz, 0.7H), 7.69 (d, J = 8.1 Hz, 0.3H), 4.08 (s, 2.1H), | |
| | 4.03 (s, 0.9H), 3.93-3.80 (m, 0.7H), 3.76-3.66 (m, 0.3H), 3.52 (s, 0.9H), 3.44 (s, | |
| | 2.1H), 3.42 (s, 2.1H), 3.35 (s, 0.9H), 3.21-3.11 (m, 0.7H), 3.07-2.97 (m, 0.3H), | |
| | 2.03-1.95 (m, 1.4H), 1.94-1.86 (m, 0.6H), 1.19 (t, J = 7.4 Hz, 2.1H), 1.14 (t, J = | |
| | 7.4 Hz, 0.9H). | |
| 1-101 | ¹H NMR (400 MHz, DMSO-d₆ rotameric) δ 8.42 (d, J = 8.2 Hz, 0.5H), 8.35 (d, | |
| | J = 8.2 Hz, 0.5H), 8.19 (d, J = 8.2 Hz, 0.5H), 8.00 (d, J = 8.2 Hz, 0.5H), 4.11 (s, | |
| | 1.5H), 4.03 (s, 1.5H), 3.77-3.69 (m, 1H), 3.65 (s, 1.5H), 3.64-3.59 (m, 1H), 3.58 | |
| | (s, 1.5H), 3.47 (s, 1.5H), 3.27 (s, 1.5H), 1.96-1.90 (m, 1H), 1.76 (q, J = 7.5 Hz, 1H), | |
| | 1.06 (t, J = 7.4 Hz, 1.5H), 1.01 (t, J = 7.4 Hz, 1.5H). | |
| 1-102 | ¹H NMR (400 MHz, CDCl3-d, rotameric) δ 8.24 (d, J = 8.1 Hz, 0.7H), 8.02 (d, J = | |
| | 8.1 Hz, 0.3H), 7.61 (d, J = 8.1 Hz, 0.7H), 7.49 (d, J = 8.2 Hz, 0.3H), 4.03-3.92 (m, | |
| | 3H), 3.86-3.75 (m, 0.7H), 3.66-3.59 (m, 0.3H), 3.46-3.39 (m, 3H), 3.30 (s, 3H), | |
| | 1.27-1.23 (m, 4.2H), 1.09 (d, J = 6.7 Hz, 1.8H). | |
| 1-103 | ¹H NMR (400 MHz, CDCl3-d, rotameric) δ 8.39-8.34 (m, 0.7H), 8.15 (d, J = | |
| | 8.0 Hz, 0.3H), 7.81-7.69 (m, 1H), 4.40-3.89 (m, 4H), 3.60-3.24 (m, 6H), 1.71- | |
| | 0.98 (m, 6H). | |
| 1-106 | ¹H NMR (400 MHz, DMSO-d₆, rotameric) δ 8.51 (d, J = 8.3 Hz, 0.5H), 8.37 (d, | |
| | J = 8.3 Hz, 0.5H), 8.28 (d, J = 8.3 Hz, 0.5H), 8.02 (d, J = 8.3 Hz, 0.5H), 4.21 (p, J = | |
| | 6.8 Hz, 0.5H), 4.13-4.01 (m, 3.5H), 3.63 (s, 1.5H), 3.56 (s, 1.5H), 3.46 (s, 1.5H), | |
| | 3.27 (s, 1.5H), 1.39 (d, 3H), 1.10-1.02 (m, 3H). | |
| 1-112 | ¹H NMR (400 MHz, CDCl3-d, rotameric) δ 8.27 (d, J = 8.1 Hz, 0.7H), 8.06 (d, J = | |
| | 8.2 Hz, 0.3H), 7.56 (d, J = 8.1 Hz, 0.7H), 7.49 (d, J = 8.1 Hz, 0.3H), 4.02 (s, 2.1H), | |
| | 3.95 (s, 0.9H), 3.71-3.61 (m, 0.7H), 3.52-3.36 (m, 3.3H), 3.31 (s, 3H), 1.72-1.38 | |
| | (m, 2H), 1.20-1.16 (m, 3H), 1.04-0.87 (m, 3H). | |
| 1-115 | ¹H NMR (400 MHz, CDCl3-d, rotameric) δ 8.36 (d, J = 8.6 Hz, 0.7H), 8.14 (d, J = | |
| | 8.2 Hz, 0.3H), 7.81 (dd, J = 8.1, 4.1 Hz, 0.7H), 7.72 (dd, J = 8.2, 5.1 Hz, 0.3H), | |
| | 4.06 (d, J = 13.8 Hz, 4H), 3.52 (s, 0.9H), 3.48-3.29 (m, 5.1H), 2.30-0.79 (m, 8H). | |
| 1-120 | ¹H NMR (400 MHz, CDCl3-d, rotameric) δ 8.59 (d, J = 8.2 Hz, 0.7H), 8.39 (d, J = | |
| | 8.3 Hz, 0.3H), 7.87 (d, J = 8.2 Hz, 0.7H), 7.81 (d, J = 8.2 Hz, 0.3H), 4.15-4.00 (m, | |
| | 3.7H), 3.96-3.86 (m, 0.3H), 3.60 (s, 2.1H), 3.51 (d, J = 5.0 Hz, 1.8H), 3.40 (s, 2.1H), | |
| | 2.13-0.94 (m, 8H). | |
| 1-128 | ¹H NMR (400 MHz, CDCl₃-d, rotameric) δ 8.31 (d, J = 8.1 Hz, 0.7H), 8.09 (d, J = | |
| | 8.2 Hz, 0.3H), 7.63 (d, J = 8.1 Hz, 0.7H), 7.55 (d, J = 8.2 Hz, 0.3H), 4.15-3.96 (m, | |
| | 3H), 3.55-3.49 (m, 3H), 3.45-3.37 (m, 3H), 3.02 (d, J = 7.4 Hz, 1.4H), 2.85 (d, J = | |
| | 7.4 Hz, 0.6H), 1.19-0.89 (m, 1H), 0.63-0.50 (m, 2H), 0.34-0.10 (m, 2H). | |
| 1-129 | ¹H NMR (400 MHz, CDCl₃-d, rotameric) δ 8.25 (d, J = 8.0 Hz, 0.7H), 8.03 (d, | |
| | J = 8.2 Hz, 0.3H), 7.75 (d, J = 8.1 Hz, 0.7H), 7.68 (d, J = 8.1 Hz, 0.3H), 4.10-4.01 | |
| | (m, 3H), 3.91-3.68 (m, 1H), 3.58-3.28 (m, 6H), 2.99-2.70 (m, 1H), 1.44-1.30 | |
| | (m, 1H), 0.92-0.65 (m, 2H), 0.60-0.40 (m, 2H). | |
| 1-132 | ¹H NMR (400 MHz, CDCl3-d, rotameric) δ 8.50 (d, J = 8.2 Hz, 0.7H), 8.28 (d, | |
| | J = 8.2 Hz, 0.3H), 7.90 (d, J = 8.2 Hz, 0.7H), 7.80 (d, J = 8.2 Hz, 0.3H), 4.09-4.01 | |
| | (m, 3H), 3.69-3.60 (m, 3.5H), 3.55-3.50 (m, 2.4H), 3.39 (s, 2.1H), 1.45-1.30 (m, | |
| | 1H), 0.82-0.61 (m, 2H), 0.51-0.33 (m, 2H). | |
| 1-133 | ¹H NMR (400 MHz, CDCl3-d, rotameric) δ 8.31 (d, J = 8.1 Hz, 0.7H), 8.09 (d, J = | |
| | 8.2 Hz, 0.3H), 7.63 (d, J = 8.1 Hz, 0.7H), 7.56 (d, J = 8.2 Hz, 0.3H), 4.09 (s, 2.1H), | |
| | 4.00 (s, 0.9H), 3.52 (s, 0.9H), 3.40 (d, J = 2.5 Hz, 4.2H), 3.30 (s, 0.9H), 2.81-2.42 | |
| | (m, 1H), 1.00-0.64 (m, 4H). | |
| 1-134 | ¹H NMR (400 MHz, CDCl3-d, rotameric) δ 8.46-8.22 (m, 0.7H), 8.12 (d, J = | |
| | 8.1 Hz, 0.3H), 7.73 (dd, J = 18.7, 8.1 Hz, 1H), 4.16-3.97 (m, 4H), 3.53 (s, 0.9H), | |
| | 3.46-3.42 (m, 4.2H), 3.35 (s, 0.9H), 1.37-1.17 (m, 4H). | |
| 1-137 | ¹H NMR (400 MHz, CDCl3-d, rotameric) δ 8.56 (d, J = 8.1 Hz, 0.7H), 8.34 (d, J = | |
| | 8.2 Hz, 0.3H), 7.82 (dd, J = 21.1, 8.2 Hz, 1H), 4.20-3.97 (m, 4H), 3.69-3.35 (m, | |
| | 6H), 1.28-1.24 (m, 4H). | |
| 1-138 | ¹H NMR (400 MHz, CDCl3-d, rotameric) δ 8.31 (d, J = 8.1 Hz, 0.7H), 8.09 (d, J = | |
| | 8.2 Hz, 0.3H), 7.60 (d, J = 8.1 Hz, 0.7H), 7.53 (d, J = 8.1 Hz, 0.3H), 4.12-3.77 (m, | |
| | 4H), 3.58-3.41 (m, 3H), 3.40-3.35 (m, 3H), 2.47-1.60 (m, 6H). | |
| 1-139 | ¹H NMR (400 MHz, CDCl3-d, rotameric) δ 8.25 (d, J = 8.0 Hz, 0.7H), 8.05 (d, J = | |
| | 8.1 Hz, 0.3H), 7.77 (d, J = 8.1 Hz, 0.7H), 7.68 (d, J = 8.1 Hz, 0.3H), 4.60-4.27 (m, | |
| | 1H), 4.09-4.01 (m, 3H), 3.55-3.25 (m, 6H), 3.06-2.75 (m, 2H), 2.61-1.90 (m, 4H). | |
| 1-142 | ¹H NMR (400 MHz, DMSO-d₆, rotameric) δ 8.45 (d, J = 8.2 Hz, 0.5H), 8.34 (d, | |
| | J = 8.2 Hz, 0.5H), 8.23 (d, J = 8.3 Hz, 0.5H), 7.99 (d, J = 8.3 Hz, 0.5H), 4.84-4.55 | |
| | (m, 1H), 4.17-3.94 (m, 3H), 3.75-3.53 (m, 3H), 3.41-3.24 (m, 3H), 2.76-2.61 | |
| | (m, 2H), 2.37-1.79 (m, 4H). | |
| 1-143 | ¹H NMR (400 MHz, CDCl3-d, rotameric) δ 8.32 (d, J = 8.1 Hz, 0.7H), 8.10 (d, J = | |
| | 8.2 Hz, 0.3H), 7.65 (d, J = 8.1 Hz, 0.7H), 7.55 (d, J = 8.2 Hz, 0.3H), 4.14-4.00 (m, | |
| | 3H), 4.00-3.72 (m, 1H), 3.58-3.43 (m, 3H), 3.43-3.28 (m, 3H), 2.11-1.40 (m, 8H). | |
| 1-144 | ¹H NMR (400 MHz, CDCl3-d, rotameric) δ 8.34 (d, J = 7.9 Hz, 0.7H), 8.13 (d, J = | |
| | 8.1 Hz, 0.3H), 7.79 (d, J = 8.1 Hz, 0.7H), 7.71 (d, J = 8.1 Hz, 0.3H), 4.53-4.19 (m, | |
| | 1H), 4.16-3.96 (m, 3H), 3.60-3.24 (m, 6H), 2.46-2.14 (m, 2H), 1.96-1.41 (m, 6H). | |
| 1-147 | ¹H NMR (400 MHz, DMSO-d₆, rotameric) δ 8.51 (d, J = 8.3 Hz, 0.5H), 8.36 (d, J = | |
| | 8.3 Hz, 0.5H), 8.31-8.25 (m, 0.5H), 8.02 (d, J = 8.3 Hz, 0.5H), 4.53-4.41 (m, | |
| | 0.5H), 4.41-4.28 (m, 0.5H), 4.10 (s, 1.5H), 4.03 (s, 1.5H), 3.63 (s, 1.5H), 3.56 (s, | |
| | 1.5H), 3.47 (s, 1.5H), 3.27 (s, 1.5H), 2.25-1.54 (m, 8H). | |
| 1-168 | ¹H NMR (400 MHz, CDCl₃-d) δ 8.31 (d, J = 8.1 Hz, 0.6H), 8.07 (d, J = 8.2 Hz, | |
| | 0.4H), 7.63 (d, J = 8.1 Hz, 0.6H), 7.46 (d, J = 8.2 Hz, 0.4H), 4.35-4.16 (m, 1H), 4.07 | |
| | (s, 2H), 3.90 (s, 1H), 3.89-3.64 (m, 3H), 3.48 (s, 1.8H), 3.46-3.42 (m, 0.5H), 3.38 | |
| | (s, 1.2H), 3.35-3.27 (m, 0.5H), 3.24 (s, 1.2H), 3.21 (s, 1.8H), 1.40-1.29 (m, 4H), | |
| | 1.21 (d, J = 6.7 Hz, 2H). | |
| 1-169 | ¹H NMR (400 MHz, DMSO-d₆) δ 8.21 (d, J = 8.1 Hz, 1H), 8.09-7.95 (m, | |
| | 0.5H), 7.89 (d, J = 8.1 Hz, 0.5H), 4.18-4.06 (m, 1.5H), 4.03 (d, J = 7.3 Hz, 3H), 3.86- | |
| | 3.77 (m, 1H), 3.77-3.67 (m, 0.5H), 3.65-3.55 (m, 1H), 3.49 (d, J = 31.4 Hz, 3H), | |
| | 3.41-3.36 (m, 1H), 3.19-3.08 (m, 3H), 1.50-1.37 (m, 3H), 1.26-1.12 (m, 3H). | |
| 1-172 | ¹H NMR (400 MHz, DMSO-d₆) δ 8.48 (d, J = 8.3 Hz, 0.6H), 8.36 (d, J = 8.3 Hz, | |
| | 0.6H), 8.27 (d, J = 8.3 Hz, 0.4H), 8.04 (d, J = 8.3 Hz, 0.4H), 4.28-4.12 (m, 1H), | |
| | 4.12-4.05 (m, 0.8H), 4.03 (d, J = 11.0 Hz, 3H), 3.85-3.73 (m, 1.2H), 3.61-3.56 (m, | |
| | 0.8H), 3.59 (d, J = 31.1 Hz, 3H), 3.38-3.35 (m, 1.2H), 3.14 (d, J = 23.4 Hz, 3H), | |
| | 1.39 (dd, J = 6.8, 3.4 Hz, 3.6H), 1.31-1.18 (m, 2.4H). | |
| 1-173 | ¹H NMR (400 MHz, DMSO-d₆) δ 11.98 (s, 1H), 8.15 (d, J = 8.1 Hz, 1H), 7.99 | |
| | (d, J = 8.1 Hz, 1H), 4.03 (s, 3H), 3.58 (s, 3H), 3.11 (d, J = 7.6 Hz, 2H), 2.60-2.52 | |
| | (m, 1H), 2.12-2.00 (m, 2H), 1.90-1.65 (m, 4H). | |
| 1-174 | ¹H NMR (400 MHz, DMSO-d₆) δ 12.07 (s, 1H), 8.17-8.10 (m, 2H), 4.02 (s, | |
| | 3H), 3.93-3.85 (m, 1H), 3.54 (s, 3H), 3.27-3.19 (m, 1H), 2.93-2.80 (m, 1H), 2.26- | |
| | 2.09 (m, 2H), 2.00-1.90 (m, 4H). | |
| 1-177 | ¹H NMR (400 MHz, DMSO-d₆) δ 11.98 (s, 1H), 8.39 (d, J = 8.1 Hz, 1H), 8.29 | |
| | (d, J = 8.2 Hz, 1H), 4.02 (s, 3H), 3.84 (d, J = 7.3 Hz, 2H), 3.65 (s, 3H), 3.03-2.91 | |
| | (m, 1H), 2.20-2.05 (m, 2H), 2.00-1.91 (m, 4H). | |
| 1-178 | ¹H NMR (400 MHz, CDCl₃-d) δ 11.22 (s, 1H), 8.20 (d, J = 8.1 Hz, 1H), 7.74 (d, J = | |
| | 8.1 Hz, 1H), 4.14 (s, 3H), 3.50 (s, 3H), 3.01 (d, J = 7.4 Hz, 2H), 2.30-2.12 (m, 1H), | |
| | 1.96-1.72 (m, 2H), 1.73-1.52 (m, 4H), 1.40-1.25 (m, 2H). | |
| 1-179 | ¹H NMR (400 MHz, CDCl₃-d) δ 11.55 (s, 1H), 8.18 (d, J = 6.6 Hz, 1H), 7.86 (d, J = | |
| | 7.3 Hz, 1H), 4.12 (s, 3H), 4.02-3.95 (m, 1H), 3.45 (s, 3H), 3.10-3.00 (m, 1H), 2.54- | |
| | 2.49 (m, 1H), 2.06-1.89 (m, 2H), 1.83-1.59 (m, 4H), 1.57-1.46 (m, 1H), 1.43- | |
| | 1.31 (m, 1H). | |
| 1-182 | ¹H NMR (400 MHz, CDCl₃-d) δ 11.26 (s, 1H), 8.36 (d, J = 7.8 Hz, 1H), 7.95 (d, J = | |
| | 7.9 Hz, 1H), 4.10 (s, 3H), 3.69 (d, J = 7.0 Hz, 2H), 3.61 (s, 3H), 2.71-2.57 (m, 1H), | |
| | 2.10-2.01 (m, 2H), 1.78-1.56 (m, 4H), 1.49-1.36 (m, 2H). | |
| 1-183 | ¹H NMR (400 MHz, DMSO-d₆) δ 12.00 (s, 1H), 8.19 (d, J = 8.1 Hz, 1H), 8.03 | |
| | (d, J = 7.7 Hz, 1H), 4.03 (s, 3H), 3.92-3.83 (m, 1H), 3.57 (s, 3H), 3.13-2.97 (m, | |
| | 2H), 2.94-2.75 (m, 2H). | |
| 1-184 | ¹H NMR (400 MHz, DMSO-d₆) δ 12.01 (s, 1H), 8.19-8.10 (m, 2H), 4.32- | |
| | 4.26 (m, 1H), 4.02 (d, J = 4.4 Hz, 3H), 3.56 (s, 3H), 3.09-2.87 (m, 4H). | |
| 1-187 | ¹H NMR (400 MHz, DMSO-d₆) δ 12.01 (s, 1H), 8.19-8.10 (m, 2H), 4.32- | |
| | 4.26 (m, 1H), 4.02 (d, J = 4.4 Hz, 3H), 3.56 (s, 3H), 3.09-2.87 (m, 4H). | |
| 2-2 | ¹H NMR (400 MHz, CDCl₃-d) δ 8.18 (d, J = 8.1 Hz, 1H), 7.86-7.80 (m, 2H), | |
| | 4.03-3.73 (m, 4H), 3.44 (s, 3H), 3.17-3.05 (m, 1H), 2.04-1.90 (m, 2H), 1.17 (t, J = | |
| | 7.4 Hz, 3H). | |
| 2-5 | ¹H NMR (400 MHz, DMSO-d₆) δ 11.54 (s, 1H), 8.38 (d, J = 8.2 Hz, 1H), 8.26 | |
| | (d, J = 8.2 Hz, 1H), 7.96 (s, 1H), 3.80 (s, 3H), 3.74-3.63 (m, 5H), 1.89-1.82 (m, | |
| | 2H), 1.06 (t, J = 7.4 Hz, 3H). | |
| 2-6 | ¹H NMR (400 MHz, DMSO-d₆) δ 11.47 (s, 1H), 8.16 (d, J = 7.9 Hz, 1H), 8.01- | |
| | 7.87 (m, 2H), 3.90-3.81 (m, 1H), 3.80 (s, 3H), 3.56 (s, 3H), 1.26 (d, J = 6.5 Hz, 6H). | |
| 2-7 | ¹H NMR (400 MHz, DMSO-d₆) δ 11.53 (s, 1H), 8.21 (d, J = 7.9 Hz, 1H), 8.14 | |
| | (d, J = 3.5 Hz, 1H), 7.93 (s, 1H), 4.19-4.07 (m, 1H), 3.80 (s, 3H), 3.52 (s, 3H), 1.46 | |
| | (d, J = 6.7 Hz, 3H), 1.12 (d, J = 6.8 Hz, 3H). | |
| 2-8 | ¹H NMR (400 MHz, CDCl₃-d) δ 8.23 (d, J = 8.0 Hz, 1H), 7.84 (d, J = 8.0 Hz, | [α] |
| | 1H), 7.72 (s, 1H), 4.33-4.15 (m, 1H), 3.86 (s, 3H), 3.41 (s, 3H), 1.58 (d, J = 6.8 Hz, | D²⁰ = +56.09°(c |
| | 3H), 1.19 (d, J = 7.0 Hz, 3H). | 0.103, DMF) |
| 2-9 | ¹H NMR (400 MHz, CDCl₃-d) δ 8.23 (d, J = 8.0 Hz, 1H), 7.84 (d, J = 8.0 Hz, | [α] |
| | 1H), 7.72 (s, 1H), 4.32-4.14 (m, 1H), 3.86 (s, 3H), 3.41 (s, 3H), 1.58 (d, J = 6.8 Hz, | D²⁰ = −58.03°(c |
| | 3H), 1.19 (d, J = 7.0 Hz, 3H). | 0.102 , DMF) |
| 2-10 | ¹H NMR (400 MHz, DMSO-d₆) δ 11.59 (s, 1H), 8.46 (d, J = 8.1 Hz, 1H), 8.28 | |
| | (d, J = 7.8 Hz, 1H), 7.96 (s, 1H), 4.28-4.12 (m, 1H), 3.80 (s, 3H), 3.63 (s, 3H), 1.37 | |
| | (d, J = 6.4 Hz, 6H). | |
| 2-16 | ¹H NMR (400 MHz, CDCl₃) δ 8.24 (d, J = 8.1 Hz, 1H), 7.70 (d, J = 8.1 Hz, 1H), | |
| | 7.54 (s, 1H), 3.93 (s, 3H), 3.72-3.59 (m, 1H), 3.46 (s, 3H), 1.75-1.60 (m, 2H), 1.19 | |
| | (d, J = 6.6 Hz, 3H), 1.01 (t, J = 7.4 Hz, 3H). | |
| 2-19 | ¹H NMR (400 MHz, DMSO-d₆) δ 11.55 (s, 1H), 8.21 (d, J = 7.9 Hz, 1H), 8.14 | |
| | (d, J = 3.3 Hz, 1H), 7.95 (s, 1H), 3.94-3.84 (m, 1H), 3.80 (s, 3H), 3.52 (d, J = 3.6 | |
| | Hz, 3H), 2.13-2.02 (m, 0.5H), 1.84-1.68 (m, 0.5H), 1.57-1.46 (m, 1H), 1.42 (d, J = | |
| | 6.8 Hz, 1.5H), 1.07 (t, J = 7.5 Hz, 3H), 0.93 (t, J = 7.2 Hz, 1.5H). | |
| 2-24 | ¹H NMR (400 MHz, DMSO-d₆) δ 11.54 (s, 1H), 8.46 (d, J = 8.0 Hz, 1H), 8.28 | |
| | (d, J = 7.2 Hz, 1H), 7.95 (s, 1H), 4.06-3.97 (m, 1H), 3.80 (s, 3H), 3.63 (s, 3H), 2.03- | |
| | 1.93 (m, 1H), 1.74-1.58 (m, 1H), 1.35 (d, J = 6.7 Hz, 3H), 1.01 (t, J = 7.2 Hz, 3H). | |
| 2-27 | ¹H NMR (400 MHz, CDCl₃) δ 8.21 (d, J = 8.1 Hz, 1H), 7.72 (d, J = 8.1 Hz, 1H), | |
| | 7.57 (s, 1H), 3.90 (s, 3H), 3.51 (s, 3H), 2.94 (d, J = 7.4 Hz, 2H), 1.14-1.05 (m, 1H), | |
| | 0.63-0.51 (m, 2H), 0.31-0.21 (m, 2H). | |
| 2-28 | ¹H NMR (400 MHz, DMSO-d₆) δ 11.61 (s, 1H), 8.13 (d, J = 7.7 Hz, 1H), 8.09 | |
| | (d, J = 7.2 Hz, 1H), 7.95 (s, 1H), 3.89 (dd, J = 12.5, 5.1 Hz, 1H), 3.79 (s, 3H), 3.55 (s, | |
| | 3H), 2.88 (dd, J = 13.0, 9.1 Hz, 1H), 1.23 (s, 1H), 0.81-0.69 (m, 1H), 0.68-0.58 | |
| | (m, 1H), 0.55-0.44 (m, 1H), 0.39 (m, 1H). | |
| 2-29 | ¹H NMR (400 MHz, CDCl₃-d) δ 8.14 (d, J = 8.1 Hz, 1H), 7.82 (d, J = 8.1 Hz, | [α] |
| | 1H), 7.76 (s, 1H), 4.03 (dd, J = 13.0, 6.1 Hz, 1H), 3.85 (s, 3H), 3.43 (s, 3H), 2.83 (dd, | D²⁰ = +99.87°(c |
| | J = 13.1, 9.1 Hz, 1H), 1.38-1.26 (m, 1H), 0.92-0.65 (m, 2H), 0.57-0.45 (m, 2H). | 0.104, DMF) |
| 2-30 | ¹H NMR (400 MHz, CDCl₃-d) δ 8.14 (d, J = 8.1 Hz, 1H), 7.82 (d, J = 8.1 Hz, | [α] |
| | 1H), 7.76 (s, 1H), 4.03 (dd, J = 13.0, 6.1 Hz, 1H), 3.85 (s, 3H), 3.43 (s, 3H), 2.83 (dd, | D²⁰ = −98.32°(c |
| | J = 13.1, 9.1 Hz, 1H), 1.39-1.25 (m, 1H), 0.92-0.65 (m, 2H), 0.57-0.45 (m, 2H). | 0.108, DMF) |
| 2-31 | ¹H NMR (400 MHz, DMSO-d₆) δ 11.54 (s, 1H), 8.40 (d, J = 8.0 Hz, 1H), 8.28 | |
| | (d, J = 7.8 Hz, 1H), 7.94 (s, 1H), 3.80 (s, 3H), 3.69 (d, J = 7.2 Hz, 2H), 3.66 (s, 3H), | |
| | 1.27-1.21 (m, 1H), 0.63 (m, 2H), 0.37 (m, 2H). | |
| 2-32 | ¹H NMR (400 MHz, CDCl₃-d) δ 8.21 (d, J = 8.1 Hz, 1H), 7.71 (d, J = 8.1 Hz, | |
| | 1H), 7.49 (s, 1H), 3.91 (s, 3H), 3.39 (s, 3H), 2.67 (dt, J = 11.2, 5.8 Hz, 1H), 0.88 (s, 4H). | |
| 2-33 | ¹H NMR (400 MHz, DMSO-d₆) δ 11.61 (s, 1H), 8.17 (d, J = 7.8 Hz, 1H), 8.10 | |
| | (d, J = 7.1 Hz, 1H), 7.95 (s, 1H), 3.80 (s, 3H), 3.53 (s, 3H), 3.35 (s, 1H), 1.37-1.18 | |
| | (m, 2H), 1.12-0.96 (m, 2H). | |
| 2-36 | ¹H NMR (400 MHz, DMSO-d₆) δ 11.61 (s, 1H), 8.42 (d, J = 8.2 Hz, 1H), 8.26 | |
| | (d, J = 8.1 Hz, 1H), 7.96 (s, 1H), 3.80 (s, 3H), 3.65 (s, 3H), 3.51-3.43 (m, 2H), 1.58- | |
| | 1.43 (m, 2H), 1.29-1.21 (m, 2H). | |
| 2-37 | ¹H NMR (400 MHz, DMSO-d₆) δ 11.46 (s, 1H), 8.14 (d, J = 7.9 Hz, 1H), 7.93 | |
| | (d, J = 10.1 Hz, 2H), 4.02-3.92 (m, 1H), 3.80 (s, 3H), 3.56 (s, 3H), 2.40-2.29 (m, | |
| | 2H), 2.26-2.08 (m, 2H), 1.97-1.77 (m, 2H). | |
| 2-38 | ¹H NMR (400 MHz, DMSO-d₆) δ 11.53 (s, 1H), 8.13 (d, J = 7.8 Hz, 1H), 8.07 | |
| | (d, J = 7.7 Hz, 1H), 7.95 (s, 1H), 4.54-4.39 (m, 1H), 3.79 (s, 3H), 3.53 (s, 3H), 2.82- | |
| | 2.70 (m, 1H), 2.39-2.27 (m, 1H), 2.26-2.18 (m, 1H), 2.17-2.07 (m, 1H), 2.06- | |
| | 1.92 (m, 2H). | |
| 2-41 | ¹H NMR (400 MHz, DMSO-d₆) δ 11.55 (s, 1H), 8.41 (d, J = 8.1 Hz, 1H), 8.25 | |
| | (d, J = 7.5 Hz, 1H), 7.95 (s, 1H), 4.82-4.66 (m, 1H), 3.79 (s, 3H), 3.65 (s, 3H), 2.76- | |
| | 2.60 (m, 2H), 2.32-2.17 (m, 2H), 2.09-1.89 (m, 2H). | |
| 2-42 | ¹H NMR (400 MHz, DMSO-d₆) δ 11.49 (s, 1H), 8.15 (d, J = 7.9 Hz, 1H), 7.95 (s, | |
| | 2H), 3.99-3.87 (m, 1H), 3.80 (s, 3H), 3.55 (s, 3H), 1.97-1.47 (m, 8H). | |
| 2-43 | ¹H NMR (400 MHz, DMSO-d₆) δ 11.54 (s, 1H), 8.19 (d, J = 7.9 Hz, 1H), 8.12 (d, J = | |
| | 7.2 Hz, 1H), 7.94 (s, 1H), 4.46-4.30 (m, 1H), 3.80 (s, 3H), 3.53 (s, 3H), 2.26-2.13 | |
| | (m, 1H), 2.13-2.00 (m, 1H), 1.78-1.52 (m, 6H). | |
| 2-46 | ¹H NMR (400 MHz, DMSO-d₆) δ 11.72 (s, 1H), 8.45 (d, J = 8.0 Hz, 1H), 8.25 | |
| | (d, J = 5.8 Hz, 1H), 7.94 (s, 1H), 4.53-4.42 (m, 1H), 3.79 (s, 3H), 3.63 (s, 3H), 2.27- | |
| | 2.10 (m, 2H), 2.03-1.87 (m, 2H), 1.84-1.70 (m, 2H), 1.68-1.53 (m, 2H). | |
| 2-53 | ¹H NMR (400 MHz, DMSO-d₆) δ 11.53 (s, 1H), 8.18 (s, 2H), 7.94 (s, 1H), 4.84- | |
| | 4.62 (m, 1H), 4.50-4.30 (m, 1H), 3.80 (s, 3H), 3.55 (s, 3H). | |
| 2-58 | ¹H NMR (400 MHz, DMSO-d₆) δ 11.50 (s, 1H), 8.14 (d, J = 9.0 Hz, 2H), 7.94 (s, | |
| | 1H), 6.80-6.45 (m, 1H), 4.45-4.27 (m, 1H), 3.94-3.82 (m, 1H), 3.80 (s, 3H), 3.55 | |
| | (s, 3H). | |
| 2-63 | ¹H NMR (400 MHz, DMSO-d₆) δ 11.57 (s, 1H), 8.17 (s, 2H), 7.94 (s, 1H), 4.98 (d, | |
| | J = 15.8 Hz, 1H), 4.72 (d, J = 16.2 Hz, 1H), 3.80 (s, 3H), 3.56 (s, 3H). | |
| 2-67 | ¹H NMR (400 MHz, CDCl₃-d) δ 8.21 (d, J = 8.1 Hz, 1H), 7.71 (d, J = 8.1 Hz, | |
| | 1H), 7.64 (s, 1H), 5.99-5.84 (m, 1H), 5.24-4.92 (m, 2H), 3.92 (s, 3H), 3.70 (d, J = | |
| | 7.5 Hz, 2H), 3.47 (s, 3H). | |
| 2-73 | ¹H NMR (400 MHz, DMSO-d₆) δ 11.54 (s, 1H), 8.20-8.08 (m, 2H), 7.95 (s, 1H), | |
| | 4.26-4.12 (m, 2H), 4.12-4.01 (m, 1H), 3.80 (s, 3H), 3.59-3.50 (m, 4H). | |
| 2-78 | ¹H NMR (400 MHz, CDCl₃-d, rotameric) δ 8.22-8.15 (m, 0.4H), 7.94 (d, J = | |
| | 8.1 Hz, 0.6H), 7.82 (s, 0.4H), 7.67 (d, J = 8.0 Hz, 0.4H), 7.60 (s, 0.6H), 7.53 (d, J = | |
| | 8.1 Hz, 0.6H), 3.85-3.80 (m, 1.6H), 3.75-3.64 (m, 2.4H), 3.43-3.34 (m, 3H), 3.33- | |
| | 3.21 (m, 3H), 3.13-3.01 (m, 0.4H), 3.01-2.89 (m, 0.6H), 2.00-1.76 (m, 2H), | |
| | 1.16-1.03 (m, 3H). | |
| 2-82 | ¹H NMR (400 MHz, DMSO-d₆, rotameric) δ 8.21 (d, J = 8.1 Hz, 0.3H), 8.02- | |
| | 7.94 (m, 1.3H), 7.67 (d, J = 8.7 Hz, 1.4H), 3.82 (s, 2.1H), 3.81-3.79 (m, 1.2H), 3.70- | |
| | 3.64 (m, 0.7H), 3.57 (s, 0.9H), 3.47 (s, 2.1H), 3.37 (s, 2.1H), 3.16 (s, 0.9H), 1.25 (d, J = | |
| | 6.9 Hz, 1.8H), 1.10 (d, J = 6.6 Hz, 4.2H). | |
| 2-83 | ¹H NMR (400 MHz, DMSO-d₆, rotameric) δ 8.24 (d, J = 8.1 Hz, 0.3H), 8.16- | |
| | 7.98 (m, 1.3H), 7.84 (d, J = 8.1 Hz, 0.7H), 7.69 (s, 0.7H), 4.15-4.04 (m, 0.3H), 4.10- | |
| | 3.89 (s, 0.7H), 3.84 (s, 2.1H), 3.81 (s, 0.9H), 3.53 (s, 0.9H), 3.46 (s, 2.1H), 3.38 (s, | |
| | 2.1H), 3.20-3.19 (s, 0.9H), 1.46 (d, J = 6.8 Hz, 0.9H), 1.40 (d, J = 6.8 Hz, 2.1H), 1.14 | |
| | (s, 0.9H), 0.91 (d, J = 7.0 Hz, 2.1H). | |
| 2-86 | ¹H NMR (400 MHz, DMSO-d₆, rotameric) δ 8.49 (d, J = 8.3 Hz, 0.3H), 8.32- | |
| | 8.25 (m, 1H), 8.02-7.97 (m, 1H), 7.70 (s, 0.7H), 4.26-4.14 (m, 0.3H), 4.09-4.02 | |
| | (m, 0.7H), 3.85 (s, 2.1H), 3.81 (s, 0.9H), 3.63 (s, 0.9H), 3.56 (s, 2.1H), 3.39 (s, 2.1H), | |
| | 3.18 (s, 0.9H), 1.38 (d, J = 6.9 Hz, 1.8H), 1.24 (d, J = 6.6 Hz, 4.2H). | |
| 2-87 | ¹H NMR (400 MHz, CDCl₃-d, rotameric) δ 8.28 (d, J = 8.2 Hz, 0.4H), 8.02 (d, J = | |
| | 8.2 Hz, 0.6H), 7.93 (s, 0.4H), 7.68 (s, 0.6H), 7.59 (d, J = 8.2 Hz, 0.4H), 7.42 (d, J = | |
| | 8.2 Hz, 0.6H), 4.02-3.93 (m, 1.2H), 3.91 (s, 1.2H), 3.74 (s, 1.8H), 3.70-3.55 (m, | |
| | 0.8H), 3.50 (s, 1.2H), 3.40 (s, 1.8H), 3.12-2.97 (m, 0.8H), 2.92 (t, J = 7.5 Hz, 1.2H), | |
| | 1.77-1.71 (m, 0.8H), 1.66-1.59 (m, 1.2H), 1.32 (t, J = 7.2 Hz, 1.8H), 1.13 (t, J = | |
| | 7.2 Hz, 1.2H), 1.06 (t, J = 7.4 Hz, 1.2H), 1.02 (t, J = 7.4 Hz, 1.8H). | |
| 2-88 | ¹H NMR (400 MHz, CDCl₃-d, rotameric) δ 8.29-8.20 (m, 0.3H), 7.98 (d, | |
| | 0.7H), 7.93 (s, 0.3H), 7.75-7.69 (m, 1H), 7.53 (d, J = 8.1 Hz, 0.7H), 4.05-3.93 (m, | |
| | 1.4H), 3.90 (s, 0.9H), 3.86-3.79 (m, 0.6H), 3.77 (s, 2.1H), 3.74-3.65 (m, 0.7H), | |
| | 3.63-3.54 (m, 0.3H), 3.44 (d, J = 6.0 Hz, 0.9H), 3.36 (s, 2.1H), 3.21-3.09 (m, | |
| | 0.3H), 3.08-2.98 (m, 0.7H), 2.08-1.95 (m, 1.4H), 1.95-1.85 (m, 0.6H), 1.36- | |
| | 1.11 (m, 8H). | |
| 2-91 | ¹H NMR (400 MHz, CDCl₃-d, rotameric) δ 8.47 (d, J = 8.1 Hz, 0.3H), 8.19 (d, | |
| | J = 8.2 Hz, 0.7H), 7.93 (s, 0.3H), 7.84 (d, J = 8.1 Hz, 0.3H), 7.71 (s, 0.7H), 7.64 (d, J = | |
| | 8.2 Hz, 0.7H), 4.02-3.92 (m, 1.4H), 3.90 (s, 0.9H), 3.78 (s, 2.1H), 3.76-3.68 (m, | |
| | 0.6H), 3.68-3.64 (m, 0.6H), 3.62 (s, 0.9H), 3.61-3.55 (m, 1.4H), 3.54 (s, 2.1H), | |
| | 2.14-2.09 (m, 0.6H), 2.06-1.97 (m, 1.4H), 1.37-1.09 (m, 6H). | |
| 2-98 | ¹H NMR (400 MHz, DMSO-d₆, rotameric) δ 8.18-8.10 (m, 0.5H), 8.09-7.98 | |
| | (m, 1H), 7.94 (d, J = 8.2 Hz, 0.5H), 7.81-7.74 (m, 1H), 4.53-4.40 (m, 0.5H), 4.39- | |
| | 4.23 (m, 0.5H), 4.02 (t, J = 5.4 Hz, 1H), 3.79 (s, 1.5H), 3.73 (s, 1.5H), 3.71-3.58 (m, | |
| | 1H), 3.57-3.51 (m, 2.5H), 3.46 (s, 1.5H), 3.29 (q, J = 5.2, 4.2 Hz, 1H), 3.21 (s, | |
| | 1.5H), 3.11 (d, J = 5.9 Hz, 1.5H), 2.86-2.60 (m, 1H), 2.32 (q, J = 9.4, 9.0 Hz, 0.5H), | |
| | 2.25-2.07 (m, 2H), 2.05-1.91 (m, 2.5H). | |
| 3-1 | ¹H NMR (400 MHz, DMSO-d₆) δ 12.44 (s, 1H), 8.12 (d, J = 8.1 Hz, 1H), 7.89 | |
| | (d, J = 8.2 Hz, 1H), 3.57 (s, 3H), 2.98 (t, J = 7.5 Hz, 2H), 1.63 (h, J = 7.3 Hz, 2H), | |
| | 0.98 (t, J = 7.3 Hz, 3H). | |
| 3-2 | ¹H NMR (400 MHz, DMSO-d₆) δ 12.53 (s, 1H), 8.11 (d, J = 8.1 Hz, 1H), 8.02 | |
| | (d, J = 8.1 Hz, 1H), 3.78-3.68 (m, 1H), 3.53 (s, 3H), 3.10-3.02 (m, 1H), 2.48 (s, | |
| | 3H), 1.95-1.73 (m, 2H), 1.08 (t, J = 7.4 Hz, 3H). | |
| 3-3 | ¹H NMR (400 MHz, DMSO-d₆) δ 12.57 (s, 1H), 8.11 (d, J = 8.1 Hz, 1H), 8.02 | [α] |
| | (d, J = 8.1 Hz, 1H), 3.80-3.68 (m, 1H), 3.53 (s, 3H), 3.12-3.01 (m, 1H), 2.48 (s, | D²⁰ = +98.84°(c |
| | 3H), 1.95-1.73 (m, 2H), 1.08 (t, J = 7.4 Hz, 3H). | 0.108, DMF) |
| 3-4 | ¹H NMR (400 MHz, DMSO-d₆) δ 12.53 (s, 1H), 8.11 (d, J = 8.1 Hz, 1H), 8.02 | [α] |
| | (d, J = 8.1 Hz, 1H), 3.78-3.68 (m, 1H), 3.53 (s, 3H), 3.10-3.02 (m, 1H), 2.48 (s, | D²⁰ = −101.09°(c |
| | 3H), 1.95-1.73 (m, 2H), 1.08 (t, J = 7.4 Hz, 3H). | 0.102, DMF) |
| 3-5 | ¹H NMR (400 MHz, DMSO-d₆) δ 12.62 (s, 1H), 8.37 (d, J = 8.2 Hz, 1H), 8.19 | |
| | (d, J = 8.2 Hz, 1H), 3.73-3.66 (m, 2H), 3.65 (s, 3H), 2.49 (s, 3H), 1.89 (h, J = 7.5 | |
| | Hz, 2H), 1.06 (t, J = 7.4 Hz, 3H). | |
| 3-6 | ¹H NMR (400 MHz, DMSO-d₆) δ 12.48 (s, 1H), 8.14 (d, J = 8.1 Hz, 1H), 7.87 | |
| | (d, J = 8.2 Hz, 1H), 3.78 (h, J = 6.7 Hz, 1H), 3.55 (s, 3H), 2.48 (s, 3H), 1.24 (d, J = | |
| | 6.6 Hz, 6H). | |
| 3-7 | ¹H NMR (400 MHz, DMSO-d₆) δ 12.55 (s, 1H), 8.18 (d, J = 8.1 Hz, 1H), 8.05 | |
| | (d, J = 8.1 Hz, 1H), 4.23-3.93 (m, 1H), 3.51 (s, 3H), 2.48 (s, 3H), 1.45 (d, J = 6.8 | |
| | Hz, 3H), 1.11 (d, J = 7.0 Hz, 3H). | |
| 3-10 | ¹H NMR (400 MHz, DMSO-d6) δ 12.55 (s, 1H), 8.18 (d, J = 8.1 Hz, 1H), 8.05 | |
| | (d, J = 8.1 Hz, 1H), 4.16-4.01 (m, 1H), 3.51 (s, 3H), 2.48 (s, 3H), 1.45 (d, J = 6.8 | |
| | Hz, 3H), 1.11 (d, J = 7.0 Hz, 3H). | |
| 3-11 | ¹H NMR (400 MHz, DMSO-d₆) δ 12.45 (s, 1H), 8.12 (d, J = 8.1 Hz, 1H), 7.88 | |
| | (d, J = 8.1 Hz, 1H), 3.56 (s, 3H), 3.00 (t, J = 7.5 Hz, 2H), 2.48 (s, 3H), 1.60 (p, J = 7.3 | |
| | Hz, 2H), 1.40 (h, J = 7.3 Hz, 2H), 0.89 (t, J = 7.3 Hz, 3H). | |
| 3-12 | ¹H NMR (400 MHz, DMSO-d6) δ 12.52 (s, 1H), 8.11 (d, J = 8.1 Hz, 1H), 8.03 | |
| | (d, J = 8.1 Hz, 1H), 3.73 (s, 1H), 3.53 (s, 3H), 3.18-3.02 (m, 1H), 2.45 (s, 3H), 1.86- | |
| | 1.72 (m, 2H), 1.60-1.39 (m, 2H), 0.94 (t, J = 7.3 Hz, 3H). | |
| 3-15 | ¹H NMR (400 MHz, DMSO-d₆) δ 12.55 (s, 1H), 8.37 (d, J = 8.2 Hz, 1H), 8.19 | |
| | (d, J = 8.2 Hz, 1H), 3.74-3.67 (m, 2H), 3.65 (s, 3H), 2.49 (s, 3H), 1.89-1.78 (m, | |
| | 2H), 1.47 (h, J = 7.4 Hz, 2H), 0.91 (t, J = 7.3 Hz, 3H). | |
| 3-16 | ¹H NMR (400 MHz, CDCl3-d) δ 8.23 (d, J = 8.2 Hz, 1H), 7.74 (d, J = 8.1 Hz, | |
| | 1H), 3.75-3.64 (m, 1H), 3.45 (s, 3H), 2.53 (s, 3H), 1.74-1.64 (m, 2H), 1.24 (t, J = | |
| | 7.0 Hz, 3H), 1.02 (t, J = 7.4 Hz, 3H). | |
| 3-19 | ¹H NMR (400 MHz, CDCl₃-d) δ 8.26 (d, J = 8.1 Hz, 1H), 7.85 (dd, J = 8.1, 3.8 | |
| | Hz, 1H), 4.23-3.95 (m, 1H), 3.42 (d, J = 3.1 Hz, 3H), 2.54 (s, 3H), 2.28-2.11 (m, | |
| | 0.5H), 1.93-1.78 (m, 0.5H), 1.64-1.48 (m, 2H), 1.26-1.23 (m, 1H), 1.18-1.11 | |
| | (m, 3H), 0.98 (t, J = 7.4 Hz, 1H). | |
| 3-24 | ¹H NMR (400 MHz, CDCl3-d) δ 8.49 (d, J = 8.3 Hz, 1H), 7.90 (d, J = 8.2 Hz, | |
| | 1H), 4.08-3.97 (m, 1H), 3.58 (s, 3H), 2.53 (s, 3H), 1.88-1.69 (m, 2H), 1.44 (d, J = | |
| | 6.9 Hz, 3H), 1.05 (t, J = 7.4 Hz, 3H). | |
| 3-25 | ¹H NMR (400 MHz, CDCl₃-d) δ 8.48 (d, J = 8.2 Hz, 1H), 7.90 (d, J = 8.2 Hz, | [α] |
| | 1H), 4.06-3.97 (m, 1H), 3.58 (s, 3H), 2.53 (s, 3H), 2.15-2.07 (m, 1H), 1.84-1.72 | D²⁰ = +1.97°(c |
| | (m, 1H), 1.45 (d, J = 6.9 Hz, 3H), 1.06 (t, J = 7.5 Hz, 3H). | 0.102, DMF) |
| 3-26 | ¹H NMR (400 MHz, CDCl₃-d) δ 8.48 (d, J = 8.1 Hz, 1H), 7.90 (d, J = 8.2 Hz, | [α] |
| | 1H), 4.08-3.95 (m, 1H), 3.58 (s, 3H), 2.52 (s, 3H), 2.14-2.06 (m, 1H), 1.84-1.68 | D20 = −1.97°(c |
| | (m, 1H), 1.43 (d, J = 6.9 Hz, 3H), 1.05 (t, J = 7.5 Hz, 3H). | 0.102, DMF) |
| 3-27 | ¹H NMR (400 MHz, DMSO-d₆) δ 12.44 (s, 1H), 8.10 (d, J = 8.1 Hz 1H), 7.86 | |
| | (d, J = 8.1 Hz, 1H), 3.56 (s, 3H), 2.88 (d, J = 6.8 Hz, 2H), 2.47 (s, 3H), 2.05-1.86 | |
| | (m, 1H), 1.02 (d, J = 6.6 Hz, 6H). | |
| 3-28 | ¹H NMR (400 MHz, DMSO-d₆) δ 12.49 (s, 1H), 8.11 (d, J = 8.1 Hz, 1H), 8.02 | |
| | (d, J = 8.1 Hz, 1H), 3.78-3.70 (m, 1H), 3.54 (s, 3H), 2.89-2.79 (m, 1H), 2.49 (s, | |
| | 3H), 2.32-2.18 (m, 1H), 1.15-1.07 (m, 6H). | |
| 3-31 | 1H NMR ( 400 MHz, DMSO-d₆) δ 12.53 (s, 1H), 8.35 (d, J = 8.2 Hz, 1H), 8.19 | |
| | (d, J = 8.3 Hz, 1H), 3.65 (s, 3H), 3.59 (d, J = 6.5 Hz, 2H), 2.48-2.42 (m, 4H), 1.11 | |
| | (d, J = 6.7 Hz, 6H). | |
| 3-32 | ¹H NMR (400 MHz, DMSO-d₆) δ 12.50 (s, 1H), 8.18 (d, J = 8.1 Hz, 1H), 7.89 | |
| | (d, J = 8.3 Hz, 1H), 3.48 (s, 3H), 2.47 (s, 3H), 1.41 (s, 9H). | |
| 3-37 | ¹H NMR (400 MHz, DMSO-d₆) δ 12.47 (s, 1H), 8.11 (d, J = 8.1 Hz, 1H), 7.86 | |
| | (d, J = 8.1 Hz, 1H), 3.60 (s, 3H), 2.97 (d, J = 7.3 Hz, 2H), 2.48 (s, 3H), 1.10-0.95 | |
| | (m, 1H), 0.57-0.48 (m, 2H), 0.27-0.18 (m, 2H). | |
| 3-38 | ¹H NMR (400 MHz, DMSO-d₆) δ 12.11 (s, 1H), 8.13 (d, J = 8.0 Hz, 1H), 8.00 | |
| | (d, J = 8.1 Hz, 1H), 3.91 (dd, J = 13.1, 6.0 Hz, 1H), 3.57 (s, 3H), 2.91 (dd, J = 13.1, | |
| | 8.9 Hz, 1H), 2.50 (s, 3H), 1.25-1.16 (m, 1H), 0.78-0.64 (m, 2H), 0.52 (dt, J = 9.5, | |
| | 4.7 Hz, 1H), 0.43 (dt, J = 9.6, 4.8 Hz, 1H). | |
| 3-41 | ¹H NMR (400 MHz, DMSO-d₆) δ 12.60 (s, 1H), 8.38 (d, J = 8.2 Hz, 1H), 8.20 | |
| | (d, J = 8.3 Hz, 1H), 3.74-3.60 (m, 5H), 2.50 (s, 3H), 1.23-1.08 (m, 1H), 0.71- | |
| | 0.55 (m, 2H), 0.41-0.30 (m, 2H). | |
| 3-52 | ¹H NMR (400 MHz, DMSO-d₆) δ 12.44 (s, 1H), 8.12 (d, J = 8.1 Hz, 1H), 7.89 | |
| | (d, J = 8.1 Hz, 1H), 3.57 (s, 3H), 3.01 (t, J = 7.7 Hz, 2H), 2.49 (s, 3H), 1.74-1.59 | |
| | (m, 1H), 1.56-1.46 (m, 2H), 0.93 (d, J = 6.5 Hz, 6H). | |
| 3-53 | ¹H NMR (400 MHz, DMSO-d₆) δ 12.54 (s, 1H), 8.11 (d, J = 8.0 Hz, 1H), 8.02 | |
| | (d, J = 8.1 Hz, 1H), 3.78-3.68 (m, 1H), 3.53 (s, 3H), 3.17-3.05 (m, 1H), 2.48 (s, | |
| | 3H), 1.84-1.58 (m, 3H), 0.93 (dd, J = 6.3, 2.0 Hz, 6H). | |
| 3-67 | ¹H NMR (400 MHz, DMSO-d₆) δ 12.52 (s, 1H), 8.12 (d, J = 8.1 Hz, 1H), 7.91 | |
| | (d, J = 8.2 Hz, 1H), 7.32 (q, J = 6.8, 6.3 Hz, 5H), 4.25 (s, 2H), 3.30 (s, 3H), 2.50 (s, 3H). | |
| 3-68 | ¹H NMR (400 MHz, CDCl3-d) δ 8.22 (d, J = 8.1 Hz, 1H), 7.89 (d, J = 8.1 Hz, | |
| | 1H), 7.51-7.45 (m, 2H), 7.44-7.37 (m, 3H), 5.05 (d, J = 12.6 Hz, 1H), 4.48 (d, J = | |
| | 12.6 Hz, 1H), 3.43 (s, 3H), 2.52 (s, 3H). | |
| 3-71 | ¹H NMR (400 MHz, CDCl3-d) δ 8.41 (d, J = 8.2 Hz, 1H), 7.91 (d, J = 8.2 Hz, | |
| | 1H), 7.42 (s, 5H), 4.93 (s, 2H), 3.57 (d, J = 2.2 Hz, 3H), 2.55 (s, 3H). | |
| 3-77 | ¹H NMR (400 MHz, DMSO-d₆) δ 12.50 (s, 1H), 8.11 (d, J = 8.3 Hz, 1H), 7.88 | |
| | (d, J = 8.0 Hz, 1H), 3.48 (s, 3H), 2.67 (s, 1H), 2.48 (s, 3H), 0.98-0.84 (m, 2H), 0.82- | |
| | 0.65 (m, 2H). | |
| 3-78 | ¹H NMR (400 MHz, CDCl₃-d) δ 8.16 (d, J = 8.1 Hz, 1H), 7.79 (d, J = 8.1 Hz, | |
| | 1H), 3.49-3.38 (m, 1H), 3.35 (s, 3H), 2.47 (s, 3H), 1.40-1.29 (m, 1H), 1.28-1.12 | |
| | (m, 1H), 1.01-0.89 (m, 1H), 0.80-0.73 (m, 1H). | |
| 3-81 | ¹H NMR (400 MHz, DMSO-d₆) δ 12.59 (s, 1H), 8.41 (d, J = 8.2 Hz, 1H), 8.19 | |
| | (d, J = 8.2 Hz, 1H), 3.64 (s, 3H), 3.48-3.45 (m, 1H), 2.49 (s, 3H), 1.51-1.44 (m, | |
| | 2H), 1.33-1.18 (m, 2H). | |
| 3-82 | ¹H NMR (400 MHz, CDCl3-d) δ 8.18 (d, J = 8.2 Hz, 1H), 7.71 (d, J = 8.2 Hz, | |
| | 1H), 3.95 (p, J = 8.3 Hz, 1H), 3.44 (d, J = 0.9 Hz, 3H), 2.53 (s, 3H), 2.43-2.17 (m, | |
| | 4H), 2.02-1.77 (m, 2H). | |
| 3-83 | ¹H NMR (400 MHz, CDCl3-d) δ 8.16 (d, J = 8.1 Hz, 1H), 7.83 (d, J = 8.1 Hz, | |
| | 1H), 4.56 (p, J = 8.6 Hz, 1H), 3.42-3.38 (brs, 3H), 2.89 (dt, J = 19.9, 9.6 Hz, 1H), | |
| | 2.53 (s, 3H), 2.52-2.41 (m, 1H), 2.39-2.27 (m, 1H), 2.26-2.14 (m , 1H), 2.09- | |
| | 2.05 (m, 2H). | |
| 3-86 | ¹H NMR (400 MHz, CDCl3-d) δ 8.42 (d, J = 8.2 Hz, 1H), 7.90 (d, J = 8.2 Hz, | |
| | 1H), 4.68 (p, J = 8.7 Hz, 1H), 3.60 (s, 3H), 2.88 (dq, J = 12.3, 9.4 Hz, 2H), 2.54 (s, | |
| | 3H), 2.35-2.23 (m, 2H), 2.10-2.02 (m, 2H). | |
| 3-87 | ¹H NMR (400 MHz, DMSO-d₆) δ 12.45 (s, 1H), 8.13 (d, J = 8.1 Hz, 1H), 7.88 | |
| | (d, J = 8.1 Hz, 1H), 3.92 (d, J = 6.8 Hz, 1H), 3.54 (s, 3H), 1.93-1.88 (m, 2H), 1.75- | |
| | 1.66 (m, 2H), 1.59-1.54 (m, 4H). | |
| 3-88 | ¹H NMR (400 MHz, DMSO-d₆) δ 12.57 (s, 1H), 8.17 (d, J = 8.1 Hz, 1H), 8.04 | |
| | (d, J = 8.1 Hz, 1H), 4.44-4.32 (m, 1H), 3.52 (s, 3H), 2.49 (s, 3H), 2.24-2.01 (m, | |
| | 2H), 1.75-1.50 (m, 6H). | |
| 3-91 | ¹H NMR (400 MHz, DMSO-d₆) δ 12.55 (s, 1H), 8.44 (d, J = 8.2 Hz, 1H), 8.20 | |
| | (d, J = 8.3 Hz, 1H), 4.52-4.40 (m, 1H), 3.63 (s, 3H), 2.49 (s, 3H), 2.20-2.09 (m, | |
| | 2H), 1.95-1.85 (m, 2H), 1.80-1.70 (m, 2H), 1.67-1.57 (m, 2H). | |
| 3-92 | ¹H NMR (400 MHz, DMSO-d₆) δ 12.42 (s, 1H), 8.14 (d, J = 8.1 Hz, 1H), 7.87 | |
| | (d, J = 8.1 Hz, 1H), 3.55 (s, 3H), 3.52-3.45 (m, 1H), 2.49 (s, 3H), 1.86-1.77 (m, | |
| | 2H), 1.77-1.69 (m, 2H), 1.52-1.38 (m, 2H), 1.34-1.15 (m, 4H). | |
| 3-93 | ¹H NMR (400 MHz, DMSO-d₆) δ 12.54 (s, 1H), 8.18 (d, J = 8.1 Hz, 1H), 8.04 | |
| | (d, J = 8.1 Hz, 1H), 3.89 (s, 1H), 3.50 (s, 3H), 2.48 (s, 3H), 1.89-1.71 (m, 2H), 1.69- | |
| | 1.51 (m, 2H), 1.44 (s, 2H), 1.27-1.16 (m, 4H). | |
| 3-96 | ¹H NMR (400 MHz, DMSO-d₆) δ 12.52 (s, 1H), 8.44 (d, J = 8.2 Hz, 1H), 8.20 | |
| | (d, J = 8.2 Hz, 1H), 4.08-3.91 (m, 1H), 3.63 (s, 3H), 2.50 (s, 3H), 2.03-1.95 (m, | |
| | 2H), 1.90-1.80 (m, 2H), 1.70-1.62 (m, 2H), 1.35-1.14 (m, 4H). | |
| 3-97 | ¹H NMR (400 MHz, DMSO-d₆) δ 12.46 (s, 1H), 8.15 (d, J = 8.1 Hz, 1H), 7.96 | |
| | (d, J = 8.1 Hz, 1H), 4.04 (q, J = 10.5 Hz, 2H), 3.54 (s, 3H), 2.50 (s, 3H). | |
| 3-98 | ¹H NMR (400 MHz, DMSO-d₆) δ 12.58 (s, 1H), 8.15 (d, J = 8.1 Hz, 1H), 8.11 (d, J = | |
| | 8.3 Hz, 1H), 4.73 (m, 1H), 4.49-4.20 (m, 1H), 3.55 (s, 3H), 2.50 (s, 3H). | |
| 3-101 | ¹H NMR (400 MHz, DMSO-d₆) δ 12.76 (s, 1H), 8.40 (d, J = 8.2 Hz, 1H), 8.27 | |
| | (d, J = 8.2 Hz, 1H), 5.11 (q, J = 9.8 Hz, 2H), 3.66 (s, 3H), 2.49 (s, 3H). | |
| 3-102 | ¹H NMR (400 MHz, CDCl3-d) δ 8.23 (d, J = 8.2 Hz, 1H), 7.80 (d, J = 8.2 Hz, | |
| | 1H), 6.30-5.92 (m, 1H), 3.49-3.36 (m, 5H), 2.51 (s, 3H). | |
| 3-103 | ¹H NMR (400 MHz, DMSO-d₆) δ 12.56 (s, 1H), 8.14 (d, J = 8.1 Hz, 1H), 8.07 | |
| | (d, J = 8.1 Hz, 1H), 6.89-6.37 (m, 1H), 4.44-4.23 (m, 1H), 4.00-3.74 (m, 1H), | |
| | 3.55 (s, 3H), 2.49 (s, 3H). | |
| 3-106 | ¹H NMR (400 MHz, DMSO-d₆) δ 12.62 (s, 1H), 8.38 (d, J = 8.2 Hz, 1H), 8.23 | |
| | (d, J = 8.3 Hz, 1H), 6.65 (tt, J = 54.1, 4.4 Hz, 1H), 4.65-4.52 (m, 2H), 3.65 (s, 3H), | |
| | 2.49 (s, 3H). | |
| 3-112 | ¹H NMR (400 MHz, DMSO-d₆) δ 12.43 (s, 1H), 8.11 (d, J = 8.1 Hz, 1H), 7.88 | |
| | (d, J = 8.1 Hz, 1H), 5.97-5.82 (m, 1H), 5.19-5.00 (m, 2H), 3.72 (d, J = 7.4 Hz, | |
| | 2H), 3.55 (s, 3H), 2.49 (s, 3H). | |
| 3-113 | ¹H NMR (400 MHz, DMSO-d₆) δ 12.53 (s, 1H), 8.13 (d, J = 8.1 Hz, 1H), 8.04 (d, J = | |
| | 8.1 Hz, 1H), 5.95 (dt, J = 17.3, 8.7 Hz, 1H), 5.49-5.40 (m, 2H), 4.46-4.36 (m, 1H), | |
| | 4.06-3.97 (m, 1H), 3.52 (s, 3H), 2.50 (d, J = 1.9 Hz, 3H). | |
| 3-116 | ¹H NMR (400 MHz, DMSO-d₆) δ 8.37 (d, J = 8.2 Hz, 1H), 8.21-8.12 (m, 1H), | |
| | 5.94 (dq, J = 17.0, 7.9 Hz, 1H), 5.49 (dd, J = 13.5, 10.0 Hz, 2H), 4.51 (d, J = 7.3 Hz, | |
| | 2H), 3.64 (s, 3H), 2.48 (s, 3H). | |
| 3-142 | ¹H NMR (400 MHz, CDCl3-d) δ 8.20 (d, J = 8.2 Hz, 1H), 7.75 (d, J = 8.1 Hz, | |
| | 1H), 4.88 (dt, J = 3.5, 1.7 Hz, 2H), 3.65 (s, 2H), 3.47 (d, J = 1.4 Hz, 3H), 2.52 (s, 3H), | |
| | 1.92 (d, J = 1.5 Hz, 3H). | |
| 3-143 | ¹H NMR (400 MHz, DMSO-d₆) δ 12.53 (s, 1H), 8.13 (d, J = 8.1 Hz, 1H), 8.04 | |
| | (d, J = 8.0 Hz, 1H), 5.19 (s, 1H), 5.12 (s, 1H), 4.39 (d, J = 12.4 Hz, 1H), 3.81 (d, J = | |
| | 12.3 Hz, 1H), 3.55 (s, 3H), 2.50 (s, 3H), 1.93 (s, 3H). | |
| 3-146 | ¹H NMR (400 MHz, DMSO-d₆) δ 8.37 (d, J = 8.2 Hz, 1H), 8.11 (d, J = 8.2 Hz, | |
| | 1H), 5.27 (s, 1H), 5.11 (s, 1H), 4.40 (s, 2H), 3.62 (s, 3H), 2.48 (s, 3H), 1.99 (s, 3H). | |
| 3-147 | ¹H NMR (400 MHz, DMSO-d₆) δ 12.46 (s, 1H), 8.14 (d, J = 8.1 Hz, 1H), 7.93 | |
| | (d, J = 8.1 Hz, 1H), 3.94 (d, J = 2.7 Hz, 2H), 3.59 (s, 3H), 3.25 (t, J = 2.7 Hz, 1H), | |
| | 2.49 (s, 3H). | |
| 3-148 | ¹H NMR (400 MHz, CDCl₃-d) δ 8.22 (d, J = 8.1 Hz, 1H), 7.91 (d, J = 8.1 Hz, | |
| | 1H), 4.68 (dd, J = 15.7, 2.7 Hz, 1H), 4.19 (dd, J = 15.8, 2.8 Hz, 1H), 3.46 (s, 3H), | |
| | 2.54 (s, 3H), 2.48 (t, J = 2.7 Hz, 1H). | |
| 3-151 | ¹H NMR (400 MHz, DMSO-d₆) δ 12.60 (s, 1H), 8.40 (d, J = 8.2 Hz, 1H), 8.25 (d, J = | |
| | 8.2 Hz, 1H), 4.85 (d, J = 2.8 Hz, 2H), 3.70-3.62 (m, 4H), 2.49 (s, 3H). | |
| 3-172 | ¹H NMR (400 MHz, CDCl₃-d) δ 11.86 (s, 1H), 8.22 (d, J = 8.1, 1.3 Hz, 1H), 7.82 (d, | |
| | J = 8.1 Hz, 1H), 3.69 (t, J = 17.3 Hz, 2H), 3.47 (s, 3H), 2.50 (s, 3H). | |
| 3-173 | ¹H NMR (400 MHz, CDCl₃-d) δ 8.32-8.16 (m, 1H), 7.99 (d, J = 8.1 Hz, 1H), | |
| | 3.83-3.67 (m, 1H), 3.48 (s, 3H), 2.53 (s, 3H), 2.22 (t, J = 7.7 Hz, 1H). | |
| 3-192 | ¹H NMR (400 MHz, DMSO-d₆) δ 12.49 (s, 1H), 8.17 (d, J = 8.1 Hz, 1H), 8.00 | |
| | (d, J = 8.1 Hz, 1H), 4.23 (s, 2H), 3.56 (s, 3H), 2.49 (s, 3H). | |
| 3-193 | ¹H NMR (400 MHz, DMSO-d₆) δ 12.70 (s, 1H), 8.41-7.98 (m, 2H), 4.97 (d, J = | |
| | 16.1 Hz, 1H), 4.71 (d, J = 16.1 Hz, 1H), 3.56 (s, 3H), 2.49 (s, 3H). | |
| 3-197 | ¹H NMR (400 MHz, DMSO-d₆) δ 12.46 (s, 1H), 8.15 (d, J = 8.1 Hz, 1H), 7.93 | |
| | (d, J = 8.1 Hz, 1H), 3.59 (s, 3H), 3.29 (t, J = 6.9 Hz, 2H), 2.90 (t, J = 6.9 Hz, 2H), | |
| | 2.50 (s, 3H). | |
| 3-198 | ¹H NMR (400 MHz, DMSO-d₆) δ 12.52 (s, 1H), 8.15 (d, J = 8.1 Hz, 1H), 8.07 | |
| | (d, J = 8.0 Hz, 1H), 3.91 (dt, J = 12.7, 6.2 Hz, 1H), 3.56 (s, 3H), 3.52-3.47 (m, 1H), | |
| | 3.23-3.09 (m, 2H), 2.50 (d, J = 2.1 Hz, 3H). | |
| 3-201 | ¹H NMR (400 MHz, DMSO-d₆) δ 12.59 (s, 1H), 8.39 (d, J = 8.2 Hz, 1H), 8.24 | |
| | (d, J = 8.2 Hz, 1H), 4.09 (t, J = 6.8 Hz, 2H), 3.66 (s, 3H), 3.18 (t, J = 6.7 Hz, 2H), | |
| | 2.50 (s, 3H). | |
| 3-202 | ¹H NMR (400 MHz, DMSO-d₆) δ 12.58 (s, 1H), 8.19 (d, J = 8.0 Hz, 1H), 8.01 | |
| | (d, J = 8.1 Hz, 1H), 4.57 (q, J = 7.1 Hz, 1H), 3.56 (s, 3H), 2.49 (s, 3H), 1.65 (d, J = | |
| | 7.2 Hz, 3H). | |
| 3-212 | ¹H NMR (400 MHz, DMSO-d₆) δ 12.42 (s, 1H), 8.14 (d, J = 8.1 Hz, 1H), 7.91 | |
| | (d, J = 8.1 Hz, 1H), 3.86 (t, J = 7.2 Hz, 2H), 3.58 (s, 3H), 3.39 (t, J = 7.2 Hz, 2H), | |
| | 2.49 (s, 2H). | |
| 3-213 | ¹H NMR (400 MHz, DMSO-d₆) δ 12.54 (s, 1H), 8.15 (d, J = 8.1 Hz, 1H), 8.06 | |
| | (d, J = 8.1 Hz, 1H), 4.26-4.18 (m, 1H), 4.14 (dt, J = 13.1, 4.0 Hz, 1H), 4.10-4.00 | |
| | (m, 1H), 3.54 (s, 3H), 3.53-3.48 (m, 1H), 2.49 (s, 3H). | |
| 3-216 | ¹H NMR (400 MHz, DMSO-d₆) δ 12.58 (brs, 1H), 8.38 (d, J = 8.2 Hz, 1H), 8.23 | |
| | (d, J = 7.9 Hz, 1H), 4.24 (t, J = 6.3 Hz, 2H), 4.12 (t, J = 6.3 Hz, 2H), 3.66 (s, 3H), | |
| | 2.50 (s, 3H). | |
| 3-262 | ¹H NMR (400 MHz, CDCl₃-d) δ 8.23 (d, J = 8.1 Hz, 1H), 7.76 (d, J = 8.2 Hz, | |
| | 1H), 5.07 (s, 2H), 3.48 (s, 3H), 3.43 (s, 3H), 2.53 (s, 3H). | |
| 3-267 | ¹H NMR (400 MHz, DMSO-d₆) δ 12.60 (s, 1H), 9.09 (s, 1H), 8.14 (d, J = 8.1 | |
| | Hz, 1H), 7.91 (d, J = 8.1 Hz, 1H), 3.57 (s, 3H), 2.99 (t, J = 7.4 Hz, 2H), 1.64 (h, J = | |
| | 7.4 Hz, 2H), 0.99 (t, J = 7.3 Hz, 3H). | |
| 3-268 | ¹H NMR (400 MHz, DMSO-d₆) δ 12.69 (s, 1H), 9.08 (s, 1H), 8.12 (d, J = 8.1 | |
| | Hz, 1H), 8.05 (d, J = 8.1 Hz, 1H), 3.79-3.66 (m, 1H), 3.54 (s, 3H), 3.15-3.01 (m, | |
| | 1H), 1.90-1.73 (m, 2H), 1.08 (t, J = 7.4 Hz, 3H). | |
| 3-271 | ¹H NMR (400 MHz, DMSO-d₆) δ 12.71 (s, 1H), 9.09 (s, 1H), 8.38 (d, J = 8.2 | |
| | Hz, 1H), 8.22 (d, J = 8.2 Hz, 1H), 3.71-3.66 (m, 2H), 3.65 (s, 3H), 1.95-1.84 (m, | |
| | 2H), 1.05 (t, J = 7.4 Hz, 3H). | |
| 3-272 | ¹H NMR (400 MHz, DMSO-d₆) δ 12.66 (s, 1H), 9.06 (s, 1H), 8.15 (d, J = 8.2 | |
| | Hz, 1H), 7.89 (d, J = 8.1 Hz, 1H), 3.80 (h, J = 6.8 Hz, 1H), 3.55 (s, 3H), 1.25 (d, J = | |
| | 6.7 Hz, 6H). | |
| 3-273 | ¹H NMR (400 MHz, DMSO-d₆) δ 12.75 (s, 1H), 9.07 (s, 1H), 8.20 (d, J = 8.1 | |
| | Hz, 1H), 8.07 (d, J = 8.1 Hz, 1H), 4.18-4.05 (m, 1H), 3.52 (s, 3H), 1.46 (d, J = 6.8 | |
| | Hz, 3H), 1.11 (d, J = 7.0 Hz, 3H). | |
| 3-276 | ¹H NMR (400 MHz, DMSO-d₆) δ 12.73 (s, 1H), 9.08 (s, 1H), 8.46 (d, J = 8.2 | |
| | Hz, 1H), 8.23 (d, J = 8.2 Hz, 1H), 4.18 (h, J = 6.7 Hz, 1H), 3.63 (s, 3H), 1.36 (d, J = | |
| | 6.8 Hz, 6H). | |
| 3-277 | ¹H NMR (400 MHz, DMSO-d₆) δ 12.62 (s, 1H), 9.08 (s, 1H), 8.13 (d, J = 8.1 | |
| | Hz, 1H), 7.90 (d, J = 8.1 Hz, 1H), 3.57 (s, 3H), 3.01 (t, J = 7.5 Hz, 2H), 1.60 (p, J = | |
| | 7.3 Hz, 2H), 1.40 (h, J = 7.4 Hz, 2H), 0.89 (t, J = 7.3 Hz, 3H). | |
| 3-278 | ¹H NMR (400 MHz, DMSO-d₆) δ 12.70 (s, 1H), 9.07 (s, 1H), 8.12 (d, J = 8.1 Hz, | |
| | 1H), 8.04 (d, J = 8.1 Hz, 1H), 3.79-3.67 (m, 1H), 3.54 (s, 3H), 3.17-3.03 (m, 1H), | |
| | 1.88-1.68 (m, 2H), 1.60-1.40 (m, 2H), 0.94 (t, J = 7.3 Hz, 3H). | |
| 3-281 | ¹H NMR (400 MHz, DMSO-d₆) δ 12.75 (s, 1H), 9.10 (s, 1H), 8.38 (d, J = 8.2 | |
| | Hz, 1H), 8.23 (d, J = 8.2 Hz, 1H), 3.75-3.67 (m, 2H), 3.66 (s, 3H), 1.83 (dt, J = 15.2, | |
| | 7.2 Hz, 2H), 1.47 (h, J = 7.4 Hz, 2H), 0.92 (t, J = 7.3 Hz, 3H). | |
| 3-282 | ¹H NMR (400 MHz, DMSO-d₆) δ 12.64 (brs, 1H), 9.08 (s, 1H), 8.16 (d, J = 8.1 | |
| | Hz, 1H), 7.90 (d, J = 8.2 Hz, 1H), 3.71-3.60 (m, 1H), 3.56 (s, 3H), 1.63 (p, J = 7.2 | |
| | Hz, 2H), 1.18 (d, J = 6.7 Hz, 3H), 0.98 (t, J = 7.4 Hz, 3H). | |
| 3-283 | ¹H NMR (400 MHz, CDCl₃-d) δ 8.26-8.17 (m, 2H), 7.72 (d, J = 8.1 Hz, 1H), | [α] |
| | 3.76-3.63 (m, 1H), 3.46 (s, 3H), 1.79-1.56 (m, 2H), 1.22 (d, J = 6.7 Hz, 3H), 1.01 | D²⁰ = +12.91°(c |
| | (t, J = 7.4 Hz, 3H). | 0.115, DMF) |
| 3-284 | ¹H NMR (400 MHz, CDCl₃-d) δ 8.27-8.20 (m, 2H), 7.70 (d, J = 8.2 Hz, 1H), | [α] |
| | 3.76-3.63 (m, 1H), 3.46 (s, 3H), 1.76-1.60 (m, 2H), 1.23 (d, J = 6.8 Hz, 3H), 1.02 | D²⁰ = −13.71°(c |
| | (t, J = 7.4 Hz, 3H). | 0.108, DMF) |
| 3-285 | ¹H NMR (400 MHz, DMSO-d₆) δ 12.73 (s, 1H), 9.08 (s, 1H), 8.19 (d, J = 8.1 | |
| | Hz, 1H), 8.07 (d, J = 8.2 Hz, 1H), 4.09-3.79 (m, 1H), 3.52 (d, J = 4.0 Hz, 3H), 2.13- | |
| | 2.00 (m, 1H), 1.82-1.68 (m, 1H), 1.54-1.45 (m, 1H), 1.41 (d, J = 6.9 Hz, 1H), | |
| | 1.10-1.00 (m, 3H), 0.92 (t, J = 7.4 Hz, 1H). | |
| 3-286 | ¹H NMR (400 MHz, CDCl₃-d) δ 8.27 (d, J = 8.1 Hz, 1H), 8.20 (s, 1H), 7.86 (d, J = | [α] |
| | 8.1 Hz, 1H), 4.08-3.98 (m, 1H), 3.40 (s, 3H), 1.63-1.53 (m, 2H), 1.52 (d, J = 6.9 | D²⁰ = +78.73°(c |
| | Hz, 3H), 0.97 (t, J = 7.4 Hz, 3H). | 0.105, DMF) |
| 3-287 | ¹H NMR (400 MHz, CDCl-d) δ 8.26 (d, J = 8.1 Hz, 1H), 8.19 (s, 1H), 7.87 (d, J = | [α] |
| | 8.1 Hz, 1H), 4.15 (s, 1H), 3.40 (s, 3H), 2.26-2.15 (m, 1H), 1.91-1.77 (m, 1H), | D²⁰ = −77.65°(c |
| | 1.17-1.09 (m, 6H). | 0.109, DMF) |
| 3-288 | ¹H NMR (400 MHz, CDCl₃-d) δ 8.37-8.15 (m, 2H), 7.87 (d, J = 7.7 Hz, 1H), | [α] |
| | 4.16-4.11 (m, 1H), 3.40 (s, 3H), 2.22-2.12 (m, 1H), 1.90-1.77 (m, 1H), 1.15- | D²⁰ = +65.05°(c |
| | 1.08 (m, 6H). | 0.104, DMF) |
| 3-289 | ¹H NMR (400 MHz, CDCl₃-d) δ 8.26 (d, J = 7.7 Hz, 1H), 8.20 (s, 1H), 7.86 (d, J = | [α] |
| | 7.9 Hz, 1H), 4.06-4.00 (m, 1H), 3.40 (s, 3H), 1.63-1.54 (m, 2H), 1.52 (d, J = 6.9 | D²⁰ = −80.21°(c |
| | Hz, 3H), 0.97 (t, J = 7.4 Hz, 3H). | 0.105, DMF) |
| 3-290 | ¹H NMR (400 MHz, DMSO-d₆) δ 8.97 (s, 1H), 8.42 (d, J = 8.3 Hz, 1H), 8.15 (d, | |
| | J = 8.1 Hz, 1H), 4.06-3.94 (m, 1H), 3.62 (s, 3H), 2.02-1.87 (m, 1H), 1.74-1.58 | |
| | (m, 1H), 1.34 (d, J = 6.9 Hz, 3H), 1.00 (t, J = 7.4 Hz, 3H). | |
| 3-297 | ¹H NMR (400 MHz, DMSO-d₆) δ 12.73 (s, 1H), 9.08 (s, 1H), 8.36 (d, J = 8.2 | |
| | Hz, 1H), 8.21 (d, J = 8.2 Hz, 1H), 3.66 (s, 3H), 3.60 (d, J = 6.5 Hz, 2H), 2.50-2.43 | |
| | (m, 1H), 1.13 (s, 3H), 1.12 (d, J = 6.7 Hz, 6H). | |
| 3-298 | ¹H NMR (400 MHz, DMSO-d₆) δ 12.58 (s, 1H), 9.05 (s, 1H), 8.09 (d, J = 8.1 | |
| | Hz, 1H), 7.87 (d, J = 8.1 Hz, 1H), 3.58 (s, 3H), 2.94 (d, J = 7.4 Hz, 2H), 1.05-0.93 | |
| | (m, 1H), 0.52-0.44 (m, 2H), 0.23-0.14 (m, 2H). | |
| 3-299 | ¹H NMR (400 MHz, DMSO-d₆) δ 12.70 (s, 1H), 9.08 (s, 1H), 8.12 (d, J = 8.1 | |
| | Hz, 1H), 8.05 (s, 1H), 3.86 (dd, J = 13.1, 6.0 Hz, 1H), 3.54 (s, 3H), 2.95-2.80 (m, | |
| | 1H), 1.22-1.11 (m, 1H), 0.76-0.57 (m, 2H), 0.54-0.31 (m, 2H). | |
| 3-302 | ¹H NMR (400 MHz, DMSO-d₆) δ 12.76 (s, 1H), 9.09 (s, 1H), 8.39 (d, J = 8.2 | |
| | Hz, 1H), 8.22 (d, J = 8.2 Hz, 1H), 3.79-3.60 (m, 5H), 1.22-1.12 (m, 1H), 0.68- | |
| | 0.56 (m, 2H), 0.44-0.30 (m, 2H). | |
| 3-303 | ¹H NMR (400 MHz, DMSO-d₆) δ 12.69 (s, 1H), 9.03 (s, 1H), 8.11 (d, J = 8.2 | |
| | Hz, 1H), 7.88 (d, J = 8.1 Hz, 1H), 3.48 (s, 3H), 2.75-2.60 (m, 1H), 0.96-0.86 (m, | |
| | 2H), 0.82-0.68 (m, 2H). | |
| 3-304 | ¹H NMR (400 MHz, CDCl3-d) δ 8.35-8.12 (m, 2H), 7.87 (d, J = 7.5 Hz, 1H), | |
| | 3.48-3.43 (m, 1H), 3.42 (s, 3H), 1.48-1.37 (m, 1H), 1.36-1.28 (m, 2H), 1.11- | |
| | 0.98 (m, 1H). | |
| 3-307 | ¹H NMR (400 MHz, DMSO-d₆) δ 12.77 (s, 1H), 9.08 (s, 1H), 8.42 (d, J = 8.2 | |
| | Hz, 1H), 8.21 (d, J = 8.2 Hz, 1H), 3.65 (s, 3H), 3.49-3.43 (m, 1H), 1.54-1.42 (m, | |
| | 2H), 1.27-1.16 (m, 2H). | |
| 3-309 | ¹H NMR (400 MHz, DMSO-d₆) δ 12.68 (s, 1H), 9.06 (s, 1H), 8.12 (d, J = 8.1 | |
| | Hz, 1H), 8.01 (d, J = 8.1 Hz, 1H), 4.50-4.38 (m, 1H), 3.52 (s, 3H), 2.80-2.67 (m, | |
| | 1H), 2.35-1.92 (m, 5H). | |
| 3-312 | ¹H NMR (400 MHz, DMSO-d₆) δ 12.74 (s, 1H), 9.06 (s, 1H), 8.40 (d, J = 8.2 | |
| | Hz, 1H), 8.19 (d, J = 8.2 Hz, 1H), 4.87-4.61 (m, 1H), 3.65 (s, 3H), 2.73-2.60 (m, | |
| | 2H), 2.30-2.18 (m, 2H), 2.09-1.93 (m, 2H). | |
| 3-314 | ¹H NMR (400 MHz, CDCl3-d) δ 8.29 (s, 1H), 8.21 (d, J = 7.6 Hz, 1H), 7.86 (d, | |
| | J = 7.9 Hz, 1H), 4.49-4.44 (m, 1H), 3.39 (s, 3H), 2.28-2.21 (m, 1H), 2.18-2.09 | |
| | (m, 1H), 1.92-1.67 (m, 5H), 1.64-1.57 (m, 1H). | |
| 3-317 | ¹H NMR (400 MHz, DMSO-d₆) δ 12.73 (s, 1H), 9.06 (s, 1H), 8.45 (m, 1H), 8.21 | |
| | (m, 1H), 4.45 (h, J = 7.3, 6.6 Hz, 1H), 3.63 (s, 3H), 2.20-2.11 (m, 2H), 1.94-1.89 | |
| | (m, 2H), 1.77-1.71 (m, 2H), 1.67-1.61 (m, 2H). | |
| 3-323 | ¹H NMR (400 MHz, DMSO-d₆) δ 12.65 (s, 1H), 9.08 (s, 1H), 8.16 (d, J = 8.1 | |
| | Hz, 1H), 7.98 (d, J = 8.1 Hz, 1H), 4.04 (q, J = 10.5 Hz, 2H), 3.55 (s, 3H). | |
| 3-324 | ¹H NMR (400 MHz, DMSO-d₆) δ 12.76 (s, 1H), 9.09 (s, 1H), 8.23-8.11 (m, | |
| | 2H), 4.83-4.66 (m, 1H), 4.47-4.31 (m, 1H), 3.56 (s, 3H). | |
| 3-327 | ¹H NMR (400 MHz, DMSO-d₆) δ 12.82 (s, 1H), 9.09 (s, 1H), 8.42 (d, J = 8.2 | |
| | Hz, 1H), 8.31 (d, J = 8.2 Hz, 1H), 5.11 (q, J = 9.7 Hz, 2H), 3.66 (s, 3H). | |
| 3-328 | ¹H NMR (400 MHz, CDCl3-d) δ 8.27 (d, J = 8.2 Hz, 1H), 8.16 (s, 1H), 7.81 (d, | |
| | J = 8.2 Hz, 1H), 6.13 (tt, J = 55.9, 4.4 Hz, 1H), 3.50-3.42 (m, 5H). | |
| 3-329 | ¹H NMR (400 MHz, DMSO-d₆) δ 12.71 (s, 1H), 9.08 (s, 1H), 8.15 (d, J = 8.1 | |
| | Hz, 1H), 8.10 (d, J = 8.0 Hz, 1H), 6.84-6.35 (m, 1H), 4.34 (m, 1H), 3.95-3.71 (m, | |
| | 1H), 3.55 (s, 3H). | |
| 3-338 | ¹H NMR (400 MHz, CDCl3-d) δ 8.23 (d, J = 8.1 Hz, 1H), 8.19 (s, 1H), 7.77 (d, | |
| | J = 8.1 Hz, 1H), 6.00-5.85 (m, 1H), 5.19-5.11 (m, 1H), 5.08 (d, J = 10.0 Hz, 1H), | |
| | 3.71 (d, J = 7.4 Hz, 2H), 3.47 (d, J = 1.2 Hz, 3H). | |
| 3-339 | ¹H NMR (400 MHz, CDCl3-d) δ 8.17-8.10 (m, 2H), 7.79 (d, J = 8.0 Hz, 1H), | |
| | 5.95 (m, 1H), 5.50-5.39 (m, 2H), 4.49-4.40 (m, 1H), 4.00-3.90 (m, 1H), 3.37 (s, 3H). | |
| 3-342 | ¹H NMR (400 MHz, DMSO-d₆) δ 12.78 (s, 1H), 9.09 (s, 1H), 8.39 (d, J = 8.2 | |
| | Hz, 1H), 8.23 (d, J = 8.2 Hz, 1H), 6.03-5.88 (m, 1H), 5.54-5.44 (m, 2H), 4.52 (d, J = | |
| | 7.5 Hz, 2H), 3.65 (s, 3H). | |
| 3-373 | ¹H NMR (400 MHz, DMSO-d₆) δ 12.61 (s, 1H), 9.09 (s, 1H), 8.15 (d, J = 8.1 | |
| | Hz, 1H), 7.94 (d, J = 8.1 Hz, 1H), 3.87 (t, J = 7.2 Hz, 2H), 3.59 (s, 3H), 3.40 (t, J = | |
| | 7.2 Hz, 2H). | |
| 3-378 | ¹H NMR (400 MHz, DMSO-d₆) δ 12.64 (s, 1H), 9.09 (s, 1H), 8.17 (d, J = 8.1 | |
| | Hz, 1H), 7.99 (d, J = 8.1 Hz, 1H), 4.04 (q, J = 10.6 Hz, 2H), 3.55 (s, 3H), 3.43-3.34 | |
| | (m, 2H). | |
| 3-379 | ¹H NMR (400 MHz, DMSO-d₆) δ 12.69 (s, 1H), 9.07 (s, 1H), 8.15 (d, J = 8.1 | |
| | Hz, 1H), 8.08 (d, J = 8.1 Hz, 1H), 4.27-4.16 (m, 1H), 4.19-4.08 (m, 1H), 4.11- | |
| | 3.99 (m, 1H), 3.55 (s, 3H), 3.56-3.45 (m, 1H). | |
| 3-382 | ¹H NMR (400 MHz, DMSO-d₆) δ 12.76 (s, 1H), 9.11 (s, 1H), 8.39 (d, J = 8.2 | |
| | Hz, 1H), 8.25 (d, J = 8.2 Hz, 1H), 4.24 (t, J = 6.3 Hz, 2H), 4.13 (t, J = 6.3 Hz, 2H), | |
| | 3.67 (s, 3H). | |
| 3-383 | ¹H NMR (400 MHz, CDCl3-d) δ 8.21 (d, J = 8.1 Hz, 1H), 7.72 (d, J = 8.2 Hz, | |
| | 1H), 3.49 (s, 3H), 3.01 (t, J = 7.5 Hz, 2H), 2.89 (q, J = 7.6 Hz, 2H), 1.80-1.66 (m, | |
| | 2H), 1.40 (t, J = 7.6 Hz, 3H), 1.04 (t, J = 7.3 Hz, 3H). | |
| 3-384 | ¹H NMR (400 MHz, CDCl3-d) δ 8.17 (d, J = 8.1 Hz, 1H), 7.86 (d, J = 8.1 Hz, | |
| | 1H), 3.96-3.85 (m, 1H), 3.44 (s, 3H), 3.16-3.04 (m, 1H), 2.86 (q, J = 7.6 Hz, 2H), | |
| | 2.06-1.91 (m, 2H), 1.39 (t, J = 7.6 Hz, 3H), 1.16 (t, J = 7.4 Hz, 3H). | |
| 3-387 | ¹H NMR (400 MHz, CDCl3-d) δ 8.36 (d, J = 8.1 Hz, 1H), 7.92 (d, J = 8.2 Hz, | |
| | 1H), 3.69-3.55 (m, 5H), 2.86 (q, J = 7.6 Hz, 2H), 2.06 (h, J = 7.5 Hz, 2H), 1.38 (t, | |
| | J = 7.6 Hz, 3H), 1.13 (t, J = 7.4 Hz, 3H). | |
| 3-388 | ¹H NMR (400 MHz, DMSO-d₆) δ 12.52 (s, 1H), 8.14 (d, J = 8.1 Hz, 1H), 7.88 | |
| | (d, J = 8.1 Hz, 1H), 3.85-3.75 (m, 1H), 3.55 (s, 3H), 2.91-2.77 (m, 2H), 1.29- | |
| | 1.20 (m, 9H). | |
| 3-389 | ¹H NMR (400 MHz, DMSO-d₆) δ 12.58 (s, 1H), 8.18 (d, J = 8.1 Hz, 1H), 8.05 | |
| | (d, J = 8.1 Hz, 1H), 4.16-4.00 (m, 1H), 3.51 (s, 3H), 2.92-2.77 (m, 2H), 1.45 (d, J = | |
| | 6.8 Hz, 3H), 1.25 (t, J = 7.5 Hz, 3H), 1.11 (d, J = 7.0 Hz, 3H). | |
| 3-392 | ¹H NMR (400 MHz, DMSO-d₆) δ 8.42 (d, J = 8.2 Hz, 1H), 8.16 (d, J = 8.3 Hz, | |
| | 1H), 4.25-4.11 (m, 1H), 3.62 (s, 3H), 2.82 (q, J = 7.5 Hz, 2H), 1.35 (d, J = 6.8 Hz, | |
| | 6H), 1.25 (t, J = 7.5 Hz, 3H). | |
| 3-393 | ¹H NMR (400 MHz, DMSO-d₆) δ 12.45 (s, 1H), 8.12 (d, J = 8.1 Hz, 1H), 7.88 | |
| | (d, J = 8.1 Hz, 1H), 3.56 (s, 3H), 3.00 (t, J = 7.5 Hz, 2H), 2.91-2.77 (m, 2H), 1.60 | |
| | (p, J = 7.4 Hz, 2H), 1.40 (h, J = 7.4 Hz, 2H), 1.31-1.20 (m, 3H), 0.95-0.83 (m, 3H). | |
| 3-394 | ¹H NMR (400 MHz, DMSO-d₆) δ 12.55 (s, 1H), 8.12 (d, J = 8.1 Hz, 1H), 8.02 | |
| | (d, J = 8.1 Hz, 1H), 3.78-3.69 (m, 1H), 3.53 (s, 3H), 3.15-3.06 (m, 1H), 2.85 (q, J = | |
| | 7.6 Hz, 2H), 1.89-1.68 (m, 2H), 1.60-1.40 (m, 2H), 1.26 (t, J = 7.5 Hz, 3H), 0.94 | |
| | (t, J = 7.3 Hz, 3H). | |
| 3-397 | ¹H NMR (400 MHz, DMSO-d₆) δ 12.65 (s, 1H), 8.29 (d, J = 72.5 Hz, 2H), 3.68 | |
| | (d, J = 17.7 Hz, 5H), 2.85 (s, 2H), 1.84 (t, J = 7.7 Hz, 2H), 1.47 (q, J = 7.4 Hz, 2H), | |
| | 1.27-1.19 (m, 3H), 0.91 (t, J = 7.3 Hz, 3H). | |
| 3-398 | ¹H NMR (400 MHz, DMSO-d₆) δ 12.52 (s, 1H), 8.14 (d, J = 8.1 Hz, 1H), 7.88 | |
| | (d, J = 8.1 Hz, 1H), 3.65 (p, J = 6.7 Hz, 1H), 3.55 (s, 3H), 2.84 (q, J = 7.5 Hz, 2H), | |
| | 1.63 (p, J = 7.2 Hz, 2H), 1.26 (t, J = 7.5 Hz, 3H), 1.17 (d, J = 6.7 Hz, 3H), 0.98 (t, J = | |
| | 7.3 Hz, 3H). | |
| 3-401 | ¹H NMR (400 MHz, DMSO-d₆) δ 12.56 (s, 1H), 8.18 (d, J = 8.1 Hz, 1H), 8.10 | |
| | (d, J = 8.1 Hz, 1H), 4.08-3.80 (m, 1H), 3.51 (d, J = 4.0 Hz, 3H), 2.92-2.79 (m, | |
| | 2H), 2.13-2.00 (m, 1H), 1.85-1.69 (m, 1H), 1.56-1.32 (m, 2H), 1.30-1.23 (m, | |
| | 3H), 1.07-1.02 (m, 3H), 0.92 (t, J = 7.4 Hz, 1H). | |
| 3-406 | ¹H NMR (400 MHz, DMSO-d₆) δ 12.62 (s, 1H), 8.44 (d, J = 8.2 Hz, 1H), 8.20 | |
| | (d, J = 8.2 Hz, 1H), 4.07-3.92 (m, 1H), 3.63 (s, 3H), 2.84 (q, J = 7.5 Hz, 2H), 2.04 | |
| | 1.87 (m, 1H), 1.76-1.59 (m, 1H), 1.34 (d, J = 6.9 Hz, 3H), 1.25 (t, J = 7.5 Hz, 3H), | |
| | 1.00 (t, J = 7.4 Hz, 3H). | |
| 3-413 | ¹H NMR (400 MHz, DMSO-d₆) δ 12.61 (s, 1H), 8.36 (d, J = 8.2 Hz, 1H), 8.19 | |
| | (d, J = 8.2 Hz, 1H), 3.66 (s, 3H), 3.60 (d, J = 6.5 Hz, 2H), 2.85 (m, 2H), 2.50-2.42 | |
| | (m, 1H), 1.34-1.21 (m, 3H), 1.12 (d, J = 6.7 Hz, 6H). | |
| 3-414 | ¹H NMR (400 MHz, DMSO-d₆) δ 12.45 (s, 1H), 8.11 (d, J = 8.2 Hz, 1H), 7.87 | |
| | (d, J = 8.1 Hz, 1H), 3.61 (s, 3H), 2.97 (d, J = 7.3 Hz, 2H), 2.91-2.80 (m, 2H), 1.30- | |
| | 1.23 (m, 3H), 1.10-0.96 (m, 1H), 0.60-0.46 (m, 2H), 0.29-0.15 (m, 2H). | |
| 3-415 | ¹H NMR (400 MHz, DMSO-d₆) δ 12.60 (s, 1H), 8.11 (d, J = 8.1 Hz, 1H), 8.02 | |
| | (d, J = 8.1 Hz, 1H), 3.87 (dd, J = 13.1, 6.0 Hz, 1H), 3.54 (s, 3H), 2.94-2.76 (m, 3H), | |
| | 1.25 (t, J = 7.5 Hz, 3H), 1.19-1.13 (m, 1H), 0.76-0.57 (m, 2H), 0.53-0.30 (m, 2H). | |
| 3-418 | ¹H NMR (400 MHz, DMSO-d₆) δ 12.62 (s, 1H), 8.37 (d, J = 8.2 Hz, 1H), 8.18 | |
| | (d, J = 8.2 Hz, 1H), 3.67 (d, J = 12.5 Hz, 5H), 2.84 (q, J = 7.5 Hz, 2H), 1.29-1.18 | |
| | (m, 4H), 0.68-0.55 (m, 2H), 0.43-0.30 (m, 2H). | |
| 3-419 | ¹H NMR (400 MHz, DMSO-d₆) δ 12.53 (s, 1H), 8.11 (d, J = 8.1 Hz, 1H), 7.88 | |
| | (d, J = 8.1 Hz, 1H), 3.48 (s, 3H), 2.84 (q, J = 7.5 Hz, 2H), 2.73-2.62 (m, 1H), 1.26 | |
| | (t, J = 7.5 Hz, 3H), 0.95-0.85 (m, 2H), 0.83-0.68 (m, 2H). | |
| 3-420 | ¹H NMR (400 MHz, CDCl3-d) δ 8.23 (d, J = 8.1 Hz, 1H), 7.87 (d, J = 8.1 Hz, | |
| | 1H), 3.57-3.46 (m, 1H), 3.42 (s, 3H), 2.86 (q, J = 7.5 Hz, 2H), 1.43-1.36 (m, 3H), | |
| | 1.36-1.20 (m, 3H), 1.08-0.96 (m, 1H). | |
| 3-423 | ¹H NMR (400 MHz, DMSO-d₆) δ 12.68 (s, 1H), 8.40 (d, J = 8.2 Hz, 1H), 8.18 | |
| | (d, J = 8.2 Hz, 1H), 3.64 (s, 3H), 3.48-3.44 (m, 1H), 2.84 (q, J = 7.5 Hz, 2H), 1.48 | |
| | (m, 2H), 1.30-1.17 (m, 5H). | |
| 3-425 | ¹H NMR (400 MHz, DMSO-d₆) δ 12.54 (s, 1H), 8.10 (d, J = 8.1 Hz, 1H), 7.98 | |
| | (d, J = 8.1 Hz, 1H), 4.44 (p, J = 8.3 Hz, 1H), 3.52 (s, 3H), 2.83 (q, J = 7.5 Hz, 2H), | |
| | 2.36-1.92 (m, 6H), 1.25 (t, J = 6.7 Hz, 3H). | |
| 3-428 | ¹H NMR (400 MHz, DMSO-d₆) δ 12.55 (s, 1H), 8.39 (d, J = 8.2 Hz, 1H), 8.18 | |
| | (d, J = 8.2 Hz, 1H), 4.73 (p, J = 8.5 Hz, 1H), 3.65 (s, 3H), 2.94-2.79 (m, 2H), 2.73- | |
| | 2.58 (m, 2H), 2.32-2.17 (m, 2H), 2.10-1.92 (m, 2H), 1.33-1.23 (m, 3H). | |
| 3-430 | ¹H NMR (400 MHz, DMSO-d₆) δ 12.54 (brs, 1H), 8.16 (d, J = 7.6 Hz, 1H), 8.03 | |
| | (d, J = 7.9 Hz, 1H), 4.48-4.22 (m, 1H), 3.52 (s, 3H), 2.94-2.72 (m, 2H), 2.20-2.12 | |
| | (m, 1H), 2.12-2.02 (m, 1H), 1.71-1.64 (m, 5H), 1.61-1.50 (m, 1H), 1.26 (t, J = | |
| | 7.4 Hz, 3H). | |
| 3-433 | ¹H NMR (400 MHz, DMSO-d₆) δ 8.44 (d, J = 8.2 Hz, 1H), 8.18 (d, J = 8.3 Hz, | |
| | 1H), 4.48-4.44 (m, 1H), 3.63 (s, 3H), 2.87-2.81 (m, 2H), 2.15-2.13 (m, 2H), 2.03- | |
| | 1.83 (m, 2H), 1.78-1.74 (m, 2H), 1.66-1.61 (m, 2H), 1.25 (t, J = 7.4 Hz, 3H). | |
| 3-439 | ¹H NMR (400 MHz, DMSO-d₆) δ 12.53 (s, 1H), 8.21 (d, J = 8.1 Hz, 1H), 8.02 | |
| | (d, J = 8.1 Hz, 1H), 4.09 (q, J = 10.6 Hz, 2H), 3.60 (s, 3H), 2.97-2.84 (m, 2H), 1.40- | |
| | 1.26 (m, 3H). | |
| 3-440 | ¹H NMR (400 MHz, DMSO-d₆) δ 12.57 (s, 1H), 8.20-8.06 (m, 2H), 8.11 (d, J = | |
| | 8.2 Hz, 1H), 4.81-4.67 (m, 1H), 4.47-4.30 (m, 1H), 3.55 (s, 3H), 2.93-2.78 (m, | |
| | 2H), 1.28-1.21 (m, 3H). | |
| 3-441 | ¹H NMR (400 MHz, DMSO-d₆) δ 12.67 (s, 1H), 8.18-8.07 (m, 2H), 4.81- | [α] |
| | 4.65 (m, 1H), 4.50-4.23 (m, 1H), 3.55 (s, 3H), 2.89-2.79 (m, 2H), 1.25-1.23 (m, 3H). | D²⁰ = +64.66°(c |
| | | 0.126, DMF) |
| 3-442 | ¹H NMR (400 MHz, DMSO-d₆) δ 12.59 (s, 1H), 8.18-8.07 (m, 2H), 4.81- | [α] |
| | 4.65 (m, 1H), 4.47-4.21 (m, 1H), 3.55 (s, 3H), 2.88-2.81 (m, 1H), 1.26-1.21 (m, 3H). | D²⁰ = −67.30°(c |
| | | 0.106 , DMF) |
| 3-443 | ¹H NMR (400 MHz, DMSO-d₆) δ 12.70 (s, 1H), 8.40 (d, J = 8.2 Hz, 1H), 8.28 | |
| | (d, J = 8.2 Hz, 1H), 5.11 (q, J = 9.8 Hz, 2H), 3.66 (s, 3H), 2.92-2.78 (m, 2H), 1.31- | |
| | 1.21 (m, 3H). | |
| 3-444 | ¹H NMR (400 MHz, CDCl3-d) δ 8.22 (d, J = 8.2 Hz, 1H), 7.83 (d, J = 8.2 Hz, | |
| | 1H), 6.12 (tt, J = 55.9, 4.4 Hz, 1H), 3.47-3.34 (m, 5H), 2.84 (q, J = 7.6 Hz, 2H), | |
| | 1.38 (t, J = 7.6 Hz, 3H). | |
| 3-445 | ¹H NMR (400 MHz, DMSO-d₆) δ 12.60 (s, 1H), 8.13 (d, J = 8.1 Hz, 1H), 8.07 | |
| | (d, J = 8.0 Hz, 1H), 6.86-6.33 (m, 1H), 4.42-4.29 (m, 1H), 3.91-3.75 (m, 1H), | |
| | 3.54 (s, 3H), 2.84 (q, J = 7.5 Hz, 2H), 1.28-1.17 (m, 3H). | |
| 3-454 | ¹H NMR (400 MHz, CDCl3-d) δ 8.19 (d, J = 8.2, 1H), 7.77 (d, J = 8.1 Hz, 1H), | |
| | 6.00-5.85 (m, 1H), 5.18-5.10 (m, 1H), 5.07 (d, J = 9.9 Hz, 1H), 3.70 (d, J = 7.4 | |
| | Hz, 2H), 3.46 (s, 3H), 2.90-2.80 (m, 2H), 1.38 (t, J = 7.6 Hz, 3H). | |
| 3-455 | ¹H NMR (400 MHz, CDCl3-d) δ 8.45-8.02 (m, 1H), 7.99-7.68 (m, 1H), 6.20- | |
| | 5.85 (m, 1H), 5.77-5.37 (m, 2H), 4.72-4.35 (m, 1H), 4.15-3.79 (m, 1H), 3.45 | |
| | (s, 3H), 3.06-2.52 (m, 2H), 1.41-1.36 (m, 3H). | |
| 3-458 | ¹H NMR (400 MHz, DMSO-d₆) δ 12.43 (brs, 1H), 8.37 (d, J = 8.2 Hz, 1H), 8.19 | |
| | (d, J = 8.2 Hz, 1H), 6.01-5.86 (m, 1H), 5.54-5.44 (m, 2H), 4.52 (d, J = 7.4 Hz, | |
| | 2H), 3.64 (s, 3H), 2.84 (q, J = 7.6 Hz, 2H), 1.25 (t, J = 7.5 Hz, 3H). | |
| 3-494 | ¹H NMR (400 MHz, DMSO-d₆) δ 12.43 (s, 1H), 8.14 (d, J = 8.1 Hz, 1H), 7.92 | |
| | (d, J = 8.1 Hz, 1H), 3.87 (t, J = 7.2 Hz, 2H), 3.63-3.55 (m, 3H), 3.39 (t, J = 7.2 Hz, | |
| | 2H), 2.85 (d, J = 8.0 Hz, 2H), 1.27 (s, 3H). | |
| 3-495 | ¹H NMR (400 MHz, DMSO-d₆) δ 12.54 (s, 1H), 8.14 (d, J = 8.1 Hz, 1H), 8.06 | |
| | (d, J = 8.1 Hz, 1H), 4.25-4.18 (m, 1H), 4.18-4.10 (m, 1H), 4.09-4.01 (m, 1H), | |
| | 3.55 (s, 3H), 3.53-3.47 (m, 1H), 2.92-2.78 (m, 2H), 1.33-1.24 (m, 3H). | |
| 3-498 | ¹H NMR (400 MHz, DMSO-d₆) δ 12.57 (s, 1H), 8.38 (d, J = 8.2 Hz, 1H), 8.22 | |
| | (d, J = 8.2 Hz, 1H), 4.24 (t, J = 6.4 Hz, 2H), 4.12 (t, J = 6.3 Hz, 2H), 3.66 (s, 3H), | |
| | 2.94-2.79 (m, 2H), 1.27 (s, 3H). | |
| 3-499 | ¹H NMR (400 MHz, CDCl₃-d) δ 8.18 (d, J = 8.2 Hz, 1H), 7.74 (d, J = 8.1 Hz, | |
| | 1H), 3.47 (s, 3H), 3.22-3.07 (m, 1H), 2.99 (t, J = 7.5 Hz, 2H), 1.78-1.65 (m, 2H), | |
| | 1.39 (d, J = 7.0 Hz, 6H), 1.03 (t, J = 7.4 Hz, 3H). | |
| 3-500 | ¹H NMR (400 MHz, CDCl3-d) δ 8.17 (d, J = 8.1 Hz, 1H), 7.88 (d, J = 8.1 Hz, | |
| | 1H), 3.97-3.86 (m, 1H), 3.44 (s, 3H), 3.21-3.04 (m, 2H), 2.16-1.87 (m, 2H), 1.40 | |
| | (d, J = 7.0 Hz, 6H), 1.16 (t, J = 7.4 Hz, 3H). | |
| 3-503 | ¹H NMR (400 MHz, CDCl3-d) δ 8.39 (d, J = 8.2 Hz, 1H), 7.94 (d, J = 8.2 Hz, | |
| | 1H), 3.67-3.57 (m, 5H), 3.22-3.07 (m, 1H), 2.14-2.02 (m, 2H), 1.41 (d, J = 7.0 | |
| | Hz, 6H), 1.14 (t, J = 7.4 Hz, 3H). | |
| 3-504 | ¹H NMR (400 MHz, DMSO-d₆) δ 12.50 (s, 1H), 8.14 (d, J = 8.1 Hz, 1H), 7.88 | |
| | (d, J = 8.1 Hz, 1H), 3.85-3.73 (m, 1H), 3.55 (s, 3H), 3.25-3.10 (m, 1H), 1.33- | |
| | 1.21 (m, 12H). | |
| 3-505 | ¹H NMR (400 MHz, DMSO-d₆) δ 12.62 (s, 1H), 8.18 (d, J = 8.1 Hz, 1H), 8.05 | |
| | (d, J = 8.1 Hz, 1H), 4.15-4.03 (m, 1H), 3.51 (s, 3H), 3.22-3.11 (m, 1H), 1.45 (d, | |
| | J = 6.8 Hz, 3H), 1.33-1.24 (m, 6H), 1.11 (d, J = 7.0 Hz, 3H). | |
| 3-508 | ¹H NMR (400 MHz, CDCl3-d) δ 8.48 (dd, J = 8.3, 0.9 Hz, 1H), 7.92 (d, J = 8.2 | |
| | Hz, 1H), 4.29-4.18 (m, 1H), 3.58 (s, 3H), 3.18-3.09 (m, 1H), 1.52-1.45 (m, 6H), | |
| | 1.39 (d, J = 6.9 Hz, 6H). | |
| 3-517 | ¹H NMR (400 MHz, DMSO-d₆) δ 12.57 (s, 1H), 8.18 (d, J = 8.1 Hz, 1H), 8.05 | |
| | (d, J = 8.3 Hz, 1H), 4.10-3.77 (m, 1H), 3.51 (d, J = 4.0 Hz, 3H), 3.18 (s, 1H), 2.14- | |
| | 2.00 (m, 1H), 1.84-1.67 (m, 1H), 1.55-1.35 (m, 2H), 1.34-1.22 (m, 6H), 1.07- | |
| | 1.01 (m, 3H), 0.92 (t, J = 7.4 Hz, 1H). | |
| 3-522 | ¹H NMR (400 MHz, DMSO-d₆) δ 12.57 (s, 1H), 8.44 (d, J = 8.2 Hz, 1H), 8.20 | |
| | (d, J = 8.3 Hz, 1H), 4.09-3.94 (m, 1H), 3.62 (s, 3H), 3.26-3.10 (m, 1H), 2.06- | |
| | 1.90 (m, 1H), 1.75-1.60 (m, 1H), 1.35 (d, J = 6.9 Hz, 3H), 1.33-1.24 (m, 6H), 1.01 | |
| | (t, J = 7.4 Hz, 3H). | |
| 3-534 | ¹H NMR (400 MHz, CDCl3-d) δ 8.38 (d, J = 8.2 Hz, 1H), 7.93 (d, J = 8.2 Hz, | |
| | 1H), 3.64-3.58 (m, 5H), 3.22-3.07 (m, 1H), 1.43-1.31 (m, 7H), 0.78-0.65 (m, | |
| | 2H), 0.49-0.40 (m, 2H). | |
| 3-535 | ¹H NMR (400 MHz, DMSO-d₆) δ 12.55 (s, 1H), 8.10 (d, J = 8.1 Hz, 1H), 7.88 | |
| | (d, J = 8.1 Hz, 1H), 3.47 (s, 3H), 3.22-3.10 (m, 1H), 2.72-2.62 (m, 1H), 1.29 (d, | |
| | J = 6.9 Hz, 6H), 0.93-0.86 (m, 2H), 0.81-0.73 (m, 2H). | |
| 3-536 | ¹H NMR (400 MHz, CDCl3-d) δ 8.22 (d, J = 8.1 Hz, 1H), 7.89 (d, J = 8.1 Hz, | |
| | 1H), 3.57-3.49 (m, 1H), 3.42 (s, 3H), 3.20-3.07 (m, 1H), 1.44-1.28 (m, 9H), 1.06- | |
| | 0.97 (m, 1H). | |
| 3-539 | ¹H NMR (400 MHz, DMSO-d₆) δ 8.40 (d, J = 8.2 Hz, 1H), 8.18 (d, J = 8.2 Hz, | |
| | 1H), 3.64 (s, 3H), 3.47-3.46 (m, 1H), 3.20-3.11 (m, 1H), 1.52-1.44 (m, 2H), 1.29 | |
| | (d, J = 6.9 Hz, 6H), 1.25-1.18 (m, 2H). | |
| 3-541 | ¹H NMR (400 MHz, DMSO-d₆) δ 12.55 (s, 1H), 8.10 (d, J = 8.1 Hz, 1H), 8.00 | |
| | (d, J = 8.1 Hz, 1H), 4.44 (p, J = 8.4 Hz, 1H), 3.52 (s, 3H), 3.23-3.11 (m, 1H), 2.80- | |
| | 2.66 (m, 1H), 2.35-2.16 (m, 2H), 2.16-2.06 (m, 1H), 2.06-1.96 (m, 1H), 1.97- | |
| | 1.88 (m, 1H), 1.37-1.26 (m, 6H). | |
| 3-542 | ¹H NMR (400 MHz, CDCl3-d) δ 8.15 (d, J = 8.1 Hz, 1H), 7.83 (d, J = 8.1 Hz, | [α] |
| | 1H), 4.54 (p, J = 8.5 Hz, 1H), 3.41 (s, 3H), 3.15 (p, J = 7.0 Hz, 1H), 2.94-2.43 (m, | D²⁰ = +57.50°(c |
| | 2H), 2.37-2.16 (m, 2H), 2.08-1.99 (m, 2H), 1.40 (d, J = 7.0 Hz, 6H). | 0.102, DMF) |
| 3-543 | ¹H NMR (400 MHz, CDCl3-d) δ 8.15 (d, J = 8.1 Hz, 1H), 7.83 (d, J = 8.1 Hz, | [α] |
| | 1H), 4.54 (p, J = 8.5 Hz, 1H), 3.41 (s, 3H), 3.15 (p, J = 7.0 Hz, 1H), 2.94-2.43 (m, | D²⁰ = −70.69°(c |
| | 2H), 2.37-2.16 (m, 2H), 2.08-1.99 (m, 2H), 1.40 (d, J = 7.0 Hz, 6H). | 0.101 , DMF) |
| 3-544 | ¹H NMR (400 MHz, DMSO-d₆) δ 12.61 (s, 1H), 8.39 (d, J = 8.2 Hz, 1H), 8.18 | |
| | (d, J = 8.2 Hz, 1H), 4.72 (p, J = 8.5 Hz, 1H), 3.65 (s, 3H), 3.23-3.10 (m, 1H), 2.73- | |
| | 2.60 (m, 2H), 2.32-2.17 (m, 2H), 2.03-1.89 (m, 2H), 1.35-1.25 (m, 6H). | |
| 3-546 | ¹H NMR (400 MHz, CDCl3-d) δ 8.22 (d, J = 8.1 Hz, 1H), 7.87 (d, J = 8.2 Hz, | |
| | 1H), 4.56-4.48 (m, 1H), 3.41 (s, 3H), 3.19-3.08 (m, 1H), 2.37-2.25 (m, 1H), 2.22- | |
| | 2.11 (m, 1H), 1.88-1.70 (m, 5H), 1.64-1.58 (m, 1H), 1.40 (d, J = 6.9 Hz, 6H). | |
| 3-549 | ¹H NMR (400 MHz, DMSO-d₆) δ 8.44 (d, J = 8.2 Hz, 1H), 8.19 (d, J = 8.2 Hz, | |
| | 1H), 4.52-4.39 (m, 1H), 3.63 (s, 3H), 3.22-3.11 (m, 1H), 2.22-2.09 (m, 2H), 1.99- | |
| | 1.86 (m, 2H), 1.78-1.69 (m, 2H), 1.67-1.60 (m, 2H), 1.28 (d, J = 6.9 Hz, 6H). | |
| 3-571 | ¹H NMR (400 MHz, DMSO-d₆) δ 12.59 (s, 1H), 8.13 (d, J = 8.1 Hz, 1H), 8.05 | |
| | (d, J = 8.2 Hz, 1H), 6.04-5.89 (m, 1H), 5.49-5.39 (m, 2H), 4.46-4.37 (m, 1H), | |
| | 4.06-3.97 (m, 1H), 3.53 (s, 3H), 3.24-3.01 (m, 1H), 1.30 (d, J = 7.0 Hz, 6H). | |
| 3-574 | ¹H NMR (400 MHz, DMSO-d₆) δ 8.36 (d, J = 8.2 Hz, 1H), 8.18 (d, J = 8.2 Hz, | |
| | 1H), 6.01-5.86 (m, 1H), 5.54-5.44 (m, 2H), 4.51 (d, J = 7.4 Hz, 2H), 3.64 (s, 3H), | |
| | 3.23-3.10 (m, 1H), 1.28 (d, J = 6.9 Hz, 6H). | |
| 3-615 | ¹H NMR (400 MHz, CDCl3-d) δ 8.19 (d, J = 8.2 Hz, 1H), 7.76 (d, J = 8.2 Hz, | |
| | 1H), 3.46 (s, 3H), 3.05-2.97 (m, 2H), 2.27-2.17 (m, 1H), 1.79-1.62 (m, 2H), 1.19 | |
| | (d, J = 8.4 Hz, 4H), 1.04 (t, J = 7.4 Hz, 3H). | |
| 3-616 | ¹H NMR (400 MHz, DMSO-d₆) δ 12.49 (s, 1H), 8.11 (d, J = 8.1 Hz, 1H), 8.01 | |
| | (d, J = 8.2 Hz, 1H), 3.77-3.66 (m, 1H), 3.53 (s, 3H), 3.12-3.00 (m, 1H), 2.20 (s, | |
| | 1H), 2.26-2.12 (m, 1H), 1.90-1.72 (m, 1H), 1.36-1.21 (m, 4H), 1.08 (t, J = 7.4 | |
| | Hz, 3H). | |
| 3-619 | ¹H NMR (400 MHz, CDCl3-d) δ 8.36 (d, J = 8.2 Hz, 1H), 7.92 (d, J = 8.2 Hz, | |
| | 1H), 3.67-3.56 (m, 5H), 2.16-2.00 (m, 3H), 1.21 (d, J = 8.5 Hz, 4H), 1.14 (t, J = | |
| | 7.4 Hz, 3H). | |
| 3-620 | ¹H NMR (400 MHz, DMSO-d₆) δ 12.45 (s, 1H), 8.14 (d, J = 8.1 Hz, 1H), 7.87 | |
| | (d, J = 8.2 Hz, 1H), 3.85-3.73 (m, 1H), 3.55 (s, 3H), 2.30-2.13 (m, 1H), 1.24 (d, J = | |
| | 6.6 Hz, 6H), 1.17-1.07 (m, 2H), 1.05-0.90 (m, 2H). | |
| 3-621 | ¹H NMR (400 MHz, DMSO-d₆) δ 12.58 (s, 1H), 8.17 (d, J = 8.1 Hz, 1H), 8.02 | |
| | (d, J = 8.2 Hz, 1H), 4.15-4.02 (m, 1H), 3.51 (s, 3H), 2.24-2.13 (m, 1H), 1.45 (d, J = | |
| | 6.8 Hz, 3H), 1.16-1.03 (m, 5H), 1.01-0.89 (m, 2H). | |
| 3-622 | ¹H NMR (400 MHz, DMSO-d₆) δ 12.54 (s, 1H), 8.18 (d, J = 8.1 Hz, 1H), 8.04 | [α] |
| | (d, J = 7.9 Hz, 1H), 4.21-4.05 (m, 1H), 3.51 (s, 3H), 2.19 (d, J = 7.4 Hz, 1H), 1.45 | D²⁰ = +51.53°(c |
| | (d, J = 6.8 Hz, 3H), 1.11 (d, J = 6.9 Hz, 5H), 0.97 (s, 2H). | 0.111, DMF) |
| 3-623 | ¹H NMR (400 MHz, DMSO-d₆) δ 12.57 (s, 1H), 8.18 (d, J = 7.9 Hz, 1H), 8.03 | [α] |
| | (d, J = 8.0 Hz, 1H), 4.17-4.01 (m, 1H), 3.51 (s, 3H), 2.19 (s, 1H), 1.45 (d, J = 6.8 | D²⁰ = −58.27°(c |
| | Hz, 3H), 1.10 (d, J = 7.0 Hz, 5H), 0.96 (s, 2H). | 0.101, DMF) |
| 3-624 | ¹H NMR (400 MHz, DMSO-d₆) δ 12.58 (s, 1H), 8.43 (d, J = 8.2 Hz, 1H), 8.18 | |
| | (d, J = 8.2 Hz, 1H), 4.17 (p, J = 6.8 Hz, 1H), 3.62 (s, 3H), 2.24-2.13 (m, 1H), 1.35 | |
| | (d, J = 6.8 Hz, 6H), 1.15-1.04 (m, 2H), 1.01-0.90 (m, 2H). | |
| 3-633 | ¹H NMR (400 MHz, DMSO-d₆) δ 12.52 (s, 1H), 8.18 (d, J = 8.1 Hz, 1H), 8.04 | |
| | (d, J = 7.8 Hz, 1H), 4.07-3.80 (m, 1H), 3.51 (d, J = 3.9 Hz, 3H), 2.27-2.14 (m, | |
| | 1H), 2.12-1.98 (m, 1H), 1.84-1.65 (m, 1H), 1.56-1.37 (m, 2H), 1.37-1.15 (m, | |
| | 2H), 1.07-1.02 (m, 3H), 1.01-0.95 (m, 2H), 0.92 (t, J = 7.4 Hz, 1H). | |
| 3-638 | ¹H NMR (400 MHz, DMSO-d₆) δ 12.53 (s, 1H), 8.44 (d, J = 8.2 Hz, 1H), 8.19 | |
| | (d, J = 8.3 Hz, 1H), 4.08-3.91 (m, 1H), 3.62 (s, 3H), 2.28-2.10 (m, 1H), 2.03- | |
| | 1.89 (m, 1H), 1.75-1.56 (m, 1H), 1.34 (d, J = 6.9 Hz, 3H), 1.17-1.07 (m, 2H), 1.05- | |
| | 0.88 (m, 5H). | |
| 3-650 | ¹H NMR (400 MHz, DMSO-d₆) δ 12.60 (s, 1H), 8.37 (d, J = 8.2 Hz, 1H), 8.17- | |
| | 8.11 (m, 1H), 3.69-3.61 (m, 5H), 2.20-2.16 (m, 1H), 1.26-1.16 (m, 1H), 1.15- | |
| | 1.09 (m, 2H), 1.00-0.95 (m, 2H), 0.66-0.60 (m, 2H), 0.40-0.34 (m, 2H). | |
| 3-651 | ¹H NMR (400 MHz, DMSO-d₆) δ 12.39 (s, 1H), 8.11 (d, J = 8.1 Hz, 1H), 7.87 | |
| | (d, J = 8.2 Hz, 1H), 3.48 (s, 3H), 2.72-2.62 (m, 1H), 2.24-2.16 (m, 1H), 1.18- | |
| | 1.07 (m, 2H), 1.04-0.85 (m, 4H), 0.82-0.66 (m, 2H). | |
| 3-652 | ¹H NMR (400 MHz, CDCl3-d) δ 8.22 (d, J = 8.1 Hz, 1H), 7.86 (d, J = 8.1 Hz, | |
| | 1H), 3.57-3.48 (m, 1H), 3.42 (s, 3H), 2.15-2.06 (m, 1H), 1.45-1.25 (m, 6H), 1.07- | |
| | 0.98 (m, 1H), 0.91-0.80 (m, 1H). | |
| 3-655 | ¹H NMR (400 MHz, DMSO-d₆) δ 12.43 (brs, 1H), 8.40 (d, J = 8.2 Hz, 1H), 8.16 | |
| | (d, J = 8.3 Hz, 1H), 3.69-3.60 (m, 4H), 2.25-2.13 (m, 1H), 1.51-1.42 (m, 2H), | |
| | 1.27-1.18 (m, 2H), 1.16-1.05 (m, 2H), 1.00-0.88 (m, 2H). | |
| 3-657 | ¹H NMR (400 MHz, DMSO-d₆) δ 12.51 (s, 1H), 8.10 (d, J = 8.1 Hz, 1H), 7.98 | |
| | (d, J = 8.1 Hz, 1H), 4.44 (p, J = 8.3 Hz, 1H), 3.52 (s, 3H), 2.80-2.66 (m, 1H), 2.34 | |
| | 1.92 (m, 4H), 1.37-1.21 (m, 2H), 1.21-1.08 (m, 2H), 1.05-0.90 (m, 2H). | |
| 3-660 | ¹H NMR (400 MHz, DMSO-d₆) δ 12.56 (s, 1H), 8.38 (d, J = 8.2 Hz, 1H), 8.16 | |
| | (d, J = 8.2 Hz, 1H), 4.72 (p, J = 8.5 Hz, 1H), 3.64 (s, 3H), 2.76-2.58 (m, 2H), 2.31- | |
| | 2.12 (m, 3H), 2.09-1.92 (m, 2H), 1.28-1.20 (m, 2H), 1.17-1.06 (m, 2H). | |
| 3-662 | ¹H NMR (400 MHz, DMSO-d₆) δ 12.58 (s, 1H), 8.16 (d, J = 8.1 Hz, 1H), 8.02 | |
| | (d, J = 8.1 Hz, 1H), 4.52-4.22 (m, 1H), 3.52 (s, 3H), 2.26-1.95 (m, 3H), 1.79- | |
| | 1.48 (m, 6H), 1.17-1.05 (m, 2H), 1.02-0.90 (m, 2H). | |
| 3-665 | ¹H NMR (400 MHz, DMSO-d₆) δ 12.55 (s, 1H), 8.40 (d, J = 8.2 Hz, 1H), 8.16- | |
| | 8.06 (m, 1H), 4.45 (p, J = 7.5 Hz, 1H), 3.61 (s, 3H), 2.19-2.10 (m, 3H), 1.94-1.88 | |
| | (m, 2H), 1.77-1.71 (m, 2H), 1.67-1.59 (m, 2H), 1.13-1.04 (m, 2H), 0.95-0.91 | |
| | (m, 2H). | |
| 3-687 | ¹H NMR (400 MHz, DMSO-d₆) δ 12.55 (brs, 1H), 8.12 (d, J = 8.1 Hz, 1H), 8.01 | |
| | (d, J = 8.1 Hz, 1H), 6.03-5.84 (m, 1H), 5.48-5.41 (m, 2H), 4.46-4.36 (m, 1H), | |
| | 4.05-3.96 (m, 1H), 3.52 (s, 3H), 2.22-2.16 (m, 1H), 1.11 (m, 2H), 1.14-1.07 (m, 2H). | |
| 3-690 | ¹H NMR (400 MHz, DMSO-d₆) δ 12.64 (brs, 1H), 8.36 (d, J = 8.2 Hz, 1H), 8.20- | |
| | 8.11 (m, 1H), 6.00-5.86 (m, 1H), 5.53-5.43 (m, 2H), 4.51 (d, J = 7.4 Hz, 2H), | |
| | 3.63 (s, 3H), 2.26-2.09 (m, 1H), 1.16-1.02 (m, 2H), 0.98-0.93 (m, 2H). | |
| 3-731 | ¹H NMR (400 MHz, DMSO-d₆) δ 12.49 (s, 1H), 8.12 (d, J = 8.1 Hz, 1H), 7.89 | |
| | (d, J = 8.2 Hz, 1H), 3.57 (s, 3H), 2.99 (t, J = 7.4 Hz, 2H), 2.81 (t, J = 7.2 Hz, 2H), | |
| | 1.77-1.55 (m, 4H), 0.98 (t, J = 7.4 Hz, 6H). | |
| 3-732 | ¹H NMR (400 MHz, DMSO-d₆) δ 12.49 (s, 1H), 8.12 (d, J = 8.1 Hz, 1H), 7.89 | |
| | (d, J = 8.2 Hz, 1H), 3.57 (s, 3H), 2.99 (t, J = 7.4 Hz, 2H), 2.81 (t, J = 7.2 Hz, 2H), | |
| | 1.77-1.55 (m, 4H), 0.98 (t, J = 7.4 Hz, 6H). | |
| 3-735 | ¹H NMR (400 MHz, DMSO-d₆) δ 12.66 (s, 1H), 8.37 (d, J = 8.2 Hz, 1H), 8.20 (d, J = | |
| | 8.2 Hz, 1H), 3.73-3.63 (m, 5H), 2.80 (t, J = 7.3 Hz, 2H), 1.95-1.81 (m, 2H), 1.78- | |
| | 1.61 (m, 2H), 1.06 (t, J = 7.4 Hz, 3H), 0.96 (t, J = 7.4 Hz, 3H). | |
| 3-736 | ¹H NMR (400 MHz, DMSO-d₆) δ 12.49 (s, 1H), 8.14 (d, J = 8.1 Hz, 1H), 7.88 | |
| | (d, J = 8.1 Hz, 1H), 3.85-3.72 (m, 1H), 3.55 (s, 3H), 2.87-2.73 (m, 2H), 1.79- | |
| | 1.62 (m, 2H), 1.24 (d, J = 6.6 Hz, 6H), 0.96 (t, J = 6.9 Hz, 3H). | |
| 3-737 | ¹H NMR (400 MHz, DMSO-d₆) δ 12.65 (s, 1H), 8.18 (d, J = 8.1 Hz, 1H), 8.03 | |
| | (d, J = 8.1 Hz, 1H), 4.17-4.02 (m, 1H), 3.51 (s, 3H), 2.79 (t, J = 7.2 Hz, 2H), 1.77- | |
| | 1.62 (m, 2H), 1.45 (d, J = 6.8 Hz, 3H), 1.11 (d, J = 7.0 Hz, 3H), 0.96 (t, J = 7.4 Hz, 3H). | |
| 3-740 | ¹H NMR (400 MHz, DMSO-d₆) δ 12.60 (s, 1H), 8.44 (d, J = 8.2 Hz, 1H), 8.20 | |
| | (d, J = 8.2 Hz, 1H), 4.26-4.09 (m, 1H), 3.63 (s, 3H), 2.80 (t, J = 7.0 Hz, 2H), 1.77- | |
| | 1.61 (m, 2H), 1.35 (d, J = 6.8 Hz, 6H), 0.95 (t, J = 7.3 Hz, 3H). | |
| 3-741 | ¹H NMR (400 MHz, CDCl₃-d) δ 8.17 (d, J = 8.2 Hz, 1H), 7.74 (d, J = 8.2 Hz, | |
| | 1H), 3.47 (s, 3H), 2.99 (t, J = 7.5 Hz, 2H), 1.78-1.64 (m, 2H), 1.41 (s, 9H), 1.03 (t, J = | |
| | 7.3 Hz, 3H). | |
| 3-742 | ¹H NMR (400 MHz, CDCl₃-d) δ 8.16 (d, J = 7.9 Hz, 1H), 7.88 (d, J = 8.0 Hz, | |
| | 1H), 3.97-3.86 (m, 1H), 3.44 (s, 3H), 3.15-3.03 (m, 1H), 2.10-1.89 (m, 2H), 1.43 | |
| | (s, 9H), 1.16 (t, J = 7.4 Hz, 3H). | |
| 3-745 | ¹H NMR (400 MHz, CDCl₃-d) δ 8.36 (d, J = 8.2 Hz, 1H), 7.94 (d, J = 8.2 Hz, | |
| | 1H), 3.66-3.54 (m, 5H), 2.12-1.98 (m, 2H), 1.42 (s, 9H), 1.13 (t, J = 7.4 Hz, 3H). | |
| 3-746 | ¹H NMR (400 MHz, CDCl₃-d) δ 8.20 (d, J = 8.1 Hz, 1H), 7.74 (d, J = 8.2 Hz, 1H), | |
| | 3.89-3.78 (m, 1H), 3.45 (s, 3H), 1.40 (s, 9H), 1.29 (d, J = 6.7 Hz, 6H). | |
| 3-747 | ¹H NMR (400 MHz, DMSO-d₆) δ 12.59 (s, 1H), 8.19 (d, J = 8.1 Hz, 1H), 8.04 | |
| | (d, J = 8.1 Hz, 1H), 4.23-3.86 (m, 1H), 3.50 (s, 3H), 1.46 (d, J = 6.8 Hz, 3H), 1.34 | |
| | (s, 9H), 1.11 (d, J = 7.0 Hz, 3H). | |
| 3-750 | ¹H NMR (400 MHz, CDCl₃-d) δ 8.48 (d, J = 8.3 Hz, 1H), 7.92 (d, J = 8.2 Hz, 1H), | |
| | 4.31-4.16 (m, 1H), 3.58 (s, 3H), 1.47 (d, J = 6.9 Hz, 6H), 1.42 (s, 9H). | |
| 3-751 | ¹H NMR (400 MHz, CDCl₃-d) δ 8.09-8.03 (m, 1H), 7.68 (d, J = 8.1 Hz, 1H), 3.47 | |
| | (s, 3H), 2.97 (t, J = 7.4 Hz, 2H), 1.76-1.64 (m, 2H), 1.04 (t, J = 7.3 Hz, 3H). | |
| 3-752 | ¹H NMR (400 MHz, CDCl₃-d) δ 8.28-7.71 (m, 2H), 3.92-3.77 (m, 1H), 3.40 (s, | |
| | 3H), 3.21-3.03 (m, 1H), 1.97-1.91 (m, 2H), 1.14 (t, J = 7.2 Hz, 3H). | |
| 3-755 | ¹H NMR (400 MHz, CDCl₃-d) δ 11.19 (s, 1H), 8.39 (d, J = 8.1 Hz, 1H), 7.93 (d, | |
| | J = 8.2 Hz, 1H), 3.69-3.52 (m, 5H), 2.12-1.98 (m, 2H), 1.14 (t, J = 7.4 Hz, 3H). | |
| 3-756 | ¹H NMR (400 MHz, CDCl₃-d) δ 8.12 (d, J = 8.1 Hz, 1H), 7.69 (d, J = 8.2 Hz, | |
| | 1H), 3.91-3.76 (m, 1H), 3.46 (s, 3H), 1.29 (d, J = 6.7 Hz, 6H). | |
| 3-757 | ¹H NMR (400 MHz, CDCl₃-d) δ 8.24 (d, J = 8.0 Hz, 1H), 7.85 (d, J = 8.0 Hz, | |
| | 1H), 4.41-4.12 (m, 1H), 3.34 (s, 3H), 1.57 (d, J = 6.8 Hz, 3H), 1.18 (d, J = 7.0 Hz, 3H). | |
| 3-760 | ¹H NMR (400 MHz, CDCl₃-d) δ 8.48 (d, J = 8.1 Hz, 1H), 7.92 (d, J = 8.2 Hz, | |
| | 1H), 4.27-4.19 (m, 1H), 3.57 (s, 3H), 1.45 (d, J = 6.9 Hz, 6H). | |
| 3-761 | ¹H NMR (400 MHz, DMSO-d₆) δ 12.51 (s, 1H), 8.14 (d, J = 8.1 Hz, 1H), 7.88 | |
| | (d, J = 8.0 Hz, 1H), 3.85-3.69 (m, 2H), 3.55 (s, 3H), 2.46-2.17 (m, 4H), 2.12- | |
| | 1.85 (m, 2H), 1.24 (d, J = 6.7 Hz, 6H). | |
| 3-762 | ¹H NMR (400 MHz, DMSO-d₆) δ 12.61 (s, 1H), 8.18 (d, J = 8.1 Hz, 1H), 8.06 | |
| | (d, J = 8.1 Hz, 1H), 4.14-4.05 (m, 1H), 3.79-3.67 (m, 1H), 3.52 (s, 3H), 2.42- | |
| | 2.23 (m, 4H), 2.13-1.91 (m, 2H), 1.45 (d, J = 6.8 Hz, 3H), 1.11 (d, J = 7.0 Hz, 3H). | |
| 3-765 | ¹H NMR (400 MHz, DMSO-d₆) δ 12.62 (s, 1H), 8.44 (d, J = 8.2 Hz, 1H), 8.20 | |
| | (d, J = 8.1 Hz, 1H), 4.25-4.11 (m, 1H), 3.80-3.67 (m, 1H), 3.63 (s, 3H), 2.44- | |
| | 2.20 (m, 4H), 2.13-1.93 (m, 2H), 1.35 (d, J = 6.8 Hz, 6H). | |
| 3-766 | ¹H NMR (400 MHz, DMSO-d₆) δ 12.81 (s, 1H), 8.16 (d, J = 8.1 Hz, 1H), 8.05 | |
| | (s, 1H), 7.91 (d, J = 8.1 Hz, 1H), 7.28 (s, 1H), 6.83-6.77 (m, 1H), 3.83-3.76 (m, | |
| | 1H), 3.56 (s, 3H), 1.25 (d, J = 6.0 Hz, 6H). | |
| 3-767 | ¹H NMR (400 MHz, DMSO-d₆) δ 12.88 (s, 1H), 8.20 (d, J = 8.1 Hz, 1H), 8.14- | |
| | 8.02 (m, 2H), 7.28 (s, 1H), 6.83-6.77 (m, 1H), 4.17-4.03 (m, 1H), 3.52 (s, 3H), | |
| | 1.46 (d, J = 6.8 Hz, 3H), 1.12 (d, J = 7.0 Hz, 3H). | |
| 3-770 | ¹H NMR (400 MHz, DMSO-d₆) δ 12.87 (s, 1H), 8.46 (d, J = 8.2 Hz, 1H), 8.24 | |
| | (d, J = 8.2 Hz, 1H), 8.05 (s, 1H), 7.28 (s, 1H), 6.85-6.77 (m, 1H), 4.24-4.14 (m, | |
| | 1H), 3.64 (s, 3H), 1.36 (d, J = 6.8 Hz, 6H). | |
| 3-771 | ¹H NMR (400 MHz, DMSO-d₆) δ 12.80 (s, 1H), 8.17 (d, J = 8.1 Hz, 1H), 8.04- | |
| | 7.85 (m, 3H), 7.66-7.58 (m, 3H), 3.87-3.75 (m, 1H), 3.56 (s, 3H), 1.26-1.22 (m, 6H). | |
| 3-772 | ¹H NMR (400 MHz, DMSO-d₆) δ 12.89 (s, 1H), 8.21 (d, J = 8.1 Hz, 1H), 8.10 | |
| | (d, J = 8.1 Hz, 1H), 7.99-7.87 (m, 2H), 7.67-7.57 (m, 3H), 4.20-4.05 (m, 1H), | |
| | 3.53 (s, 3H), 1.46 (d, J = 6.8 Hz, 3H), 1.12 (d, J = 7.0 Hz, 3H). | |
| 3-775 | ¹H NMR (400 MHz, DMSO-d₆) δ 12.86 (s, 1H), 8.47 (d, J = 8.2 Hz, 1H), 8.25 | |
| | (d, J = 8.2 Hz, 1H), 8.09-7.85 (m, 2H), 7.72-7.56 (m, 3H), 4.25-4.14 (m, 1H), | |
| | 3.64 (s, 3H), 1.36 (d, J = 6.8 Hz, 6H). | |
| 3-776 | ¹H NMR (400 MHz, DMSO-d₆) δ 12.85 (s, 1H), 8.17 (d, J = 8.2 Hz, 1H), 8.02- | |
| | 7.95 (m, 2H), 7.93 (d, J = 8.1 Hz, 1H), 7.46 (t, J = 8.7 Hz, 2H), 3.87--3.76 (m, 1H), | |
| | 3.57 (s, 3H), 1.25 (d, J = 6.6 Hz, 6H). | |
| 3-777 | ¹H NMR (400 MHz, DMSO-d₆) δ 12.89 (s, 1H), 8.22 (d, J = 8.1 Hz, 1H), 8.10 | |
| | (d, J = 8.1 Hz, 1H), 7.99 (s, 2H), 7.47 (t, J = 8.8 Hz, 2H), 4.16-4.05 (m, 1H), 3.53 (s, | |
| | 3H), 1.46 (d, J = 6.8 Hz, 3H), 1.12 (d, J = 7.0 Hz, 3H). | |
| 3-780 | ¹H NMR (400 MHz, DMSO-d₆) δ 12.90 (s, 1H), 8.48 (d, J = 8.2 Hz, 1H), 8.25 | |
| | (d, J = 8.2 Hz, 1H), 7.98 (t, J = 6.9 Hz, 2H), 7.46 (t, J = 8.6 Hz, 2H), 4.26-4.14 (m, | |
| | 1H), 3.65 (s, 3H), 1.36 (d, J = 6.8 Hz, 6H). | |
| 3-781 | ¹H NMR (400 MHz, DMSO-d₆) δ 13.09 (s, 1H), 8.16 (d, J = 8.1 Hz, 1H), 7.91 | |
| | (d, J = 8.1 Hz, 1H), 4.42 (q, J = 7.1 Hz, 2H), 3.87-3.73 (m, 1H), 3.56 (s, 3H), 1.34 | |
| | (t, J = 7.1 Hz, 3H), 1.25 (d, J = 6.7 Hz, 6H). | |
| 3-782 | ¹H NMR (400 MHz, DMSO-d₆) δ 13.18 (s, 1H), 8.21 (d, J = 8.1 Hz, 1H), 8.08 (d, J = | |
| | 8.1 Hz, 1H), 4.42 (q, J = 7.1 Hz, 2H), 4.18-4.03 (m, 1H), 3.53 (s, 3H), 1.46 (d, J = | |
| | 6.8 Hz, 3H), 1.34 (t, J = 7.1 Hz, 3H), 1.12 (d, J = 7.0 Hz, 3H). | |
| 3-785 | ¹H NMR (400 MHz, DMSO-d₆) δ 13.17 (s, 1H), 8.47 (d, J = 8.2 Hz, 1H), 8.24 | |
| | (d, J = 8.1 Hz, 1H), 4.42 (q, J = 7.0 Hz, 2H), 4.26-4.12 (m, 1H), 3.64 (s, 3H), 1.43- | |
| | 1.28 (m, 9H). | |
| 3-786 | ¹H NMR (400 MHz, CDCl₃-d) δ 8.19 (d, J = 8.1 Hz, 1H), 7.52 (d, J = 8.1 Hz, | |
| | 1H), 3.58 (s, 3H), 3.45 (s, 3H), 2.94 (t, J = 7.5 Hz, 2H), 2.37 (s, 3H), 1.73-1.61 (m, | |
| | 2H), 1.02 (t, J = 7.3 Hz, 3H). | |
| 3-787 | ¹H NMR (400 MHz, CDCl3-d) δ 8.15 (d, J = 8.1 Hz, 1H), 7.64 (d, J = 8.1 Hz, | |
| | 1H), 3.80-3.76 (m, 1H), 3.58 (s, 3H), 3.41 (s, 3H), 3.11-2.99 (m, 1H), 2.42 (s, 3H), | |
| | 2.07-1.83 (m, 2H), 1.14 (t, J = 7.4 Hz, 3H). | |
| 3-788 | ¹H NMR (400 MHz, CDCl₃-d) δ 8.15 (d, J = 8.1 Hz, 1H), 7.64 (d, J = 8.1 Hz, | [α] |
| | 1H), 3.87-3.68 (m, 1H), 3.58 (s, 3H), 3.41 (s, 3H), 3.11-2.99 (m, 1H), 2.42 (s, 3H), | D²⁰ = +143.41°(c |
| | 2.09-1.83 (m, 2H), 1.14 (t, J = 7.4 Hz, 3H). | 0.099, DMF) |
| 3-789 | ¹H NMR (400 MHz, CDCl₃-d) δ 8.15 (d, J = 8.1 Hz, 1H), 7.64 (d, J = 8.1 Hz, | [α] |
| | 1H), 3.86-3.70 (m, 1H), 3.58 (s, 3H), 3.41 (s, 3H), 3.11-2.99 (m, 1H), 2.42 (s, | D²⁰ = −144.20°(c |
| | 3H), 2.11-1.83 (m, 2H), 1.14 (t, J = 7.4 Hz, 3H). | 0.097, DMF) |
| 3-790 | ¹H NMR (400 MHz, DMSO-d₆) δ 8.32 (d, J = 8.0 Hz, 1H), 8.08 (d, J = 8.2 Hz, | |
| | 1H), 3.69-3.60 (m, 5H), 3.49 (s, 3H), 2.33 (s, 3H), 1.93-1.77 (m, 2H), 1.02 (t, J = | |
| | 7.4 Hz, 3H). | |
| 3-792 | ¹H NMR (400 MHz, DMSO-d₆) δ 12.60 (s, 1H), 8.29 (d, J = 8.1 Hz, 1H), 8.14 | |
| | (d, J = 8.2 Hz, 1H), 6.55-6.07 (m, 1H), 3.49 (s, 3H), 2.51 (s, 3H). | |
| 3-795 | ¹H NMR (400 MHz, CDCl₃-d) δ 8.20-8.14 (m, 2H), 7.64 (d, J = 8.1 Hz, 1H), | |
| | 3.84-3.72 (m, 1H), 3.65 (s, 3H), 3.42 (s, 3H), 3.12-3.00 (m, 1H), 2.10-1.83 (m, | |
| | 2H), 1.14 (t, J = 7.4 Hz, 3H). | |
| 3-798 | ¹H NMR (400 MHz, CDCl₃-d) δ 8.16 (d, J = 8.1 Hz, 1H), 7.63 (d, J = 8.1 Hz, | |
| | 1H), 3.85-3.74 (m, 1H), 3.61 (s, 3H), 3.41 (s, 3H), 3.10-3.00 (m, 1H), 2.81-2.66 | |
| | (m, 2H), 2.05-1.90 (m, 2H), 1.24-1.16 (m, 3H), 1.14 (t, J = 7.4 Hz, 3H). | |
| 3-801 | ¹H NMR (400 MHz, CDCl₃-d) δ 8.16 (d, J = 8.1 Hz, 1H), 7.63 (d, J = 8.1 Hz, | |
| | 1H), 3.94-3.72 (m, 1H), 3.63 (s, 3H), 3.40 (s, 3H), 3.10-2.96 (m, 2H), 2.10-1.84 | |
| | (m, 2H), 1.21-1.11 (m, 9H). | |
| 3-804 | ¹H NMR (400 MHz, DMSO-d₆) δ 9.03 (s, 1H), 8.22-8.08 (m, 1H), 7.97 (d, J = | |
| | 8.1 Hz, 1H), 4.12-3.78 (m, 1H), 3.52 (d, J = 8.9 Hz, 6H), 1.41 (d, J = 6.8 Hz, 3H), | |
| | 1.05 (d, J = 7.0 Hz, 3H). | |
| 3-807 | ¹H NMR (400 MHz, DMSO-d₆) δ 8.22-7.83 (m, 2H), 4.18-3.84 (m, 1H), 3.52- | |
| | 3.48 (m, 6H), 2.32 (s, 3H), 1.42 (d, J = 6.8 Hz, 3H), 1.05 (d, J = 7.0 Hz, 3H). | |
| 3-810 | ¹H NMR (400 MHz, DMSO-d₆) δ 8.14 (s, 1H), 7.97 (s, 1H), 4.15-3.87 (m, | |
| | 1H), 3.57-3.46 (m, 6H), 2.65 (s, 2H), 1.41 (d, J = 6.9 Hz, 3H), 1.11-0.95 (m, 6H). | |
| 3-812 | ¹H NMR (400 MHz, CDCl₃-d) δ 8.22 (d, J = 8.1 Hz, 1H), 7.66 (d, J = 8.1 Hz, | |
| | 1H), 3.59 (s, 3H), 3.46-3.31 (m, 4H), 2.40 (s, 3H), 1.44-1.36 (m, 1H), 1.25-1.17 | |
| | (m, 2H), 1.05-0.92 (m, 1H). | |
| 3-813 | ¹H NMR (400 MHz, CDCl₃-d) δ 8.22 (d, J = 8.1 Hz, 1H), 7.66 (d, J = 8.1 Hz, | |
| | 1H), 3.61 (s, 3H), 3.45-3.34 (m, 4H), 2.78-2.61 (m, 2H), 1.46-1.35 (m, 1H), 1.29- | |
| | 1.15 (m, 5H), 1.06-0.93 (m, 1H). | |
| 3-815 | ¹H NMR (400 MHz, CDCl₃-d) δ 8.15 (d, J = 8.1 Hz, 1H), 7.64 (d, J = 8.1 Hz, 1H), | |
| | 3.83-3.73 (m, 1H), 3.58 (s, 3H), 3.41 (s, 3H), 3.15-3.03 (m, 1H), 2.42 (s, 3H), 2.02- | |
| | 1.79 (m, 2H), 1.60-1.44 (m, 2H), 0.98 (t, J = 7.3 Hz, 3H). | |
| 3-820 | ¹H NMR (400 MHz, CDCl₃-d) δ 8.24 (d, J = 8.0 Hz, 1H), 7.65 (d, J = 8.5 Hz, | |
| | 1H), 4.21-3.75 (m, 1H), 3.59 (s, 3H), 3.40 (d, J = 7.2 Hz, 3H), 2.40 (s, 3H), 2.24- | |
| | 2.11 (m, 1H), 1.90-1.78 (m, 1H), 1.49 (d, J = 6.9 Hz, 2H), 1.11 (q, J = 7.5 Hz, 3H), | |
| | 0.98 (t, J = 7.4 Hz, 1H). | |
| 3-829 | ¹H NMR (400 MHz, CDCl₃-d) δ 8.15 (d, J = 8.1 Hz, 1H), 7.64 (d, J = 8.1 Hz, | |
| | 1H), 3.85-3.78 (m, 1H), 3.58 (s, 3H), 3.42 (s, 3H), 2.88-2.76 (m, 1H), 2.44-2.32 | |
| | (m, 3H), 1.36-1.23 (m, 1H), 1.20 (d, J = 6.6 Hz, 3H), 1.14 (d, J = 6.8 Hz, 3H). | |
| 3-832 | ¹H NMR (400 MHz, CDCl₃-d) δ 8.21 (d, J = 7.6 Hz, 1H), 7.71 (d, J = 8.1 Hz, | |
| | 1H), 4.81-4.47 (m, 1H), 3.81-3.71 (m, 1H), 3.59 (s, 3H), 3.44 (s, 3H), 2.46 (s, 3H). | |
| 3-835 | ¹H NMR (400 MHz, CDCl₃-d) δ 8.19 (d, J = 8.1 Hz, 1H), 7.67 (d, J = 8.1 Hz, | |
| | 1H), 4.28-4.13 (m, 1H), 4.13-3.90 (m, 2H), 3.59 (s, 3H), 3.46-3.42 (m, 3H), 2.44 | |
| | (s, 3H). | |
| 3-852 | ¹H NMR (400 MHz, DMSO-d₆) δ 12.43 (s, 1H), 8.11 (d, J = 8.1 Hz, 1H), 7.87 | |
| | (d, J = 8.1 Hz, 1H), 3.56 (s, 3H), 3.10 (d, J = 7.6 Hz, 2H), 2.59-2.52 (m, 1H), 2.49 | |
| | (s, 3H), 2.13-1.99 (m, 2H), 1.87-1.68 (m, 4H). | |
| 3-853 | ¹H NMR (400 MHz, DMSO-d₆) δ 12.54 (s, 1H), 8.11 (d, J = 8.1 Hz, 1H), 8.02 | |
| | (d, J = 8.1 Hz, 1H), 3.87 (dd, J = 12.7, 5.9 Hz, 1H), 3.52 (s, 3H), 3.19-3.15 (m, 1H), | |
| | 2.91-2.81 (m, 1H), 2.49 (s, 3H), 2.26-2.08 (m, 2H), 1.98-1.85 (m, 4H). | |
| 3-856 | ¹H NMR (400 MHz, DMSO-d₆) δ 12.56 (s, 1H), 8.36 (d, J = 8.2 Hz, 1H), 8.19 | |
| | (d, J = 8.2 Hz, 1H), 3.83 (d, J = 7.3 Hz, 2H), 3.64 (s, 3H), 3.03-2.92 (m, 1H), 2.49 | |
| | (s, 3H), 2.17-2.06 (m, 2H), 2.03-1.84 (m, 4H). | |
| 3-857 | ¹H NMR (400 MHz, CDCl₃-d) δ 11.78 (s, 1H), 8.16 (d, J = 8.2 Hz, 1H), 7.70 (d, J = | |
| | 8.1 Hz, 1H), 3.49 (s, 3H), 3.00 (d, J = 7.4 Hz, 2H), 2.52 (s, 3H), 2.27-2.13 (m, 1H), | |
| | 1.93-1.81 (m, 2H), 1.74-1.51 (m, 4H), 1.42-1.18 (m, 2H). | |
| 3-858 | ¹H NMR (400 MHz, CDCl₃-d) δ 11.90 (s, 1H), 8.16 (d, J = 7.9 Hz, 1H), 7.82 (d, | |
| | J = 8.0 Hz, 1H), 4.08-3.99 (m, 1H), 3.45 (s, 3H), 3.06-2.96 (m, 1H), 2.64-2.44 | |
| | (m, 4H), 2.07-1.89 (m, 2H), 1.77-1.56 (m, 4H), 1.56-1.45 (m, 1H), 1.43-1.31 | |
| | (m, 1H). | |
| 3-861 | ¹H NMR (400 MHz, CDCl₃-d) δ 8.28 (d, J = 8.1 Hz, 1H), 7.83 (d, J = 8.1 Hz, | |
| | 1H), 3.61 (d, J = 7.0 Hz, 2H), 3.54 (s, 3H), 2.66-2.51 (m, 1H), 2.46 (s, 3H), 2.04- | |
| | 1.92 (m, 2H), 1.66-1.49 (m, 4H), 1.41-1.31 (m, 2H). | |
| 3-862 | ¹H NMR (400 MHz, DMSO-d₆) δ 12.44 (s, 1H), 8.15 (d, J = 8.1 Hz, 1H), 7.92 | |
| | (d, J = 8.3 Hz, 1H), 3.91-3.82 (m, 1H), 3.56 (s, 3H), 3.12-2.99 (m, 2H), 2.90- | |
| | 2.74 (m, 2H), 2.49 (s, 3H), . | |
| 3-863 | ¹H NMR (400 MHz, DMSO-d₆) δ 9.17 (d, J = 7.8 Hz, 1H), 9.07 (d, J = 8.3 Hz, 1H), | |
| | 5.36-5.28 (m, 1H), 4.59 (s, 3H), 4.08-3.92 (m, 4H), 3.53 (s, 3H). | |
| 3-866 | ¹H NMR (400 MHz, DMSO-d₆) δ 12.61 (s, 1H), 8.42 (d, J = 8.2 Hz, 1H), 8.22 | |
| | (d, J = 8.2 Hz, 1H), 4.73-4.65 (m, 1H), 3.66 (s, 3H), 3.30-3.24 (m, 2H), 3.15- | |
| | 3.06 (m, 2H), 2.49 (s, 3H). | |
| 4-1 | ¹H NMR (400 MHz, CDCl3-d) δ 9.02 (s, 1H), 7.91 (d, J = 8.1 Hz, 1H), 7.61 (d, | |
| | J = 8.2 Hz, 1H), 3.37 (s, 3H), 2.89 (t, J = 7.5 Hz, 2H), 2.45 (s, 3H), 1.64 (h, J = 7.4 | |
| | Hz, 2H), 0.98 (t, J = 7.4 Hz, 3H). | |
| 4-2 | ¹H NMR (400 MHz, CDCl3-d) δ 9.61 (s, 1H), 8.16 (d, J = 8.1 Hz, 1H), 7.85 (d, | |
| | J = 8.1 Hz, 1H), 3.88-3.78 (m, 1H), 3.41 (s, 3H), 3.15-3.03 (m, 1H), 2.51 (s, 3H), | |
| | 2.07-1.85 (m, 2H), 1.15 (t, J = 7.4 Hz, 3H). | |
| 4-3 | ¹H NMR (400 MHz, CDCl₃-d) δ 8.11 (d, J = 8.1 Hz, 1H), 7.80 (d, J = 8.1 Hz, | [α] |
| | 1H), 3.88-3.75 (m, 1H), 3.37 (s, 3H), 3.14-3.03 (m, 1H), 2.44 (s, 3H), 2.07-1.81 | D²⁰ = +100.18°(c |
| | (m, 2H), 1.12 (t, J = 7.4 Hz, 3H). | 0.108, DMF) |
| 4-4 | ¹H NMR (400 MHz, CDCl₃-d) δ 8.19 (d, J = 8.1 Hz, 1H), 7.83 (d, J = 8.1 Hz, | [α] |
| | 1H), 3.98-3.83 (m, 1H), 3.42 (s, 3H), 3.20-3.03 (m, 1H), 2.50 (s, 3H), 2.09-1.90 | D²⁰ = −111.11°(c |
| | (m, 2H), 1.18 (t, J = 7.4 Hz, 3H). | 0.121, DMF) |
| 4-5 | ¹H NMR (400 MHz, DMSO-d₆) δ 8.39 (d, J = 8.2 Hz, 1H), 8.20 (d, J = 8.2 Hz, | |
| | 1H), 3.72-3.64 (m, 5H), 2.40 (s, 3H), 1.92 (q, J = 7.5 Hz, 2H), 1.08 (t, J = 7.4 Hz, 3H). | |
| 4-6 | ¹H NMR (400 MHz, CDCl₃) δ 8.68 (s, 1H), 8.11 (d, J = 8.1 Hz, 1H), 7.72 (d, J = | |
| | 8.1 Hz, 1H), 3.85 (dq, J = 13.4, 6.7 Hz, 1H), 3.44 (s, 3H), 2.53 (s, 3H), 1.31 (s, 3H), | |
| | 1.29 (s, 3H). | |
| 4-7 | ¹H NMR (400 MHz, CDCl₃) δ 9.19 (s, 1H), 8.26 (d, J = 8.1 Hz, 1H), 7.88 (d, J = | |
| | 8.1 Hz, 1H), 4.30-4.14 (m, 1H), 3.38 (s, 3H), 2.52 (s, 3H), 1.56 (d, J = 6.8 Hz, 3H), | |
| | 1.20 (d, J = 7.0 Hz, 3H). | |
| 4-10 | ¹H NMR (400 MHz, CDCl₃) δ 9.41 (s, 1H), 8.39 (d, J = 8.2 Hz, 1H), 7.93 (d, J = | |
| | 8.1 Hz, 1H), 4.27-4.15 (m, 1H), 3.55 (s, 3H), 2.49 (s, 3H), 1.44 (s, 3H), 1.42 (s, 3H). | |
| 4-16 | ¹H NMR (400 MHz, DMSO-d₆) δ 11.67 (s, 1H), 8.17 (d, J = 8.1 Hz, 1H), 7.94 | |
| | (d, J = 8.0 Hz, 1H), 3.65 (dq, J = 12.8, 6.5 Hz, 1H), 3.56 (s, 3H), 2.41 (s, 3H), 1.69- | |
| | 1.55 (m, 2H), 1.18 (d, J = 6.6 Hz, 3H), 0.98 (t, J = 7.3 Hz, 3H). | |
| 4-19 | ¹H NMR (400 MHz, DMSO-d₆) δ 11.72 (s, 1H), 8.21 (s, 1H), 8.13 (s, 1H), 4.02 (s, | |
| | 0.5H), 3.89 (s, 0.5H), 3.53 (s, 3H), 2.41 (s, 3H), 2.06 (s, 0.5H), 1.91 (s, 0.5H), 1.76 (s, | |
| | 0.5H), 1.50 (s, 0.5H), 1.42 (s, 1.5H), 1.07 (s, 3H), 0.93 (s, 1.5H). | |
| 4-24 | ¹H NMR (400 MHz, DMSO-d₆) δ 11.69 (s, 1H), 8.47 (d, J = 7.3 Hz, 1H), 8.27 | |
| | (d, J = 7.5 Hz, 1H), 4.10-3.93 (m, 1H), 3.64 (s, 3H), 2.41 (s, 3H), 2.05-1.88 (m, | |
| | 1H), 1.75-1.57 (m, 1H), 1.36 (d, J = 5.7 Hz, 3H), 1.01 (t, J = 6.3 Hz, 3H). | |
| 4-32 | ¹H NMR (400 MHz, CDCl₃-d)) δ 8.72 (s, 1H), 8.08 (d, J = 8.1 Hz, 1H), 7.72 (d, | |
| | J = 8.0 Hz, 1H), 3.49 (s, 3H), 2.95 (d, J = 7.5 Hz, 2H), 2.53 (s, 3H), 1.09-1.0.5 (m, | |
| | 1H), 0.65-0.54 (m, 2H), 0.33-0.21 (m, 2H). | |
| 4-33 | ¹H NMR (400 MHz, CDCl₃-d)) δ 10.24 (s, 1H), 8.07 (d, J = 8.1 Hz, 1H), 7.83 | |
| | (d, J = 8.1 Hz, 1H), 3.96 (dd, J = 13.2, 6.0 Hz, 1H), 3.40 (s, 3H), 2.82 (dd, J = 13.2, | |
| | 9.1 Hz, 1H), 2.45 (s, 3H), 2.01 (d, J = 17.2 Hz, 2H), 1.24-1.20 (m, 1H), 0.82-0.68 | |
| | (m, 2H). | |
| 4-36 | ¹H NMR (400 MHz, CDCl₃-d) δ 9.04 (s, 1H), 8.32 (d, J = 8.1 Hz, 1H), 7.93 (d, | |
| | J = 8.2 Hz, 1H), 3.60 (s, 2H), 3.58 (s, 3H), 2.50 (s, 3H), 1.36-1.19 (m, 1H), 0.80-0.66 | |
| | (m, 2H), 0.52-0.37 (m, 2H). | |
| 4-37 | ¹H NMR (400 MHz, DMSO-d₆) δ 11.68 (s, 1H), 8.14 (d, J = 8.1 Hz, 1H), 7.95 | |
| | (d, J = 7.9 Hz, 1H), 3.49 (s, 3H), 2.74-2.61 (m, 1H), 2.41 (s, 3H), 0.96-0.85 (m, | |
| | 2H), 0.80 (s, 2H). | |
| 4-38 | ¹H NMR (400 MHz, DMSO-d₆) δ 11.72 (s, 1H), 8.19 (d, J = 8.1 Hz, 1H), 8.10 | |
| | (d, J = 8.1 Hz, 1H), 3.53 (s, 3H), 3.37 (dd, J = 8.4, 4.3 Hz, 1H), 2.41 (s, 3H), 1.37- | |
| | 1.17 (m, 2H), 1.14-0.98 (m, 2H). | |
| 4-41 | ¹H NMR (400 MHz, DMSO-d₆) δ 11.71 (s, 1H), 8.44 (d, J = 8.2 Hz, 1H), 8.26 | |
| | (d, J = 8.2 Hz, 1H), 3.66 (s, 3H), 3.52-3.43 (m, 1H), 2.42 (s, 3H), 1.58-1.44 (m, | |
| | 2H), 1.23 (dd, J = 7.7, 2.3 Hz, 2H). | |
| 4-42 | ¹H NMR (400 MHz, DMSO-d₆) δ 11.64 (s, 1H), 8.14 (d, J = 8.1 Hz, 1H), 7.92 | |
| | (d, J = 8.1 Hz, 1H), 4.02-3.91 (m, 1H), 3.56 (s, 3H), 2.41 (s, 3H), 2.39-2.29 (m, | |
| | 2H), 2.24-2.11 (m, 2H), 1.95-1.79 (m, 2H). | |
| 4-43 | ¹H NMR (400 MHz, CDCl₃ -d) δ 8.12 (d, J = 8.0 Hz, 1H), 7.82 (d, J = 8.0 Hz, | |
| | 1H), 4.47 (p, J = 8.5 Hz, 1H), 3.38 (s, 3H), 2.90-2.70 (m, 1H), 2.47 (s, 3H), 2.44- | |
| | 2.34 (m, 1H), 2.32-2.13 (m, 2H), 2.11-2.03 (m, 2H). | |
| 4-44 | ¹H NMR (400 MHz, CDCl₃ -d) δ 8.12 (d, J = 8.0 Hz, 1H), 7.82 (d, J = 8.0 Hz, | [α] |
| | 1H), 4.47 (p, J = 8.5 Hz, 1H), 3.38 (s, 3H), 2.90-2.70 (m, 1H), 2.47 (s, 3H), 2.44- | D²⁰ = +94.99°(c |
| | 2.34 (m, 1H), 2.32-2.13 (m, 2H), 2.11-2.03 (m, 2H). | 0.099, DMF) |
| 4-45 | ¹H NMR (400 MHz, CDCl₃ -d) δ 8.12 (d, J = 8.0 Hz, 1H), 7.82 (d, J = 8.0 Hz, | [α] |
| | 1H), 4.47 (p, J = 8.5 Hz, 1H), 3.38 (s, 3H), 2.90-2.70 (m, 1H), 2.47 (s, 3H), 2.44- | D²⁰ = −92.68°(c |
| | 2.34 (m, 1H), 2.32-2.13 (m, 2H), 2.11-2.03 (m, 2H). | 0.108, DMF) |
| 4-46 | ¹H NMR (400 MHz, DMSO-d₆) δ 11.69 (s, 1H), 8.42 (d, J = 8.2 Hz, 1H), 8.25 | |
| | (d, J = 8.2 Hz, 1H), 4.78-4.70 (m, 1H), 3.66 (s, 3H), 2.72-2.61 (m, 2H), 2.40 (s, | |
| | 3H), 2.30-2.19 (m, 2H), 2.10-1.91 (m, 2H). | |
| 4-47 | ¹H NMR (400 MHz, CDCl3-d) δ 8.59 (s, 1H), 8.16 (d, J = 8.1 Hz, 1H), 7.73 (d, | |
| | J = 8.1 Hz, 1H), 3.95-3.86 (m, 1H), 3.44 (s, 3H), 2.53 (s, 3H), 2.06-1.90 (m, 2H), | |
| | 1.89-1.73 (m, 2H), 1.72-1.63 (m, 2H), 1.28-1.23 (m, 2H). | |
| 4-48 | ¹H NMR (400 MHz, CDCl3-d) δ 9.28 (s, 1H), 8.24 (d, J = 8.1 Hz, 1H), 7.86 (d, J = | |
| | 8.1 Hz, 1H), 4.50-4.42 (m, 1H), 3.38 (s, 3H), 2.52 (s, 3H), 2.36-1.96 (m, 3H), 1.90- | |
| | 1.68 (m, 5H). | |
| 4-49 | ¹H NMR (400 MHz, CDCl3-d) δ 9.98 (s, 1H), 8.17 (d, J = 8.1 Hz, 1H), 7.85 (d, | [α] |
| | J = 8.1 Hz, 1H), 4.51-4.43 (m, 1H), 3.35 (s, 3H), 2.49 (s, 3H), 2.30-1.95 (m, 2H), | D²⁰ = +42.99°(c |
| | 1.87-1.49 (m, 6H). | 0.100, DMF) |
| 4-50 | ¹H NMR (400 MHz, CDCl3-d) δ 10.14 (s, 1H), 8.18 (d, J = 8.1 Hz, 1H), 7.85 (d, | [α] |
| | J = 8.0 Hz, 1H), 4.51-4.43 (m, 1H), 3.36 (s, 3H), 2.49 (s, 3H), 2.29-2.08 (m, 2H), | D²⁰ = −44.09°(c |
| | 1.88-1.55 (m, 6H). | 0.104, DMF) |
| 4-51 | ¹H NMR (400 MHz, CDCl₃-d) δ 9.46 (s, 1H), 8.28 (d, J = 8.2 Hz, 1H), 7.84 (d, J = | |
| | 8.2 Hz, 1H), 4.33 (p, J = 7.9 Hz, 1H), 3.47 (s, 3H), 2.40 (s, 3H), 2.21-2.08 (m, 2H), | |
| | 1.95-1.84 (m, 2H), 1.83-1.69 (m, 2H), 1.69-1.56 (m, 2H). | |
| 4-58 | ¹H NMR (400 MHz, DMSO-d₆) δ 11.71 (s, 1H), 8.28-8.11 (m, 2H), 4.82- | |
| | 4.67 (m, 1H), 4.50-4.32 (m, 1H), 3.56 (s, 3H), 2.41 (s, 3H). | |
| 4-61 | ¹H NMR (400 MHz, DMSO-d₆) δ 11.73 (s, 1H), 8.43 (d, J = 8.2 Hz, 1H), 8.34 | |
| | (d, J = 8.2 Hz, 1H), 5.23-5.03 (m, 2H), 3.66 (s, 3H), 2.41 (s, 3H). | |
| 4-63 | ¹H NMR (400 MHz, DMSO-d₆) δ 11.68 (s, 1H), 8.23-8.09 (m, 2H), 6.75-6.44 (m, | |
| | 1H), 4.45-4.28 (m, 1H), 3.95-3.76 (m, 1H), 3.55 (s, 3H), 2.41 (s, 3H). | |
| 4-66 | ¹H NMR (400 MHz, DMSO-d₆) δ 11.72 (s, 1H), 8.41 (d, J = 8.2 Hz, 1H), 8.31 (d, J = | |
| | 8.2 Hz, 1H), 6.82-6.51 (m, 1H), 4.73-4.52 (m, 2H), 3.66 (s, 3H), 2.41 (s, 3H). | |
| 4-76 | ¹H NMR (400 MHz, DMSO-d₆) δ 11.71 (s, 1H), 8.40 (d, J = 8.2 Hz, 1H), 8.27 | |
| | (d, J = 8.2 Hz, 1H), 6.02-5.87 (m, 1H), 5.55-5.45 (m, 2H), 4.53 (d, J = 7.3 Hz, | |
| | 2H), 3.65 (s, 3H), 2.41 (s, 3H). | |
| 4-103 | ¹H NMR (400 MHz, DMSO-d₆) δ 11.75 (s, 1H), 8.18 (s, 2H), 4.98 (d, J = 16.1 | |
| | Hz, 1H), 4.72 (d, J = 16.1 Hz, 1H), 3.56 (s, 3H), 2.41 (s, 3H). | |
| 4-113 | ¹H NMR (400 MHz, DMSO-d₆) δ 11.67 (s, 1H), 8.17 (d, J = 8.1 Hz, 1H), 8.12 | |
| | (d, J = 8.1 Hz, 1H), 4.24-4.03 (m, 3H), 3.56 (s, 3H), 3.55-3.48 (m, 1H), 2.41 (s, 3H). | |
| 4-116 | ¹H NMR (400 MHz, DMSO-d₆) δ 11.71 (s, 1H), 8.41 (d, J = 8.2 Hz, 1H), 8.28 | |
| | (d, J = 8.1 Hz, 1H), 4.27-4.22 (m, 2H), 4.17-4.12 (m, 2H), 3.67 (s, 3H), 2.42 (s, 3H). | |
| 4-117 | ¹H NMR (400 MHz, DMSO-d₆) δ 11.58 (s, 1H), 8.15 (d, J = 8.1 Hz, 1H), 7.93 (d, J = | |
| | 8.1 Hz, 1H), 3.58 (s, 3H), 3.00 (t, J = 7.4 Hz, 2H), 2.82 (t, J = 7.5 Hz, 2H), 1.65 (h, J = | |
| | 7.3 Hz, 2H), 1.28 (t, J = 7.5 Hz, 3H), 0.99 (t, J = 7.3 Hz, 3H). | |
| 4-118 | ¹H NMR (400 MHz, DMSO-d₆) δ 11.63 (s, 1H), 8.15 (d, J = 8.1 Hz, 1H), 8.08 | |
| | (d, J = 8.1 Hz, 1H), 3.80-3.70 (m, 1H), 3.54 (s, 3H), 3.12-3.04 (m, 1H), 2.82 (q, J = | |
| | 7.5 Hz, 2H), 1.92-1.76 (m, 2H), 1.28 (t, J = 7.5 Hz, 3H), 1.09 (t, J = 7.4 Hz, 3H). | |
| 4-121 | ¹H NMR (400 MHz, DMSO-d₆) δ 11.65 (s, 1H), 8.39 (d, J = 8.2 Hz, 1H), 8.23 | |
| | (d, J = 8.2 Hz, 1H), 3.73-3.67 (m, 2H), 3.66 (s, 3H), 2.81 (q, J = 7.5 Hz, 2H), 1.94- | |
| | 1.86 (m, 2H), 1.28 (t, J = 7.5 Hz, 3H), 1.06 (t, J = 7.4 Hz, 3H). | |
| 4-122 | ¹H NMR (400 MHz, CDCl₃-d) δ 8.69 (s, 1H), 8.12 (d, J = 8.1 Hz, 1H), 7.72 (d, J = | |
| | 8.1 Hz, 1H), 3.90-3.79 (m, 1H), 3.44 (s, 3H), 2.98-2.88 (m, 2H), 1.41 (t, J = 7.5 | |
| | Hz, 3H), 1.30 (d, J = 6.7 Hz, 6H). | |
| 4-123 | ¹H NMR (400 MHz, CDCl₃-d) δ 9.70 (s, 1H), 8.23 (d, J = 8.1 Hz, 1H), 7.86 (d, J = | |
| | 8.1 Hz, 1H), 4.32-4.09 (m, 1H), 3.37 (s, 3H), 2.90 (q, J = 7.5 Hz, 2H), 1.55 (d, J = | |
| | 6.8 Hz, 3H), 1.39 (t, J = 7.5 Hz, 3H), 1.19 (d, J = 7.0 Hz, 3H). | |
| 4-126 | ¹H NMR (400 MHz, CDCl₃-d) δ 9.30 (s, 1H), 8.35 (d, J = 8.2 Hz, 1H), 7.92 (d, J = | |
| | 8.2 Hz, 1H), 4.25-4.14 (m, 1H), 3.54 (s, 3H), 2.92-2.82 (m, 2H), 1.45-1.34 (m, 9H). | |
| 4-127 | ¹H NMR (400 MHz, DMSO-d₆) δ 8.18 (d, J = 7.9 Hz, 1H), 7.92 (d, J = 7.5 Hz, | |
| | 1H), 3.90-3.76 (m, 1H), 3.57 (s, 3H), 3.29-3.18 (m, 2H), 1.29 (dd, J = 23.2, 6.4 | |
| | Hz, 12H). | |
| 4-128 | ¹H NMR (400 MHz, DMSO-d₆) δ 11.59 (s, 1H), 8.23 (d, J = 7.8 Hz, 1H), 8.11 (d, J = | |
| | 7.9 Hz, 1H), 4.19-4.06 (m, 1H), 3.53 (s, 3H), 3.30-3.19 (m, 1H), 1.47 (d, J = 6.5 | |
| | Hz, 3H), 1.32 (d, J = 6.7 Hz, 6H), 1.14 (d, J = 6.5 Hz, 3H). | |
| 4-131 | ¹H NMR (400 MHz, DMSO-d₆) δ 11.57 (s, 1H), 8.49 (d, J = 8.2 Hz, 1H), 8.26 | |
| | (d, J = 8.2 Hz, 1H), 4.30-4.12 (m, 1H), 3.64 (s, 3H), 3.30-3.17 (m, 1H), 1.38 (d, J = | |
| | 6.8 Hz, 6H), 1.32 (d, J = 6.9 Hz, 6H). | |
| 4-132 | ¹H NMR (400 MHz, DMSO-d₆) δ 11.53 (s, 1H), 8.18 (d, J = 8.1 Hz, 1H), 7.92 | |
| | (d, J = 8.1 Hz, 1H), 3.71-3.61 (m, 1H), 3.56 (s, 3H), 3.30-3.21 (m, 1H), 1.70- | |
| | 1.59 (m, 2H), 1.31 (d, J = 6.9 Hz, 6H), 1.18 (d, J = 6.7 Hz, 3H), 0.98 (t, J = 7.3 Hz, 3H). | |
| 4-135 | ¹H NMR (400 MHz, DMSO-d₆) δ 11.58 (s, 1H), 8.22 (d, J = 8.1 Hz, 1H), 8.15- | |
| | 8.05 (m, 1H), 4.09-3.84 (m, 1H), 3.52 (d, J = 3.9 Hz, 3H), 3.30-3.19 (m, 1H), 2.15- | |
| | 2.00 (m, 1H), 1.82-1.71 (m, 1H), 1.56-1.25 (m, 9H), 1.09-1.04 (m, 3H). | |
| 4-140 | ¹H NMR (400 MHz, DMSO-d₆) δ 11.53 (s, 1H), 8.47 (d, J = 8.2 Hz, 1H), 8.25 | |
| | (d, J = 8.2 Hz, 1H), 4.08-3.98 (m, 1H), 3.63 (s, 3H), 3.28-3.19 (m, 1H), 2.03- | |
| | 1.91 (m, 1H), 1.74-1.61 (m, 1H), 1.36 (d, J = 6.9 Hz, 3H), 1.31 (d, J = 6.9 Hz, 6H), | |
| | 1.01 (t, J = 7.4 Hz, 3H). | |
| 4-143 | ¹H NMR (400 MHz, DMSO-d₆) δ 11.63 (s, 1H), 8.18 (d, J = 8.1 Hz, 1H), 7.93 | |
| | (d, J = 8.1 Hz, 1H), 3.87-3.73 (m, 1H), 3.56 (s, 3H), 2.80 (t, J = 7.5 Hz, 2H), 1.79- | |
| | 1.67 (m, 2H), 1.26 (d, J = 6.7 Hz, 6H), 0.96 (t, J = 7.4 Hz, 3H). | |
| 4-144 | ¹H NMR (400 MHz, DMSO-d₆) δ 11.61 (s, 1H), 8.48 (d, J = 8.2 Hz, 1H), 8.26 | |
| | (d, J = 8.2 Hz, 1H), 4.26-4.14 (m, 1H), 3.64 (s, 3H), 2.79 (t, J = 7.5 Hz, 2H), 1.77- | |
| | 1.65 (m, 2H), 1.37 (d, J = 6.9 Hz, 6H), 0.96 (t, J = 7.4 Hz, 3H). | |
| 4-147 | ¹H NMR (400 MHz, DMSO-d₆) δ 11.63 (s, 1H), 8.23 (d, J = 8.1 Hz, 1H), 8.11 | |
| | (d, J = 8.1 Hz, 1H), 4.18-4.04 (m, 1H), 3.53 (s, 3H), 2.80 (t, J = 7.5 Hz, 2H), 1.79- | |
| | 1.65 (m, 2H), 1.46 (d, J = 6.8 Hz, 3H), 1.14 (d, J = 7.0 Hz, 3H), 0.96 (t, J = 7.4 Hz, 3H). | |
| 4-148 | ¹H NMR (400 MHz, CDCl₃-d) δ 8.56 (s, 1H), 8.07 (d, J = 8.1 Hz, 1H), 7.65 (d, J = | |
| | 8.1 Hz, 1H), 3.66-3.58 (m, 1H), 3.38 (s, 3H), 2.85-2.77 (m, 2H), 1.84-1.70 (m, | |
| | 2H), 1.65-1.55 (m, 1H), 1.30-1.08 (m, 4H), 1.02-0.92 (m, 6H). | |
| 4-151 | ¹H NMR (400 MHz, DMSO-d₆) δ 11.64 (s, 1H), 8.22 (d, J = 8.1 Hz, 1H), 8.11 | |
| | (dd, J = 8.1, 4.1 Hz, 1H), 4.09-3.81 (m, 1H), 3.55-3.49 (m, 3H), 2.83-2.75 (m, | |
| | 2H), 2.13-1.94 (m, 1H), 1.84-1.65 (m, 3H), 1.58-1.37 (m, 2H), 1.15-0.86 (m, 7H). | |
| 4-156 | ¹H NMR (400 MHz, CDCl₃-d) δ 9.06 (s, 1H), 8.40 (d, J = 7.9 Hz, 1H), 7.92 (d, J = | |
| | 8.1 Hz, 1H), 4.03-3.98 (m, 1H), 3.55 (s, 3H), 2.87-2.80 (m, 2H), 1.91-1.61 (m, | |
| | 4H), 1.41 (d, J = 6.9 Hz, 3H), 1.10-0.99 (m, 6H). | |
| 4-159 | ¹H NMR (400 MHz, CDCl₃-d) δ 7.95 (d, J = 8.1 Hz, 1H), 7.71 (d, J = 8.1 Hz, | |
| | 1H), 3.47 (s, 3H), 2.95 (t, J = 7.5 Hz, 2H), 1.78-1.60 (m, 2H), 1.05 (t, J = 7.3 Hz, 3H). | |
| 4-160 | ¹H NMR (400 MHz, CDCl₃-d) δ 10.98 (s, 1H), 8.15 (d, J = 8.1 Hz, 1H), 7.89 (d, | |
| | J = 8.1 Hz, 1H), 3.88-3.77 (m, 1H), 3.38 (s, 3H), 3.17-3.05 (m, 1H), 2.09-1.83 | |
| | (m, 2H), 1.14 (t, J = 7.4 Hz, 3H). | |
| 4-163 | ¹H NMR (400 MHz, CDCl₃-d) δ 9.79 (s, 1H), 8.28 (d, J = 8.2 Hz, 1H), 7.95 (d, | |
| | J = 8.2 Hz, 1H), 3.66-3.54 (m, 5H), 2.08-1.94 (m, 2H), 1.14 (t, J = 7.4 Hz, 3H). | |
| 4-164 | ¹H NMR (400 MHz, DMSO-d₆) δ 12.82 (s, 1H), 8.21 (d, J = 8.2 Hz, 1H), 7.93 (d, J = | |
| | 8.1 Hz, 1H), 3.86-3.77 (m, 1H), 3.58 (s, 3H), 1.27 (d, J = 6.7 Hz, 6H). | |
| 4-165 | ¹H NMR (400 MHz, DMSO-d₆) δ 12.86 (s, 1H), 8.26 (d, J = 8.1 Hz, 1H), 8.09 | |
| | (d, J = 8.1 Hz, 1H), 4.17-4.07 (m, 1H), 3.54 (s, 3H), 1.47 (d, J = 6.8 Hz, 3H), 1.13 (d, | |
| | J = 7.0 Hz, 3H). | |
| 4-168 | ¹H NMR (400 MHz, CDCl₃ -d) δ 9.74 (s, 1H), 8.38 (d, J = 8.2 Hz, 1H), 7.97 (d, J = | |
| | 8.2 Hz, 1H), 4.27-4.14 (m, 1H), 3.57 (s, 3H), 1.44 (d, J = 6.9 Hz, 6H). | |
| 4-215 | ¹H NMR (400 MHz, DMSO-d₆) δ 11.63 (s, 1H), 8.15 (d, J = 8.1 Hz, 1H), 7.95 (d, J = | |
| | 8.1 Hz, 1H), 3.58 (s, 3H), 3.00 (t, J = 7.4 Hz, 2H), 2.11-2.04 (m, 1H), 1.71-1.59 | |
| | (m, 2H), 1.17-1.11 (m, 2H), 1.03-0.93 (m, 5H). | |
| 4-216 | ¹H NMR (400 MHz, DMSO-d₆) δ 11.69 (s, 1H), 8.15 (d, J = 8.1 Hz, 1H), 8.09 | |
| | (d, J = 8.1 Hz, 1H), 3.81-3.69 (m, 1H), 3.55 (s, 3H), 3.14-3.02 (m, 1H), 2.13- | |
| | 2.03 (m, 1H), 1.94-1.75 (m, 2H), 1.18-1.11 (m, 2H), 1.09 (t, J = 7.4 Hz, 3H), 1.00- | |
| | 0.94 (m, 2H). | |
| 4-219 | ¹H NMR (400 MHz, DMSO-d₆) δ 11.69 (s, 1H), 8.40 (d, J = 8.2 Hz, 1H), 8.27 | |
| | (d, J = 8.2 Hz, 1H), 3.73-3.67 (m, 2H), 3.66 (s, 3H), 2.10-2.02 (m, 1H), 1.97- | |
| | 1.84 (m, 2H), 1.18-1.11 (m, 2H), 1.06 (t, J = 7.4 Hz, 3H), 1.01-0.94 (m, 2H). | |
| 4-220 | ¹H NMR (400 MHz, DMSO-d₆) δ 11.66 (s, 1H), 8.18 (d, J = 8.1 Hz, 1H), 7.96 | |
| | (d, J = 8.1 Hz, 1H), 3.88-3.72 (m, 1H), 3.57 (s, 3H), 2.14-2.03 (m, 1H), 1.27 (d, J = | |
| | 6.6 Hz, 6H), 1.17-1.11 (m, 2H), 1.01-0.94 (m, 2H). | |
| 4-221 | ¹H NMR (400 MHz, DMSO-d₆) δ 8.22 (d, J = 8.1 Hz, 1H), 8.11 (d, J = 8.1 Hz, | |
| | 1H), 4.12 (dt, J = 13.4, 6.5 Hz, 1H), 3.52 (s, 3H), 2.12-2.02 (m, 1H), 1.47 (d, J = 6.8 | |
| | Hz, 3H), 1.18-1.07 (m, 5H), 1.00-0.93 (m, 2H). | |
| 4-224 | ¹H NMR (400 MHz, DMSO-d₆) δ 11.69 (s, 1H), 8.48 (d, J = 8.2 Hz, 1H), 8.28 (d, J = | |
| | 8.2 Hz, 1H), 4.20 (dt, J = 13.4, 6.6 Hz, 1H), 3.64 (s, 3H), 2.14-2.01 (m, 1H), 1.37 (d, | |
| | J = 6.8 Hz, 6H), 1.17-1.11 (m, 2H), 1.01-0.94 (m, 2H). | |
| 4-271 | ¹H NMR (400 MHz, DMSO-d₆, rotameric) δ 8.18 (d, J = 8.1 Hz, 0.5H), 8.03- | |
| | 7.95 (m, 1H), 7.74 (d, J = 8.2 Hz, 0.5H), 3.57 (s, 1.5H), 3.51 (s, 1.5H), 3.44 (s, 1.5H), | |
| | 3.25 (s, 1.5H), 3.02 (t, J = 7.4 Hz, 1H), 2.91-2.77 (m, 1H), 2.46 (s, 1.5H), 2.41 (s, | |
| | 1.5H), 1.70-1.58 (m, 1H), 1.51-1.33 (m, 1H), 0.98 (t, J = 7.3 Hz, 1.5H), 0.91 (t, J = | |
| | 7.3 Hz, 1.5H). | |
| 4-272 | ¹H NMR (400 MHz, CDCl₃-d, rotameric) δ 8.18 (s, 0.6H), 8.01 (d, J = 8.2 Hz, | |
| | 0.4H), 7.65 (d, J = 8.0 Hz, 0.6H), 7.56 (d, J = 8.1 Hz, 0.4H), 3.89-3.75 (m, 0.6H), | |
| | 3.70-3.56 (m, 0.4H), 3.42 (s, 1.2H), 3.37 (s, 1.8H), 3.31 (s, 1.8H), 3.29 (s, 1.2H), | |
| | 3.14-3.01 (m, 0.6H), 2.98-2.87 (m, 0.4H), 2.39 (s, 1.8H), 2.36 (s, 1.2H), 2.07- | |
| | 1.96 (m, 0.8H), 1.90-1.76 (m, 1.2H), 1.12 (t, J = 7.4 Hz, 1.8H), 1.06 (t, J = 7.4 Hz, | |
| | 1.2H). | |
| 4-275 | ¹H NMR (400 MHz, CDCl₃-d, rotameric) δ 8.48 (d, J = 8.2 Hz, 0.6H), 8.31 (d, J = | |
| | 8.2 Hz, 0.4H), 7.83 (d, J = 8.2 Hz, 0.6H), 7.74 (d, J = 8.2 Hz, 0.4H), 3.67 (td, J = | |
| | 7.2, 2.6 Hz, 2H), 3.62 (s, 3H), 3.56 (s, 2H), 3.53 (s, 1H), 3.49 (s, 2H), 3.35 (s, 3H), | |
| | 2.46 (s, 3H), 2.44 (s, 2H), 2.15-2.10 (m, 1H), 2.03-1.91 (m, 2H), 1.17 (t, J = 7.4 | |
| | Hz, 3H), 1.11 (t, J = 7.4 Hz, 2H). | |
| 4-286 | ¹H NMR (400 MHz, CDCl₃-d, rotameric) δ 8.31 (d, J = 8.1 Hz, 0.5H), 8.11 (d, J = 8.1 | |
| | Hz, 0.5H), 7.60 (d, J = 8.1 Hz, 0.5H), 7.47 (d, J = 8.2 Hz, 0.5H), 4.09-3.78 (m, | |
| | 1.5H), 3.77-3.55 (m, 1.5H), 3.49 (s, 1.5H), 3.39 (s, 1.5H), 2.45 (s, 1.5H), 2.37 (s, | |
| | 1.5H), 1.42-1.27 (m, 4.5H), 1.26-1.14 (m, 4.5H). | |
| 4-287 | ¹H NMR (400 MHz, CDCl₃-d, rotameric) δ 8.33 (d, J = 9.9 Hz, 0.5H), 8.15 (d, J = | |
| | 8.1 Hz, 0.5H), 7.73 (d, J = 8.1 Hz, 0.5H), 7.60 (d, J = 8.2 Hz, 0.5H), 4.39-3.53 (m, | |
| | 3H), 3.52-3.24 (m, 3H), 2.53-2.30 (m, 3H), 1.79-1.46 (m, 3H), 1.40-1.01 (m, 6H). | |
| 4-290 | ¹H NMR (400 MHz, CDCl₃-d, rotameric) δ 8.56 (d, J = 8.3 Hz, 0.5H), 8.38 (d, J = | |
| | 8.2 Hz, 0.5H), 7.87 (d, J = 8.2 Hz, 0.5H), 7.70 (d, J = 8.3 Hz, 0.5H), 4.35-4.20 (m, | |
| | 0.5H), 4.20-4.08 (m, 0.5H), 4.08-3.98 (m, 0.5H), 3.87-3.82 (m, 0.5H), 3.67- | |
| | 3.61 (m, 1H), 3.60 (s, 1.5H), 3.52 (s, 1.5H), 2.42 (d, J = 16.1 Hz, 3H), 1.55-1.46 (m, | |
| | 3H), 1.43-1.35 (m, 3H), 1.32 (t, J = 7.1 Hz, 1.5H), 1.21 (t, J = 7.1 Hz, 1.5H). | |
| 4-291 | ¹H NMR (400 MHz, CDCl₃-d, rotameric) δ 8.25 (d, J = 8.2 Hz, 0.6H), 8.08 (d, J = | |
| | 8.2 Hz, 0.4H), 7.60 (d, J = 8.2 Hz, 0.6H), 7.43 (d, J = 8.2 Hz, 0.4H), 4.05-3.79 (m, | |
| | 1H), 3.79-3.61 (m, 2H), 3.60-3.31 (m, 5H), 3.27-3.21 (m, 3H), 2.50-1.81 (m, 9H). | |
| 4-292 | ¹H NMR (400 MHz, CDCl₃-d, rotameric) δ 8.25-8.16 (m, 0.6H), 8.05 (d, J = 8.1 Hz, | |
| | 0.4H), 7.73 (d, J = 8.1 Hz, 0.6H), 7.55 (d, J = 8.1 Hz, 0.4H), 4.62-3.32 (m, 8H), 3.27- | |
| | 3.23 (m, 3H), 3.06-2.74 (m, 1H), 2.58-1.93 (m, 8H). | |
| 4-295 | ¹H NMR (400 MHz, CDCl₃-d, rotameric) δ 8.46 (d, J = 8.2 Hz, 0.6H), 8.31 (d, J = 8.2 | |
| | Hz, 0.4H), 7.85 (d, J = 8.2 Hz, 0.6H), 7.65 (d, J = 8.2 Hz, 0.4H), 4.78-4.53 (m, 1H), | |
| | 4.24-3.48 (m, 7H), 3.28-3.24 (m, 3H), 2.99-2.70 (m, 2H), 2.54-1.97 (m, 7H). | |

### Biological Activity Evaluation

### Test Example 1: Greenhouse Herbicidal Activity Assay

The herbicidal activity testing method for the compounds of the present invention is as follows:

Pre-emergence: Seeds of gramineous weeds (Echinochloa crusgalli, Echinochloa crusgalli var. zelayensis, Echinochloa phyllopogon, Eleusine indica, Digitaria sanguinalis, Leptochloa chinensis, Setaria viridis, Alopecurus japonicus Steud, Beckmannia syzigachne, Polypogon fugax, Alopecurus aequalis, Lolium multiflorum, Poa annua, Aegilops tataschii, Avena fatua, Alopecurus myosuroides, Bromus japonicus), broadleaf weeds (Eclipta prostrata, Amaranthus retroflexus, Rorippa indica, Myosoton aquaticum, Veronica didyma, Galium aparine, Solanum nigrum, Xanthium sibiricum, Conyza canadensis, Sesbania cannabina, Sagittaria trifolia), and Cyperus iria (Cyperus iria, Scirpus juncoides, Cyperus difformis, etc.) were sown in plastic pots (7 cm diameter) with nutrient soil and bottom holes. Crop seeds (including Oryza sativa subsp. indica, Oryza sativa subsp. japonica, Oryza sativa L. var. Glutinosa matsum, Triticum aestivum, Zea mays, Sorghum bicolor, Setaria italica, Glycine max, Gossypium hirsutum, Brassica napus, Arachis hypogaea) were sown in plastic pots (10 cm diameter) with nutrient soil and bottom holes. After sowing, the seeds were lightly covered with soil, and the soil was moistened via bottom water absorption. The pots were placed in a constant-temperature light-controlled growth chamber for 24 hours before soil spraying. Spraying was performed using a 3WP-2000 walking-type spray tower (produced by the Nanjing Agricultural Mechanization Institute, Ministry of Agriculture) with a spindle speed of 96 mm/r, spray height of 300 mm, effective spray width of 350 mm, spray area of 0.35 m², and nozzle flow rate of 390 mL/min.

After treatment, the test materials were air-dried naturally in the laboratory and then transferred to the growth chamber. Results were assessed 14-30 days later.

Post-emergence: Seeds of gramineous weeds (Echinochloa crusgalli, Echinochloa crusgalli var. zelayensis, Echinochloa phyllopogon, Eleusine indica, Digitaria sanguinalis, Leptochloa chinensis, Setaria viridis, Alopecurus japonicus Steud, Beckmannia syzigachne, Polypogon fugax, Alopecurus aequalis, Lolium multiflorum, Poa annua, Aegilops tataschii, Avena fatua, Alopecurus myosuroides, Bromus japonicus), broadleaf weeds (Eclipta prostrata, Amaranthus retroflexus, Rorippa indica, Myosoton aquaticum, Veronica didyma, Galium aparine, Solanum nigrum, Xanthium sibiricum, Conyza canadensis, Sesbania cannabina, Sagittaria trifolia), and Cyperus iria (Cyperus iria, Scirpus juncoides, Cyperus difformis, etc.) were sown in plastic pots (7 cm diameter) with nutrient soil and bottom holes. Crop seeds (including Oryza sativa subsp. indica, Oryza sativa subsp. japonica, Oryza sativa L. var. Glutinosa matsum, Triticum aestivum, Zea mays, Sorghum bicolor, Setaria italica, Glycine max, Gossypium hirsutum, Brassica napus, Arachis hypogaea) were sown in plastic pots (10 cm diameter) with nutrient soil and bottom holes. After sowing, the pots were placed in the growth chamber until the plants reached the 3-6 leaf stage for foliar spraying. Post-treatment, the materials were air-dried naturally and returned to the growth chamber. Results were assessed 14-21 days later.

### Control Efficacy Grading:

A: Fresh weight inhibition rate≥80% to 100%;
B: Fresh weight inhibition rate≥260% to <80%;
C: Fresh weight inhibition rate≥40% to <60%;
D: Fresh weight inhibition rate≥20% to <40%;
E: Fresh weight inhibition rate<20%;
NT: Not tested.

Test results showed that the compounds of the present invention exhibited superior herbicidal efficacy at 150 g a.i./ha, with representative data listed in Table 3.

TABLE 3
Post-emergence Test (Rate: 150 g a.i./hm²)

| Compound | Echinochloa | Digitaria | Leptochloa | Eclipta | Cyperus |
|---|---|---|---|---|---|
| NO. | crusgalli | sanguinalis | chinensis | prostrata | iria |
| 1-1 | A | A | A | A | A |
| 1-2 | A | A | A | A | A |
| 1-3 | A | A | A | A | A |
| 1-4 | A | A | A | A | A |
| 1-5 | A | A | A | A | A |
| 1-6 | A | A | A | A | A |
| 1-7 | A | A | A | A | A |
| 1-8 | A | A | A | A | A |
| 1-10 | A | A | A | A | A |
| 1-11 | A | A | A | A | A |
| 1-12 | A | A | A | A | A |
| 1-16 | A | NT | A | A | B |
| 1-19 | A | A | A | A | NT |
| 1-24 | A | B | A | A | A |
| 1-27 | A | A | A | A | A |
| 1-28 | A | A | A | A | A |
| 1-31 | A | A | A | A | A |
| 1-32 | A | A | A | A | A |
| 1-33 | A | A | A | A | A |
| 1-36 | A | A | A | A | A |
| 1-37 | A | NT | NT | A | A |
| 1-38 | A | A | B | A | A |
| 1-41 | A | NT | A | A | A |
| 1-42 | B | NT | A | A | A |
| 1-43 | A | NT | A | A | A |
| 1-46 | A | NT | NT | A | A |
| 1-47 | B | NT | A | A | A |
| 1-48 | A | A | A | A | A |
| 1-52 | A | A | A | A | A |
| 1-56 | A | A | A | A | A |
| 1-58 | A | A | A | A | B |
| 1-63 | A | B | A | A | A |
| 1-67 | A | A | A | A | A |
| 1-68 | A | A | A | A | A |
| 1-71 | A | A | A | A | A |
| 1-78 | A | A | A | A | A |
| 1-83 | A | A | A | A | A |
| 1-88 | A | A | A | A | A |
| 1-93 | A | A | A | A | A |
| 1-97 | NT | B | NT | NT | A |
| 1-98 | A | NT | A | NT | A |
| 1-101 | NT | NT | B | A | NT |
| 2-1 | A | A | A | A | A |
| 2-2 | A | A | A | A | A |
| 2-5 | B | B | A | A | A |
| 2-6 | A | B | A | A | A |
| 2-7 | A | A | A | A | A |
| 2-8 | A | A | A | A | A |
| 2-9 | A | A | A | A | A |
| 2-10 | A | A | A | A | A |
| 2-12 | A | A | A | A | A |
| 2-16 | NT | NT | A | A | A |
| 2-19 | A | A | A | A | A |
| 2-24 | A | A | A | A | A |
| 2-27 | A | A | A | A | A |
| 2-28 | A | A | A | A | A |
| 2-29 | A | A | A | A | A |
| 2-30 | A | A | A | A | A |
| 2-31 | A | NT | A | A | A |
| 2-32 | A | A | A | A | NT |
| 2-33 | A | A | A | A | A |
| 2-37 | A | B | A | A | A |
| 2-38 | A | A | A | A | A |
| 2-41 | A | A | NT | A | A |
| 2-42 | NT | NT | A | A | A |
| 2-43 | A | A | A | A | A |
| 2-46 | A | A | A | A | A |
| 2-48 | A | A | A | A | A |
| 2-67 | B | B | B | A | A |
| 2-68 | A | A | A | A | A |
| 2-71 | A | A | A | A | A |
| 2-78 | A | NT | NT | NT | NT |
| 3-1 | A | A | A | A | A |
| 3-2 | A | A | A | A | A |
| 3-5 | A | A | A | A | A |
| 3-6 | A | A | A | A | A |
| 3-7 | A | A | A | A | A |
| 3-10 | A | A | A | A | A |
| 3-11 | B | A | A | A | A |
| 3-12 | A | A | A | A | A |
| 3-15 | B | B | B | A | B |
| 3-16 | A | A | A | A | A |
| 3-19 | A | A | A | A | A |
| 3-24 | A | A | A | A | A |
| 3-25 | A | A | A | A | A |
| 3-27 | A | A | A | A | A |
| 3-28 | A | A | A | A | A |
| 3-31 | B | A | A | A | A |
| 3-32 | A | B | A | B | A |
| 3-37 | A | A | A | A | A |
| 3-38 | A | A | A | A | A |
| 3-41 | A | A | A | A | A |
| 3-52 | B | B | A | A | A |
| 3-53 | B | A | A | A | A |
| 3-67 | NT | NT | NT | B | A |
| 3-68 | NT | B | A | A | A |
| 3-71 | NT | NT | A | A | A |
| 3-77 | A | A | A | A | A |
| 3-78 | A | A | A | A | A |
| 3-81 | A | A | A | A | A |
| 3-82 | A | A | A | A | A |
| 3-83 | A | A | A | A | A |
| 3-86 | A | A | A | A | A |
| 3-87 | A | A | A | A | A |
| 3-88 | A | A | A | A | A |
| 3-91 | A | A | A | A | A |
| 3-92 | NT | NT | NT | NT | A |
| 3-93 | A | A | A | A | A |
| 3-96 | NT | NT | A | A | A |
| 3-97 | A | A | A | A | A |
| 3-98 | A | A | A | A | A |
| 3-101 | A | A | A | A | A |
| 3-102 | A | A | A | A | A |
| 3-103 | A | A | A | A | A |
| 3-112 | A | A | A | A | A |
| 3-113 | A | A | A | A | A |
| 3-116 | A | A | A | A | C |
| 3-142 | A | A | A | A | A |
| 3-143 | A | A | A | A | A |
| 3-146 | NT | B | A | A | A |
| 3-147 | B | A | A | A | B |
| 3-148 | NT | NT | A | B | NT |
| 3-151 | B | B | A | A | A |
| 3-172 | NT | A | A | A | A |
| 3-173 | NT | NT | A | NT | NT |
| 3-192 | A | A | A | A | A |
| 3-193 | A | B | A | A | A |
| 3-197 | A | A | A | A | A |
| 3-198 | NT | NT | A | A | A |
| 3-201 | NT | NT | A | A | A |
| 3-202 | A | B | A | B | B |
| 3-212 | A | A | A | A | A |
| 3-213 | A | A | A | B | A |
| 3-216 | NT | NT | A | NT | NT |
| 3-262 | A | A | A | A | A |
| 3-267 | A | A | A | A | A |
| 3-268 | A | A | A | A | A |
| 3-271 | A | A | A | A | A |
| 3-272 | A | C | A | A | A |
| 3-273 | A | A | A | A | A |
| 3-276 | A | A | A | A | A |
| 3-277 | A | C | A | A | A |
| 3-278 | A | A | A | A | A |
| 3-281 | NT | NT | A | A | A |
| 3-282 | A | B | A | A | A |
| 3-283 | A | A | A | A | A |
| 3-285 | A | A | A | A | A |
| 3-290 | A | A | A | A | A |
| 3-293 | A | A | A | A | A |
| 3-297 | NT | NT | B | A | A |
| 3-298 | A | A | A | A | A |
| 3-299 | A | A | A | A | A |
| 3-302 | A | A | A | A | A |
| 3-303 | A | A | A | A | B |
| 3-304 | A | A | A | A | A |
| 3-307 | A | A | A | A | A |
| 3-308 | A | A | A | A | A |
| 3-309 | A | A | A | A | A |
| 3-312 | A | A | A | A | A |
| 3-313 | A | A | A | A | A |
| 3-314 | A | A | A | A | A |
| 3-317 | A | A | A | A | A |
| 3-318 | A | A | A | A | A |
| 3-319 | A | A | A | A | A |
| 3-323 | A | A | A | A | A |
| 3-324 | A | A | A | A | A |
| 3-327 | A | B | A | A | A |
| 3-328 | A | A | A | A | A |
| 3-329 | A | A | A | A | A |
| 3-338 | A | C | A | A | A |
| 3-339 | A | A | A | A | A |
| 3-342 | A | A | A | A | A |
| 3-373 | A | A | A | A | A |
| 3-377 | C | NT | A | A | A |
| 3-378 | A | B | A | A | A |
| 3-379 | A | A | A | A | A |
| 3-382 | NT | NT | A | B | NT |
| 3-383 | A | B | B | A | A |
| 3-384 | A | A | A | A | A |
| 3-387 | A | A | A | A | A |
| 3-388 | A | NT | A | NT | A |
| 3-389 | A | A | A | A | A |
| 3-392 | A | A | A | A | A |
| 3-393 | NT | NT | B | B | A |
| 3-394 | A | A | A | A | A |
| 3-397 | NT | NT | NT | A | A |
| 3-398 | B | NT | A | B | A |
| 3-401 | A | A | A | A | A |
| 3-406 | A | A | A | A | A |
| 3-410 | C | A | A | A | A |
| 3-413 | NT | NT | A | A | A |
| 3-414 | A | A | A | A | A |
| 3-415 | A | A | A | A | A |
| 3-418 | A | A | A | A | A |
| 3-419 | A | A | A | A | A |
| 3-420 | A | A | A | A | A |
| 3-423 | A | A | A | A | A |
| 3-424 | A | A | A | A | A |
| 3-425 | A | A | A | A | A |
| 3-428 | A | A | A | A | A |
| 3-430 | A | A | A | A | A |
| 3-433 | A | A | A | A | A |
| 3-435 | A | A | A | A | A |
| 3-439 | A | A | A | A | A |
| 3-440 | A | A | A | A | A |
| 3-443 | NT | NT | A | A | A |
| 3-444 | A | B | A | B | NT |
| 3-445 | A | A | A | A | A |
| 3-454 | A | A | A | B | A |
| 3-455 | A | A | A | A | A |
| 3-458 | A | A | A | A | A |
| 3-494 | A | A | A | A | A |
| 3-495 | A | A | A | A | A |
| 3-498 | NT | NT | A | NT | NT |
| 3-499 | NT | NT | NT | NT | A |
| 3-500 | A | A | A | A | A |
| 3-503 | A | A | A | A | A |
| 3-504 | NT | NT | NT | B | NT |
| 3-505 | NT | NT | A | A | A |
| 3-508 | A | A | A | A | A |
| 3-517 | NT | NT | A | A | B |
| 3-522 | A | C | A | A | A |
| 3-531 | A | A | A | A | A |
| 3-534 | B | B | B | A | A |
| 3-535 | B | NT | B | B | A |
| 3-536 | A | B | A | A | A |
| 3-539 | B | B | A | B | B |
| 3-541 | C | B | A | A | A |
| 3-544 | B | NT | A | A | A |
| 3-546 | B | NT | A | A | A |
| 3-549 | NT | NT | A | A | A |
| 3-551 | B | NT | A | A | A |
| 3-556 | B | B | A | A | A |
| 3-570 | NT | NT | B | A | A |
| 3-571 | NT | NT | NT | A | A |
| 3-574 | A | B | A | A | A |
| 3-615 | NT | A | A | A | A |
| 3-616 | A | A | A | A | A |
| 3-619 | A | A | A | A | A |
| 3-620 | A | NT | A | B | B |
| 3-621 | A | A | A | A | A |
| 3-622 | A | A | A | A | A |
| 3-623 | A | A | A | A | A |
| 3-624 | A | A | A | A | A |
| 3-626 | A | A | A | A | A |
| 3-633 | A | A | A | A | A |
| 3-638 | A | B | A | A | A |
| 3-647 | A | A | A | A | A |
| 3-650 | B | A | A | A | A |
| 3-651 | A | A | A | A | A |
| 3-652 | NT | A | A | A | A |
| 3-655 | A | A | A | A | A |
| 3-657 | B | A | A | A | A |
| 3-660 | NT | NT | A | A | A |
| 3-662 | NT | A | A | A | A |
| 3-665 | NT | NT | A | A | B |
| 3-667 | C | A | A | A | A |
| 3-687 | B | A | A | A | A |
| 3-690 | A | B | A | A | A |
| 3-692 | A | A | A | A | A |
| 3-727 | A | A | A | A | A |
| 3-786 | B | NT | NT | NT | NT |
| 3-787 | A | NT | NT | NT | NT |
| 3-788 | A | NT | A | NT | NT |
| 3-795 | A | NT | NT | NT | NT |
| 3-798 | A | NT | NT | NT | NT |
| 3-804 | A | NT | NT | NT | NT |
| 3-807 | A | NT | NT | NT | NT |
| 3-808 | B | NT | NT | NT | NT |
| 3-809 | B | NT | NT | NT | NT |
| 3-810 | B | NT | NT | NT | NT |
| 3-812 | B | NT | B | A | NT |
| 3-813 | NT | NT | B | A | B |
| 3-815 | B | NT | NT | NT | NT |
| 3-820 | A | NT | NT | NT | NT |
| 3-829 | A | NT | B | NT | NT |
| 3-832 | A | NT | NT | NT | NT |
| 3-835 | A | NT | NT | NT | NT |
| 4-1 | NT | A | A | A | A |
| 4-2 | A | A | A | A | A |
| 4-3 | A | A | A | A | A |
| 4-4 | A | A | A | A | A |
| 4-5 | NT | NT | NT | A | A |
| 4-6 | NT | NT | A | B | NT |
| 4-7 | A | B | A | A | NT |
| 4-10 | A | B | A | B | A |
| 4-12 | A | A | B | A | A |
| 4-16 | NT | NT | NT | NT | A |
| 4-19 | NT | NT | A | A | A |
| 4-24 | NT | A | A | A | A |
| 4-32 | NT | NT | NT | NT | A |
| 4-33 | A | A | A | A | A |
| 4-36 | NT | NT | NT | NT | A |
| 4-38 | A | A | A | A | A |
| 4-43 | A | A | A | A | A |
| 4-272 | NT | NT | NT | A | A |

### Test Example 2: Greenhouse Herbicidal Activity and Crop Safety Comparison

Using the above method, the herbicidal activity and crop safety of the present compounds were compared with structurally similar known compounds (CK1, CK2, CK3, CK4: compounds 4-756, 4-759, 1-557, 1-563 from WO2012028579A1; CK5: compound 290-1 from WO2013087577A1; CK6: compound 1-129 from WO2011035874A1). Results are shown in Tables 4-8.

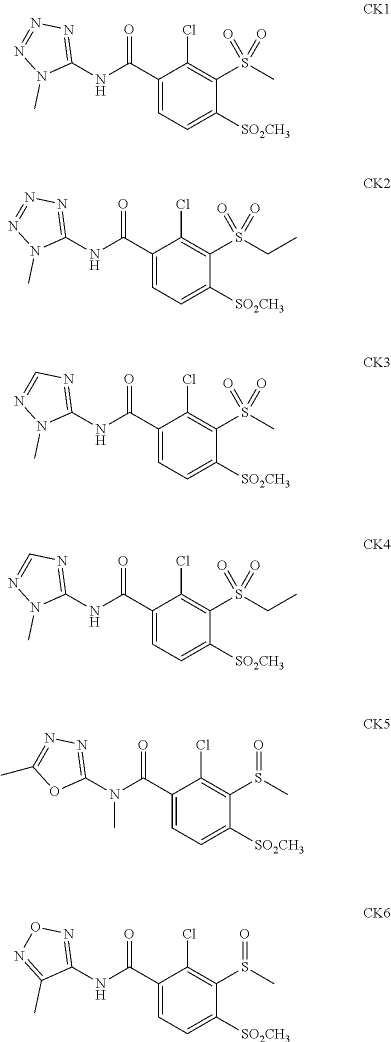

TABLE 4

| Compound | Rate | Oryza sativa | Oryza sativa | Echinochloa |
|---|---|---|---|---|
| NO. | (g/ha) | subsp. indica | subsp. japonica | crusgalli |
| 1-5 | 150 | E | E | A |
| 1-10 | 150 | E | E | A |
| 1-24 | 150 | E | E | A |
| 1-31 | 150 | E | E | A |
| 1-36 | 150 | E | E | A |
| 1-41 | 150 | E | E | A |
| 1-46 | 150 | E | E | A |
| 1-71 | 250 | E | E | A |
| 1-91 | 150 | E | E | A |
| 1-96 | 150 | E | E | A |
| CK1 | 150 | B | B | A |
| | 75 | C | C | A |
| | 18.75 | C | C | B |
| CK2 | 150 | B | B | A |
| | 75 | C | C | A |
| | 18.75 | C | C | B |

TABLE 5

| Compound | Rate | Oryza sativa | Oryza sativa | Zea | Echinochloa | Digitaria |
|---|---|---|---|---|---|---|
| NO. | (g/ha) | subsp. indica | subsp. japonica | mays | crusgalli | sanguinalis |
| 2-10 | 150 | E | E | E | A | A |
| | 75 | E | E | E | A | A |
| 2-41 | 150 | E | E | E | A | A |
| | 75 | E | E | E | A | B |
| 2-46 | 150 | E | E | E | A | A |
| | 75 | E | E | E | A | B |
| CK3 | 150 | B | C | D | A | D |
| | 75 | D | D | E | C | E |
| CK4 | 150 | B | C | D | B | C |
| | 75 | C | C | E | C | D |

TABLE 6

| Compound | Rate | Oryza sativa | Oryza sativa | Echinochloa | Leptochloa |
|---|---|---|---|---|---|
| NO. | (g/ha) | subsp. indica | subsp. japonica | crusgalli | chinensis |
| 3-5 | 150 | E | E | A | A |
| 3-10 | 150 | E | E | A | A |
| 3-15 | 250 | E | E | A | A |
| 3-24 | 150 | E | E | A | A |
| 3-31 | 250 | E | E | A | A |
| 3-41 | 150 | E | E | A | A |
| 3-71 | 150 | E | E | C | A |
| 3-81 | 150 | E | E | A | A |
| 3-86 | 150 | E | E | A | A |
| 3-91 | 150 | E | E | A | A |
| 3-96 | 150 | E | E | C | A |
| 3-101 | 150 | E | E | A | A |
| 3-116 | 150 | E | E | A | A |
| 3-151 | 150 | E | E | B | A |
| 3-201 | 150 | E | E | C | A |
| 3-216 | 150 | E | E | NT | A |
| 3-271 | 150 | E | E | A | A |
| 3-276 | 150 | E | E | A | A |
| 3-290 | 150 | E | E | A | A |
| 3-302 | 150 | E | E | A | A |
| 3-307 | 150 | E | E | A | A |
| 3-312 | 150 | E | E | A | A |
| 3-317 | 150 | E | E | A | A |
| 3-327 | 150 | E | E | A | A |
| 3-342 | 150 | E | E | A | A |
| 3-387 | 150 | E | E | A | A |
| 3-392 | 150 | E | E | A | A |
| 3-406 | 150 | E | E | A | A |
| 3-418 | 150 | E | E | A | A |
| 3-423 | 150 | E | E | A | A |
| 3-428 | 150 | E | E | A | A |
| 3-433 | 150 | E | E | A | A |
| 3-443 | 150 | E | E | C | A |
| 3-458 | 150 | E | E | A | A |
| 3-503 | 150 | E | E | A | A |
| 3-508 | 150 | E | E | A | A |
| 3-522 | 150 | E | E | A | A |
| 3-534 | 150 | E | E | B | A |
| 3-539 | 150 | E | E | B | A |
| 3-544 | 150 | E | E | B | A |
| 3-549 | 150 | E | E | NT | A |
| 3-574 | 150 | E | E | A | A |
| 3-619 | 150 | E | E | A | A |
| 3-624 | 150 | E | E | A | A |
| 3-638 | 150 | E | E | A | A |
| 3-650 | 150 | E | E | B | B |
| 3-655 | 150 | E | E | A | A |
| 3-660 | 150 | E | E | C | A |
| 3-665 | 150 | E | E | NT | A |
| 3-690 | 150 | E | E | A | A |
| CK7 | 150 | C | C | A | A |
| | 75 | D | D | B | A |
| | 18.75 | E | E | C | C |

TABLE 7

| Compound | Rate | Oryza sativa | Oryza sativa | Echinochloa |
|---|---|---|---|---|
| NO. | (g/ha) | subsp. indica | subsp. japonica | crusgalli |
| 3-787 | 150 | E | E | A |
| | 75 | E | E | A |
| 3-788 | 150 | E | E | A |
| | 75 | E | E | A |
| 3-795 | 150 | E | E | A |
| 3-798 | 150 | E | E | A |
| 3-801 | 150 | E | E | A |
| 3-804 | 150 | E | E | B |
| 3-807 | 150 | E | E | B |
| | 75 | E | E | B |
| 3-810 | 150 | E | E | B |
| CK5 | 150 | D | E | D |
| | 75 | E | E | E |
| | 18.75 | E | E | E |

TABLE 8

| Compound | Rate | | Digitaria |
|---|---|---|---|
| NO. | (g/ha) | Zea mays | sanguinalis |
| 4-2 | 150 | E | A |
| | 75 | E | A |
| | 18.75 | E | B |
| 4-3 | 150 | E | A |
| | 75 | E | A |
| | 18.75 | E | A |
| 4-4 | 150 | E | A |
| | 75 | E | A |
| | 18.75 | E | B |
| CK6 | 150 | D | C |
| | 75 | D | C |
| | 18.75 | E | E |

NT: Not tested.

### Test Example 3: Field Plot Trials

Field trials were conducted at the Zhuanghang Base and Chonggu Base of the Shanghai Academy of Agricultural Sciences, and in Xinzhou City, Shanxi Province.

### 1. Methods

Foliar spray: Applied at the 3-leaf stage of crops and 2-3-leaf stage of weeds, using 450 L/ha spray volume.

Granule application: Applied 5-7 days after rice transplantation via soil mixing (20-30 kg moist soil or fertilizer per mu), with 3-4 cm water layer maintained for 5-7 days.

Wind speed, temperature, and weather conditions were recorded during application.

### 2. Test Targets

Rice varieties: Huanghuazhan, Xiangzaoxian 45, Huaidao 5, Huiliangyou Simiao, Nanjing 9108, Nanjing 46, Jinjing 616, Liannuo 1;
Sorghum variety: Hongyingzi;
Weeds in paddy fields: Echinochloa crusgalli, Leptochloa chinensis, Ammannia baccifera, Ludwigia prostrata, Cyperus difformis, Cyperus iria;
Weeds in dry fields: Echinochloa crusgalli, Digitaria sanguinalis, Chenopodium album, Acalypha australis, Solanum nigrum, Portulaca oleracea.

### 3. Plot Design

Randomized block design with 40 m² plots and 4 replicates per treatment.

### 4. Observations

Three visual investigations will be conducted according to the following schedule:
3-7 days post-application: Observe weed intoxication symptoms and assess crop phytotoxicity or beneficial effects.
14 days post-application: Evaluate weed control efficacy through visual inspection and monitor recovery from crop phytotoxicity.
28 days post-application: Record weed population count and fresh weight, while collecting rice plants from previously observed phytotoxicity plots to measure plant height, root length, and fresh biomass (above-ground and underground parts).

A total of three surveys will be performed. Adjustments to the schedule may be made in case of rainy weather or other exceptional circumstances.

### 5. Data Analysis

Weed control efficacy:

Plant control (%)=[1−(Treated weed count/Control weed count)]×100%

Fresh weight control (%)=[1−(Treated weed biomass/Control weed biomass)]×100%

Crop safety: Growth inhibition rates for plant height, root length, and biomass were calculated similarly.

Results demonstrated excellent weed control and crop safety for the present compounds.

### Test Example 4: Algal Toxicity Evaluation

(1) Test Organism: Pseudokirchneriella subcapitata (formerly Selenastrum capricornutum), provided by the Freshwater Algae Culture Collection of the Institute of Hydrobiology, Chinese Academy of Sciences.

(2) Test Conditions: The experiment was conducted at a temperature of 21-24° C. under continuous uniform illumination with a light intensity of 4,440-8,880 Lux.

The cultivation and testing of Pseudokirchneriella subcapitata utilized BG11 medium, with preparation methods detailed in Table 9.

(3) Algal Cultivation: Using aseptic techniques, the test algae were inoculated into Erlenmeyer flasks containing BG11 medium. Subculturing was performed every 96 hours for 2-3 cycles to achieve synchronized growth, which served as the test algae. Microscopic observations were conducted during each subculture to monitor algal growth status.

(4) Test Solution Preparation: A precise amount of the raw chemical was weighed using an electronic balance and fully dissolved in an organic solvent (DMSO or acetone) to prepare a stock solution.

(5) Preliminary Test: A series of widely spaced concentrations were tested under formal experimental conditions to determine the lowest concentration causing algal growth inhibition and the highest concentration showing no inhibition. Formal test concentrations were set within this range.

(6) Formal Test: Based on preliminary results, five geometrically spaced concentrations were tested. A BG11 medium blank control and a solvent control (BG11 with organic solvent) were included. Under sterile conditions, Pseudokirchneriella subcapitata was cultured in 250 mL Erlenmeyer flasks. Each flask received 50 mL of test solution and 50 mL of diluted algal suspension (initial cell density: 2.10×10⁴ cells/mL), resulting in a final algal concentration of 1.05×10⁴ cells/mL. Flasks were incubated statically in a light incubator, gently shaken once every 24 hours. Three replicates were prepared for each treatment and control group. Temperature and pH were measured at 0 h and 72 h. Algal growth was microscopically observed, and cell density was quantified using a hemocytometer at 24 h, 48 h, and 72 h.

(7) Toxicity Assessment: According to the national standard Test guidelines on environmental safety assessment for chemical pesticides-Part 14: Alga growth inhibition test (GB/T 31270.14-2014), pesticide toxicity to algae is classified as:
High toxicity: 72 h EC₅₀≤0.3 mg a.i./L
Moderate toxicity: 0.3<EC₅₀≤3.0 mg a.i./L
Low toxicity: EC₅₀>3.0 mg a.i./L

TABLE 9
BG11 Medium formulation

| | | | Mother | Mother |
|---|---|---|---|---|
| | | | liquor | liquor |
| Entry | | Component | concentration | dosage |
| 1 | | NaNO₃ | 15 g/100 mL dH₂O | 10 mL |
| 2 | | K₂HPO₄ | 2 g/500 mL dH₂O | 10 mL |
| 3 | | MgSO₄•7H₂O | 3.75 g/500 mL dH₂O | 10 mL |
| 4 | | CaCl₂•2H₂O | 1.8 g/500 mL dH₂O | 10 mL |
| 5 | | Citric Acid (C₆H₈O₇) | 0.3 g/500 mL dH₂O | 10 mL |
| 6 | | FeC₆H₅O₇•NH₄OH | 0.3 g/500 mL dH₂O | 10 mL |
| 7 | | EDTANa₂ | 0.05 g/500 mL dH₂O | 10 mL |
| 8 | | Na₂CO₃ | 1.0 g/500 mL dH₂O | 10 mL |
| | | H₃BO₃ | 2.86 g/L dH₂O | |
| 9 | A5 | MnCl₂•4H₂O | 1.86 g/L dH₂O | 1 mL |
| | 1 mL/L | ZnSO₄•7H₂O | 0.22 g/L dH₂O | |
| | | Na₂MoO₄•2H₂O | 0.39 g/L dH₂O | |
| | | CuSO₄•5H₂O | 0.08 g/L dH₂O | |
| | | Co(NO₃)₂•6H₂O | 0.05 g/L dH₂O | |

Prepare each component into corresponding stock solutions at specified concentrations. Following the indicated order, sequentially transfer the designated volumes of each stock solution into a 1000 mL volumetric flask. Bring the solution to volume, then sterilize by autoclaving (121° C. for 15 minutes). Seal the container and affix proper labeling before storing at 4° C. refrigerator, where it remains valid for two months. Prior to use, dilute the medium 10-fold with distilled water that has been sterilized by autoclaving (121° C. for 15 minutes).

Results indicated low algal toxicity for most compounds.

### Test Case 5: Fish Toxicity Evaluation

### (1) Materials and Conditions

The test fish were zebrafish (Brachydanio rerio), healthy and disease-free, with body lengths controlled at 2-2.5 cm per fish. Prior to testing, the fish were acclimated for 14 days under environmental conditions identical to those during the test, with minimal daily feeding and 16 hours of light per day. A fully equipped aquarium filtration system was maintained. Feeding was halted 24 hours before the test.

### (2) Test Water

Tap water aerated for over 24 hours using an air pump was utilized. The test was conducted at a temperature of 21° C.-25° C., with a 16 h:8 h light-dark cycle. Water parameters included hardness of 10-250 mg/L (as CaCO₃), pH 6.0-8.5, and dissolved oxygen levels not less than 60% of air saturation.

### (3) Test Method

A static test method was employed.

### (4) Test Solution Preparation

A specified quantity of the test compound was weighed using an electronic balance and fully dissolved in an organic solvent (DMSO or acetone) to prepare a stock solution.

### (5) Preliminary Test

Under formal test conditions, multiple concentration groups with wide intervals were established. Each treatment used 5 fish without replicates. Poisoning symptoms and mortality were observed and recorded over 96 hours. The preliminary test determined the maximum concentration with 100% survival and the minimum concentration with 100% lethality. Formal test concentrations were set within this range.

### (6) Formal Test

Based on preliminary results, five concentrations were established at defined intervals, including a blank control group. Acclimated zebrafish were placed into test tanks. Each treatment included one replicate, with 500 mL of solution and 5 zebrafish per replicate. At test initiation, temperature, pH, and dissolved oxygen ratio (test solution dissolved oxygen/air saturation value) were measured for all treatment concentrations and the blank control.

### (7) Observation and Recording

Zebrafish poisoning symptoms and mortality were observed and recorded at 6 h, 24 h, 48 h, 72 h, and 96 h post-exposure. Dead fish were immediately removed. Death was defined as the absence of visible movement (e.g., gill motion, no response to tail touch). Poisoning criteria included abnormal behaviors such as side-swimming, lying on the tank bottom, or significant body curvature.

### (8) Toxicity Assessment

According to the national standard Test guidelines on environmental safety assessment for chemical pesticides-Part 12: Fish acute toxicity test (GB/T 31270.12-2014), acute toxicity to zebrafish is classified into four levels:
Extremely toxic: LC₅₀ (96 h) (mg a.i./L)≤0.1;
Highly toxic: 0.1<LC₅₀ (96 h) (mg a.i./L)≤1.0;
Moderately toxic: 1.0<LC₅₀ (96 h) (mg a.i./L)≤10;
Low toxicity: LC₅₀ (96 h) (mg a.i./L)>10.

Test results indicated that most compounds in this invention exhibit low fish toxicity. The above description represents only preferred embodiments of the invention. It should be noted that those skilled in the art may make various modifications and improvements without departing from the inventive concept of the invention, all of which fall within the scope of protection of the invention.

1. A 4-methylsulfonylbenzamide compound of formula (I), stereoisomer thereof or agriculturally acceptable salt thereof 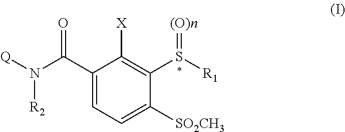
wherein
Q represents Q1, Q2, Q3 or Q4, as shown below: 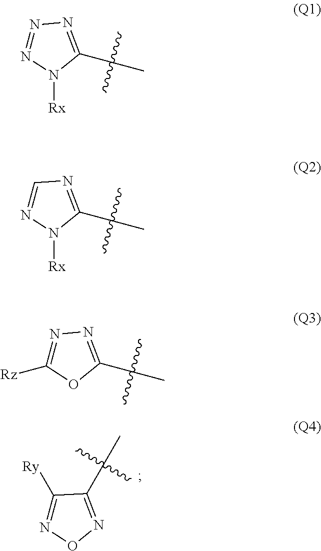
X represents fluorine, chlorine or bromine;
R₁ represents C₃₋₆ alkyl, C₃₋₆ cycloalkyl, C₃₋₆ cycloalkyl-C₁₋₆ alkyl, halogenated C₁₋₆ alkyl and not halo-C₁ alkyl, C₂₋₆ alkenyl and not C₂ alkenyl, C₂₋₆ alkynyl and not C₂ alkynyl or NC—C₁₋₆ alkyl;
R₂ represents hydrogen, C₁₋₆ alkyl or RaO—C₁₋₆ alkyl;
Rx represents hydrogen, C₁₋₆ alkyl or C₃₋₇ cycloalkyl;
Ry represents hydrogen, C₁₋₆ alkyl or C₃₋₆ cycloalkyl;
Rz represents hydrogen, C₁₋₆ alkyl or C₃₋₇ cycloalkyl;
Ra represents C₁₋₆ alkyl
n represents 1 or 2;
when n represents 1, the sulfur atom connected thereto may be selected from either an R configuration or an S configuration, or a mixture of the two, and the ratio of R to S in the mixture is 1:99 to 99:1.

2. The 4-methylsulfonylbenzamide compound, stereoisomer thereof or agriculturally acceptable salt thereof according to claim 1 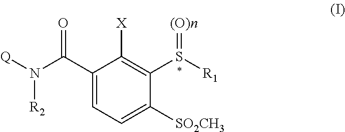
wherein
Q represents Q1, Q2, Q3 or Q4, as shown below: 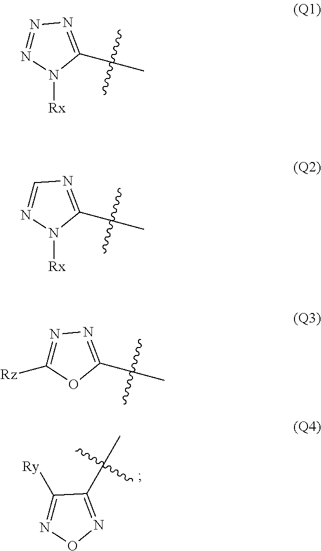
X represents chlorine;
R₁ represents C₃₋₆ alkyl, C₃₋₆ cycloalkyl, C₃₋₆ cycloalkylmethyl, halogenated C₃₋₄ alkyl, C₂₋₄ alkenyl and not C₂ alkenyl, C₂₋₄ alkynyl and not C₂ alkynyl, NC—C₁₋₄ alkyl;
R₂ represents hydrogen, C₁₋₃ alkyl or C₁₋₆ alkyl-O—C₁₋₆ alkyl;
Rx represents hydrogen, C₁₋₃ alkyl or cyclopropy;
Ry represents hydrogen, C₁₋₃ alkyl or cyclopropyl;
Rz represents hydrogen, C₁₋₃ alkyl or cyclopropyl;
n represents 1 or 2;
when n represents 1, the sulfur atom connected thereto may be selected from either an R configuration or an S configuration, or a mixture of the two, and the ratio of R to S in the mixture is 1:99 to 99:1.

3. The 4-methylsulfonylbenzamide compound, stereoisomer thereof or agriculturally acceptable salt thereof according to claim 2 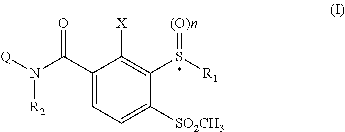
wherein
Q represents Q1, Q2, Q3 or Q4, as shown below: 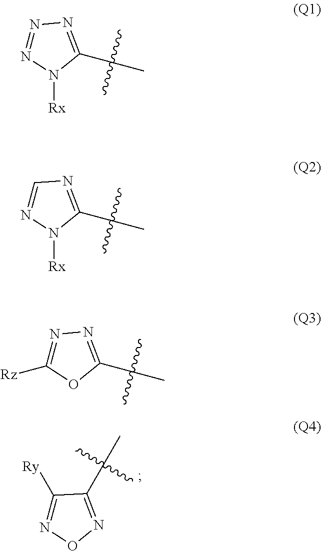
X represents chlorine;
R₁ represents CH₂CH₂CH₃, CH(CH₃)₂, CH₂CH₂CH₂CH₃, CH(CH₃)CH₂CH₃, cyclopropylmethyl, cyclopropyl, cyclobutyl, cyclopentyl, cyclohexyl, CH₂CF₃, CH₂CH═CH₂, CH₂CH₂Cl, CH₂CH(CH₃)₂, C(CH₃)₃, CH₂CH₂CH₂CH₂CH₃, CH(CH₃)CH₂CH₂CH₃, CH₂CH₂CH(CH₃)₂, C(CH₃)₂CH₂CH₃, CH₂CH(CH₃)CH₂CH₃, CH₂C₆H₅, CH(CH₃)C₆H₅, CH₂CF₂H, CH₂CFH₂, CH(CH₃)CH═CH₂, (E)-CH₂CH═CHCH₃, (Z)—CH₂CH═CHCH₃, CH₂CH═C(CH₃)₂, propargyl, but-3-en-2-yl, but-2-en-1-yl, CF₂CF₃, CF₂CF₂CF₃, CH₂CF₂CF₃, CH₂CH₂CF₃, CH₂CH₂CF₂H, CH₂CH₂CFH₂, CF₂CFHCF₃, CH₂CN, CH₂CH₂CN, CH(CH₃)CN, CH(CH₃)CH₂CN, CHClCH₃, CH₂CH₂OCF₃, CH₂CH₂OCF₂H, CH₂CH₂OCFH₂, CH₂OCH₃, CH₂CF₂H, or CH₂CFH₂;
R₂ represents hydrogen, methyl, ethyl, CH(CH₃)₂, or C₁₋₆ alkyl-O—C₁₋₆ alkyl;
Rx represents hydrogen, methyl, or ethyl;
Ry represents hydrogen, methyl, ethyl, isopropyl, cyclopropyl, or CH₂CH₂CH₃;
Rz represents hydrogen, methyl, ethyl, isopropyl, cyclopropyl or CH₂CH₂CH₃;
n represents 1 or 2;
when n represents 1, the sulfur atom connected thereto may be selected from either an R configuration or an S configuration, or a mixture of the two, and the ratio of R to S in the mixture is 1:99 to 99:1.

4. A method for preparing the 4-methylsulfonylbenzamide compound, stereoisomer thereof or agriculturally acceptable salt thereof according to claim 1, comprising the following steps:
(1) conducting a substitution reaction of a compound of formula (VI) with different substituted thiols or thiolates to give a compound of formula (V);
(2) conducting a condensation reaction between the compound of formula (V) and a compound of formula (IV) to give a compound of formula (III);
(3) conducting an oxidation reaction of the compound of formula (III) to give a compound of formula (II); and
(4) conducting a substitution reaction of the compound of formula (II) to give the 4-methylsulfonylbenzamide compound of formula (I); 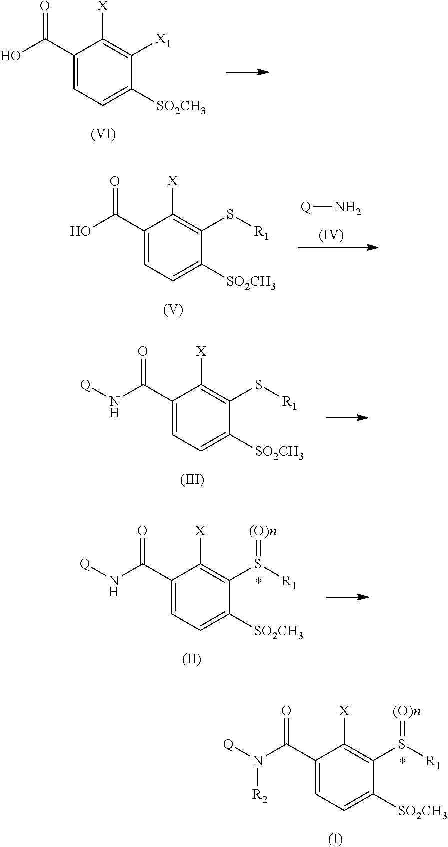
wherein
X₁ is selected from fluorine or chlorine;
R₁, R₂, X, and Q have the meaning according to claim 1, and n represents 1 or 2.

5. A method for preparing the 4-methylsulfonylbenzamide compound, stereoisomer thereof or agriculturally acceptable salt thereof according to claim 1, comprising the following steps:
(1) conducting a substitution reaction of a compound of formula (VI) with hydrosulfide salt to give a thiophenol compound of formula (VII);
(2) conducting a substitution reaction of the compound of formula (VII) with a compound of formula (VIII) to give a compound of formula (V);
(3) conducting a condensation reaction between the compound of formula (V) and a compound of formula (IV) to give a compound of formula (III);
(4) conducting an oxidation reaction of the compound of formula (III) to give a compound of formula (II); and
(5) conducting a substitution reaction of the compound of formula (II) to give the 4-methylsulfonylbenzamide compound of formula (I); 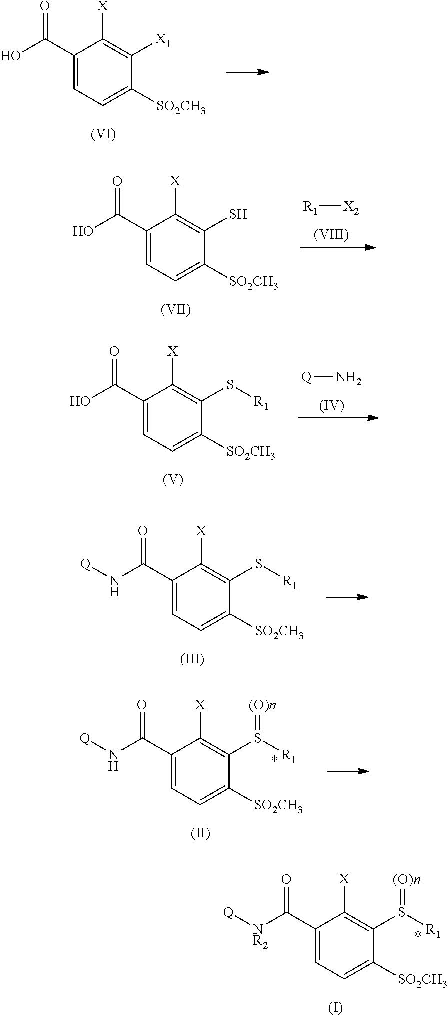
wherein
X₁ is selected from fluorine or chlorine;
X₂ is selected from chlorine, bromine, iodine, MsO (methanesulfonyloxy), TfO (trifluoromethanesulfonyloxy) or TsO (p-toluenesulfonyloxy);
R₁, R₂, X, and Q have the meaning according to claim 1.

6. A herbicidal composition, comprising at least one of the 4-methylsulfonylbenzamide compound, stereoisomer thereof or salt thereof according to claim 1 with the compound of formula (I) as an active ingredient, wherein the weight percentage of the active ingredient in the composition is 0.1% to 99.9%.

7. The herbicidal composition according to claim 6, further comprising a formulation adjuvant.

8. The herbicidal composition according to claim 7, comprising at least one further active compound selected from insecticide, acaricide, herbicide, fungicide, safener, and/or growth regulator.

9. The herbicidal composition according to claim 7 comprising a safener.

10. The herbicidal composition according to claim 9, wherein the safener is selected from mefenpyr-diethyl, cyprosulfamide, isoxadifen-ethyl, cloquintocet-mexyl, benoxacor, fenclorim, furilazole, dichlormid or Metcamifen.

11. A method for controlling harmful plants, comprising applying an effective amount of at least one compound of formula (I) according to claim 1, to a plant or a site of harmful vegetation.

12. A method for controlling harmful plants, comprising applying an effective amount of the herbicidal composition according to claim 6, to a plant or a site of harmful vegetation.

13. Use of the compound of formula (I) according to claim 1 for controlling harmful plants.

14. Use of the herbicidal composition according to claim 6 for controlling harmful plants.

15. The use according to claim 13, wherein the compound of formula (I) containing the compound of formula (I) is used for controlling harmful plants in crops of useful plants.

16. The use according to claim 15, wherein the useful plants are transgenic plants or plants processed by genome editing technology.

17. The use according to claim 13, wherein the herbicidal composition containing the compound of formula (I) is used for controlling harmful plants in crops of useful plants.

18. The use according to claim 17, wherein the useful plants are transgenic plants or plants processed by genome editing technology.
